(19) United States
(12) Patent Application Publication
(10) Pub. No.: US 2022/0390464 A1
(43) Pub. Date: Dec. 8, 2022

(54) METHODS AND COMPOSITIONS FOR CHARACTERIZING INFLAMMATORY BOWEL DISEASE

(71) Applicant: Children's Medical Center Corporation, Boston, MA (US)

(72) Inventors: Scott B. SNAPPER, Boston, MA (US); Vanessa MITSIALIS, Boston, MA (US); Liza KONNIKOVA, Boston, MA (US)

(73) Assignee: Children's Medical Center Corporation, Boston, MA (US)

(21) Appl. No.: 17/631,365

(22) PCT Filed: Jul. 27, 2020
(86) PCT No.: PCT/US20/43736

§ 371 Date: Jan. 28, 2022

Related U.S. Application Data
(60) Provisional application No. 62/880,479, filed on Jul. 30, 2019.
(60) Provisional application No. 63/005,086, filed on Apr. 3, 2020.

(51) Int. Cl. G01N 33/68

(52) U.S. Cl. CPC G01N 33/6893

(57) ABSTRACT

The invention features compositions and methods for characterizing inflammatory bowel disease and inflammatory bowel disease subtypes, such as well Crohn's disease and ulcerative colitis. In one aspect, the invention provides a panel for characterizing inflammatory bowel disease, the panel including two or more (e.g., 2, 3, 4, 5, 6, 7, 8, 9, 10, 11, 12, 13, 14, 15, 20, 25) capture molecules each bound to a substrate, wherein each capture molecule specifically binds a marker polypeptide selected from one or more of the following: CD3, CD4, CDS, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-10, IL-17A, IL-21, IL-22, CD11c, IL23p19, CD66b, CD163, CD44, ckit, CD16, NKp46, AHR, and TNFα, or a polynucleotide encoding said marker polypeptide.

### CROSS REFERENCE TO RELATED APPLICATIONS

The present application claims priority to U.S. App. Nos. 62/880,479 filed Jul. 30, 2019 and 63/005,086, filed Apr. 3, 2020, the entire contents of which are hereby incorporated by reference in their entirety.

### BACKGROUND OF THE INVENTION

Inflammatory bowel disease is a chronic disease characterized by long-lasting intestinal inflammation that affects over one million people in the U.S. The two main subtypes of inflammatory bowel disease are Crohn's disease and ulcerative colitis. Crohn's disease involves transmural inflammation at any location in the gastrointestinal tract and can be associated with fistulizing or stenotic complications. Ulcerative colitis is characterized by contiguous superficial mucosal inflammation of the colon. In some cases, the two subtypes can be differentiated by macroscopic inflammation patterns, but obtaining such data requires invasive techniques. Even with macroscopic inflammation data, differentiating between Crohn's disease or ulcerative colitis can remain difficult or impossible in some cases, which can lead to a less specific diagnosis of “unclassified” inflammatory bowel disease. While several pharmaceutical options are available, the inflammatory bowel disease is heterogenous, and an accurate diagnosis is needed for proper treatment. More accurate diagnostic techniques would allow for more personalized and effective treatments. Thus, there is a demonstrated and urgent need for improved methods for differentiating inflammatory bowel disease subtypes.

### SUMMARY OF THE INVENTION

As described below, the present invention features methods and compositions for differentiating and characterizing the different subtypes of inflammatory bowel disease.

In one aspect, the invention provides a panel for characterizing inflammatory bowel disease, the panel including two or more (e.g., 2, 3, 4, 5, 6, 7, 8, 9, 10, 11, 12, 13, 14, 15, 20, 25) capture molecules each bound to a substrate, wherein each capture molecule specifically binds a marker polypeptide selected from one or more of the following: CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα, or a polynucleotide encoding said marker polypeptide.

In some embodiments of the above aspect, the panel includes the following: CD3, CD4, CD8, CD127, CD25, CD27, CXCR3, CCR6, CCR7, CD45RA, CD45RO, IFNγ, TNFα, IL-1β, IL-17A, IL-21, IL-22, HLA-DR, and CD38; or CD38, HLA-DR, FOXP3, IL-17A, CCR4, CTLA-4, TNFα, IL-1β, CD161, IFNγ, CCR6, CCR4, CD45RO CD161, CD24, CCR7, CD44, IL-21, IL-22, CD45RA, CXCR3, and CD45RA.

In another aspect, the invention provides a panel for characterizing inflammatory bowel disease, the panel including two or more capture molecules each bound to a substrate, wherein each capture molecule specifically binds a marker polypeptide selected from one or more of the following: CD45, CD3, CD4, CD8, FOXP3, CD127, CD25, CD27, CD56, CXCR3, CCR6, CCR7, CD45RA, CD45RO, CTLA-4, CD161, IFNγ, TNFα, IL-1β, IL-17A, IL-21, IL-22, HLA-DR, CD38, or a polynucleotide encoding said marker polypeptide.

In another aspect, the invention provides a method for detecting a marker in a sample derived from a subject suspected of having inflammatory bowel disease, the method involving detecting two or more marker polypeptides selected from one or more of the following: CD45, CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα in the sample.

In another aspect, the invention provides a method for distinguishing active ulcerative colitis from a non-inflammatory bowel disease state, the method involving detecting in a T cell derived from the mucosa of a subject having active ulcerative colitis an increase in a marker polypeptide selected from one or more of the following CD38, HLA-DR, FOXP3, IL-17A, CCR4, and CTLA-4 and a decrease in TNFα relative to a reference level.

In another aspect, the invention provides a method for distinguishing active ulcerative colitis from a non-inflammatory bowel disease state, the method involving detecting in a T cell derived from the mucosa of a subject having active ulcerative colitis an increase in a marker polypeptide selected from one or more of the following: IL-17A, HLA-DR, FOXP3, CTLA-4, CD45RO, CD45RA, CD38, CD27, CD25, CD24, CD161, CCR7, CCR6, and CCR4 and a decrease in TNFα relative to a reference level. In some embodiments of the above aspect, the method further involves detecting an increase in a marker polypeptide selected from one or more of the following: CD45RO, CD45RA, CD38, CD27, CD25, CD24, CD161, CCR7, and CCR6 and a decrease in CD44 and CD27 relative to a reference level.

In another aspect, the invention provides a method for distinguishing active ulcerative colitis from a non-inflammatory bowel disease state, the method involving detecting in a T cell derived from the mucosa or peripheral blood of a subject an increase or decrease in a marker polypeptide selected from any one or more of a marker polypeptide present in FIG. 7C or 18A.

In another aspect, the invention provides a method for distinguishing active Crohn's disease from a non-inflammatory bowel disease state, the method involving detecting in a T cell derived from the mucosa of a subject having active Crohn's disease an increase in a marker polypeptide selected from any one or more of CD38, HLA-DR, and FOXP3.

In an embodiment of the above aspect, the method involves detecting in a T cell derived from the mucosa of a subject having active Crohn's disease an increase in a marker polypeptide selected from any one or more of IL-1β, IL-17, IFNγ, CTLA-4, CD45RA, CD24, CCR7, CCR6, and CCR4 relative to a reference level. In an embodiment of the above aspect, the method further involves detecting a decrease in TNFα. In an embodiment of the above aspect, the method further involves detecting an increase in marker polypeptide CCR4 in a B cell derived from the mucosa of the subject relative to a reference level. In an embodiment of the above aspect, the method further involves detecting an increase in IL-1β, CTLA-4, CD38, and CD161 and a decrease in TNFα in a B cell derived from the mucosa of the subject relative to a reference level. In an embodiment of the above aspect, the method further involves detecting an increase in IL-1β, IFNγ, and CD45RO and a decrease in TNFα in an innate immune cell derived from the mucosa of the subject relative to a reference level. In an embodiment of the above aspect, the method further involves detecting an increase in marker polypeptides IL-1β, IFNγ, FOXP3, CD45RO, CD38, CCR4, CD24, and CD161 and a decrease in marker polypeptides IL-17 and CXCR3 in an innate immune cell derived from the mucosa of the subject relative to a reference level. In an embodiment of the above aspect, the method further involves detecting in a B cell derived from the peripheral blood of the subject an increase in CXCR3 relative to a reference level. In an embodiment of the above aspect, the method further involves detecting in a B cell derived from the peripheral blood of the subject an increase in marker polypeptides CXCR3, CD45RO, CD38, and CCR6 and a decrease in marker polypeptides CD24 and TNFα. In an embodiment of the above aspect, the method further involves detecting an increase in marker polypeptides CD45RO, CD38, and CCR6 and a decrease in marker polypeptides CD24 and TNFα relative to a reference level. In an embodiment of the above aspect, the method further involves detecting in a T cell derived from peripheral blood of the subject an increase or decrease in a polypeptide marker present in FIG. 7C. In an embodiment of the above aspect, the method further involves detecting in an innate immune cell derived from peripheral blood of the subject an increase or decrease in a polypeptide marker present in FIG. 18A. In an embodiment of the above aspect, the method further involves detecting in a T cell derived from peripheral blood of the subject an increase in marker polypeptide IL-22, IL-21, IL-17, CTLA-4, and CD45RA and a decrease in marker polypeptide CD161. In an embodiment of the above aspect, the method further involves detecting in an innate immune cell derived from peripheral blood of the subject an increase in IL-1β and HLA-DR and decrease in CD161.

In another aspect, the invention provides a method for distinguishing active Crohn's disease from active ulcerative colitis, the method involving detecting in a T cell derived from the mucosa of a subject an increase in marker polypeptide IL-1β and a decrease in IL-17A relative to a reference level. In an embodiment of the above aspect, the method further involves detecting in a T cell derived from the mucosa of a subject an increase in marker polypeptide TNFα and IFNγ or an increase in HLA-DR, CD38, CD27, CD24, and CD161 relative to a reference level. In an embodiment of the above aspect, the method further involves detecting an increase in IL-1β and a decrease in IL-17A in a B cell derived from the mucosa of the subject relative to a reference level. In an embodiment of the above aspect, the method further involves detecting an increase in IL-1β and CTLA-4 and a decrease in TNFα, IL-17A, CD45RA, and CCR7 in a B cell derived from the mucosa of the subject relative to a reference level. In an embodiment of the above aspect, the method further involves detecting an increase in IL-1β, TNFα, and IL-22 and a decrease in CD38 in an innate immune cell derived from the mucosa of the subject relative to a reference level. In an embodiment of the above aspect, the method further involves detecting an increase in polypeptide markers TNFα, IL-22, and IL-1β and a decrease in polypeptide markers HLA-DR, CD44, CD38, and CCR4 in an innate immune cell derived from the mucosa of the subject relative to a reference level. In an embodiment of the above aspect, the method further involves detecting an increase in polypeptide markers CCR7, CD24, and IL-22 in a T cell derived from the peripheral blood of the subject relative to a reference level. In an embodiment of the above aspect, the method further involves detecting an increase in polypeptide markers IL-21, CD45RA, and CCR4 and a decrease in polypeptide marker IFNγ in a T cell derived from the peripheral blood of the subject relative to a reference level. In an embodiment of the above aspect, the method further involves detecting an increase in polypeptide markers IL-1β and CD38 and a decrease in polypeptide marker CTLA-4 and CD45RA in a B cell derived from the peripheral blood of the subject relative to a reference level. In an embodiment of the above aspect, the method further involves detecting an increase in polypeptide marker IL-1β in a B cell derived from the peripheral blood of the subject relative to a reference level. In an embodiment of the above aspect, the method further involves detecting an increase in polypeptide marker CD38 and a decrease in polypeptide markers CTLA-4 and CD45RA. In an embodiment of the above aspect, the method further involves detecting an increase in polypeptide markers IL-1β, CXCR3, TNFα, and HLA-DR and a decrease in CD45RA in an innate immune cell derived from the peripheral blood of the subject relative to a reference level. In an embodiment of the above aspect, the method further involves detecting an increase in TNFα, and a decrease in CD24.

In another aspect, the invention provides a method for distinguishing active ulcerative colitis from inactive ulcerative colitis, the method involving detecting in a T cell derived from the mucosa of a subject an increase in HLA-DR, CD38, and CTLA-4 relative to a reference level. In an embodiment of the above aspect, the method further involves detecting an increase in marker polypeptides CD45RO, CD45RA, CD44, CD27, CD25, CCR7, and CCR4 and a decrease in marker polypeptides TNFα and IFNγ. In an embodiment of the above aspect, the method further involves detecting an increase in marker polypeptides HLA-DR, CD38, and CTLA-4, CD45RO, CD45RA, CD44, CD27, CD25, CCR7, and CCR4 and a decrease in TNFα and IFNγ in a B cell derived from the mucosa of the subject relative to a reference level. In an embodiment of the above aspect, the method further involves detecting an increase in IL-17A and a decrease in TNFα in an innate immune cell derived from the mucosa of the subject relative to a reference level. In an embodiment of the above aspect, the method further involves detecting an increase in IL-17A, CTLA-4, CD45RO, CD45RA, CD44, and CD38 and a decrease in TNFα and CXCR3 in an innate immune cell derived from the mucosa of the subject relative to a reference level.

In another aspect, the invention provides a method for distinguishing active Crohn's Disease from inactive Crohn's Disease, the method involving detecting in a T cell derived from the mucosa of a subject an increase in polypeptide markers IL-1β and a decrease in CD161 relative to a reference level. In an embodiment of the above aspect, the method further involves detecting an increase in TNFα, CD45RA, and CD38 and a decrease in CD27, CD45RO, and IL-21 relative to a reference level. In an embodiment of the above aspect, the method further involves detecting a decrease in polypeptide markers CCR7, CD25, HLA-DR, and TNFα in a B cell derived from the mucosa of the subject relative to a reference level. In an embodiment of the above aspect, the method further involves detecting an increase in polypeptide markers IL-1β, CTLA-4, and CD24 and a decrease in polypeptide markers CD161, HLA-DR, CCR7, and CXCR3 in an innate immune cell derived from the mucosa of the subject relative to a reference level. In an embodiment of the above aspect, the method further involves detecting an increase in in polypeptide marker CCR4 in a T cell derived from the peripheral blood of the subject relative to a reference level. In an embodiment of the above aspect, the method further involves detecting an increase in polypeptide markers IL-1β, IL-22, and CTLA-4 relative to a reference level. In an embodiment of the above aspect, the method further involves detecting an increase in polypeptide markers IL-1β and a decrease in CD161 in an innate immune cell derived from the mucosa of the subject relative to a reference level. In an embodiment of the above aspect, the method further involves detecting an increase in in polypeptide markers CTLA-4 and CD24 and a decrease in polypeptide markers HLA-DR, CCR7, and CXCR3 in an innate immune cell derived from the mucosa of the subject relative to a reference level. In an embodiment of the above aspect, the method further involves detecting an increase in IL-17A and CCR4 in a B cell derived from the peripheral blood of the subject relative to a reference level. In an embodiment of the above aspect, the method further involves detecting an increase in CD45RO and a decrease in CTLA-4 in an innate cell derived from the peripheral blood of the subject relative to a reference level.

In another aspect, the invention provides a method for distinguishing inactive Crohn's Disease from non-inflammatory bowel disease, the method involving detecting in a T cell derived from the mucosa of a subject an increase in polypeptide marker IL-17A relative to a reference level. In an embodiment of the above aspect, the method further involves detecting an increase in polypeptide markers IL-21, IFNγ, FOXP3, HLA-DR, CTLA-4, CXCR3, CD45RO, CD45RA, CD38, CD27, CD25, CD24, CD161, CCR7, CCR6, and CCR4 relative to a reference level. In an embodiment of the above aspect, the method further involves detecting an increase in polypeptide markers CCR7, CD45RO, IFNγ, and HLA-DR in a B cell derived from the mucosa of the subject relative to a reference level. In an embodiment of the above aspect, the method further involves detecting an increase in polypeptide markers FOXP3, IL-21, CD38, CD25, CD24, CD161, and CCR4 and a decrease in polypeptide marker CD27 relative to a reference level. In an embodiment of the above aspect, the method further involves detecting an increase in polypeptide markers IL-21, IFNγ, HLA-DR, FOXP3, CD45RO, CD38, CD24, CD161, CCR7, CCR6, and CCR4 and decrease in polypeptide marker IL-17A in an innate cell derived from the mucosa of the subject relative to a reference level. In an embodiment of the above aspect, the method further involves detecting an increase in CD44 in a T cell derived from the peripheral blood of the subject relative to a reference level. In an embodiment of the above aspect, the method further involves detecting an increase in CD38 in a B cell derived from the peripheral blood of the subject relative to a reference level. In an embodiment of the above aspect, the method further involves detecting an increase in polypeptide markers HLA-DR and CTLA-4 and a decrease in polypeptide marker CCR4 in an innate cell derived from the peripheral blood of the subject relative to a reference level.

In another aspect, the invention provides a method for distinguishing active ulcerative colitis or Crohn's disease from non-inflammatory bowel disease, the method involving detecting in a B cell derived from the peripheral blood of the subject an increase in polypeptide marker CXCR3 relative to a reference level.

In another aspect, the invention provides a method for distinguishing inactive ulcerative colitis from non-inflammatory bowel disease, the method involving detecting in a T cell derived from the mucosa of a subject an increase in polypeptide markers IL-17A, CD45RA, CD38, CD24, CD161, and CCR4 relative to a reference level. In an embodiment of the above aspect, the method further involves detecting an increase in polypeptide markers CD45RO and CCR4 and a decrease in polypeptide marker CD27 in a B cell derived from the mucosa of the subject relative to a reference level. In an embodiment of the above aspect, the method further involves detecting an increase in polypeptide markers IL-21, CD38, and CD161 in an innate cell derived from the mucosa of the subject relative to a reference level.

In an embodiment of the above aspect, the method further involves detecting a decrease in polypeptide markers TNFα, IFNγ, CTLA-4, CD45RO, CD161, CCR6, and CCR4 in a T cell derived from the peripheral blood of the subject relative to a reference level. In an embodiment of the above aspect, the method further involves detecting a decrease in polypeptide markers CD161 and CCR4 in a B cell derived from the peripheral blood of the subject relative to a reference level. In an embodiment of the above aspect, the method further involves detecting an increase in CD27 and decrease in polypeptide markers IL-22, CD161, CCR6, and CCR4 in an innate cell derived from the peripheral blood of the subject relative to a reference level.

In another aspect, the invention provides a method for distinguishing active ulcerative colitis from active Crohn's disease, the method involving detecting an increase in polypeptide markers CCR7, CD24, and IL-22 in a T cell derived from the peripheral blood of the subject relative to a reference level. In an embodiment of the above aspect, the method further involves detecting a decrease in IL-1β and CD38 and an increase in CTLA-4 and CD45RA in a B cell derived from the peripheral blood of the subject relative to a reference level. In an embodiment of the above aspect, the method further involves detecting an increase in polypeptide markers IL-1β, CXCR3, and HLA-DR and a decrease in polypeptide marker CD45RA in an innate immune cell of the subject relative to a reference level.

In another aspect, the invention provides a method for distinguishing active ulcerative colitis from inactive ulcerative colitis, the method involving detecting an increase in polypeptide markers CD24, CD45RA, and CCR4 in a T cell derived from the peripheral blood of the subject relative to a reference level.

In another aspect, the invention provides a method for distinguishing active ulcerative colitis from inactive ulcerative colitis, the method involving detecting an increase in polypeptide markers IL-22, HLA-DR, CD45RA, CD24, CCR7, CCR6, and CCR4 in a T cell derived from the peripheral blood of the subject relative to a reference level.

In another aspect, the invention provides a method of treating ulcerative colitis in a selected subject, the method involving administering an agent that inhibits IL-17 or CD38 to a subject, wherein the subject is selected as having increased levels of IL-17A or CD38 in a T cell derived from the mucosa of the subject relative to a reference.

In another aspect, the invention provides a method of treating Crohn's disease in a selected subject, the method involving administering an agent that inhibits IL-1β blockade to a selected subject, wherein the subject is selected as having increased levels of IL-1β in a T cell derived from the mucosa of the subject relative to a reference.

In another aspect, the invention provides a method of characterizing disease progression in a subject having or suspected of having inflammatory bowel disease, the method involving:

detecting two or more markers selected from any one or more of CD45, CD3, CD4, CD8, CD24, CD25, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, and TNFα in a first sample derived from a subject at a first point in time; and

detecting the same markers in a second sample derived from the subject at a second point in time; thereby characterizing disease progression.

In another aspect, the invention provides a method of distinguishing active ulcerative colitis from other forms of inflammatory bowel disease, the method involving detecting in a sample derived from the mucosa of a subject having active ulcerative colitis an increased B cell to T cell ratio.

In another aspect, the invention provides a method of distinguishing active ulcerative colitis from a non-inflammatory bowel disease state, the method involving detecting an increase in CD4⁺ T cells in a sample derived from a subject having active ulcerative colitis relative to a reference level. In an embodiment of the above aspect, the method further involves detecting a decrease in CD4⁻CD8⁻ T cells in the sample derived from the subject having active ulcerative colitis relative to a reference level.

In another aspect, the invention provides a method of distinguishing active ulcerative colitis, active Crohn's disease, or inactive Crohn's disease from a non-inflammatory bowel disease state, the method involving detecting an increased in HLA-DR⁺CD38⁺ T cells in a sample derived from a subject having or suspected of having active ulcerative colitis, active Crohn's disease, or inactive Crohn's disease relative to a reference. In an embodiment of the above aspect, the method further involves detecting in the HLA-DR⁺CD38⁺ T cells an increase in expression of one of the following markers selected from any one or more of CD45RO, CD45RA, CCR4, CCR6, CCR7, CXCR3, and IL-1β. In an embodiment of the above aspect, the method further involves detecting decreased expression levels of TNFα and IFNγ in the sample derived from the mucosa of the subject having or suspected of having active ulcerative colitis, active Crohn's disease, or inactive Crohn's disease relative to reference levels. In an embodiment of the above aspect, the method further involves detecting increased expression of memory T cell markers, activation markers, and pro-inflammatory cytokines. In an embodiment of the above aspect, the pro-inflammatory cytokines contain IL-1β. In an embodiment of the above aspect, the memory T cell markers contain CD45RO and CD161.

In another aspect, the invention provides a method of distinguishing active ulcerative colitis, active Crohn's disease, or inactive Crohn's disease from a non-inflammatory bowel disease state, the method involving detecting increased number of FOXP3⁺ regulatory T cells in a sample derived from active ulcerative colitis, active Crohn's disease, or inactive Crohn's disease relative to a reference. In an embodiment of the above aspect, the FOXP3⁺ regulatory T cells have an increased expression level of CD45RA and CD24 relative to a reference level. In another embodiment, the FOXP3⁺ regulatory T cells have an increased expression level of IL-17A relative to a reference level.

In another aspect, the invention provides a method of distinguishing Crohn's disease from ulcerative colitis and from a non-inflammatory bowel disease state, the method involving detecting increased expression of IL-1β in FOXP3⁺ regulatory T cells in a mucosa sample derived from a subject having Crohn's disease relative to a reference level.

In another aspect, the invention provides a method of distinguishing active inflammatory bowel disease from a non-inflammatory bowel disease state, the method involving detecting in a mucosa sample derived from a subject having inflammatory bowel disease an increased FOXP3⁺ expression level in non-regulatory T cells relative to a reference level. In an embodiment of the above aspect, the non-regulatory T cells are CD25⁻CD4⁺ or CD25-CD8⁺ T cells; or are CD25⁻CD4⁺ and CD8⁺ T cells.

In another aspect, the invention provides a method of distinguishing active ulcerative colitis from active Crohn's disease and inactive ulcerative colitis, the method involving detecting an increased IL-22 expression level in FOXP3⁺ regulatory T cells in peripheral blood relative to a reference level.

In another aspect, the invention provides a method of distinguishing active ulcerative colitis and inactive Crohn's disease from a non-inflammatory bowel disease state, the method involving detecting a decreased number of conventional T cells in a mucosa sample derived from a subject having active ulcerative colitis or inactive Crohn's disease compared to a non-inflammatory bowel disease reference number. In an embodiment of the above aspect, the method further involves characterizing T helper subsets. In another embodiment, increased ratios relative to reference ratios of TH17 to TH1 cells and TH1-17 to TH1 cells is indicative of active ulcerative colitis. In an embodiment of the above aspect, the method further involves detecting expression of IFNγ and IL-17A in T helper cells, wherein the expression of IFNγ and IL-17A is increased in active ulcerative colitis relative to active Crohn's disease and the non-inflammatory bowel disease state.

In another aspect, the invention provides a method of distinguishing active ulcerative colitis from a non-inflammatory bowel disease state, the method involving detecting one or more of increased CCR4 expression in all CD4⁺ T cell subsets; increased CTLA-4 expression in T cell subset excluding FOXP⁺ regulatory T cells; increased CD27 expression in CD8⁺ T cells; and decreased TNFα expression in TH1, T1-17, and double negative T cells relative to a reference level.

In another aspect, the invention provides a method of distinguishing active inflammatory bowel disease from a non-inflammatory bowel disease state, the method involving detecting an increased number of plasmablasts and regulatory B cells in the mucosa sample derived from a subject having active inflammatory bowel disease relative to a reference number.

In another aspect, the invention provides a method of distinguishing active inflammatory bowel disease from a non-inflammatory bowel disease state, the method involving detecting an increased number of CD123⁺ innate cells relative to a reference number in a mucosa sample derived from a subject having active inflammatory bowel disease.

In another aspect, the invention provides a method of distinguishing active ulcerative colitis from inactive ulcerative colitis, the method involving detecting an increased number of dendritic cells relative to a reference number in a mucosa sample derived from a subject having active ulcerative colitis.

In another aspect, the invention provides a method of distinguishing active Crohn's disease from inactive Crohn's disease and from a non-inflammatory bowel disease state, the method involving detecting an increased proportion of plasmacytoid dendritic cells relative to a reference proportion. In an embodiment, the detecting is by mass cytometry Time-of-Flight (CyTOF), flow cytometry, bulk RNA sequencing, or single cell RNA sequencing.

In another aspect, the invention provides a method of distinguishing active ulcerative colitis from a non-inflammatory bowel disease state and active Crohn's disease, the method involving detecting an increased number of effector memory T cells in a mucosa sample derived from a subject having active ulcerative colitis compared to a non-inflammatory bowel disease state or Crohn's disease reference number, wherein the effector memory T cells are defined as of CD45RA⁺CD45RO⁺CCR7⁺CD27⁺CD3⁺CD45⁺CD4⁻CD8α⁻ and also express CD161, IL17A, TNFα, and IFNγ. In another embodiment, the effector memory T cells also express HLA-DR and CD38.

In another aspect, the invention provides a method of distinguishing active ulcerative colitis from a non-inflammatory bowel disease state, inactive ulcerative colitis, inactive Crohn's disease, or active Crohn's disease, the method involving detecting a decreased number of effector memory T cells in a mucosa sample derived from a subject having active ulcerative colitis compared to a non-inflammatory bowel disease state, inactive ulcerative colitis, or inactive or active Crohn's disease reference number, wherein the effector memory T cells are defined as of CD45RA⁺CD45RO⁺CCR7⁺CD27⁺CD3⁺CD45⁺ and also express CD8α, TNFα, and IFNγ. In an embodiment, the effector memory T cells also express HLA-DR and CD38.

In another aspect, the invention provides a method of distinguishing active ulcerative colitis from a non-inflammatory bowel disease state, inactive ulcerative colitis, inactive Crohn's disease, or active Crohn's disease, the method involving detecting a decreased number of effector memory T cells in a mucosa sample derived from a subject having active ulcerative colitis compared to a non-inflammatory bowel disease state, inactive ulcerative colitis, or inactive or active Crohn's disease reference number, wherein the effector memory T cells are defined as of CD45RA⁺CD45RO⁺CCR7⁺CD27⁺CD3⁺CD45⁺ and also express CD56, TNFα, and IFNγ.

In another aspect, the invention provides a method of distinguishing active Crohn's disease from active ulcerative colitis, the method involving detecting an increased number of IL-1β⁺HLA-DR⁺CD38⁺CD45⁺CD3⁺ T cells in a mucosa sample derived from a subject having active Crohn's disease compared to an active ulcerative colitis reference number.

In another aspect, the invention provides a method of distinguishing active ulcerative colitis and inactive Crohn's disease, the method involving detecting an increased number of FoxP3⁺HLA-DR⁺CD38⁺CTLA-4⁺CD45RO⁺ T regulatory cells in a mucosa sample derived from a subject having active ulcerative colitis compared to a non-inflammatory bowel disease state and active Crohn's disease reference number.

In another aspect, the invention provides a method of distinguishing inflammatory bowel disease from a non-inflammatory bowel disease state, the method involving detecting an increased number of FoxP3^{lo}HLA-DR⁺CD38⁺CTLA-4⁺CD45RO⁺IFNγ⁺TNFα⁺ T regulatory cells in a mucosa sample derived from a subject having inflammatory bowel disease compared to a non-inflammatory bowel disease state reference number.

In another aspect, the invention provides a method of distinguishing active ulcerative colitis from a non-inflammatory bowel disease state, inactive Crohn's disease and active Crohn's disease, the method involving detecting an increased number of FoxP3^{lo}HLA-DR⁺CD38⁺CTLA-4⁺CD45RO⁺IFNγ⁺TNFα⁺IL17A⁺ T regulatory cells in a mucosa sample derived from a subject having inflammatory bowel disease compared to a non-inflammatory bowel disease state reference number.

In another aspect, the invention provides a method of distinguishing active Crohn's disease from active ulcerative colitis, the method involving detecting an increased number of CD45⁺CD3⁺CD25⁺CD127⁺CCR7⁺CD27⁺CD45RA⁺CD45RO⁺ central memory T cells regulatory cells that also express IL-1β and CXCR3 in a peripheral blood sample derived from a subject having active Crohn's disease compared to an active ulcerative colitis reference number.

In another aspect, the invention provides a method of distinguishing active Crohn's disease from a non-inflammatory bowel disease state, inactive ulcerative colitis, and active ulcerative colitis, the method involving detecting an increased number of IL-1β⁺TNFα⁺IFNγ⁺ naïve B cells in a mucosa sample derived from a subject having active Crohn's disease compared to a non-inflammatory bowel disease state, inactive ulcerative colitis, or active ulcerative colitis reference number.

In another aspect, the invention provides a method of distinguishing active ulcerative colitis, inactive Crohn's disease, and active Crohn's disease from a non-inflammatory bowel disease state, the method involving detecting an increased number of CXCR3⁺ plasmablasts in a mucosa sample derived from a subject having active ulcerative colitis, inactive Crohn's disease, or active Crohn's disease compared to a non-inflammatory bowel disease state, reference number.

In another aspect, the invention provides a method of distinguishing active ulcerative colitis from a non-inflammatory bowel disease state, active Crohn's disease, inactive Crohn's disease, and inactive ulcerative colitis, the method involving detecting an increased number of CD56⁺ granulocytes in a mucosa sample derived from a subject having active ulcerative colitis compared to a non-inflammatory bowel disease state, active Crohn's disease, inactive Crohn's disease, and inactive ulcerative colitis reference number, wherein the granulocytes are CD45⁺CXCR3⁺CD44⁺CCR4⁺CCR7⁺CD66b⁺CD56⁺CD3⁻CD19⁻.

In another aspect, the invention provides a method of distinguishing active Crohn's disease from a non-inflammatory bowel disease state, inactive Crohn's disease, inactive ulcerative colitis, and active ulcerative colitis, the method involving detecting an increased number of dendritic cells or plasmacytoid dendritic cells in a mucosa sample derived from a subject having active Crohn's disease compared to a non-inflammatory bowel disease state, inactive ulcerative colitis, or active ulcerative colitis reference number, wherein the dendritic cells or plasmacytoid dendritic cells are CD11c⁺CD123⁺HLA-DR⁺IL-1β⁺.

In another aspect, the invention provides a method of distinguishing active Crohn's disease from active ulcerative colitis, the method involving detecting an increased number of dendritic cells or plasmacytoid dendritic cells in a peripheral blood sample derived from a subject having active Crohn's disease compared to an active ulcerative colitis reference number, wherein the dendritic cells or plasmacytoid dendritic cells are CD11c⁺CD123⁺HLA-DR⁺IL-1β⁺.

In another aspect, the invention provides a method of distinguishing active Crohn's disease from a non-inflammatory bowel disease state and active ulcerative colitis, the method involving detecting a decreased number of Group 1 innate lymphoid cells in a peripheral blood sample derived from a subject having active Crohn's disease compared to a non-inflammatory bowel disease state or active ulcerative colitis reference number, wherein the Group 1 innate lymphoid cells are CD45⁺CD3⁻CD19⁻CD38⁺CD45RA⁺IFNγ⁺Tbet⁺CD161⁺. In another aspect, the invention provides a method of distinguishing active Crohn's disease from active ulcerative colitis, the method involving detecting an increased number of innate lymphoid cells (ILC) type 1 and ILC-like cells in a mucosa sample derived from a subject having active Crohn's disease compared to an active ulcerative colitis reference number, wherein the innate lymphoid cells (ILC) type 1 and ILC-like cells are CD45⁺CD3⁻CD19⁻IFNγ⁺TNFα⁺Tbet^{+/-}CD8α^{+/-}.

In another aspect, the invention provides a method of distinguishing active ulcerative colitis from a non-inflammatory bowel disease state, the method involving detecting a decreased number of innate lymphoid cells (ILC) type 1 and ILC-like cells in a mucosa sample derived from a subject having active ulcerative colitis compared to a non-inflammatory bowel disease state reference number, wherein the innate lymphoid cells (ILC) type 1 and ILC-like cells are CD45⁺CD3⁻CD19⁻IFNγ⁺TNFα⁺Tbet^{+/-}CD8α^{+/-}.

In another aspect, the invention provides a method of distinguishing active Crohn's disease from active ulcerative colitis and non-inflammatory bowel disease state, the method involving detecting a decreased number of innate lymphoid cells (ILC) type 1 and ILC-like cells in a peripheral blood sample derived from a subject having active Crohn's disease compared to active ulcerative colitis or non-inflammatory bowel disease state reference number, wherein the innate lymphoid cells (ILC) type 1 and ILC-like cells are CD45⁺CD3⁻CD19⁻IFNγ⁺TNFα⁺Tbet^{+/-}CD8α^{+/-}.

In another aspect, the invention provides a method of distinguishing active ulcerative colitis from a non-inflammatory bowel disease state, inactive ulcerative colitis, active Crohn's disease, and inactive Crohn's disease, the method involving detecting a decreased number of innate lymphoid cells type 3 in a mucosa sample derived from a subject having active ulcerative colitis compared to a non-inflammatory bowel disease state, inactive ulcerative colitis, active Crohn's disease, or inactive Crohn's disease reference number, wherein the innate lymphoid cells type 3 are CD45⁺CD3⁻CD19⁻CD161⁺ CD127⁺CCR6⁺ckit⁺TNFα⁺.

In another aspect, the invention provides a method of distinguishing active inflammatory bowel disease from inactive inflammatory bowel disease and a non-inflammatory bowel disease state, the method involving detecting an increased number of macrophages/monocytes in a mucosa sample derived from a subject having active inflammatory bowel disease compared to inactive inflammatory bowel disease and a non-inflammatory bowel disease state reference number, wherein the macrophages/monocytes are CD45⁺CD3⁻CD19⁻CD14⁺CD11c⁺HLA-DR⁺CD14⁺.

In another aspect, the invention provides a method of distinguishing active Crohn's disease from active ulcerative colitis and a non-inflammatory bowel disease state, the method involving detecting an increased number of macrophages/monocytes in a peripheral blood sample derived from a subject having active Crohn's disease compared to active ulcerative colitis or a non-inflammatory bowel disease state reference number, wherein the macrophages/monocytes are CD45⁺CD3⁻CD19⁻CD14⁺CD11c⁺HLA-DR⁺CD14⁺.

In various embodiments of the above aspects, the macrophages/monocytes are CD45⁺CD3⁻CD19⁻CD14⁺CD11c⁺HLA-DR⁺CD14⁺IL-1β⁺. In various embodiments of the above aspects, the capture molecule is a polypeptide, polynucleotide probe, or fragment thereof. In various embodiments of the above aspects, the polypeptide is an antibody that specifically binds the marker polypeptide. In various embodiments of the above aspects, the polynucleotide is an aptamer that specifically binds the marker polypeptide. In various embodiments of the above aspects, the capture molecule is a polynucleotide probe having a nucleic acid sequence at least partially complementary to a polynucleotide encoding the marker polypeptide. In various embodiments of the above aspects, the substrate is a bead or planar surface. In various embodiments of the above aspects, the planar surface is a membrane, filter, chip, glass slide, or other solid support. In various embodiments of the above aspects, the method further involves detecting an increase in CCR4 in a B cell derived from the mucosa of the subject relative to a reference level. In various embodiments of the above aspects, the method involved detecting in a B cell derived from the mucosa of the subject an increase in marker polypeptides IL-17A, HLA-DR, CD45RO, CD45RA, CD38, CD25, CD24, CD161, CCR7, and CCR4 and a decrease in CD44 and CD27. In an embodiment of the above aspect, the method further involves detecting an increase in marker polypeptides IL-1β, CD38, CD44, HLA-DR, and IL-21 and a decrease in TNFα in an innate immune cell derived from the mucosa of the subject relative to a reference level. In an embodiment of the above aspect, the method further involves detecting an increase in a marker polypeptide selected from any one or more of CD45RO, CD45RA, CD161, CCR4, and IFNγ and a decrease in CXCR3 in an innate immune cell derived from the mucosa of the subject relative to a reference level. In an embodiment of the above aspect, the method further involves detecting in a B cell derived from the peripheral blood of the subject an increase in marker polypeptide CXCR3 relative to a reference level. In an embodiment of the above aspect, the method further involves detecting in a B cell derived from the peripheral blood of the subject an increase in a marker polypeptide selected from any one or more of CLTA-4, CD27, and CCR4 and a decrease in CCR6 relative to a reference level. In an embodiment of the above aspect, the method further involves detecting in a T cell derived from peripheral blood of the subject an increase in a marker polypeptide selected from any one or more of IFNγ, CTLA-4, and CD38 and decrease in CD24, CD161, and CCR4. In an embodiment of the above aspect, the method further involves detecting in an innate immune cell derived from peripheral blood of the subject an increase in a marker polypeptide selected from any one or more of FOXP3, CTLA-4, CD45RA, and CD44 and a decrease in TFNα, IFNγ, HLA-DR, and CD161.

In various embodiments of any of the above methods, the detecting is by mass cytometry Time-of-Flight (CyTOF), flow cytometry, bulk RNA sequencing, or single cell RNA sequencing. The present disclosure provides methods and compositions for detecting, characterizing, and differentiating inflammatory bowel disease subtypes. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant not having or suspected of having a disease and/or not being derived from a diseased source. For example, a control sample is a sample derived from a subject that exhibit no signs of a disease (e.g., inflammatory bowel disease, Crohn's disease, ulcerative colitis).

By “decreases” is meant a reduction by at least about 5% relative to a reference level. A decrease may be by 5%, 10%, 15%, 20%, 25% or 50%, or even by as much as 75%, 85%, 95% or more.

“Detect” refers to identifying the presence, absence, or amount of the analyte to be detected.

By “detectable label” is meant a composition that when linked to a molecule of interest renders the latter detectable, via spectroscopic, photochemical, biochemical, immunochemical, or chemical means. For example, useful labels include rare earth metals, radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include inflammatory bowel disease, Crohn's disease, and ulcerative colitis.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. The term “biomarker” may be used interchangeably with the term “marker.”

By “mucosa” is meant the inner lining of the intestinal tract including epithelium and lamina propria.

By “non-inflammatory bowel disease” or “a non-inflammatory bowel disease state” is meant the absence of inflammatory bowel disease such as active and inactive Crohn's disease and ulcerative colitis, as well as undefined inflammatory bowel disease.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In one embodiment, a reference is a corresponding healthy cell or an a corresponding untreated cell.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustrating cell types associated with Crohn's disease and ulcerative colitis.

FIG. 2 is a schematic of the experimental protocol used to characterize samples derived from subjects having Crohn's disease or ulcerative colitis relative to each other and to control samples derived from subjects without inflammatory bowel disease.

FIGS. 3A to 3C illustrate histological evaluation of inflammatory bowel disease. FIG. 3A is a series of images of the colonic mucosa scored (0-4) using the Nancy Histological Index as described in Marchal-Bressenot et al., Gut, 65:1919-20 (2016). The box-and-whisker plots depict the median Nancy score (thick horizontal line) in each group, and points are “jittered” for visual discrimination although the underlying value of each point remains on the 0-4 integer scale. FIG. 3B consists of two graphs depicting the Nancy Histological Index scores of mucosa mononuclear cells (MC) and peripheral blood mononuclear cells (PBMC) for inactive and active forms of ulcerative colitis (UC) and Crohn's disease (CD). FIG. 3C is a graph comparing inflammatory bowel disease diagnoses made by assessing patient samples using the Nancy Histological Index or determined by the clinical assessment of an endoscopist.

FIGS. 4A to 4D provide analysis of T cells that demonstrates UC- and CD-specific cytokine signatures. FIG. 4A comprises box-and-whisker plots demonstrating proportion of manually-gated immune cell subsets (total T cells, total B cells, total innate cells, T regulatory cells (Tregs)) out of all CD45⁺ cells (for Tregs, out of total T cells) in the mucosa and periphery as well as the B:T cell ratio across subject groups. FIG. 4B comprises flow cytometry data and box-and-whisker plots demonstrating the percentage of total T cells, total B cells (out of total CD45⁺ cells) and B:T ratio of an independent cohort, with representative biaxial gating plots. FIG. 4C is an image showing the gating strategy to define T cells (CD3⁺CD45⁺) which were exported and subjected to dimensionality reduction (t-SNE) and clustering (FlowSOM) as depicted in labeled t-SNE cluster plot (right). FIG. 4D is a CyTOF marker heatmap (rows are clusters, columns are markers, tile colors represent mean metal intensity per Color Key and Histogram legend scaled across all tiles). On the right are manually-labeled cluster identities and a schematic of cluster abundance in mucosa vs. periphery. A hierarchical clustering dendrogram is provided to the left of the heatmap, with nodes numbered and selected nodes highlighted. Selected node branches are outlined on heatmap and labeled on the right. T cell memory subsets were assigned and color-coded (naïve: CD45RA⁺CD45RO⁻CCR7⁺CD27⁺; central memory (CM): CD45RA⁻CD45RO⁺CCR7⁺CD27⁺; effector memory (EM): CD45RA⁻CD45RO⁺CCR7⁻CD27^{+/-}; terminally differentiated effector memory cells re-expressing CD45RA (TEMRA): CD45RA⁺CD45RO^{+/-}CCR7⁻CD27^{+/-}; and a subtype external to these established categories, CD45RO⁺CD45RA⁺).

FIGS. 5A-5D illustrate that IL-17A⁺⁺CD161⁺ EM T cells are enriched while IFNγ⁺TNFα⁺ EM T cell subsets are diminished in UCa mucosa. FIG. 5A comprises a box- and whisker plot of Node 4(+) showing the percentage of T cells in mucosa samples from different subsets of patients. Node 4(+) is labeled on a t-SNE plot, with a IL-17A heatmap for reference. FIG. 5B comprises t-SNE plot of nodes 11(+) and 12(−) t-SNE plot, IFNγ/TNFα/CD8α heatmaps, and box- and whisker plots of these nodes. FIG. 5C shows flow cytometry data from an independent cohort demonstrating proportion of IFNγ⁺ T cells (out of total T cells) and the ratio of IL-17A to IFN γ⁺ T cells across subject groups with representative biaxial plots shown. FIG. 5D shows scRNA-seq data of UCa subjects from CyTOF cohort (n=5 samples) represented by t-SNE plot colored by cluster (left; total cells analyzed=3,979) with cluster identity labeled according to differential gene expression. Data was subsetted on T cells, which were then re-clustered (right; total cells analyzed=718) with generation of 9 clusters labeled “0” through “8”); cluster 1, corresponding to IL17A⁺ MAIT cells or IL17A⁺CD8αα intraepithelial lymphocytes (IEL), is circled. FIG. 5E comprises violin plots of selected transcripts, with expression profile of cluster 1 outlined. KLRB1=CD161; ITGAE=CD103; GNLY=granulysin. All violin plots generated using standard Seurat v3.1 implementation; y-axis represents normalized and log-transformed expression data (log(scaled transcript counts+1)).

FIGS. 6A to 6L illustrate that IBD mucosa is characterized by an abundance of HLA-DR+CD38+ T cell populations. FIG. 6A is a series of graphs illustrating the abundance of HLA-DR⁺CD38⁺CD4⁺ T cells, HLA-DR⁺CD38⁺ conventional T cells (TCONS), HLA-DR⁺CD38⁺FOXP3⁺ regulatory T cells (TREG), HLA-DR⁺CD38⁺CD8⁺ T cells, HLA-DR⁺CD38⁺CD4⁻CD8⁻ T cells, and HLA-DR⁺CD38⁺ natural killer (NK) T cells in mucosa and peripheral blood (PB) samples derived from non-IBD controls, from subjects with active and inactive ulcerative colitis (UC), and from subjects with and active and inactive Crohn's disease (CD). FIG. 6B comprises box- and whisker plots, a t-SNE plot of Node 4(−) (from FIG. 4D), and HLA-DR/CD38/CXCR3 heatmaps. FIG. 6C provides flow cytometry data demonstrating an increase in HLA-DR⁺CD38⁺ T cells (out of total T cells) in active IBD mucosa of an independent cohort with representative biaxial gating plots. FIG. 6D is a chart illustrating the expression pattern of various protein markers characterizing HLA-DR⁺CD38⁺ T cell populations. “T CONS” refers to conventional T cells; “TREGS” refers to regulatory T cells; and “NK T cells” refers to natural killer cells T cells. FIGS. 6E to 6H illustrate differential expression patterns of potential biomarkers in samples derived from subjects with inflammatory bowel disease. FIG. 6E depicts differential abundance of specific T cell populations between IBD and non-IBD controls. “IBD” refers to inflammatory bowel disease. FIG. 6F is a comparison of the protein expression profiles of samples derived from subjects with and without inflammatory bowel disease in T cells. FIG. 6G is a series of T-distributed Stochastic Neighbor Embedding (T-SNE) density heatmaps of mucosa T cells (CD3⁺CD45⁺) of pooled subjects (total inflammatory bowel disease (IBD), total non-IBD, total active IBD, total inactive IBD, active/inactive ulcerative colitis (UC) and active/inactive Crohn's disease (CD)). FIG. 6H is a series of T-SNE marker heat maps depicting expression levels of protein markers. FIG. 6I is a gating strategy defining HLA-DR⁺CD38⁺ T cells (left) with labeled t-SNE cluster plot (right) from dedicated clustering analysis. FIG. 6J comprises a CyTOF marker heatmap, cluster dendrogram, cluster abundance and identity for HLA-DR⁺CD38⁺ T cell analysis with labeling strategy as per FIG. 4D. FIG. 6L comprises an abundance plot, t-SNE, and IL-1β heatmap of Node 11(+) (from FIG. 6J).

FIGS. 7A to 7F illustrate that FOXP3⁺ Tregs are increased in abundance in active IBD mucosa and demonstrate increased expression of pro-inflammatory cytokines (IL-17A, IL-1β) FIGS. 7A and 7B illustrate the abundance of FOXP3⁺ regulatory T cells and conventional T cells (TCONS) in IBD and non-IBD subjects. FIG. 7A is a series of graphs showing the abundance of FOXP3⁺ regulatory T cells and TCONS out of total CD4⁺ T cells in mucosa samples from controls (non-IBD) and from subjects having active or inactive ulcerative colitis (UC) or Crohn's disease (CD). FIG. 7B is a series of graphs illustrating expression of markers in FOXP3⁺ regulatory T cells and conventional T cells in mucosa samples from controls (non-IBD) and from subjects having active or inactive ulcerative colitis (UC) or Crohn's disease (CD). FIG. 7C is a series of protein expression profiles in T cells that compare expression in active ulcerative colitis and Crohn's disease samples and non-IBD samples. FIG. 7D is a series of graphs illustrating the proportions of various manually gated T cell subsets out of total T cells in lamina propria and peripheral blood. Specifically, the abundance of CD4+ T cells, conventional T cells (TCONS), FOXP3+ regulatory T cells (TREGS), CD8+ T cells, CD4−CD8− T cells, and natural killer (NK) T cells were measured in lamina propria (LP) and peripheral blood (PB) of non-IBD controls, active and inactive ulcerative colitis (UC), and active and inactive Crohn's disease (CD). FIG. 7E is a series of protein expression profiles that compare the expression of certain markers in active and inactive IBD (e.g., inactive Crohn's disease and inactive ulcerative colitis). FIG. 7F is a graph illustrating the abundance of IL-22⁺ T cells in the peripheral blood across subject groups.

FIGS. 8A-8G illustrate that T regulatory cell (Treg) analysis demonstrates expansion of pro-inflammatory memory Treg subsets in IBD. FIG. 8A provides a gating strategy for defining Tregs cells (left) with labeled t-SNE cluster plot (right) from dedicated clustering analysis. FIG. 8B comprises a CyTOF marker heatmap, cluster dendrogram, cluster abundance and identity for Treg analysis with the labeling strategy as per FIG. 4D. FIG. 8C comprises abundance plots, t-SNE, and selected Treg marker heatmaps for Node 5(−) and Node 1(+) (from FIG. 3B). FIG. 8D. comprises abundance plots, t-SNE, and TNFα/IFNγ/IL-17A marker heatmaps for clusters 7 and 8 (from Node 1(+) in FIG. 8B). FIG. 8E comprises abundance plot, t-SNE, and IL-1β marker heatmaps cluster 2 (from FIG. 8B). FIGS. 8F and 8G demonstrate T regulatory cell (Treg) expansion in IBD mucosa and identification of IL-1β⁺ Treg population with flow cytometry. FIG. 8F comprises flow cytometry data used to identify an IL-1β⁺ Treg population from an active UC patient. FIG. 8G comprises representative biaxial gating plots of Tregs (gated as CD45+CD3+CD4+FoxP3+ cells) in an independent cohort. These cells are shown to be increased in active IBD mucosa. A box-and-whisker abundance plot is shown on the right.

FIG. 9 is a series of graphs depicting the ratios of different types of T helper cells in healthy controls (HC), active and inactive ulcerative colitis, and active and inactive Crohn's disease.

FIGS. 10A and 10B illustrate differential marker expression differences in T cell subsets between IBD disease and inflammation states. FIG. 10A is a graph depicting marker expression differences between inactive ulcerative colitis and non-IBD. FIG. 10B is a graph depicting marker expression differences between Crohn's disease and ulcerative colitis.

FIGS. 11A-11C illustrate a B Cell analysis highlights unique naïve B Cell and plasmablast populations in IBD. FIG. 11A comprises a gating strategy defining B cells (left) and a labeled t-SNE cluster plot (right) from dedicated clustering analysis. FIG. 11B comprises CyTOF marker heatmap, cluster dendrogram, cluster abundance and identity (color-coded by subset) and organized as per FIG. 4D. FIG. 11C comprises abundance plots, t-SNE, and IL-1β/TNFα/CCR7 marker heatmaps of Node 8(+) (from FIG. 11B).

FIG. 12 is a series of graphs depicting the relative proportions of B cells to all CD45⁺ cells and the relative proportions of CD27⁺ B cells and plasmablasts to all B cells.

FIGS. 13A and 13B illustrate that CXCR3⁺ plasmablasts are increased in IBD mucosa. FIG. 13A comprises abundance plots, t-SNE, and selected marker heatmap for Cluster 18 (from Node 10(−) in FIG. 11B). FIG. 13B comprises biaxial plots of CyTOF data demonstrating gating strategy to define plasmablasts (CD45⁺CD19⁺CD27⁺CD24−CD38⁺⁺) and the gating strategy to define FoxP3⁺ populations in both total B cells and plasmablasts. FIG. 13B also comprises a plot comparison of the proportion of FoxP3⁺ plasmablasts (out of total plasmablasts) versus proportion of FoxP3⁺ B cells (out of total B cells) in the mucosa and in the periphery.

FIG. 14 is a series of expression profiles of various markers in manually gated B cell subsets in mucosa and peripheral blood (PB) samples. Shading (as defined in the legends for each heatmap) is depicted for differences having a p value <0.05 (Wilcoxon t-test).

FIGS. 15A-15D show that innate immune cell analysis reveals granulocyte and dendritic cell differences in UC and CD. FIG. 15A provides a gating strategy defining innate immune cells (left) with labeled t-SNE cluster plot (right) from dedicated clustering analysis. FIG. 15B comprises a CyTOF marker heatmap, cluster dendrogram, cluster abundance and identity (color-coded by subset) organized as per FIG. 4D. FIG. 15C comprises abundance plots, t-SNE, and CD38/CXCR3 marker heatmaps of Node 9(−) (from FIG. 15B). FIG. 15D comprises abundance plots (combined), t-SNE, and selected marker heatmaps Nodes 16(+) and 4(+) (from FIG. 15B).

FIG. 16 is a series of graphs depicting the proportion of various innate cell populations as a percentage of parental populations in mucosa and PB. Populations were gaited as following: innate cells (CD45⁺CD3⁻CD19⁻), macrophage/monocytes (CD14⁺ innate), dendritic cells (DCs) (CD14⁻HLA-DR⁺ innate), plasmacytoid (p) DCs (CD123⁺ DCs), CD123⁺ innate (CD123⁺ innate), natural killer (NK) (CD56⁺ innate), and innate lymphoid cells.

FIGS. 17A and 17B show that innate lymphoid cell signatures differentiate Crohn's disease from ulcerative colitis in mucosa and periphery samples. FIG. 17A comprises abundance plots, t-SNE, and selected marker heatmaps from Nodes 6(−) and 19(+) (from FIG. 15B). FIG. 17B comprises an abundance plot, t-SNE, and CCR6 marker heatmap from Cluster 30 (from FIG. 15B).

FIGS. 18A and 18B illustrate that IL-1β expression in innate immune cells is increased in mucosa and peripheral blood (PB) samples from active Crohn's disease. FIG. 18A is a series of expression profiles that depict the mean metal intensities of various markers in manually gated innate cell subsets in mucosa and PB. Shading (as defined in the legends for each heatmap) is depicted for differences having a p-value <0.05 (Wilcoxon t-test). FIG. 18B is a series of graphs depicting IL-1β expression in various innate cell populations depicted.

FIGS. 19A to 19C show that CyTOF demonstrates differential innate lymphoid cell and IL-1β-related macrophage/monocyte signatures in UC vs CD. FIG. 19A comprises an abundance plot, t-SNE, and CD14 marker heatmap for Node 4(−) (from FIG. 15B). FIG. 19B comprises an abundance plot (combined), t-SNE, and IL-1β marker heatmap for Clusters 3 and 4 (from FIG. 15B). FIG. 19C comprises a box-and-whisker plot of IL1B+CD14+ cells counted in ISH images (4 images per subject) with representative image.

FIGS. 20A to 20D illustrate that Random Forest modeling discriminates CDa vs UCa in mucosa and periphery. FIG. 20A illustrates the Random Forest modeling in mucosa and comprises receiver operator characteristic (ROC) curves (top panels) with models using top 3, top 6, and full model of input features shown. Area under the curve (AUC) for each model shown with associated p-value. Probability plots with associated confusion matrices (middle panels) demonstrating model accuracy of the full model. Top 10 features (in descending order of importance), representing isometric log-ratio of depicted nodes, with relative abundance of node branches in bar graphs and colored based on preferential abundance in UCa vs CDa (bottom panels). “In.”=“innate”. FIG. 20B illustrates the Random Forest modeling of the periphery and comprises the analogous data described for FIG. 20A. FIG. 20C provides results for Random forest (RF) classifier used to generate models differentiating non-IBD:UCa, non-IBD:CDa, UCa:UCi, and CDa:CDi in the mucosa. FIG. 20D provides results for Random forest (RF) classifier used to generate models differentiating non-IBD:UCa, non-IBD:CDa, UCa:UCi, and CDa:CDi in the periphery. The leftmost panels of FIGS. 20C and 20D depict receiver operator characteristic curves (ROC) of models using the top 3, top 6, or full model of input features. Each curve is accompanied with area under the curve (AUC) and the associated p-value. Middle panels of FIGS. 20C and 20D comprise probability plots with associated confusion matrices of prediction accuracy of the full model (prediction accuracy listed in bold in the upper left corner of the probability plots). The rightmost plots of FIGS. 20C and 20D depict the isometric log-ratio of the top 10 features (nodes derived from the automated clustering analysis of total T, total B, and total innate immune cells (FIGS. 4D, 11B, and 15B)) in descending order of importance. The barplots are color-coded by subject group comparison. The more “negative” the isometric log-ratio of a particular node, the more increased Node X(−)>Node X(+) branch abundance. The more “positive” the isometric log-ratio of a particular node, the more increased Node X(+)>Node X(−) branch abundance.

### DETAILED DESCRIPTION OF THE INVENTION

The invention features compositions and methods that are useful for characterizing inflammatory bowel disease subtype (e.g., Crohn's disease versus ulcerative colitis) as well as activity (e.g., active versus inactive disease) in colonic mucosa as well as in peripheral blood.

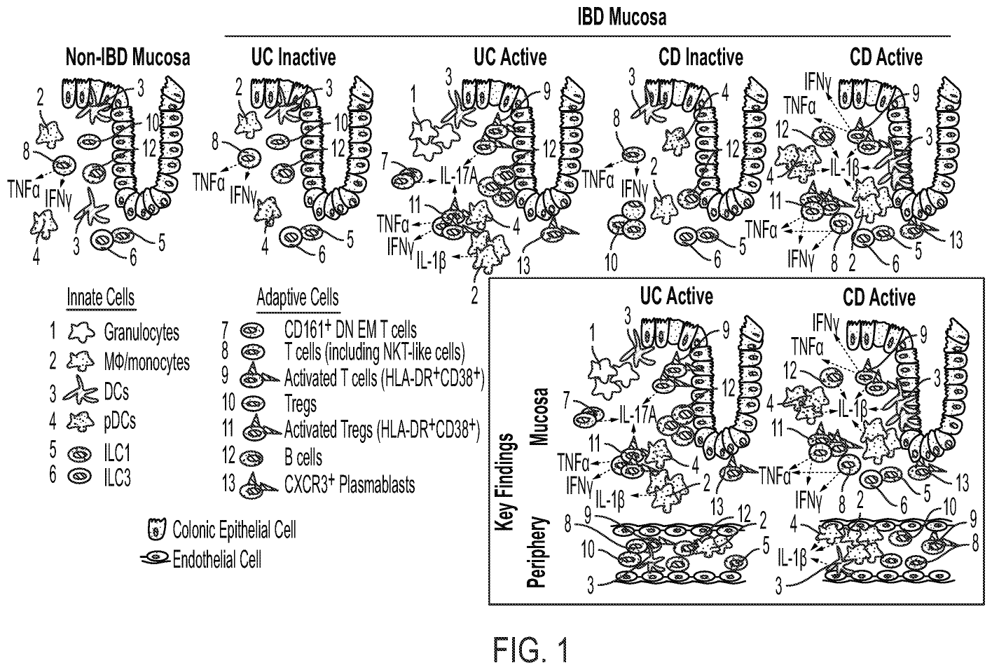

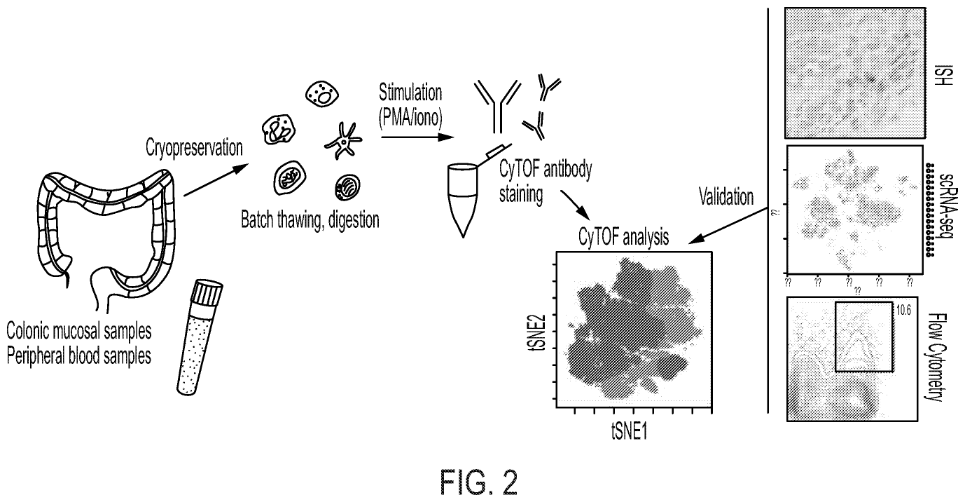

The invention is based, at least in part, on the discovery that subjects with inflammatory bowel disease have different expression levels of certain markers when compared to subjects that are not affected by the disease as well as differential abundance of certain immune cell populations in their colonic tissue and peripheral blood. As reported in detail below, mass cytometry time of flight (CyTOF) was used to profile colonic mucosa (n=87) and peripheral blood mononuclear cells (n=85) of patients with active (a) or inactive (i) ulcerative colitis (UC) or Crohn's disease (CD) and non-inflammatory bowel disease (IBD) controls. Analysis of CyTOF data identified differences in both mucosa and peripheral blood that distinguish the inflammatory bowel disease groups and non-IBD controls (FIGS. 1 and 2). In the mucosa, active inflammatory bowel disease could be distinguished from non-inflammatory bowel disease by increased abundance of (1) HLADR⁺CD38⁺ T cells; (2) FOXP3⁺ Tregs; (3) CD24⁺CD38⁺ B cells; and (4) IL-1β-expressing macrophages. Active ulcerative colitis could be distinguished from active Crohn's disease by (I) increased expression of IL-17A in active ulcerative colitis and increased expression of IL-1α, IFNγ, and INFα, in T cell subsets in active Crohn's disease and (2) increased IL-1β expression in innate cells in active Crohn's disease. In the peripheral blood, active ulcerative colitis could be distinguished from active Crohn's disease by (1) increased expression of CD24 and CCR7 in T cells in active ulcerative colitis and (2) increased IL-1β expression in B cells and innate cells in active Crohn's disease. Distinctions between mucosa and peripheral blood were identified in active ulcerative colitis and inactive ulcerative colitis, active Crohn's disease and inactive Crohn's disease, and between inactive inflammatory bowel disease and non-inflammatory bowel disease controls. The results demonstrate the strength of single-cell technology in simultaneously identifying numerous disease-specific immune signatures in both colonic tissue and the periphery that can be harnessed for targeted therapeutics and personalized medicine in inflammatory bowel disease.

Thus, Crohn's disease and ulcerative colitis, two subtypes of inflammatory bowel disease, have distinct immune cell profiles in the colonic mucosa and in peripheral blood. Methods and compositions, as disclosed herein, are directed to detecting markers comprising CD3, CD4, CD8, CD127, CD25, CD27, CXCR3, CCR6, CCR7, CD45RA, CD45RO, IFNγ, TNFα, IL-1β, IL-17A, IL-21, IL-22, HLA-DR, CD38, CD11c, IL23p19, CD66b, CD163, CD44, ckit, CD16, NKp46, AHR or any subset thereof. As demonstrated herein, these markers can be used to differentiate active and inactive Crohn's disease, active and inactive ulcerative colitis, and non-inflammatory bowel disease.

### Methods for Detecting Markers

Any suitable method can be used to detect markers associated with inflammatory bowel disease (e.g., CD3, CD4, CD8, CD127, CD25, CD27, CXCR3, CCR6, CCR7, CD45RA, CD45RO, IFNγ, TNFα, IL-1β, IL-17A, IL-21, IL-22, HLA-DR, CD38, CD11c, IL23p19, CD66b, CD163, CD44, ckit, CD16, NKp46, AHR or any subgroup thereof). The invention can be successfully practiced with one or a combination of methods that detect markers. In some embodiments, methods to detect markers further comprise steps to quantify the markers. In some embodiments, the methods disclosed herein detect markers in specific cells, such as immune cells. In some embodiments, the immune cells are conventional T cells, regulatory T cells, natural killer T cells, T helper cells, and T cells characterized by surface antigens (e.g., CD4, CD8, and CD45). In some embodiments, the immune cells are B cells. In some embodiments, the B cells are plasmablasts, B regulatory cells, and B cells characterized by surface antigens (e.g., CD27). In some embodiments, the immune cells are innate immune cells. In some embodiments, the innate immune cells are macrophages, monocytes, dendritic cells, plasmacytoid dendritic cells, CD123⁺ innate cells, natural killer (NK) cells, CD161⁺ natural killer cells, CD127⁺ innate lymphoid cells (ILCs), or CD161⁺CD127⁺ innate lymphoid cells.

Markers can be detected in a sample from a subject having or suspected of having inflammatory bowel disease. In some embodiments, the sample is a blood sample, such as a plasma, whole blood, sample. In some embodiments, the blood sample is a peripheral blood sample. In some embodiments, the sample is a tissue sample, such as a biopsy sample. In some embodiments, the tissue sample comprises mucosa tissue.

Detection methods include, without limitation, hybridization-based methods, including those employed in mass cytometry (e.g., cytometry time-of-flight (CyTOF)), a powerful proteomic technique in which antibodies are detectably labeled with rare earth metal isotopes instead of fluorophores. Such labeling allows for combining many different labeled antibodies without significant spillover between detection channels as is more common in visual (e.g., fluorescence-based) detection schemes. Labeled cells are then analyzed using high throughput mass spectrometry to quantify parameters at the single-cell level. In addition to detecting markers on a cell, CyTOF can be used to determine cell types present in a sample. For example, the technique can be used to identify the abundance of CD4⁺ T cells, conventional T cells (TCONS), FOXP3⁺ regulatory T cells (TREGS), CD8⁺ T cells, CD4⁻CD8⁻ T cells, and natural killer (NK) T cells in a sample. In some embodiments, CyTOF can be used to determine the abundance of plasmablasts, B regulatory cells, and B cells characterized by surface antigens (e.g., CD27) in a sample. In some embodiments, the sample is a peripheral blood sample or a tissue (e.g., mucosa) biopsy derived from a subject having or suspected of having inflammatory bowel disease.

Other marker detection methods include, but are not limited to, biochip arrays, fluorescence assays (e.g. sandwich immunoassay), surface plasmon resonance, ellipsometry and atomic force microscopy. Expression levels of markers (e.g., polynucleotides or polypeptides) are compared by procedures well-known in the art, such as RT-PCR, Northern blotting, Western blotting, mass spectrometry, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays.

Detection methods may include use of a biochip array. Biochip arrays contemplated in the present disclosure include protein arrays. One or more markers are captured on the biochip array and subjected to analysis to detect the level of the markers in a sample. In some embodiments, markers are captured with capture reagents immobilized to a solid support, such as a biochip, a multi-well microtiter plate, a resin, or a nitrocellulose membrane that is subsequently probed for the presence or level of a marker. Capture can be on a chromatographic surface or a biospecific surface. For example, a sample containing the markers, such as serum, is used in some embodiments to contact the active surface of a biochip for a sufficient time to allow binding. Unbound molecules are washed from the surface using a suitable eluent, such as phosphate buffered saline. In general, the more stringent the eluent, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry is used. In some embodiments, the mass spectrometry used is LC-MS/MS. In some embodiments, the mass spectrometry is SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

In some embodiments of the methods of the present invention, multiple markers are measured. The use of multiple markers increases the predictive value of the test and provides greater utility in diagnosis, toxicology, patient stratification, and patient monitoring. The process called “pattern recognition” detects patterns formed by multiple markers, which greatly improves the sensitivity and specificity of clinical proteomics for predictive medicine. Subtle variations in data from clinical samples indicate that certain patterns of protein expression indicates the presence or absence of inflammatory bowel disease, ulcerative colitis, or Crohn's disease.

### Diagnostic Assays

The present invention provides diagnostic assays for determining whether a subject with inflammatory bowel disease has Crohn's disease or ulcerative colitis. Expression levels of markers as well as differential abundance of certain cellular populations described herein are correlated with inflammatory bowel disease. These markers can be used to discriminate between ulcerative colitis and Crohn's disease in both the peripheral blood as well as colonic tissue (e.g., mucosa), as well as between active and inactive IBD. In some embodiments of the invention, a sample (e.g., blood or biopsied tissue) from a subject is analyzed to determine levels of markers associated with inflammatory bowel disease. Particular expression profiles are associated with Crohn's disease, with ulcerative colitis, or with a phenotype that indicates the absence of inflammatory bowel disease. In some embodiments, levels of the markers are indicative of an active state of Crohn's disease or ulcerative colitis. In some embodiments, a marker expression profile is indicative of an inactive state of Crohn's disease or ulcerative colitis.

In some embodiments, diagnostic assays are provided that detect active or inactive inflammatory bowel disease (e.g., ulcerative colitis, Crohn's disease) in a subject. Diagnoses made using the compositions and methods of the present invention enable a caregiver to design an appropriate or optimized therapeutic regimen. Personalized treatment regimens, rather than generic therapies, for inflammatory bowel disease can greatly improve clinical outcome for a subject with the disease.

In some embodiments, methods are provided for identifying cell types in a sample from a subject having or suspected of having inflammatory bowel disease. Based on the identified cell types, a profile of the sample can be generated for the sample and compared to a reference profile. The reference profile can be the profile of a control sample in which inflammatory bowel disease is absent, an active or inactive Crohn's disease profile, and/or an active or inactive ulcerative colitis profile.

The cell types that comprise a profile for a disease subtype (e.g., Crohn's disease or ulcerative colitis) or disease state (e.g., active or inactive) can be any cell type for which there is a significant difference between the disease subtypes and/or states. For example, a profile of active ulcerative colitis has a relative increase in T cells expressing CD38, HLA-DR, FOXP3, IL-17A, CCR4, and CTLA-4 and a decrease in TNFα compared to normal control.

In some embodiments, the cells used to generate a profile are cells that are involved in a disease-related immune response, such as inflammation. In some embodiments, T and B cells exhibit expression profiles that are characteristic for subtypes of inflammatory bowel disease (e.g., active and inactive Crohn's disease and active and inactive ulcerative colitis). In some embodiments, the T cells are conventional T cells (Tcons), regulatory T cells (Tregs), natural killer T cells, type 1 T helper cells (Th1), type 2 T helper (Th2) cells, T helper 17 (Th17) cells, or T helper 1-17 (Th1-17) cells. In some embodiments, an expression profile of a cell includes the expression of any one or combination of the following markers: CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, or TNFα.

Marker expression can be detected and/or measured at the transcript or protein level by methods well-known in the art. Detecting expressed transcripts in a sample generally involves contacting the sample with a nucleic acid molecule comprising a nucleotide sequence that is at least partially complementary to the nucleic acid sequence of the transcript to be measured under conditions suitable for hybridization. In some embodiments, the nucleic acid molecule is labeled to allow visualization of the molecule after contacting the sample. In some embodiments, the nucleic acid molecule is present on the surface of a substrate. In some embodiments, the substrate comprises a microarray.

Marker expression can also be detected, and quantified in some embodiments, at the protein level. Antibodies that specifically bind a marker described herein or any other method known in the art can be used to monitor expression of a marker of interest. Detection of an alteration relative to a normal, reference sample can be used as a diagnostic indicator of inflammatory bowel disease. Similarly, detection of alterations or similarities relative to a reference sample of Crohn's disease or ulcerative colitis can be used as a diagnostic indicator of these diseases.

The methods described herein can be used to diagnose an individual or to confirm the results of another diagnostic method. Additionally, the methods described herein, or known in the art, can be use used with any other diagnostic method described herein for a more accurate diagnosis of the presence or severity of inflammatory bowel disease. For example, a diagnosis of inflammatory bowel disease based on histological examination can be refined to a diagnosis of active or inactive Crohn's disease or ulcerative colitis using the methods described herein.

The markers detected using the methods described herein can be used individually to diagnose inflammatory bowel disease. In some embodiments, the individual markers are used in combination with other identified markers, or with other markers known in the art that are associated with inflammatory bowel disease or treatment thereof. Individual markers, or combinations thereof, are differentially expressed in a subject and, therefore, differentially present in samples from a subject having inflammatory bowel disease and from a normal subject in whom inflammatory bowel disease is undetectable.

The individual markers disclosed herein are useful in determining the status or stage of a subject's inflammatory bowel disease. A marker or combination of markers detected in a sample using the methods described herein can be compared with the marker or combination of markers in a control sample, wherein differences in the expression or amounts of the marker identifies the disease.

Analyzing a combination of markers can provide greater predictive value than a single marker in some cases. Additionally, characterizing a combination of markers can enhance the confidence in observed results. For example, detecting the expression of a plurality of markers in a sample can decrease erroneous conclusions resulting from false positives and negatives. One skilled in the art will understand that the methods disclosed herein can be adjusted or optimized to increase the sensitivity or specificity of the diagnostic assay.

Some embodiments of the present invention comprise detecting differences in expression of two or more of interleukin (IL)-1β, IL-17, IFNγ, HLA-DR, FOXP3, CTLA4, CD45RO, CD45RA, CD38, CD27, CD24, CD161, CCR7, CCR6, and CCR4 between a sample comprising T cells derived from a subject suspected of having or having inflammatory bowel disease and a sample comprising T cells derived from a subject without inflammatory bowel disease, wherein the differences are indicative of active Crohn's disease. Thus, some embodiments of the present invention comprise a panel of antibodies that specifically bind two or more of IL-1β, IL-17, IFNγ, HLA-DR, FOXP3, CTLA4, CD45RO, CD45RA, CD38, CD27, CD24, CD161, CCR7, CCR6, and CCR4. In some embodiments, the panel comprises polynucleotides that are at least partially complementary to nucleic acid sequences encoding two or more of IL-1β, IL-17, IFNγ, HLA-DR, FOXP3, CTLA4, CD45RO, CD45RA, CD38, CD27, CD24, CD161, CCR7, CCR6, and CCR4.

In some embodiments, active Crohn's disease is distinguished from inactive Crohn's disease by detecting differences in the expression of two or more of TNFα, IL-21, IL-1β, CD45RO, CD45RA, CD38, CD27, CD161, and CCR4 between samples comprising T cells derived from subjects having active and inactive Crohn's disease. Some embodiments of the present invention comprise a panel of antibodies that specifically bind two or more of TNFα, IL-21, IL-1β, CD45RO, CD45RA, CD38, CD27, CD161, and CCR4. In some embodiments, the panel comprises polynucleotides that are at least partially complementary to nucleic acid sequences encoding two or more of TNFα, IL-21, IL-1β, CD45RO, CD45RA, CD38, CD27, CD161, and CCR4.

Some embodiments of the present invention comprise detecting differences in expression of two or more of IL-21, IL-17, IFNγ, HLA-DR, CTLA4, FOXP3, CXCR3, CTLA4, CD45RO, CD45RA, CD44, CD38, CD27, CD25, CD24, CD161, CCR7, CCR6, and CCR4 between a sample comprising T cells derived from a subject having or suspected of having inflammatory bowel disease and a sample comprising T cells derived from a subject without inflammatory bowel disease, wherein the differences are indicative of inactive Crohn's disease. Thus, some embodiments of the present invention comprise a panel of antibodies that specifically bind two or more of IL-21, IL-17, IFNγ, HLA-DR, CTLA4, FOXP3, CXCR3, CTLA4, CD45RO, CD45RA, CD44, CD38, CD27, CD25, CD24, CD161, CCR7, CCR6, and CCR4. In some embodiments, the panel comprises polynucleotides that are at least partially complementary to nucleic acid sequences encoding two or more of IL-21, IL-17, IFNγ, HLA-DR, CTLA4, FOXP3, CXCR3, CTLA4, CD45RO, CD45RA, CD44, CD38, CD27, CD25, CD24, CD161, CCR7, CCR6, and CCR4.

Some embodiments of the present invention comprise detecting differences in expression of two or more of IL-17, HLA-DR, FOXP3, CTLA4, CL45RO, CL45RA, CD38, CD27, CD25, CD24, CD161, CCR7, CCR6, CCR4, IL-22, IL-21, CD3, CD4, CD8, CD45, CD56, IFNγ, and TNFα between a sample comprising T cells derived from a subject suspected of having inflammatory bowel disease and a sample comprising T cells derived from a subject without inflammatory bowel disease, wherein the differences are indicative of active ulcerative colitis. Thus, some embodiments of the present invention comprise a panel of antibodies that specifically bind two or more of IL-17, HLA-DR, FOXP3, CTLA4, CL45RO, CL45RA, CD38, CD27, CD25, CD24, CD161, CCR7, CCR6, CCR4, IL-22, IL-21, CD3, CD4, CD8, CD45, CD56, IFNγ, and TNFα. In some embodiments, the panel comprises polynucleotides that are at least partially complementary to nucleic acid sequences encoding two or more of IL-17, HLA-DR, FOXP3, CTLA4, CL45RO, CL45RA, CD38, CD27, CD25, CD24, CD161, CCR7, CCR6, CCR4, IL-22, IL-21, CD3, CD4, CD8, CD45, CD56, IFNγ, and TNFα.

Some embodiments involve detecting increased numbers of effector memory T cells between a sample derived from mucosa of a subject suspected of having active UC and a sample derived from mucosa of a subject having or suspected of having non-IBD or active CD, wherein the effector memory T cells are defined as of CD45RA⁺CD45RO⁺CCR7⁺CD27⁺CD3⁺CD45⁺CD4⁻CD8α⁻ and also express CD161, IL17A, TNFα, and IFNγ, and wherein the differences are indicative of active ulcerative colitis. Thus, some embodiments of the present invention comprise a panel of antibodies that specifically bind two or more of CD45RA, CD45RO, CCR7, CD27, CD3, CD45, CD4, CD8α, CD161, IL17A, TNFα, and IFNγ. In some embodiments, the panel comprises antibodies that specifically bind CD45RA, CD45RO, CCR7, CD27, CD3, CD45, CD4, CD8α, CD161, IL17A, TNFα, IFNγ, HLA-DR, and CD38 In some embodiments, the panel comprises polynucleotides that are at least partially complementary to nucleic acid sequences encoding two or more of CD45RA, CD45RO, CCR7, CD27, CD3, CD45, CD4, CD8α, CD161, IL17A, TNFα, and IFNγ. In some embodiments, the panel comprises polynucleotides that are at least partially complementary to nucleic acid sequences encoding two or more of CD45RA, CD45RO, CCR7, CD27, CD3, CD45, CD4, CD8α, CD161, IL17A, TNFα, IFNγ, HLA-DR, and CD38.

For example, in some embodiments, these panels can be used to detect increased effector memory T cells (specifically defined by a combination of CD45RA, CD45RO, CCR7, CD27, CD3, and CD45) that are CD4⁻CD8α⁻ (e.g., mucosal-associated invariant T cells or “innate-like” T lymphocytes) and express CD161, IL17A, TNFα, and IFNγ, thereby differentiating active UC from non-IBD and active CD. The panel can be used to detect cells expressing the same markers, in combination with HLA-DR and CD38 to define an additional population that is also increased in active UC mucosa compared to non-IBD and active CD. In some embodiments, these panels can be used to detect reduced effector memory T cells (specifically defined by a combination of CD45RA, CD45RO, CCR7, CD27, CD3, and CD45) that express CD8α, TNFα, and IFNγ, thereby differentiating active UC (mucosa, where they are diminished) compared to non-IBD, inactive UC, inactive CD, and active CD. The same markers, in combination with HLA-DR and CD38 define an additional population that is also reduced in active UC mucosa relative to non-IBD, inactive UC, inactive CD and active CD.

Some embodiments involve detecting differences in expression of two or more of biomarkers selected from the group consisting of CD45RA, CD45RO, CCR7, CD27, CD3, CD45 CD161, IL17A, TNFα, and IFNγ between a sample comprising CD4-CD8α− effector memory T cells in a mucosa sample derived from a subject suspected of having active ulcerative colitis and a sample comprising CD4-CD8α− effector memory T cells in a mucosa sample derived from a subject having a non-inflammatory bowel disease state or active Crohn's disease, wherein the differences are indicative of active ulcerative colitis. Thus, some embodiments of the present invention comprise a panel of antibodies that specifically bind two or more of CD45RA, CD45RO, CCR7, CD27, CD3, CD45 CD161, IL17A, TNFα, and IFNγ. In some embodiments, the panel comprises polynucleotides that are at least partially complementary to nucleic acid sequences encoding two or more of CD45RA, CD45RO, CCR7, CD27, CD3, CD45 CD161, IL17A, TNFα, and IFNγ. CD45RA⁺CD45RO⁺CCR7⁺CD27⁺CD3⁺CD45⁺CD161⁺IL17A⁺TNFα⁺ and IFNγ⁺ effector memory T cells are increased in active UC (mucosa) compared to non-IBD and active CD. In some embodiments, the panel also comprises HLA-DR and CD38.

In another embodiment, these panels can be used to detect reduced levels of effector memory T cells (specifically defined by a combination of CD45RA, CD45RO, CCR7, CD27, CD3, CD45) that express CD56, TNFα, IFNγ (e.g., natural killer T-like cells) to differentiate active UC (mucosa)(where they are diminished) compared to non-IBD, inactive UC, inactive CD, and active CD.

Some embodiments involve detecting differences in expression of two or more of CD45, CD3, CD25, CD127, HLA-DR, CD38, CTLA-4, CD45RO, and FoxP3 between a sample comprising T regulatory cells derived from a subject suspected of having inflammatory bowel disease and a sample comprising T cells derived from a subject without inflammatory bowel disease, wherein the differences are indicative of active ulcerative colitis or inactive Crohn's disease. Thus, some embodiments of the present invention comprise a panel of antibodies that specifically bind two or more of CD45, CD3, CD25, CD127, HLA-DR, CD38, CTLA-4, CD45RO, and FoxP3. In some embodiments, the panel comprises polynucleotides that are at least partially complementary to nucleic acid sequences encoding two or more of CD45, CD3, CD25, CD127, HLA-DR, CD38, CTLA-4, CD45RO, and FoxP3. FoxP3⁺HLA-DR⁺CD38⁺CTLA-4⁺CD45RO⁺ Tregs are increased in active UC and inactive CD (mucosa) compared to non-IBD and active CD. FoxP3^{lo}HLA-DR⁺CD38⁺CTLA-4⁺CD45RO⁺ Tregs are a hallmark of both active UC and active CD mucosa.

In some embodiments, active ulcerative colitis is distinguished from inactive ulcerative colitis by detecting differences in the expression of two or more of TNFα, IL-22, IFNγ, HLA-DR, CTLA4, CD45RO, CD45RA, CD44, CD38, CD27, CD25, CD24, CCR7, CCR6, and CCR4 between samples comprising T cells derived from subjects having active and inactive ulcerative colitis. Some embodiments of the present invention comprise a panel of antibodies that specifically bind two or more of TNFα, IL-22, IFNγ, HLA-DR, CTLA4, CD45RO, CD45RA, CD44, CD38, CD27, CD25, CD24, CCR7, CCR6, and CCR4. In some embodiments, the panel comprises polynucleotides that are at least partially complementary to nucleic acid sequences encoding two or more of TNFα, IL-22, IFNγ, HLA-DR, CTLA4, CD45RO, CD45RA, CD44, CD38, CD27, CD25, CD24, CCR7, CCR6, and CCR4.

Some embodiments of the present invention comprise detecting differences in expression of two or more of TNFα, IL-17, IFNγ, CTLA4, CD45RO, CD45RA, CD38, CD24, CD161, CCR6, and CCR4 between a sample comprising T cells derived from a subject having or suspected of having inflammatory bowel disease and a sample derived from a subject without inflammatory bowel disease, wherein the differences are indicative of inactive ulcerative colitis. Thus, some embodiments of the present invention comprise a panel of antibodies that specifically bind two or more of TNFα, IL-17, IFNγ, CTLA4, CD45RO, CD45RA, CD38, CD24, CD161, CCR6, and CCR4. In some embodiments, the panel comprises polynucleotides that are at least partially complementary to nucleic acid sequences encoding two or more of TNFα, IL-17, IFNγ, CTLA4, CD45RO, CD45RA, CD38, CD24, CD161, CCR6, and CCR4.

In some embodiments, active ulcerative colitis is distinguished from active Crohn's disease by detecting differences in the expression of two or more of TNFα, IL-22, IL-21, IL-1β, IL-17, IFNγ, HLA-DR, CD45RA, CD38, CD27, CD25, CD24, CD161, CCR7, CD3, CD45 and CCR4 between a sample comprising T cells derived from a subject having active ulcerative colitis and a sample comprising T cells derived from a subject having active Crohn's disease. Some embodiments of the present invention comprise a panel of antibodies that specifically bind two or more of TNFα, IL-22, IL-21, IL-1β, IL-17, IFNγ, HLA-DR, CD45RA, CD38, CD27, CD25, CD24, CD161, CCR7, CD3, CD45 and CCR4. In some embodiments, the panel comprises polynucleotides that are at least partially complementary to nucleic acid sequences encoding two or more of TNFα, IL-22, IL-21, IL-1β, IL-17, IFNγ, HLA-DR, CD45RA, CD38, CD27, CD25, CD24, CD161, CCR7, CD3, CD45 and CCR4. In some embodiments, these panels can be used to detect increased levels of IL-1β⁺HLA-DR⁺CD38⁺ T cells, as defined specifically by the markers CD45, CD3, IL1β, HLA-DR, and CD38 active CD mucosa compared to active UC mucosa.

In some embodiments, IBD is distinguished from non-IBD by detecting differences in the expression of two or more of CD45, CD3, CD25, CD127, HLA-DR, CD38, CTLA-4, CD45RO, FoxP3, IL-17A, IFNγ, and TNFα between a sample comprising T regulatory cells derived from a subject having IBD and a sample comprising T regulatory cells derived from a subject not having IBD. Some embodiments of the present invention comprise a panel of antibodies that specifically bind two or more of CD45, CD3, CD25, CD127, HLA-DR, CD38, CTLA-4, CD45RO, FoxP3, IL-17A, IFNγ, and TNFα. In some embodiments, the panel comprises polynucleotides that are at least partially complementary to nucleic acid sequences encoding two or more of CD45, CD3, CD25, CD127, HLA-DR, CD38, CTLA-4, CD45RO, FoxP3, IL-17A, IFNγ, and TNFα. In some embodiments, these panels can be used to detect increased levels of IL-1β⁺ HLA-DR⁺CD38⁺ T cells, as defined specifically by the markers CD45, CD3, IL1β, HLA-DR, and CD38 active CD (mucosa) compared to active UC mucosa. FoxP3^{lo}HLA-DR⁺CD38⁺CTLA-4⁺CD45RO⁺IFNγ⁺TNFα⁺ Tregs are increased in IBD mucosa compared to non-IBD (inactive UC, active UC, inactive CD, active CD) mucosa. FoxP3^{lo}HLA-DR⁺CD38⁺CTLA-4⁺CD45RO⁺IFNγ⁺TNFα⁺IL17A⁺ Tregs are increased specifically in active UC mucosa compared to non-IBD, inactive UC, inactive CD, and active CD mucosa.

In some embodiments, active CD is distinguished from active UC by detecting differences in the expression of two or more of CD45, CD3, CD25, CD127, CXCR3, CCR7, CD27, CD45RA, CD45RO, and IL-1β between a sample comprising T regulatory cells derived from peripheral blood of a subject having active CD and a sample comprising T regulatory cells derived from peripheral blood of a subject having active UC. Some embodiments of the present invention comprise a panel of antibodies that specifically bind two or more CD45, CD3, CD25, CD127, CXCR3, CCR7, CD27, CD45RA, CD45RO, and IL-1β. In some embodiments, the panel comprises polynucleotides that are at least partially complementary to nucleic acid sequences encoding two or more of CD45, CD3, CD25, CD127, CXCR3, CCR7, CD27, CD45RA, CD45RO, and IL-1β. In some embodiments, these panels can be used to detect increased levels of IL-1β⁺CXCR3⁺ T regulatory cells in peripheral blood in active CD compared to active UC.

Some embodiments of the present invention comprise detecting differences in expression of two or more of TNFα, CXCR3, CTLA4, CD45RO, CD38, CD24, CD161, and CCR6 between a sample comprising B cells derived from a subject having or suspected of having inflammatory bowel disease and a sample comprising B cells derived from a subject without inflammatory bowel disease, wherein the differences are indicative of active Crohn's disease. Thus, some embodiments of the present invention comprise a panel of antibodies that specifically bind two or more of TNFα, IL-1β, CXCR3, CTLA4, CD45RO, CD38, CD24, CD161, and CCR6. In some embodiments, the panel comprises polynucleotides that are at least partially complementary to nucleic acid sequences encoding two or more of TNFα, IL-1β, CXCR3, CTLA4, CD45RO, CD38, CD24, CD161, and CCR6.

Some embodiments of the present invention comprise detecting differences in expression of two or more of CD45, CD19, CD27, IL1β, TNFα, and IFNγ between a sample comprising naïve B cells derived from the mucosa of a subject having or suspected of having active CD and a sample comprising naïve B cells derived from the mucosa of a subject having non-IBD, inactive UC, or active UC, wherein the differences are indicative of active Crohn's disease. Thus, some embodiments of the present invention comprise a panel of antibodies that specifically bind two or more of CD45, CD19, CD27, IL1β, TNFα, and IFNγ. In some embodiments, the panel comprises polynucleotides that are at least partially complementary to nucleic acid sequences encoding two or more of CD45, CD19, CD27, IL1β, TNFα, and IFNγ. In some embodiments, IL1β⁺TNFα⁺IFNγ⁺ naïve B cells are specifically increased in active CD mucosa compared to non-IBD, inactive UC, and active UC.

Some embodiments of the present invention comprise detecting differences in expression of two or more of CD45, CD19, CD24, CD27, CD38, and CXCR3 between a sample comprising plasmablasts cells derived from the mucosa of a subject having or suspected of having active UC, inactive CD, and active CD and a sample comprising plasmablasts derived from the mucosa of a non IBD subject, wherein the differences are indicative of active UC, inactive CD, and active CD. Thus, some embodiments of the present invention comprise a panel of antibodies that specifically bind two or more of CD45, CD19, CD24, CD27, CD38, and CXCR3. In some embodiments, the panel comprises polynucleotides that are at least partially complementary to nucleic acid sequences encoding two or more of CD45, CD19, CD24, CD27, CD38, and CXCR3. In some embodiments, CXCR3⁺ plasmablasts (defined by the markers CD45, CD19, CD24, CD27, CD38, and CXCR3) are specifically increased in active UC, inactive CD, and active CD mucosa. FoxP3 expression in CXCR3⁺ plasmablasts is a marker of active IBD mucosa.

Some embodiments of the present invention comprise detecting CD45⁺CXCR3⁺CD44⁺CCR4⁺CCR7⁺CCR7⁺CD66b⁺CD56⁺CD3⁻CD19⁻ granulocytes in a sample derived from the mucosa of a subject having or suspected of having active UC, wherein CD45⁺CXCR3⁺CD44⁺CCR4⁺CCR7⁺CCR7⁺CD66b⁺CD56⁺CD3⁻CD19⁻ granulocytes are indicative of active UC. Thus, some embodiments of the present invention comprise a panel of antibodies that specifically CD45, CXCR3, CD44, CCR4, CCR7, CCR7, CD66b, CD56, CD3, and CD19. In some embodiments, the panel comprises polynucleotides that are at least partially complementary to nucleic acid sequences encoding CD45, CXCR3, CD44, CCR4, CCR7, CCR7, CD66b, CD56, CD3, and CD19.

Some embodiments of the present invention comprise detecting increased levels of IL1β⁺ dendritic cells and plasmacytoid dendritic cells in a sample derived from the mucosa or peripheral blood of a subject having or suspected of having active CD relative to a sample derived from the mucosa or peripheral blood of a subject not having active CD, wherein IL1β⁺ dendritic cells and plasmacytoid dendritic cells are indicative of active CD. Thus, some embodiments of the present invention comprise a panel of antibodies that specifically bind CD11c, CD123, HLA-DR, and IL1β. In some embodiments, the panel comprises polynucleotides that are at least partially complementary to nucleic acid sequences encoding CD11c, CD123, HLA-DR, and IUD.

Some embodiments of the present invention comprise detecting decreased levels of Group 1 innate lymphoid cells (defined as CD45⁺CD3⁻CD19⁻CD38⁺CD45RA⁺IFNγ⁺Tbet⁺CD161⁺), which include natural killer cells (NK cells) in a sample derived from the peripheral blood of a subject having or suspected of having active CD relative to a sample derived from the peripheral blood of a subject having active UC or non-IBD, wherein the Group 1 innate lymphoid cells (defined as CD45⁺CD3⁻CD19⁻CD38⁺CD45RA⁺IFNγ⁺Tbet⁺CD161⁺) are indicative of active CD. Thus, some embodiments of the present invention comprise a panel of antibodies that specifically bind CD45, CD3-CD19-CD38, CD45RA, IFNγ, Tbet, and CD161. In some embodiments, the panel comprises polynucleotides that are at least partially complementary to nucleic acid sequences encoding CD45, CD3-CD19-CD38, CD45RA, IFNγ, Tbet, and CD161.

Some embodiments of the present invention comprise detecting increased levels of Group 1 innate lymphoid cells (ILC) or ILC-like cells (defined as CD45⁺CD3⁻CD19⁻IFNγ⁺TNFα⁺CD8α^{+/-}Tbet^{+/-}), in a sample derived from mucosa of a subject having or suspected of having active CD relative to a sample derived from mucosa of a subject having active UC, wherein increased levels of the Group 1 innate lymphoid cells or ILC-like cells (defined as CD45⁺CD3⁻CD19⁻IFNγ⁺ TNFα⁺CD8α^{+/-}Tbet^{+/-}) are indicative of active CD. Some embodiments of the present invention comprise detecting decreased levels of Group 1 innate lymphoid cells (ILC) or ILC-like cells (defined as CD45⁺CD3⁻CD19⁻IFNγ⁺TNFα⁺CD8α^{+/-}Tbet^{+/-}), in a sample derived from the peripheral blood of a subject having or suspected of having active CD relative to a sample derived from the peripheral blood of a subject having active UC or non-IBD, wherein decreased levels of the Group 1 innate lymphoid cells or ILC-like cells (defined as CD45⁺CD3⁻CD19⁻IFNγ⁺ TNFα⁺CD8α^{+/-}Tbet^{+/-}) are indicative of active CD. Some embodiments of the present invention comprise detecting decreased levels of Group 1 innate lymphoid cells (ILC) or ILC-like cells (defined as CD45⁺CD3⁻CD19⁻IFNγ⁺TNFα⁺CD8α^{+/-}Tbet^{+/-}), in a sample derived from mucosa of a subject having or suspected of having active UC relative to a sample derived from mucosa of a subject having non-IBD, wherein decreased levels of the Group 1 innate lymphoid cells or ILC-like cells (defined as CD45⁺CD3⁻CD19⁻IFNγ⁺ TNFα⁺CD8α^{+/-}Tbet^{+/-}) are indicative of active CD. Thus, some embodiments of the present invention comprise a panel of antibodies that specifically bind CD45, CD3, CD19, IFNγ, TNFα, CD8α, and Tbet. In some embodiments, the panel comprises polynucleotides that are at least partially complementary to nucleic acid sequences encoding CD45, CD3, CD19, IFNγ, TNFα, CD8α, and Tbet.

Some embodiments of the present invention comprise detecting decreased levels of innate lymphoid cells (ILC) type 3 (defined as CD45⁺CD3⁻CD19⁻CD161⁺CD127⁺CCR6⁺ ckit⁺TNFα⁺), in a sample derived from mucosa of a subject having or suspected of having active CD relative to a sample derived from mucosa of a subject having non-IBD, inactive UC, inactive CD, or active CD, wherein increased levels of the innate lymphoid type 3 cells (defined as CD45⁺CD3⁻CD19⁻CD161⁺CD127⁺CCR6⁺ckit⁺TNFα⁺) are indicative of active CD. Thus, some embodiments of the present invention comprise a panel of antibodies that specifically bind CD45, CD3, CD19, CD161, CD127, CCR6, ckit, TNFα. In some embodiments, the panel comprises polynucleotides that are at least partially complementary to nucleic acid sequences encoding CD45, CD3, CD19, CD161, CD127, CCR6, ckit, TNFα.

Some embodiments of the present invention comprise detecting increased levels of CD14⁺ macrophages/monocytes (defined as CD45⁺CD3⁻CD19⁻CD14⁺CD11c⁺HLA-DR⁺) in a sample derived from mucosa of a subject having or suspected of having active IBD relative to a sample derived from mucosa of a subject having inactive IBD or non-IBD, wherein increased levels of the CD14⁺ macrophages/monocytes (defined as CD45⁺CD3⁻CD19⁻CD14⁺CD11c⁺HLA-DR⁺) are indicative of active IBD. Some embodiments of the present invention comprise detecting increased levels of CD14⁺ macrophages/monocytes (defined as CD45⁺CD3⁻CD19⁻CD14⁺CD11c⁺HLA-DR⁺) in a sample derived from peripheral blood of a subject having or suspected of having active CD relative to a sample derived from peripheral blood of a subject having active UC or non-IBD, wherein increased levels of the CD14⁺ macrophages/monocytes (defined as CD45⁺CD3⁻CD19⁻CD14⁺CD11c⁺HLA-DR⁺) are indicative of active CD. Thus, some embodiments of the present invention comprise a panel of antibodies that specifically bind CD45, CD3, CD19, CD14, CD11c, and HLA-DR. In some embodiments, the panel comprises polynucleotides that are at least partially complementary to nucleic acid sequences encoding CD45, CD3, CD19, CD14, CD11c, and HLA-DR.

Some embodiments of the present invention comprise detecting increased levels of IL-1β⁺CD14⁺ macrophages/monocytes (defined as CD45⁺CD3⁻CD19⁻CD14⁺CD11c⁺HLA-DR⁺IL-1β⁺) in a sample derived from mucosa of a subject having or suspected of having active IBD relative to a sample derived from mucosa of a subject having inactive IBD or non-IBD, wherein increased levels of the IL-1β⁺CD14⁺ macrophages/monocytes (defined as CD45⁺CD3⁻CD19⁻CD14⁺CD11c⁺HLA-DR⁻IL-1β⁺) are indicative of active IBD. Some embodiments of the present invention comprise increased levels of IL-1β⁺CD14⁺ macrophages/monocytes (defined as CD45⁺CD3⁻CD19⁻CD14⁺CD11c⁺HLA-DR⁺IL-1β⁺) in a sample derived from peripheral blood of a subject having or suspected of having active CD relative to a sample derived from peripheral blood of a subject having active UC or non-IBD, wherein increased levels of the IL-1β⁺CD14⁺ macrophages/monocytes (defined as CD45⁺CD3⁻CD19⁻CD14⁺CD11c⁺HLA-DR⁻IL-1β⁺) are indicative of active CD. Thus, some embodiments of the present invention comprise a panel of antibodies that specifically bind CD45, CD3, CD19, CD14, CD11c, HLA-DR, and IL-1β⁺. In some embodiments, the panel comprises polynucleotides that are at least partially complementary to nucleic acid sequences encoding CD45, CD3, CD19, CD14, CD11c, HLA-DR and IL-1β⁺.

In some embodiments, active Crohn's disease is distinguished from inactive Crohn's disease by detecting differences in the expression of two or more of TNFα, IL-22, IL-1β, IL-17, HLA-DR, CTLA4, CD25, and CCR7 between samples comprising B cells derived from subjects having or suspected of having active and inactive Crohn's disease. Some embodiments of the present invention comprise a panel of antibodies that specifically bind two or more of TNFα, IL-21, IL-1β, CD45RO, CD45RA, CD38, CD27, CD161, and CCR4. In some embodiments, the panel comprises polynucleotides that are at least partially complementary to nucleic acid sequences encoding two or more of TNFα, IL-21, IL-1β, CD45RO, CD45RA, CD38, CD27, CD161, and CCR4.

Some embodiments of the present invention comprise detecting differences in expression of two or more of IL-21, IFNγ, HLA-DR, FOXP3, CD45RO, CD38, CD27, CD25, CD24, CD161, and CCR7 between a sample comprising B cells derived from a subject having or suspected of having inflammatory bowel disease and a sample comprising B cells derived from a subject without inflammatory bowel disease, wherein the differences are indicative of inactive Crohn's disease. Thus, some embodiments of the present invention comprise a panel of antibodies that specifically bind two or more of IL-21, IFNγ, HLA-DR, FOXP3, CD45RO, CD38, CD27, CD25, CD24, CD161, and CCR7. In some embodiments, the panel comprises polynucleotides that are at least partially complementary to nucleic acid sequences encoding two or more of IL-21, IFNγ, HLA-DR, FOXP3, CD45RO, CD38, CD27, CD25, CD24, CD161, and CCR7.

Some embodiments of the present invention comprise detecting differences in expression of two or more of IL-17, HLA-DR, CXCR3, CTLA4, CD45RO, CD45RA CD44, CD38, CD27, CD25, CD24, CD161, CCR7, CCR6, and CCR4 between a sample comprising B cells derived from a subject having or suspected of having inflammatory bowel disease and a sample comprising B cells derived from a subject without inflammatory bowel disease, wherein the differences are indicative of active ulcerative colitis. Thus, some embodiments of the present invention comprise a panel of antibodies that specifically bind two or more of IL-17, HLA-DR, CXCR3, CTLA4, CD45RO, CD45RA CD44, CD38, CD27, CD25, CD24, CD161, CCR7, CCR6, and CCR4. In some embodiments, the panel comprises polynucleotides that are at least partially complementary to nucleic acid sequences encoding two or more of IL-17, HLA-DR, CXCR3, CTLA4, CD45RO, CD45RA CD44, CD38, CD27, CD25, CD24, CD161, CCR7, CCR6, and CCR4.

In some embodiments, active ulcerative colitis is distinguished from inactive ulcerative colitis by detecting differences in the expression of two or more of HLA-DR, CD45RO, CD45RA, CD24, CD161, and CCR4 between samples comprising B cells derived from subjects having active and inactive ulcerative colitis. Some embodiments of the present invention comprise a panel of antibodies that specifically bind two or more of HLA-DR, CD45RO, CD45RA, CD24, CD161, and CCR4. In some embodiments, the panel comprises polynucleotides that are at least partially complementary to nucleic acid sequences encoding two or more of HLA-DR, CD45RO, CD45RA, CD24, CD161, and CCR4.

Some embodiments of the present invention comprise detecting differences in expression of two or more of CD45RO, CD27, CD161, and CCR4 between a sample comprising B cells derived from a subject having or suspected of having inflammatory bowel disease and a sample comprising B cells derived from a subject without inflammatory bowel disease, wherein the differences are indicative of inactive ulcerative colitis. Thus, some embodiments of the present invention comprise a panel of antibodies that specifically bind two or more of CD45RO, CD27, CD161, and CCR4. In some embodiments, the panel comprises polynucleotides that are at least partially complementary to nucleic acid sequences encoding two or more of CD45RO, CD27, CD161, and CCR4.

In some embodiments, active ulcerative colitis is distinguished from active Crohn's disease by detecting differences in the expression of two or more of TNFα, IL-1β, IL-17, CTLA4, CD45RA, CD38, CD24, and CCR7 between a sample comprising B cells derived from a subject having active ulcerative colitis and a sample comprising B cells derived from a subject having active Crohn's disease. Some embodiments of the present invention comprise a panel of antibodies that specifically bind two or more of TNFα, IL-1β, IL-17, CTLA4, CD45RA, CD38, CD24, and CCR7. In some embodiments, the panel comprises polynucleotides that are at least partially complementary to nucleic acid sequences encoding two or more of TNFα, IL-1β, IL-17, CTLA4, CD45RA, CD38, CD24, and CCR7.

Some embodiments of the present invention comprise detecting differences in expression of two or more of IL-1β, IL-17, IFNγ, HLA-DR, FOXP3, CXCR3, CD45RO, CD38, CD24, CD161, and CDR4 between a sample comprising B cells derived from a subject having or suspected of having inflammatory bowel disease and a sample comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from a subject without inflammatory bowel disease, wherein the differences are indicative of active Crohn's disease. Thus, some embodiments of the present invention comprise a panel of antibodies that specifically bind two or more of IL-1β, IL-17, IFNγ, HLA-DR, FOXP3, CXCR3, CD45RO, CD38, CD24, CD161, CDR4. In some embodiments, the panel comprises polynucleotides that are at least partially complementary to nucleic acid sequences encoding two or more of IL-1β, IL-17, IFNγ, HLA-DR, FOXP3, CXCR3, CD45RO, CD38, CD24, CD161, CDR4.

In some embodiments, active Crohn's disease is distinguished from inactive Crohn's disease by detecting differences in the expression of two or more of IL-1β, HLA-DR, CXCR3, CTLA4, CD45RO, CD24, CD161, and CCR7 between samples comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from subjects having or suspected of having active and inactive Crohn's disease. Some embodiments of the present invention comprise a panel of antibodies that specifically bind two or more of IL-1β, HLA-DR, CXCR3, CTLA4, CD45RO, CD24, CD161, and CCR7. In some embodiments, the panel comprises polynucleotides that are at least partially complementary to nucleic acid sequences encoding two or more of IL-1β, HLA-DR, CXCR3, CTLA4, CD45RO, CD24, CD161, and CCR7.

Some embodiments of the present invention comprise detecting differences in expression of two or more of IL-21, IL-17, IFNγ, HLA-DR, FOXP3, CD45RO, CD38, CD24, CD161, CCR7, CCR6, and CCR4 between samples comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from a subject having or suspected of having inflammatory bowel disease and from a subject without inflammatory bowel disease, wherein the differences are indicative of inactive Crohn's disease. Thus, some embodiments of the present invention comprise a panel of antibodies that specifically bind two or more of IL-21, IL-17, IFNγ, HLA-DR, FOXP3, CD45RO, CD38, CD24, CD161, CCR7, CCR6, and CCR4. In some embodiments, the panel comprises polynucleotides that are at least partially complementary to nucleic acid sequences encoding two or more of IL-21, IL-17, IFNγ, HLA-DR, FOXP3, CD45RO, CD38, CD24, CD161, CCR7, CCR6, and CCR4.

Some embodiments of the present invention comprise detecting differences in expression of two or more of TNFα, IL-21, IL-1β, IL-17, IFNγ, HLA-DR, CXCR3, FOXP3, CTLA4, CD45RO, CD45RA, CD44, CD38, CD27, CD161, and CCR4 between samples comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from a subject having or suspected of having inflammatory bowel disease and from a subject without inflammatory bowel disease, wherein the differences are indicative of active ulcerative colitis. Thus, some embodiments of the present invention comprise a panel of antibodies that specifically bind two or more of TNFα, IL-21, IL-1β, IL-17, IFNγ, HLA-DR, CXCR3, FOXP3, CTLA4, CD45RO, CD45RA, CD44, CD38, CD27, CD161, and CCR4. In some embodiments, the panel comprises polynucleotides that are at least partially complementary to nucleic acid sequences encoding two or more of TNFα, IL-21, IL-1β, IL-17, IFNγ, HLA-DR, CXCR3, FOXP3, CTLA4, CD45RO, CD45RA, CD44, CD38, CD27, CD161, and CCR4.

In some embodiments, active ulcerative colitis is distinguished from inactive ulcerative colitis by detecting differences in the expression of two or more of TNFα, IL-22, IL-17, HLA-DR, CXCR3, CTLA4, CD45RO, CD45RA, CD44, CD38, CD25, CD24, CD161, CCR7, CCR6, and CCR4 between samples comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from subjects having active and inactive ulcerative colitis. Some embodiments of the present invention comprise a panel of antibodies that specifically bind two or more of TNFα, IL-22, IL-17, HLA-DR, CXCR3, CTLA4, CD45RO, CD45RA, CD44, CD38, CD25, CD24, CD161, CCR7, CCR6, and CCR4. In some embodiments, the panel comprises polynucleotides that are at least partially complementary to nucleic acid sequences encoding two or more of TNFα, IL-22, IL-17, HLA-DR, CXCR3, CTLA4, CD45RO, CD45RA, CD44, CD38, CD25, CD24, CD161, CCR7, CCR6, and CCR4.

Some embodiments of the present invention comprise detecting differences in expression of two or more of IL-21, IL-22, CD38, CD27, CD161, CCR6, and CCR4 between a sample comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from a subject having or suspected of having inflammatory bowel disease and from a subject without inflammatory bowel disease, wherein the differences are indicative of inactive ulcerative colitis. Thus, some embodiments of the present invention comprise a panel of antibodies that specifically bind two or more of IL-21, IL-22, CD38, CD27, CD161, CCR6, and CCR4. In some embodiments, the panel comprises polynucleotides that are at least partially complementary to nucleic acid sequences encoding two or more of IL-21, IL-22, CD38, CD27, CD161, CCR6, and CCR4.

In some embodiments, active ulcerative colitis is distinguished from active Crohn's disease by detecting differences in the expression of two or more of TNFα, IL-22, IL-1β, HLA-DR, CXCR3, CD45RA, CD44, CD38, CD24, CD161, and CCR4 between a sample comprising innate cells, including macrophages, monocytes, dendritic cells, plasmacytoid dendritic cells, CD123⁺ innate cells, natural killer (NK) cells, CD161⁺ natural killer cells, CD127⁺ innate lymphoid cells, and CD161⁺CD127⁺ innate lymphoid cells derived from a subject having active ulcerative colitis and from a subject having active Crohn's disease. Some embodiments of the present invention comprise a panel of antibodies that specifically bind two or more of TNFα, IL-22, IL-1β, HLA-DR, CXCR3, CD45RA, CD44, CD38, CD24, CD161, and CCR4. In some embodiments, the panel comprises polynucleotides that are at least partially complementary to nucleic acid sequences encoding two or more of TNFα, IL-22, IL-1β, HLA-DR, CXCR3, CD45RA, CD44, CD38, CD24, CD161, and CCR4.

In some embodiments, inflammatory bowel disease can be distinguished from a non-inflammatory bowel disease state by detecting differences in the absolute or relative abundances of particular cell types in a subject compared to known abundances or compared to another sample derived from a subject not having the disease or the same disease subtype. Cell types that can be used to distinguish various forms of inflammatory bowel disease or inflammatory bowel disease from a non-inflammatory bowel disease are immune cells including B cells, T cells, innate immune cells, and subtypes thereof.

### Methods of Treatment

The present disclosure provides methods of identifying subsets of patients with inflammatory bowel disease that may respond to a specific type of therapy. In some embodiments, these methods involve detecting the expression level of at least one marker as disclosed above to identify the particular subtype of inflammatory bowel disease affecting a subject. In some embodiments, the method further involves administering a therapeutically effective amount of a pharmaceutical composition that is suitable for treating the subject's particular subtype of inflammatory bowel disease. In some embodiments, the level of at least one marker (or a combination of markers) is determined for a subject before treatment commences. The marker's pretreatment level can be compared to the marker's level in the subject during and/or after treatment to monitor treatment efficacy. In some embodiments, the level of the marker is measured by any suitable method as described herein.

In other embodiment of methods for identifying a subset of subjects with inflammatory bowel disease, the methods comprise detecting the abundance of immune cells present in colonic tissue and/or a peripheral blood sample. In some embodiments, the method includes determining the abundance of T cells and subsets thereof, B cells and subsets thereof, and innate immune cells and subsets thereof. The cell-type profile of an subject can be used to identify the particular subtype of inflammatory bowel disease affecting the subject. In some embodiments, the method further comprises administering a therapeutically effective amount of a pharmaceutical composition that is suitable for treating the subject's particular subtype of inflammatory bowel disease. In some embodiments, the abundance of at least one cell type (or a combination of cell types) is determined for a subject before treatment commences. The cell type's pretreatment abundance can be compared to the cell type's abundance in the subject during and/or after treatment to monitor treatment efficacy. In some embodiments, the abundance of the cell type is measured by any suitable method as described herein.

In some embodiments, the pharmaceutical composition is a TNFα blocker.

In certain embodiments, the methods disclosed herein further comprise managing the therapeutic approach to the subject' disease. For example, the invention includes methods wherein the markers (or a specific combination or panel of markers) are measured before diagnosis and again after diagnosis, such as during or after treatment. In some embodiment of the methods, cell type abundance rather than, or in combination with, marker expression levels is measured. Using the profile of markers or cell type abundance that led to the diagnosis or a standard profile of markers or cell type abundance associated with a particular disease status, response to treatment and/or the progression, regression, or maintenance of the disease can be assessed by determining the marker and/or cell type profile for the subject after treatment has begun and/or terminated. Therapeutic options may change at different stages of the disease. The methods provided herein can surveil a subject's disease and alert the subject or caregiver to significant changes in disease status. Such monitoring allows for personalized treatment throughout the course of the disease.

In some embodiments, the pharmaceutical composition may be administered by any appropriate route for the treatment or prevention of a neoplasia. For example, administration may be accomplished by parenteral, intravenous, intra-arterial, subcutaneous, intramuscular, intraventricular, intracapsular, intraspinal, intracisternal, intraperitoneal, intranasal, by aerosol, by suppositories, or by oral administration. Pharmaceutical compositions for the treatment of an inflammatory bowel disease may be administered to humans with a pharmaceutically acceptable diluent, carrier, or excipient, in unit dosage form.

In one embodiment, the invention provides a method of monitoring treatment progress. The method includes determining an expression profile of a two or more of markers (e.g., CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR and TNFα) in a subject having, suspected of having, or susceptible to inflammatory bowel disease or symptoms thereof, in which the subject has been administered a therapeutic amount of a compound sufficient to treat the disease or symptoms thereof. The level of a marker (e.g., CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα) can be compared to known levels of the marker in either healthy normal controls or in other afflicted subjects to establish the subject's disease status. In some embodiments, a second level of the marker in the subject is determined after the determination of the first level, and the two levels are compared to monitor the course of disease or the efficacy of the therapy. In some embodiments, a pre-treatment level of a marker (e.g., CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα) in the subject is determined from a sample obtained prior to treatment; this pre-treatment marker expression level can then be compared to the level of the marker in a sample obtained from the subject after treatment commences to determine the efficacy of the treatment.

In another embodiment, the invention provides a method of monitoring treatment progress that includes determining a profile of immune cells present in colonic tissue and/or a peripheral blood sample. In some embodiments, the method includes determining the abundance of T cells and subsets thereof, B cells and subsets thereof, and innate immune cells and subsets thereof.

Kits for Characterizing Markers Associated with Inflammatory Bowel Disease

Some aspects of the present disclosure provide kits for detecting markers associated with inflammatory bowel disease in a sample. The invention provides kits for the characterizing inflammatory bowel disease. More specifically, the invention provides kits for detecting the expression levels of markers associated with inflammatory bowel disease (e.g., CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα). In one embodiment, the kit includes a capture molecule (e.g., antibody or polynucleotide probe) that binds a CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα polynucleotide or polypeptide.

In some embodiments of the kits used to characterize inflammatory bowel disease, reagents are provided for determining the abundance of particular cell types in a sample. In some embodiments, the invention provides kits for labeling cells and/or capturing cells. The labeled and/or captured cells can be quantified using any of the methods described herein or any other method known in the art.

In some embodiments, the kit comprises a sterile container. Such containers can be boxes, ampoules, bottles, vials, tubes, bags, pouches, blister-packs, or other suitable container forms known in the art. Such containers can be made of plastic, glass, laminated paper, metal foil, or other materials suitable for holding reagents.

If desired the kit is provided together with instructions for using the kit to detect the markers of interest. The instructions will generally include information about the use of the kit for the characterization of inflammatory bowel disease. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization.

In certain embodiments, a subject can be diagnosed with inflammatory bowel disease by adding a biological sample (e.g., blood or serum) from the subject to the kit, or components thereof, and detecting the relevant markers that are specifically bound by capture molecules. By way of example, the method comprises: (i) collecting a sample from the subject; (ii) adding subject's sample to the components in the kit, e.g., a holding tube or a substrate; and (iii) detecting the capture molecules to which the markers in the sample have bound.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Analysis of Biomarkers in Human Blood and Colonic Tissue to Characterize Inflammatory Bowel Disease (IBD)

Peripheral blood and/or colonic biopsy or tissue samples were collected from pediatric and adult subjects with and without inflammatory bowel disease (IBD). Subjects with IBD were originally diagnosed based on clinical, endoscopic, and histological information. To confirm inflammation status of colonic tissue specimens, the most inflamed region and the colonic area from which research biopsies were taken were scored by a pathologist using the Nancy Histological Index (0-4) (Marchal-Bressenot et al., Gut, 65:1919-20 (2016)). Grade 0 to 1 was considered inactive disease and grades 2 to 4 were considered active. For those colonic specimens used in cytometry by Time of Flight (CyTOF) analysis, disease activity status was assigned based on the Nancy Index of the nearest colonic specimen submitted for clinical histopathologic assessment.

For peripheral blood specimens used in CyTOF analysis, activity status was assigned at the time of sample collection in those patients without concomitant intestinal specimens. Assignments were based on either (1) the Nancy Index of the most inflamed available intestinal pathology specimen obtained within 1 month of peripheral blood draw or (2) the disease assessment scoring systems Pediatric Ulcerative Colitis Activity Index (PUCAI) for ulcerative colitis and Harvey-Bradshaw index (HBI) for Crohn's disease. PUCAI >10 and HBI >3 was deemed active. Chart review was performed to obtain disease phenotype, including disease extent and behavior per the Montreal Classification System (Table 1). Additionally, information including age, gender, treatment status, and relevant laboratory testing was obtained from each subject's medical records (Table 1).

TABLE 1
Demographic and Clinical Characteristics of Study Participants

| | | | Ulcerative Colitis | | | |
|---|---|---|---|---|---|---|
| | | | Blood | | Tissue | |
| | Non-IBD Control | | (n = 27) | | (n = 32) | |
| | Blood | Tissue | Inactive | Active | Inactive | Active |
| | (n = 24) | (n = 18) | (n = 5) | (n = 22) | ( n = 14) | (n = 18) |
| Gender (n %) | | | | | | |
| Female | 15 (63%) | 12 (67%) | 3 (60%) | 10 (45%) | 6 (43%) | 6 (33%) |
| Male | 9 (37%) | 6 (33%) | 2 (40%) | 12 (55%) | 8 (57%) | 12 (67%) |
| Age at collection; years (mean ± SD) | (17.1 ± 11.7) | (24.6 ± 17.3) | (14.6 ± 4.9) | (16.9 ± 4.1) | (20.3 ± 9.2) | (16.3 ± 3.0) |
| Age at diagnosis; years (mean ± SD) | (N/A) | (N/A) | (11.4 ± 3.0) | (13.4 ± 4.5) | (17.0 ± 6.9) | (12.0 ± 4.4) |
| Duration of disease; years (mean ± SD) | (N/A) | (N/A) | (3.2 ± 3.7) | (3.6 ± 3.1) | (3.3 ± 3.1) | (4.3 ± 4.6) |
| Histological disease activity | | | | | | |
| (Nancy Index), n (%) | | | | | | |
| Nancy Index 0 | N/A | N/A | 2 (40%) | 0 | 11 (79%) | 0 |
| Nancy Index 1 | N/A | N/A | 1 (20%) | 0 | 3 (21%) | 0 |
| Nancy Index 2 | N/A | N/A | 0 | 12 (54%) | 0 | 7 (39%) |
| Nancy Index 3 | N/A | N/A | 0 | 3 (14%) | 0 | 6 (33%) |
| Nancy Index 4 | N/A | N/A | 0 | 6 (27%) | 0 | 5 (28%) |
| Unavailable | N/A | N/A | 2 (40%) | 1 (5%) | 0 | 0 |
| Mean Nancy Index (mean ± SD) | N/A | N/A | (0.33 ± 0.57) | (2.71 ± 0.90) | (0.21 ± 0.43) | (2.89 ± 0.83) |
| Extent of disease | | | | | | |
| (Montreal classification) n (%) | | | | | | |
| Heal CD (L1) | N/A | N/A | N/A | N/A | N/A | N/A |
| Crohn's Colitis (L2) | N/A | N/A | N/A | N/A | N/A | N/A |
| Heo-colonic CD (L3) | N/A | N/A | N/A | N/A | N/A | N/A |
| Upper GI disease (L4) | N/A | N/A | N/A | N/A | N/A | N/A |
| Left-sided colitis | N/A | N/A | 1 (20%) | 3 (14%) | 4 (29%) | 0 |
| Pancolitis | N/A | N/A | 4 (80%) | 19 (86%) | 10 (71%) | 18 (100%) |
| Disease behavior | | | | | | |
| (Montreal classification) n (%) | | | | | | |
| Non-stenotic/non-penetrating (B1) | N/A | N/A | N/A | N/A | N/A | |
| Stenotic (B2) | N/A | N/A | N/A | N/A | N/A | N/A |
| Penetrating (B3) | N/A | N/A | N/A | N/A | N/A | N/A |
| Stenotic/penetrating (B2B3) | N/A | N/A | N/A | N/A | N/A | N/A |
| Perianal disease (p) | N/A | N/A | N/A | N/A | N/A | N/A |
| Sample location n (%) | | | | | | |
| Left Colon | N/A | 3 (17%) | N/A | N/A | 2 (14%) | 2 (11%) |
| Transverse Colon | N/A | 15 (83%) | N/A | N/A | 12 (86%) | 16 (89%) |
| CRP levels, mg/dL | | | | | | |
| Median | 0.03 | 0.04 | 0.24 | 0.88 | 0.09 | 0.65 |
| [min, max]; n patients with CRP | [0.03, 1.65]; | [0.04, 0.04]; | [0.06, 0.42]; | [0.03, 5.97]; | [0.03, 0.38]; | [0.03, 5.97]; |
| value within 10d of sample collection | 3 | 1 | 2 | 13 | 6 | 10 |
| Treatment n (%) | | | | | | |
| 5-Aminosalicylic acid | N/A | N/A | 0 | 4 (18%) | 4 (28%) | 6 (33%) |
| Corticosteroids | N/A | N/A | 1 (20%) | 11 (50%) | 2 (14%) | 9 (50%) |
| Immunomodulators (AZA, MTX, | N/A | N/A | 2 (40%) | 7 (32%) | 4 (28%) | 10 (55%) |
| 6MP, tacrolimus) | | | | | | |
| anti-TNF inhibitors | N/A | N/A | 0 | 5 (23%) | 5 (36%) | 4 (22%) |
| ustekinumab | N/A | N/A | 0 | 0 | 0 | 0 |
| vedolizumab | N/A | N/A | 0 | 1 (5.5%) | 0 | 3 (16.7%) |
| None | N/A | N/A | 2 (40%) | 5 (23%) | 3 (21%) | 1 (5%) |
| | Crohn’s Disease | | | | | |
| | Blood | | Tissue | | | |
| | (n = 34) | | (n = 37) | | | |
| | Inactive | Active | Inactive | | | |
| | (n = 12) | (n = 22) | (n = 25) | (n =1 2) | | |
| Gender (n %) | 4 (33%) | 7 (32%) | 9 (36%) | 5 (42%) | | |
| Male | 8 (67 %) | 15 (68% | 16 (64%) | 7 (58%) | | |
| Age at collection; years (mean ±SD) | (17.5 ± 4.4) | (16.7 ± 3.86) | (18.4 ± 6.6) | (20.6 ± 15.2) | | |
| Age at diagnosis; years (mean ±SD) | (12.5 ± 3.3) | (13.2 ± 3.5) | (13.2 ± 4.5) | (14.3 ± 5.4) | | |
| Duration of disease; years (mean ±SD) | (5.1 ± 2.8) | (3.5 ± 3.4) | (5.1 ± 4.7) | (6.3 ± 10.9) | | |
| Histological disease activity | 3 (25%) | 0 | 20 (80%) | 0 | | |
| (Nancy Index), n (%) | | | | | | |
| Nancy Index 0 | | | | | | |
| Nancy Index 1 | 8 (67%) | 0 | 5 (20%) | 0 | | |
| Nancy Index 2 | 0 | 12 (55%) | 0 | 8 (66%) | | |
| Nancy Index 3 | 0 | 2 (9%) | 0 | 2 (17%) | | |
| Nancy Index 4 | 0 | 7 (32%) | 0 | 2 (17%) | | |
| Unavailable | 1 (8%) | 1 (4%) | 0 | 0 | | |
| Mean Nancy Index (mean ±SD) | (0.73 ± 0.45) | (2.76 ± 0.94) | (0.2 ± 0.41) | (2.5 ± 0.80) | | |
| Extent of disease | 2 (17%) | 3 (14%) | 5 (20%) | 0 | | |
| (Montreal classification) n (%) | | | | | | |
| Ileal CD (L1) | | | | | | |
| Crohn’s Colitis (L2) | 3 (25%) | 5 (23%) | 6 (24%) | 3 (25%) | | |
| Heo-colonic CD (L3) | 7 (58%) | 14 (64%) | 14 (56%) | 10 (83) | | |
| Upper GI disease (L4) | 6 (50%) | 13 (60%) | 10 (40%) | 4 (33%) | | |
| Left-sided colitis | N/A | N/A | N/A | N/A | | |
| Pancolitis | N/A | N/A | N/A | N/A | | |
| Disease behavior | 10 (83%) | 13 (60%) | 14 (56%) | | | |
| (Montreal classification) n (%) | 2 (17%) | 6 (27%) | 7 (28%) | 2 (17%) | | |
| Non-stenotic/non-penetrating (B1) | 0 | 3 (14%) | 2 (8%) | 0 | | |
| Stenotic (B2) | 0 | 0 | 2 (8%) | 0 | | |
| Penetrating (B3) | 7 (58%) | 9 (41%) | 12 (48%) | 4 (33%) | | |
| Stenotic/penetrating (B2B3) | | | | | | |
| Perianal disease (p) | | | | | | |
| Sample location n (%) | N/A | N/A | 2 (8%) | 1 (8%) | | |
| Left Colon | N/A | N/A | 23 (92%) | 11 (92%) | | |
| Transverse Colon | | | | | | |
| CRP levels, mg/dL | 0.28 | 1.28 | 0.44 | 1.58 | | |
| Median | [0.03, 1.93]; | [0.05, 9.31]; | [0.05, 6.09]; | [0.07, 7.59]; | | |
| [min, max]; n patients with CRP | 6 | 13 | 13 | 6 | | |
| value within 10d of sample collection | | | | | | |
| Treatment n (%) | 2 (16%) | 2 (9%) | 2 (8%) | 0 | | |
| 5-Aminosalicylic acid | 3 (25%) | 4 (18%) | 3 (12%) | 1 (8%) | | |
| Corticosteroids | 1 (8%) | 8 (36%) | 5 (20%) | 2 (17%) | | |
| Immunomodulators (AZA, MTX, | 7 (58%) | 7 (32%) | 12 (48%) | 3 (25%) | | |
| 6MP, tacrolimus) | 0 | 1 (4.5%) | 1 (4.0%) | 1 (8.3%) | | |
| anti-TNF inhibitors | 0 | 0 | 0 | 0 | | |
| ustekinumab | 1 (8%) | 4 (18%) | 8 (32%) | 6 (50%) | | |
| vedolizumab | | | | | | |
| None | | | | | | |

AZA = azathioprine; MTX = methotrexate; 6MP = 6-mercaptopurine

The total numbers mucosa and peripheral blood (PB) samples derived from non-IBD controls, subjects having ulcerative colitis (UC), and subjects having Crohn's disease (CD) are listed in addition to the breakdown of gender, age at collection/diagnosis, duration of disease, histological disease activity using the Nancy Index, extent of IBD disease and behavior per the Montreal classification system, location of mucosa sample collection (left colon or transverse), C-reactive protein (CRP) levels within 10 days of sample collection, and current medical therapy of each patient listed. Corticosteroid use was defined as systemic treatment with either prednisone or intravenous steroids at time of sample collection. Anti-tumor necrosis factor (anti-TNF) inhibitor use was defined as use of any anti-TNF inhibitor within three months of sample collection.

### Example 2: Marker Analysis

To better define the immune dysregulation in IBD, mass cytometry (e.g., CyTOF) was used to analyze approximately 40 surface and intracellular markers including cytokines to colonic mucosa and peripheral blood of IBD patients and non-IBD control subjects.

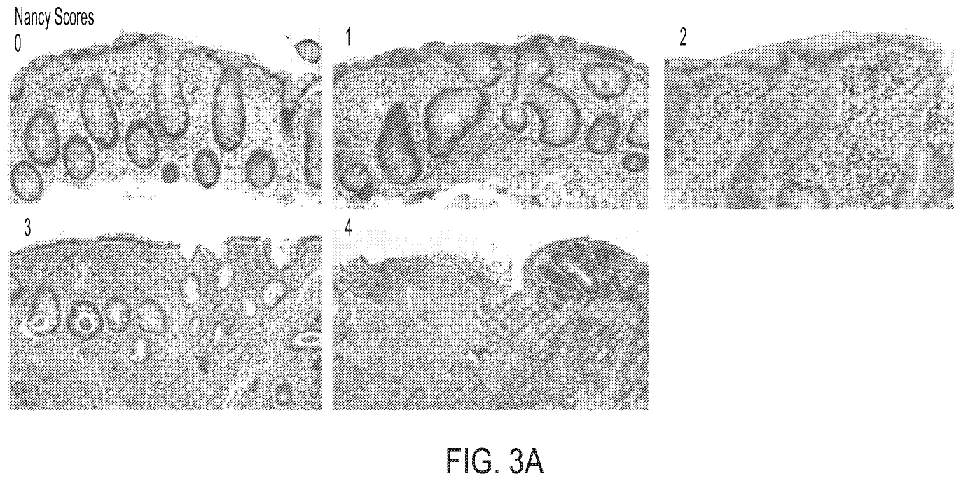

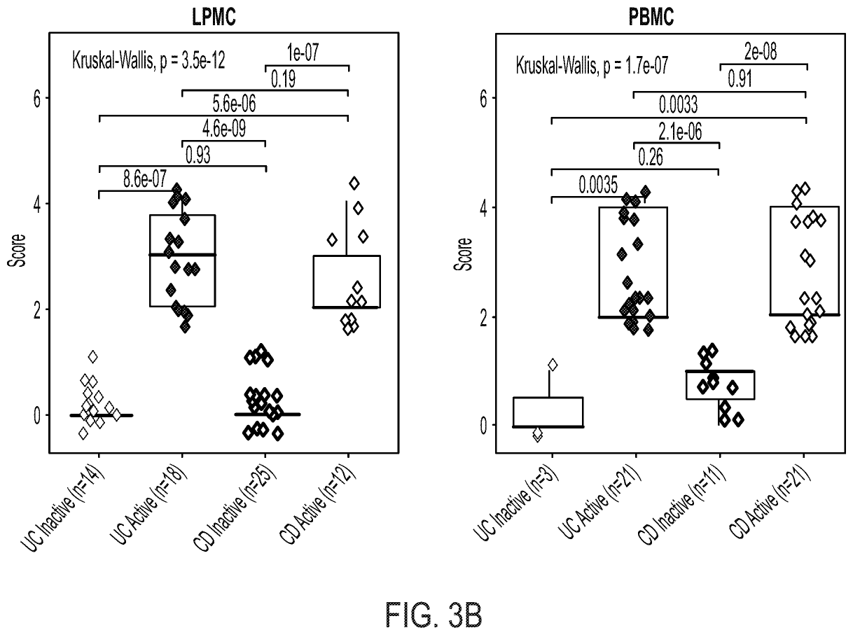

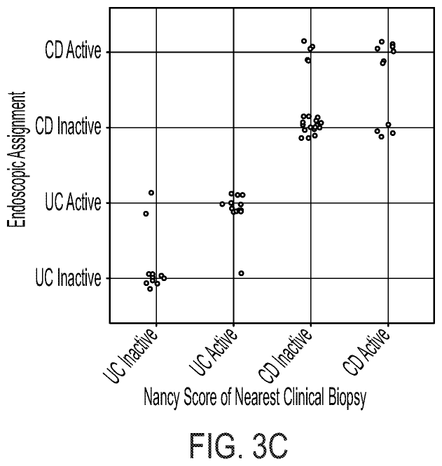

CyTOF was performed on colonic mucosa from 18 non-IBD, 32 UC (14 inactive, i, and 18 active, a) and 37 CD (i=25, a=12) subjects and peripheral blood from 24 non-IBD, 27 UC (i=5, a=22) and 34 CD (i=12, a=22) subjects (Table 1). Of these, 24 subjects had matched blood and biopsy samples. CyTOF markers included surface antigens and cytokines (Table 2), with the intent to broadly immunophenotype innate and adaptive immune cells. Mucosal analyses were performed on cryopreserved samples with robust viability as we have described (Konnikova et al., Mucosal Immunol, 11:1684-93 (2018) (FIG. 2). The inflammatory status of the IBD tissue was based on the pathology report as scored using the Nancy scoring system by an experienced pathologist who blindly reviewed diagnostic biopsies that were the closest in location to the research biopsies taken (Marchal-Bressenot et al., Gut, 65:1919-20 (2016). Using Nancy index of 0-1 as inactive and 2-4 as active disease, there was a correlation between the Nancy index and inflammatory status as determined by the clinical assessment of an endoscopist (FIGS. 3A to 3C). Table 3 provides an overview of the observed results.

TABLE 2

| | | |
|---|---|---|
| Marker | Tg | Clone |

CyTOF Panel A and B (Shared Targets)

| | | |
|---|---|---|
| CD45 | Y89 | HI3O |
| CD19 | 142Nd | HIB19 |
| HLA-DR | 143Nd | L243 |
| TNFα | 144Nd | MAb11 |
| CDBα | 146Nd | RPA-TB |
| CD45RO | 147Sm | UCHL1 |
| CD14 | 148Nd | RMO52 |
| CD25 | 149Sm | 2A3 |
| IL-22 | 150Nd | 22URTI |
| CD123 | 151Eu | 6H6 |
| CD45RA | 153Eu | HI100 |
| CD38 | 154Sm | HIT2 |
| CD27 | 155Gd | L128 |
| CD3 | 15BGd | UCHT1 |
| CCR7 | 159Tb | G043H7 |
| IFNγ | 160Gd | 4S.B3 |
| IL-1β | 162Dy | CRM56 |
| CXCR3 | 163Dy | G025H7 |
| CD161 | 164Dy | HP-3610 |
| CCRG | 168Er | G034E3 |
| IL-17A | 1697m | BL168 |
| CD127 | 171Yb | eBIoRDR5 |
| IL-21 | 172Yb | 3A3-N2 |
| CD4 | 174Yb | SK3 |
| CD5G | 176Yb | HCD56 |

CyTOF Panel A (Unique Targets)

| | | |
|---|---|---|
| CD44 | 115In | IM7 |
| ckit | 141 Pr | 104D2 |
| CD16 | 145Nd | 3G8 |
| CTLA-4 | 152Sm | L3D10 |
| CCR4 | 156Gd | L291H4 |
| AHR | 161 Dy | FF3399 |
| FoxP3 | 165Ho | PCH101 |
| CD24 | 166Er | ML5 |
| NKp46 | 173Yb | 9E2 |
| Tbet | 175Lu | 4B 10 |

CyTOF Panel B (Unique Targets)

| | | |
|---|---|---|
| CD11c | 115In | Bu15 |
| CD66b | 141Pr | G10F5 |
| IL-2319 | 161Dy | 23dcdp |
| CD163 | 165Ho | GHI/61 |

Immune cell subsets of interest were exported for dedicated automated analyses with unbiased clustering. Phenotypically similar clusters were organized into branches of hierarchical clustering dendrograms. On relevant figures, dendrogram nodes were labeled in circles while node branches are labeled as Node X(−) or Node X(+) depending on the direction of branch splitting. The abundances of cellular populations (individual clusters and/or node branches) were compared across subject groups. Finally, predictive modeling using random forest (RF) was applied to identify features that distinguish subject groups in the mucosa and periphery.

TABLE 3
Summary of Marker Analysis

| | | | | | | | |
|---|---|---|---|---|---|---|---|
| Marker | UCa vs | CDa vs | CDa vs | UCa vs | CDa vs | UCi vs | CDi vs |
| Expression | Non-IBD | Non-IBD | UCA | UCi | CDi | Non-IBD | Non-IBD |

LP

| | | | | | | | | |
|---|---|---|---|---|---|---|---|---|
| T cells | [symbol] | CD38, HLA-DR, | CD38, | IL-1β | HLA-Dr, CD38, | IL-1β | IL-17A | IL-17A, IFNγ, HLA-DR, |
| | [symbol] | FOXP3, IL-17A, | HLA-DR, | IL-17A | CTLA-4 | CD161 | | CD38, FOXP3, CCR6, |
| | | CCR4, CTLA-4 | FOXP3 | | | | | CCR4, CD45RO, CD161, |
| | | TNFα | TNFα | | | | | CD24 |
| B cells | [symbol] | CCR4 | CCR4 | IL-1β | | CCR7, HLA-DR, | | CCR7, CD45RO, |
| | [symbol] | | | IL-17A | | TFNα | | IFNγ, HLA-DR |
| Innate cells | [symbol] | IL-1β CD38, | IL-1β, | IL-1β, TNFα, | IL-17A | IL-1β | | |
| | [symbol] | CD44, HLA-DR, | IFNγ, | IL-22 | TNFα | CD161 | | |
| | | IL-21 | CD45RO | CD38 | | | | |
| | | TNFα | | | | | | |

PR

| | | | | | |
|---|---|---|---|---|---|
| T cells | [symbol] | | | CCR7, CD24, | CD24, CD45RA, |
| | [symbol] | | | IL-22 | CCR4 |
| B cells | [symbol] | CXCR3 | CXCR3 | IL-1β | |
| | [symbol] | | | IL-1β, CXCR3, | |
| Innate cells | [symbol] | | | HLA-DR | |
| | [symbol] | | | CD45RA | |

### Example 3: Active Ulcerative Colitis Mucosa is Characterized by an Increased B:T Cell Ratio

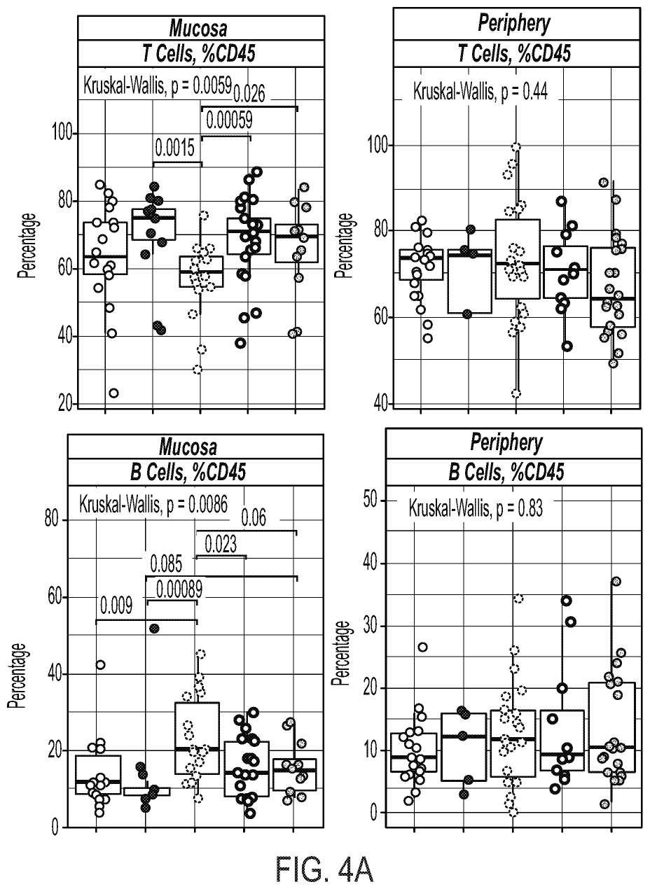

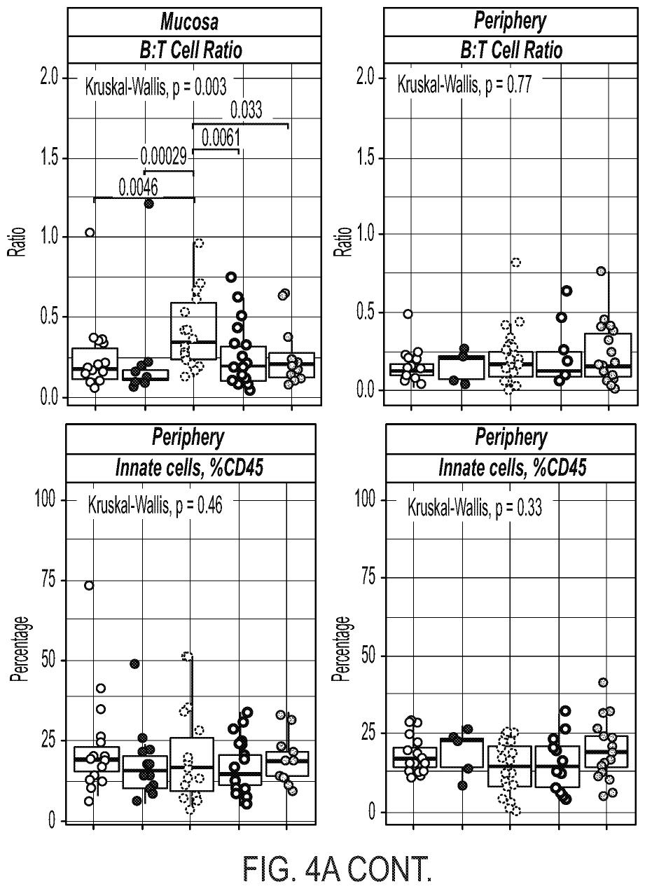

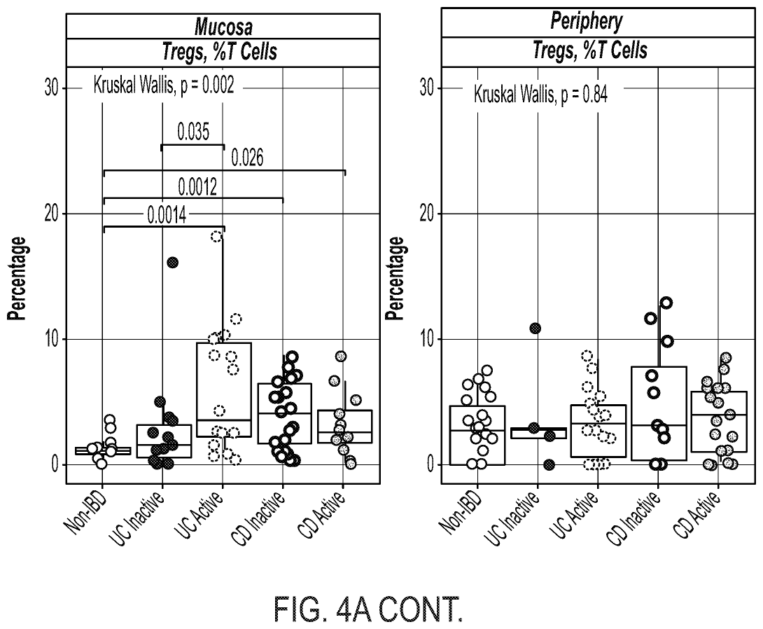

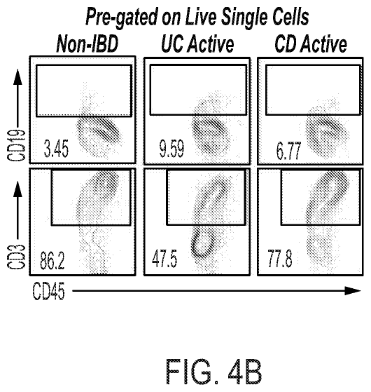

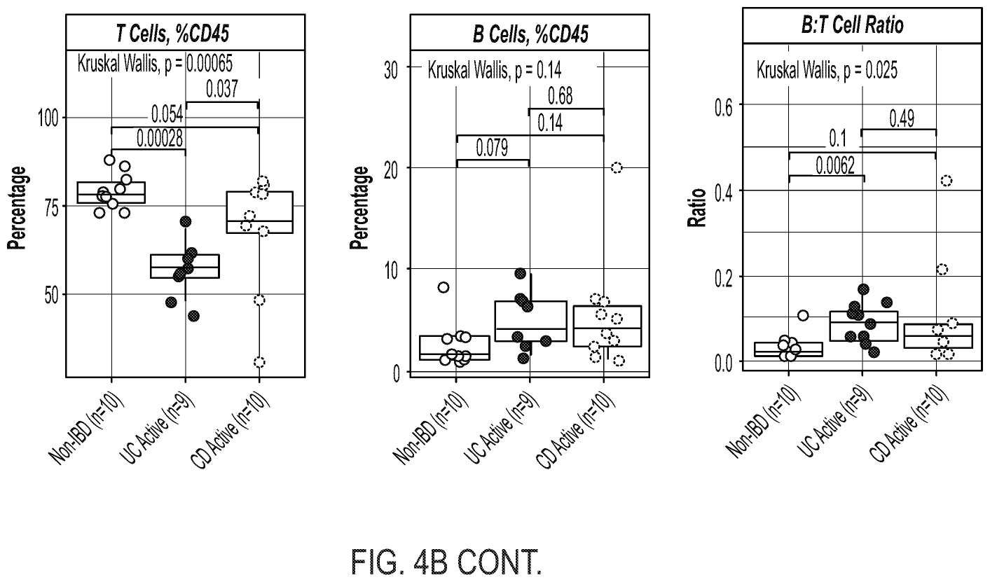

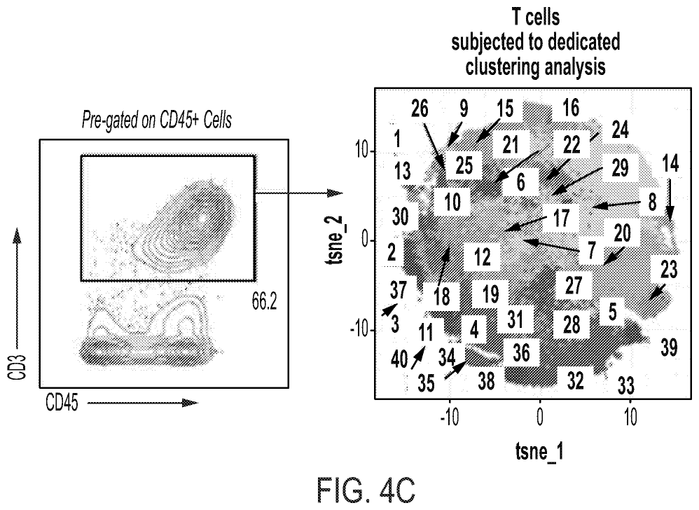

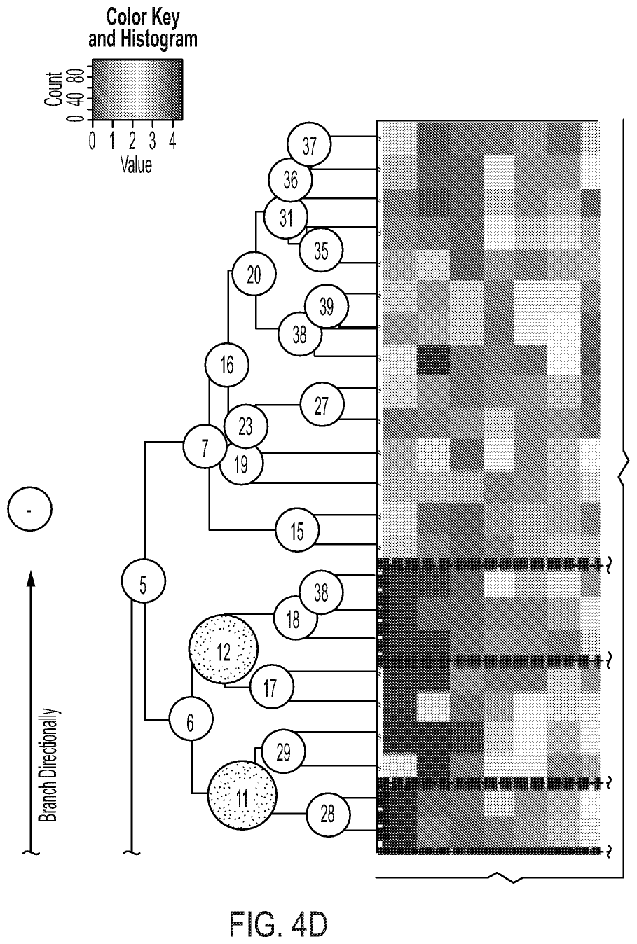

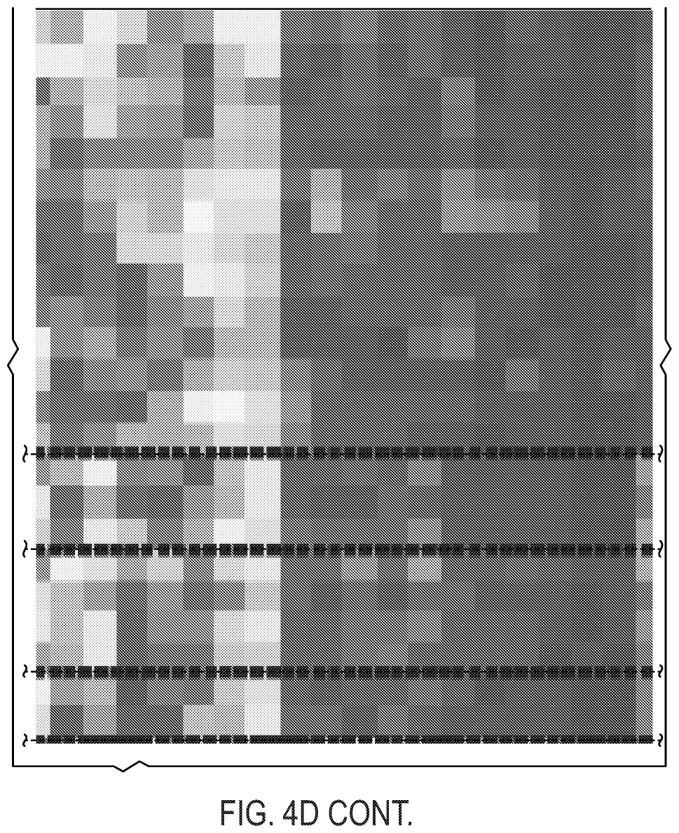

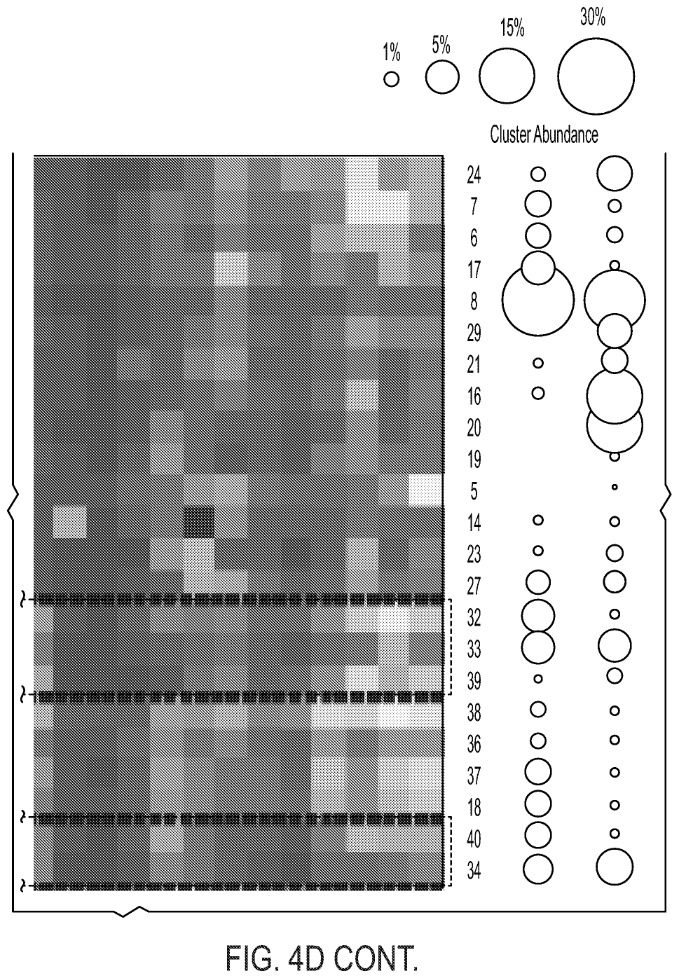

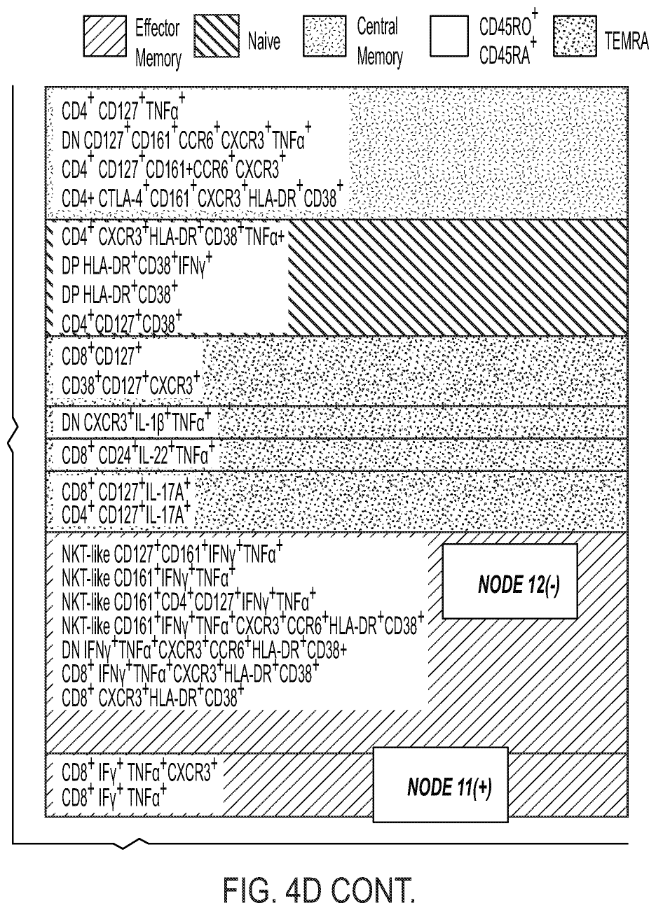

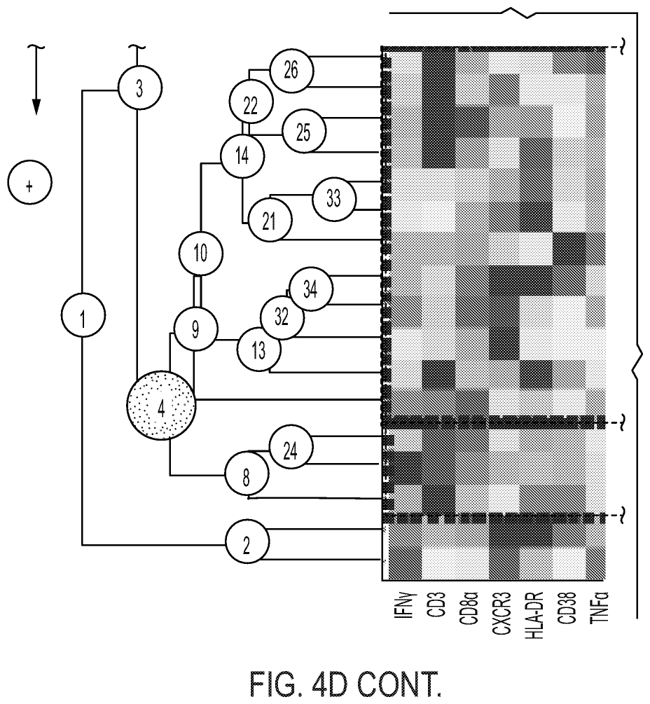

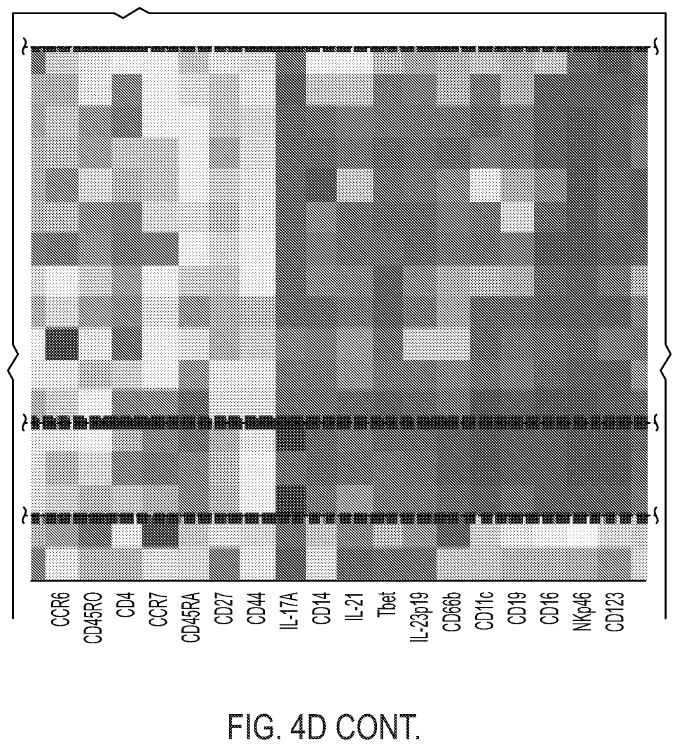

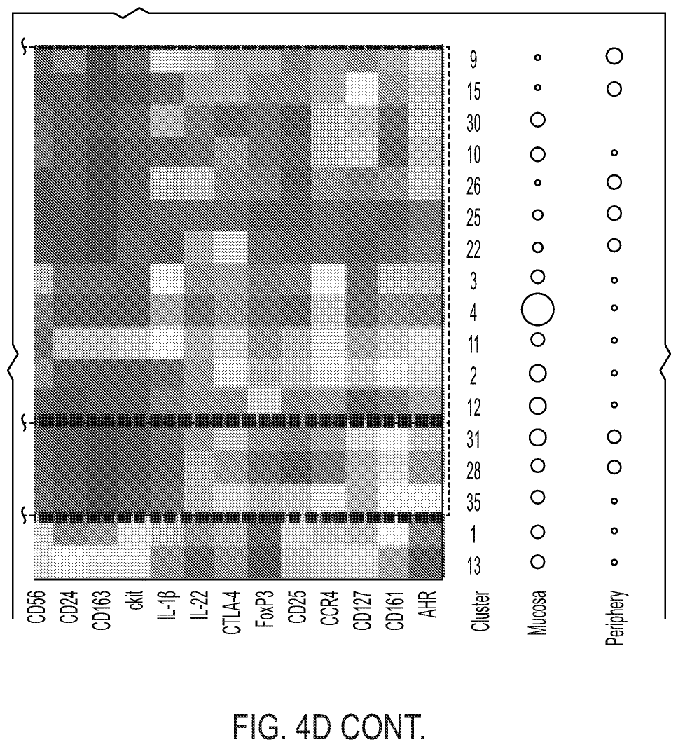

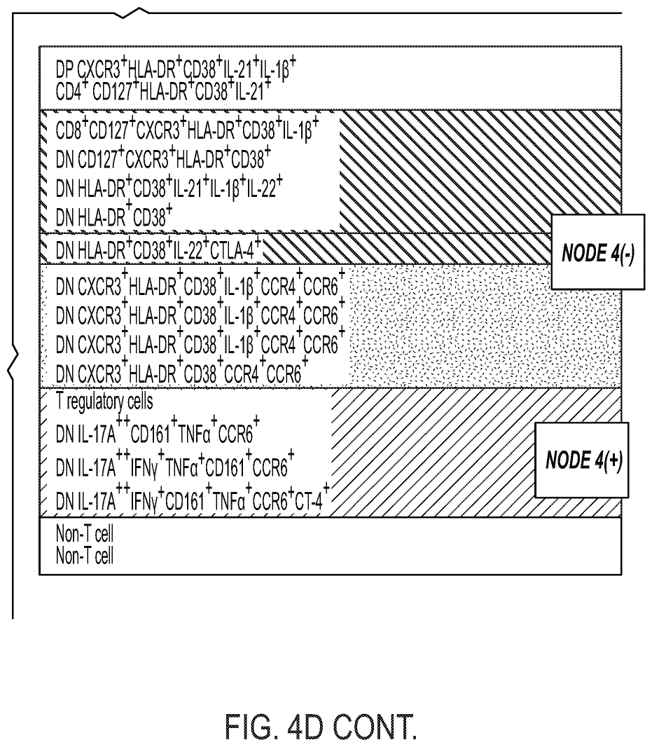

Prior to automated analyses, we assessed the relative proportions of manually-gated T cells (CD3⁺CD45⁺), B cells (CD19⁺CD45⁺) and innate immune cells (CD3⁺CD19⁺CD45⁺) across all subject groups (FIG. 4A). Active ulcerative colitis mucosa had proportionally fewer T cells compared to all other IBD groups with an enhanced B:T cell ratio (FIG. 4A). The T and B cell findings in UC compared to non-IBD mucosa were validated by flow analysis of an independent cohort (n=10 non-IBD, 9 UCa, 10 CDa) (FIG. 4B). No overall differences in the proportion of innate cells were observed (FIG. 4A).

T cells were clustered in a dedicated automated analysis (FIGS. 4C, 4D). T cell memory status (naïve, effector memory (EM), central memory (CM), terminally differentiated EM cells re-expressing CD45RA (TEMRA), and a subtype that did not clearly fall into these categories, labeled CD45RO⁺CD45RA⁺) was assigned based on differential expression of CD45RA, CD45RO, CCR7 and CD27 (FIG. 4D). A range of CD4⁺, CD8⁺, CD4⁺CD8⁺ (DP) and CD4⁻CD8⁻ (DN) T cell subtypes were identified.

### Example 4: IL-17A⁺⁺CD161⁺ EM T Cells are Enriched while IFNγ⁺ TNFα⁺ EM T Cell Subsets are Diminished in UCa Mucosa

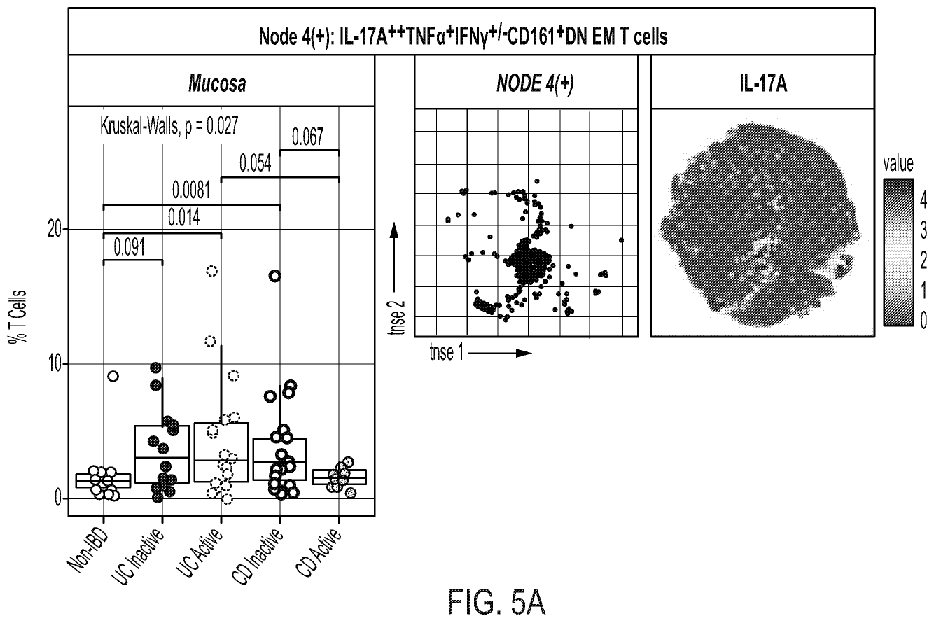

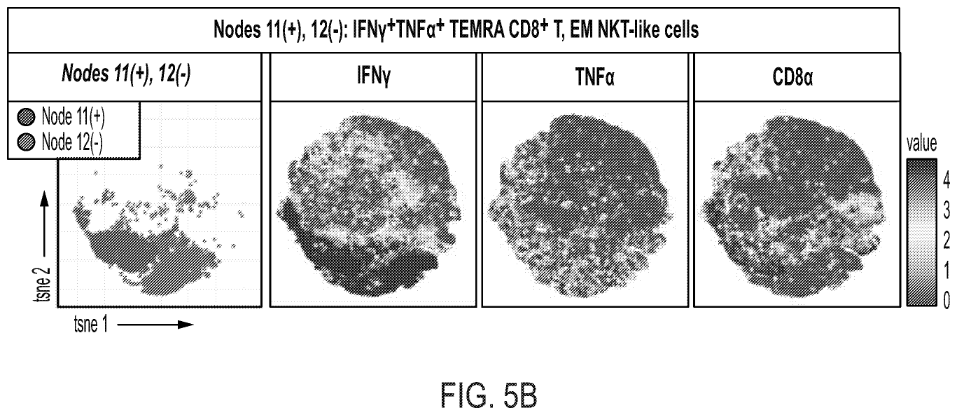

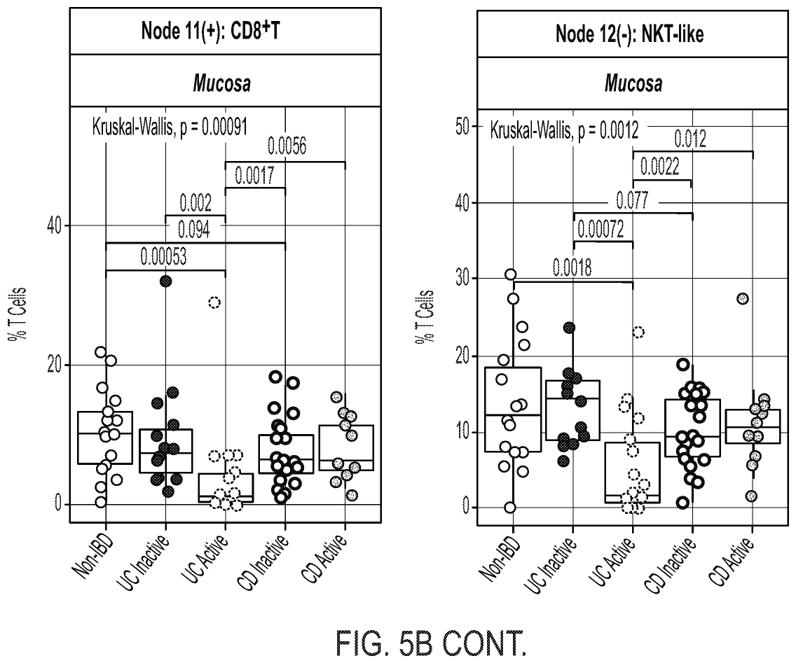

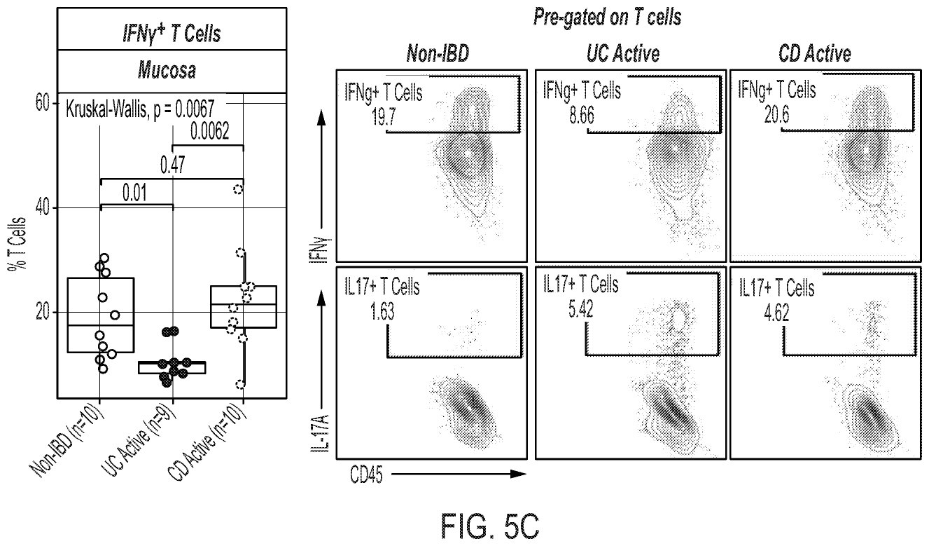

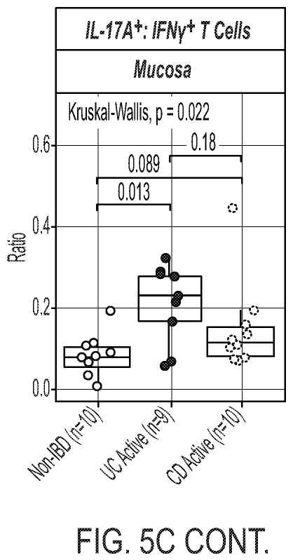

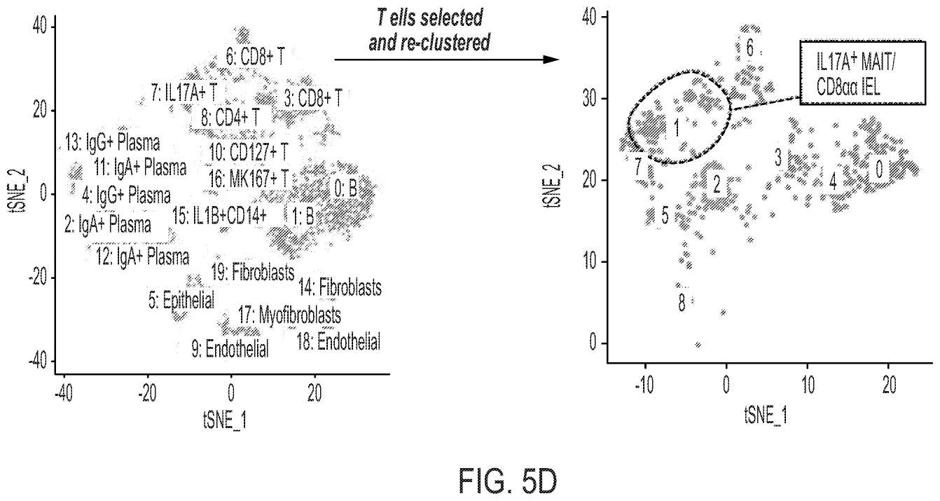

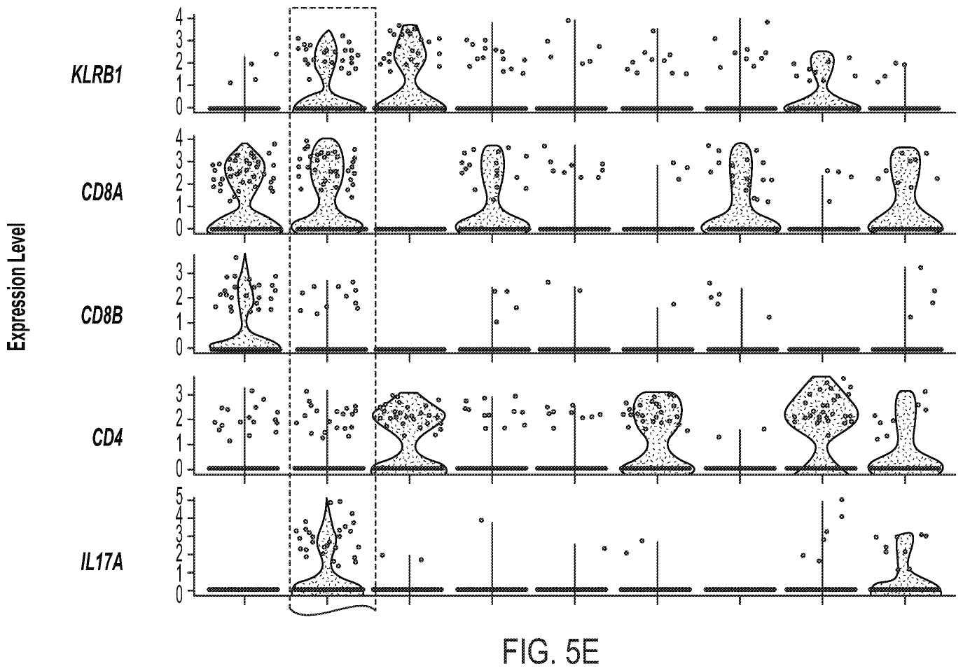

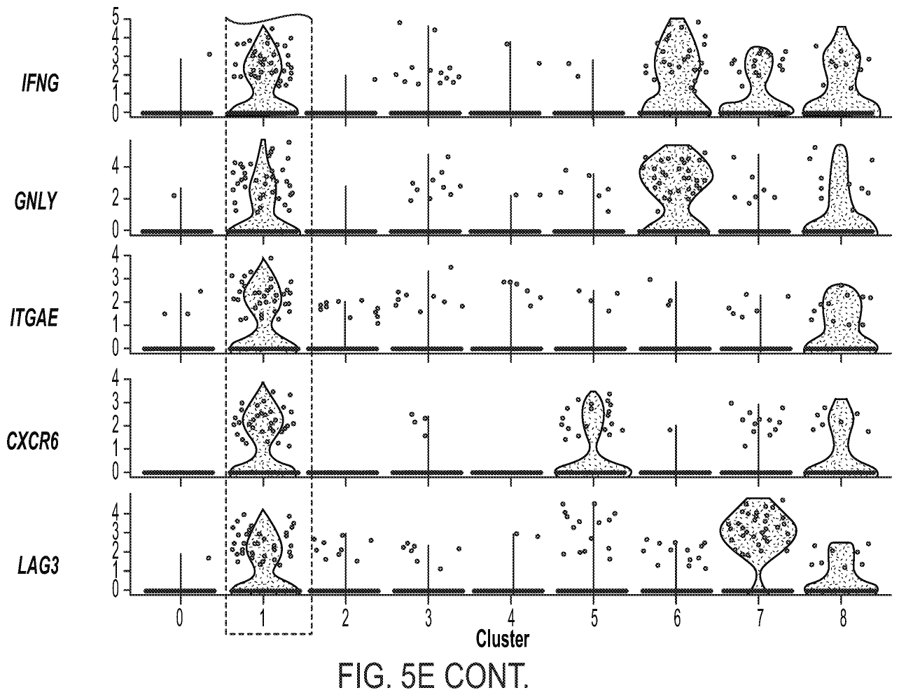

Disease-specific compositional differences were identified in T cells pertaining to cytokine-producing EM subsets. Specifically, DN IL-17A⁺⁺CD161⁺ EM T cell clusters (belonging to Node 4(+)) were increased in UCa mucosa (FIG. 5A). Given cytokine co-expression (not only IL-17A⁺⁺ but also TNFα⁺ and IFNγ⁺) as well as CD161 and CCR6 co-expression, these DN EM T cells may represent mucosal-associated invariant T cells (MAIT) (or related lymphocytes with innate characteristics), although the CyTOF panel was not equipped to capture MAIT-specific semi-invariant T cell receptors for more definitive classification.

While these clusters were increased, IFNγ⁺TNFα⁺ EM T cell subsets were decreased compared to all other subject groups in UCa mucosa, including Node 11(+) (cytotoxic CD8+ TEMRA cells) and Node 12(−), which were CD161⁺CD56⁺ EM T cells that could represent natural killer T (NKT)-like cells (FIG. 5B).

A decrease in overall IFNγ⁺ T cells in UCa compared to both non-IBD and CDa mucosa was confirmed with flow cytometry, as was an increased ratio of IL-17A⁺:IFNγ⁺ T cells in UCa mucosa compared to non-IBD (FIG. 5C).

To gain greater molecular insights into these cell subsets, scRNA-seq was performed on matched cryopreserved mucosal samples from five UCa subjects in the CyTOF cohort. All expected epithelial, stromal, and immune cell subsets were recovered and further analyses was focused on the T cell clusters (FIG. 5D). T cells were identified, subsetted and reclustered independently (FIG. 5D). A T cell cluster co-expressing IL17A, IFNG, CD8A (but not CD8B) was identified, and KLRB1 (CD161), potentially representing IL-17A⁺CD161⁺ cells of similar phenotype to those increased in UCa mucosa (Node 4(+)) identified with CyTOF (FIGS. 5D, 5E). As the sequencing data did not capture enrichment of specific TCR transcripts, such as TRAV1-2 or the specific TRAJ transcripts that make up the semi-invariant MAIT T cell receptor, these cells may represent MAIT cells or polyclonal CD8αα IELs Specific sequencing of the TCR region (www.nature.com/articles/s41590-019-0544-5) will be necessary to identify if this subset is composed of public or private TCRs. Nevertheless, the scRNA-seq analysis did highlight several MAIT-related markers in this cluster, which were not included in the CyTOF panels, including ITGAE (CD103), CXCR6, LAG3, GNLY, CCR5, and RORA, a transcription factor belonging to the same family as RORGT, associated with Th17 responses.

### Example 5: IBD Mucosa is Characterized by an Abundance of HLA⁻DR⁺CD38⁺ T Cell Populations

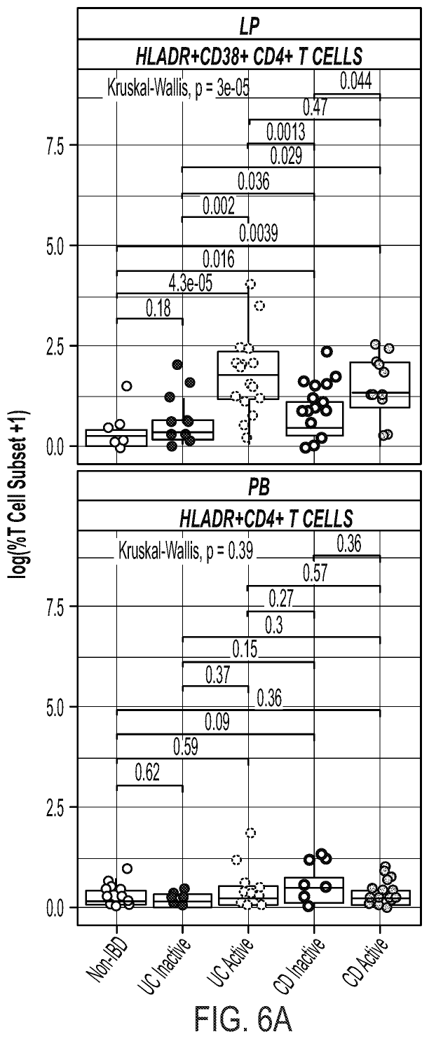

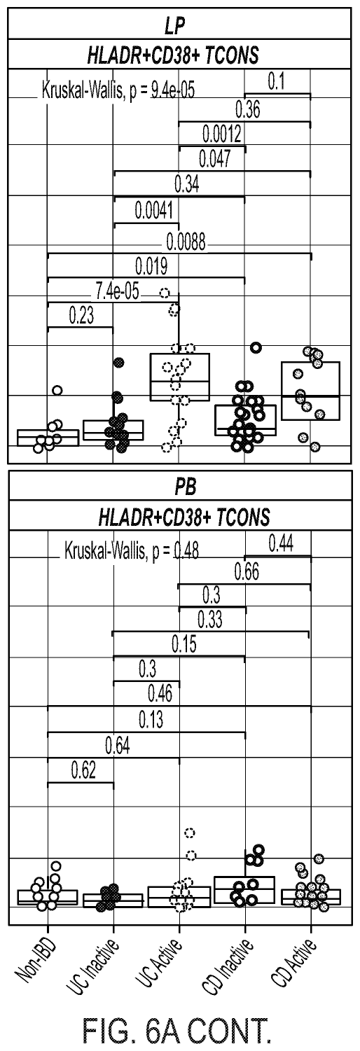

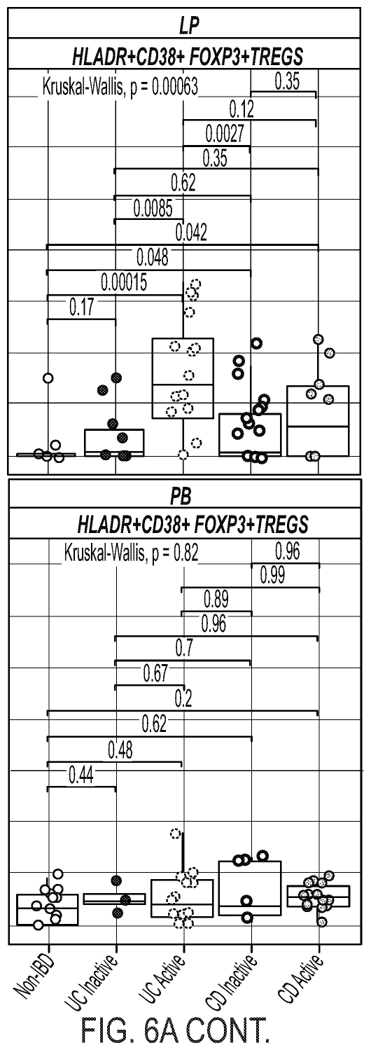

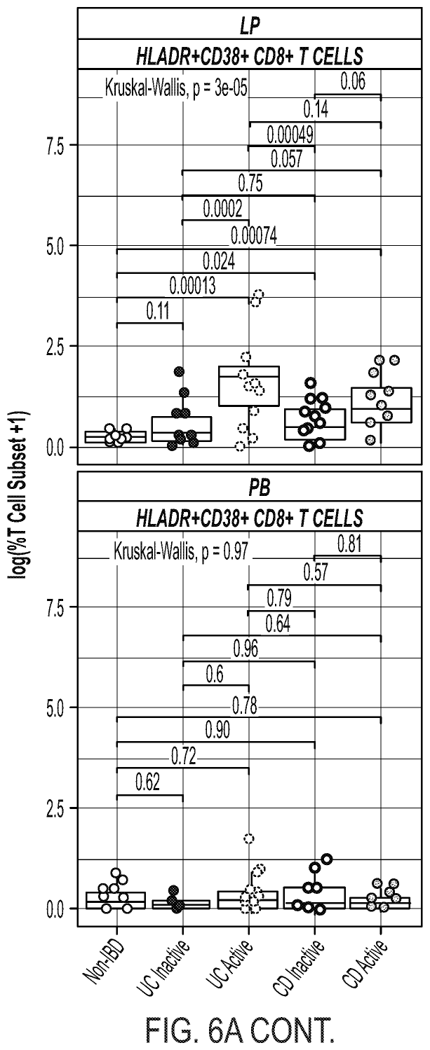

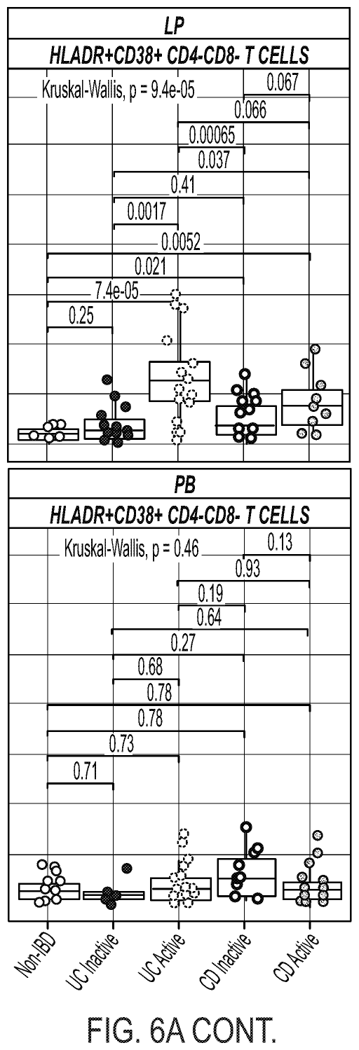

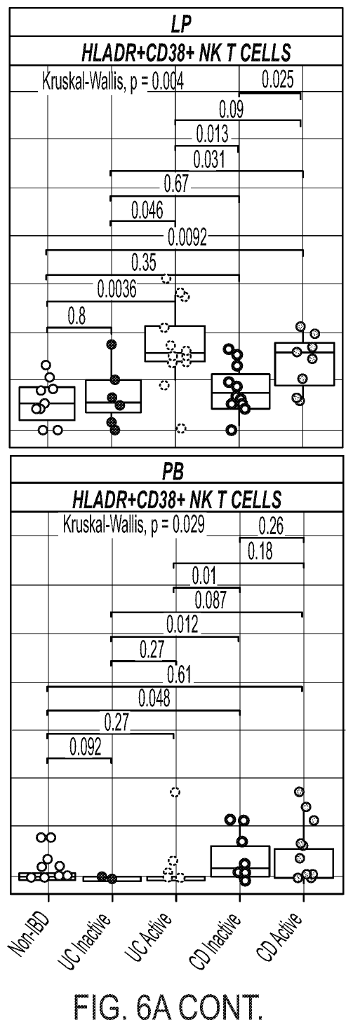

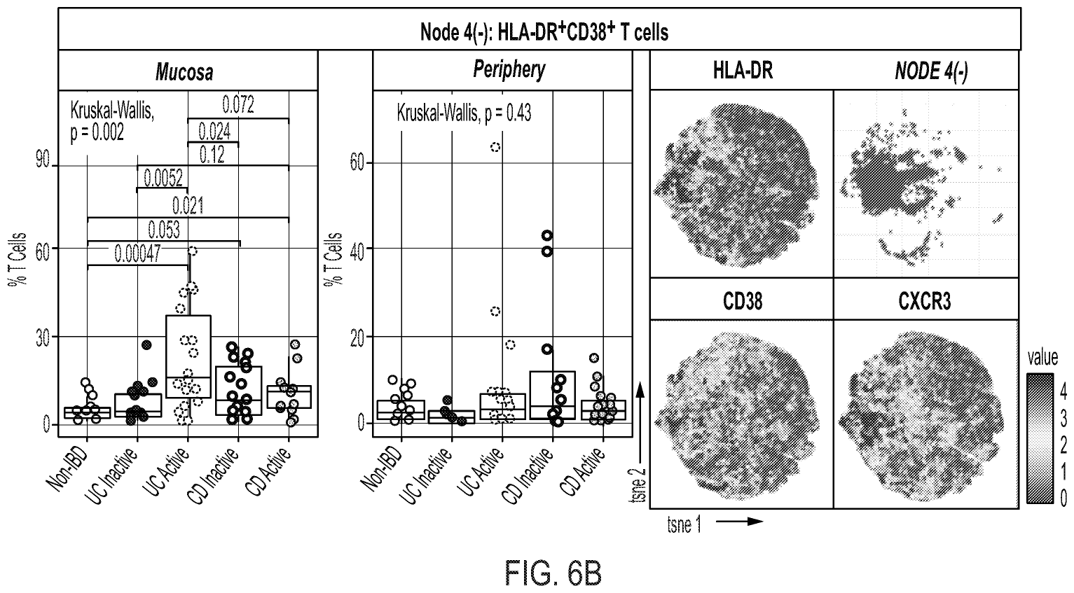

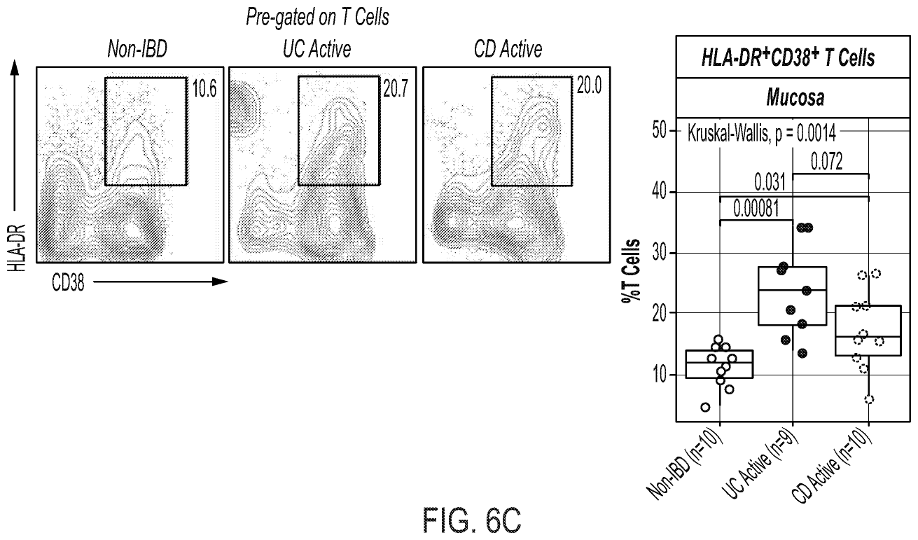

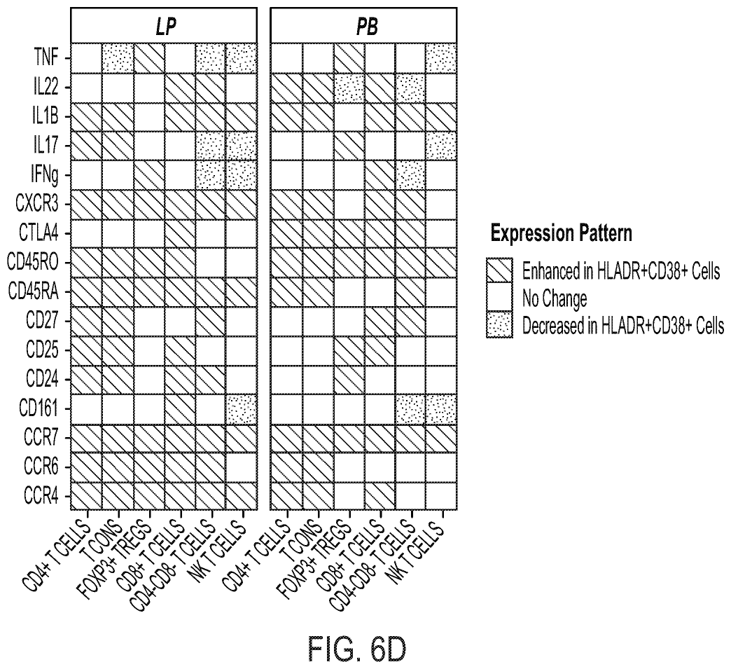

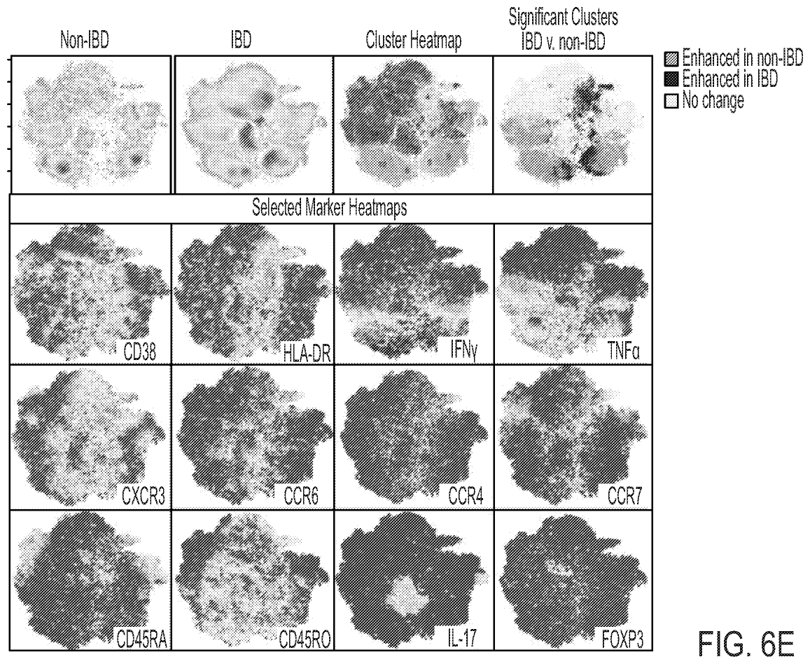

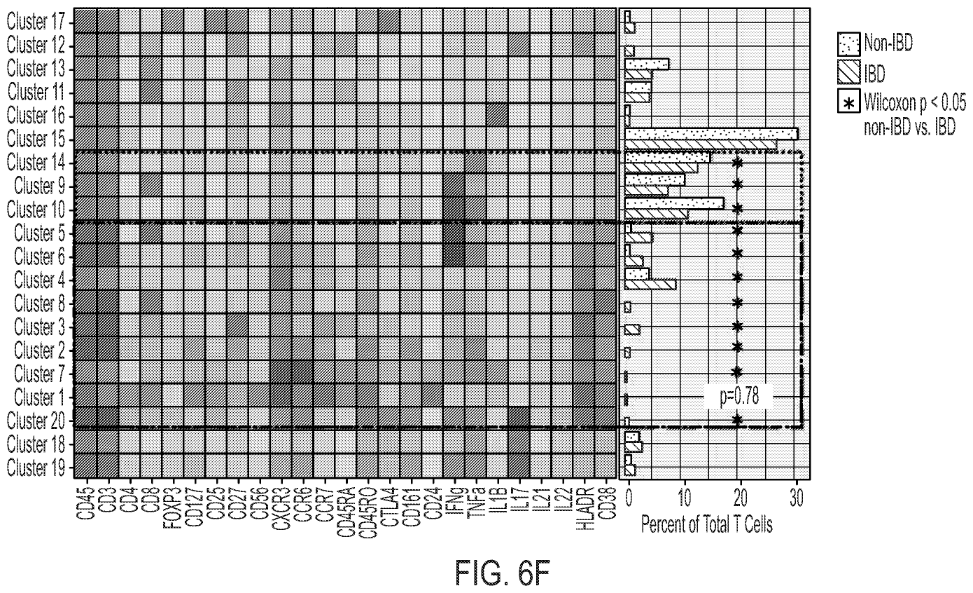

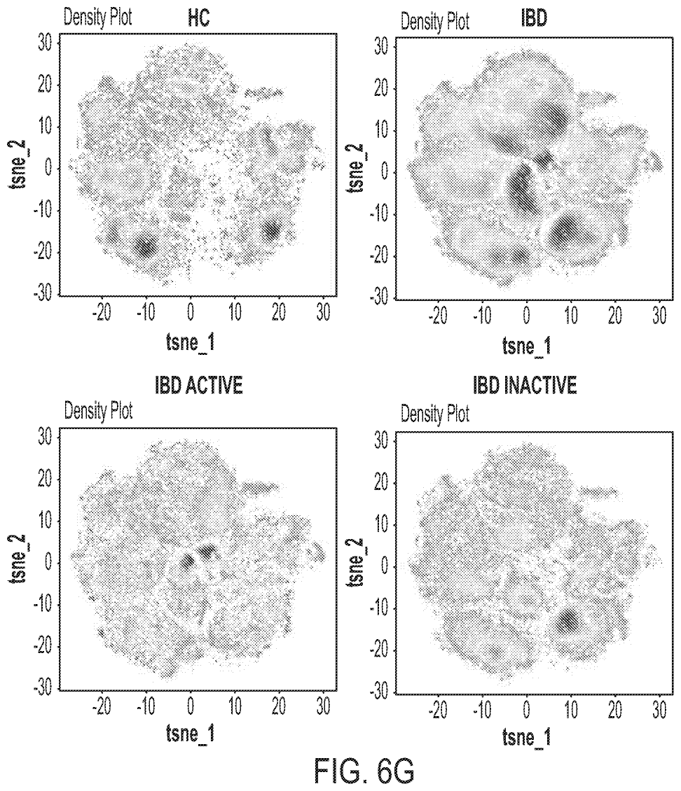

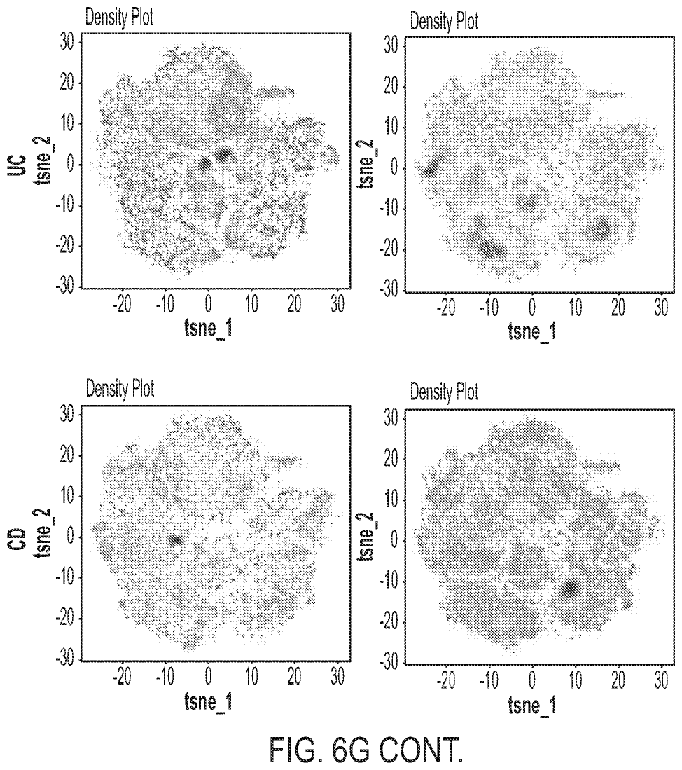

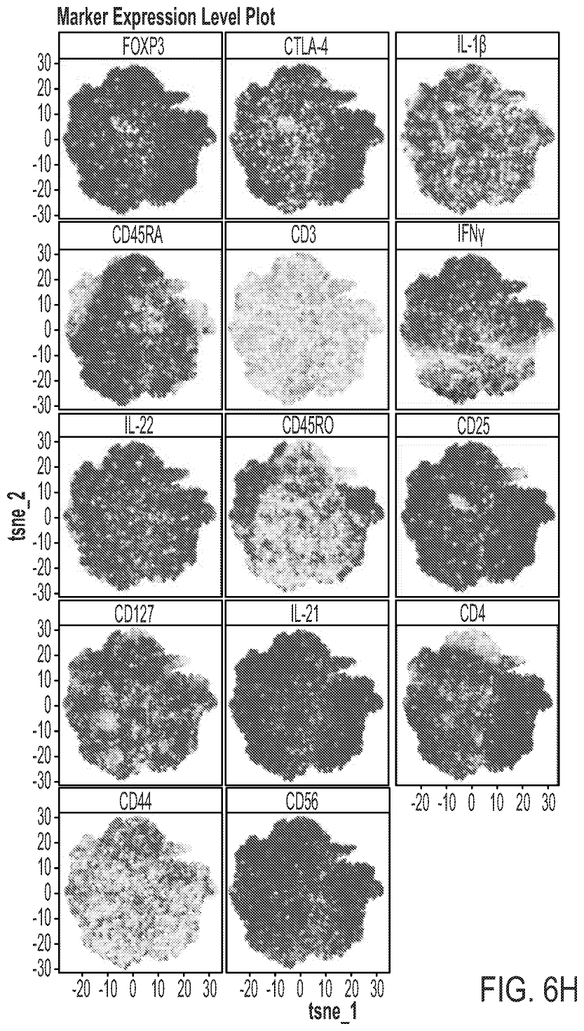

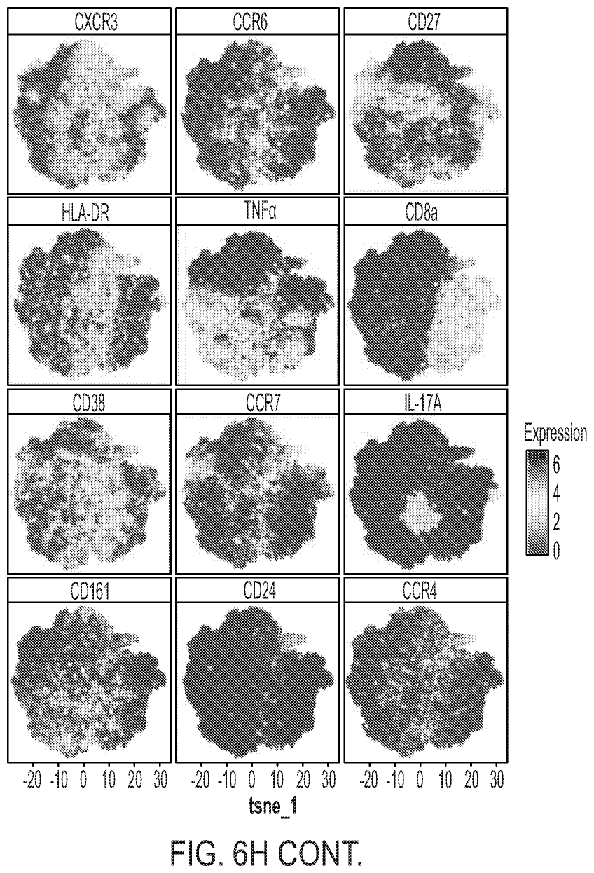

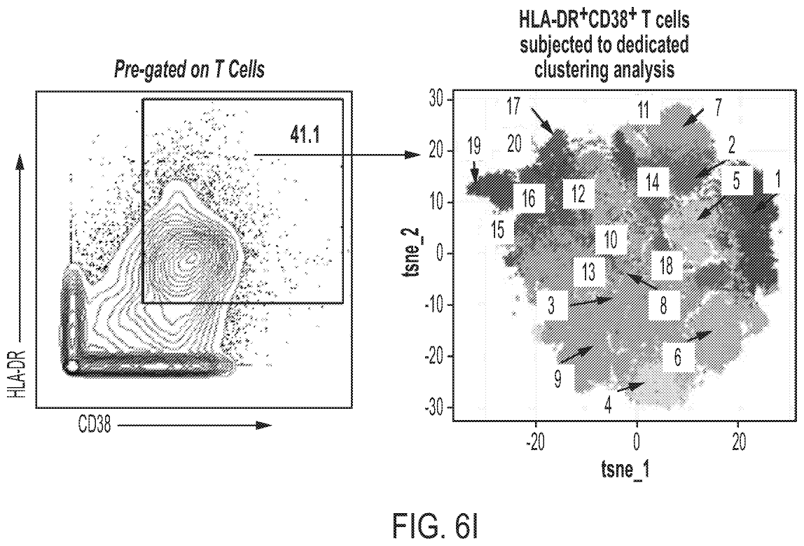

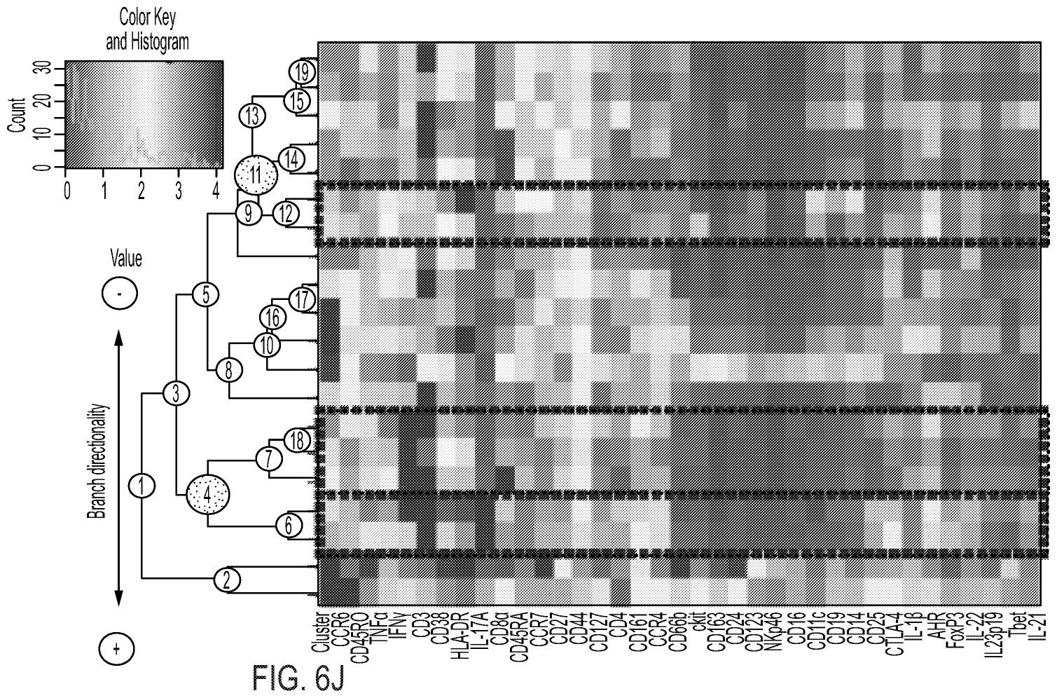

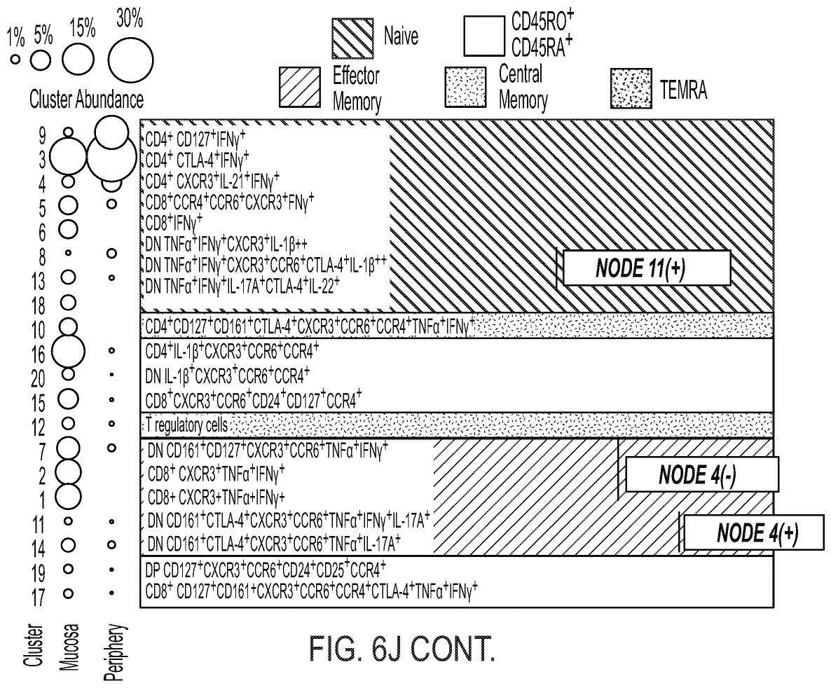

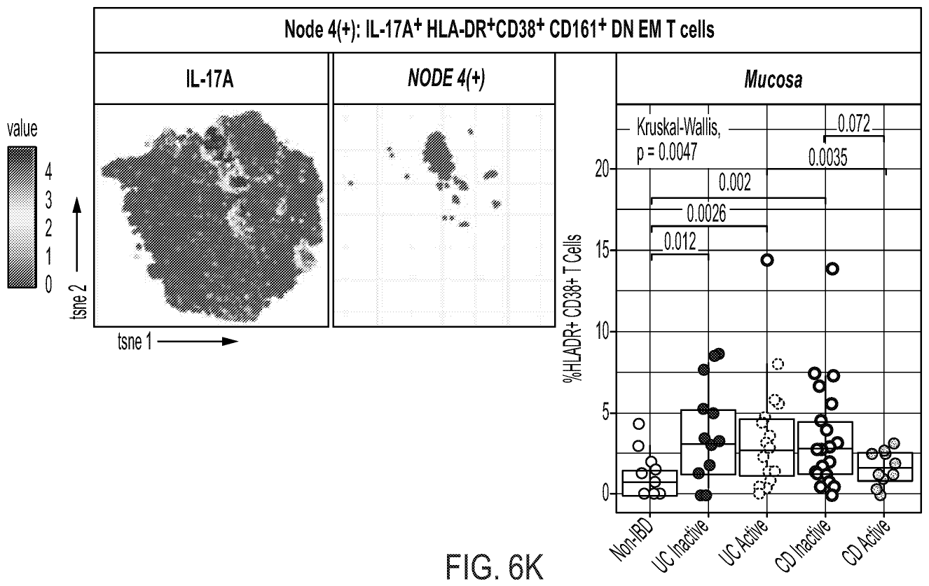

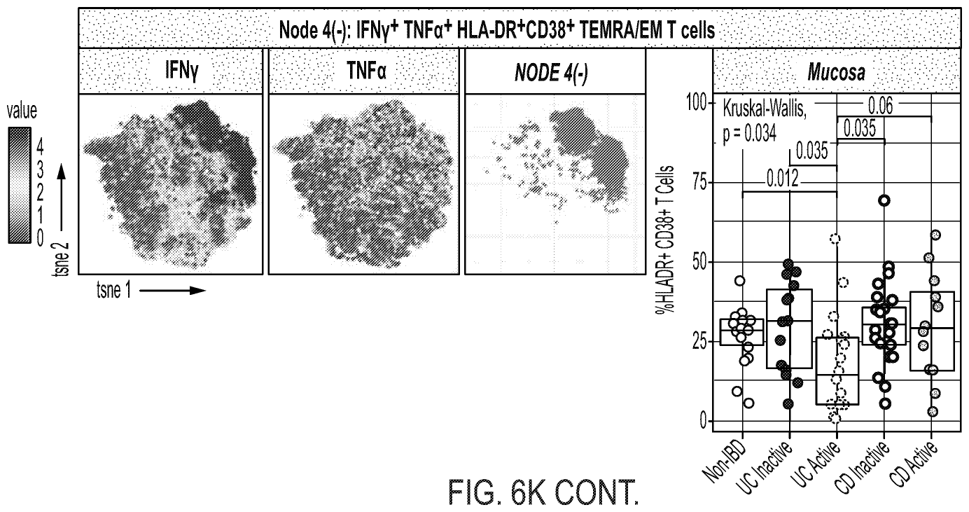

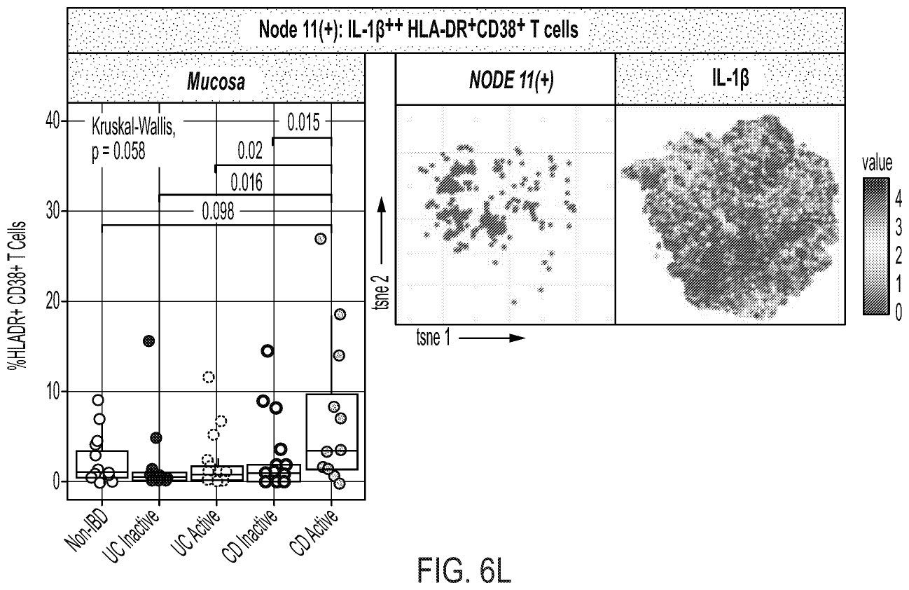

The proportions of HLA⁻DR⁺CD38⁺ T cells were increased in active IBD (active ulcerative colitis/active Crohn's disease) and inactive Crohn's disease mucosa compared to control samples across all examined T cell subsets including CD4⁺ T cells, conventional T cells (Tcons), CD8⁺ T cells, FOXP3⁺ regulatory T cells (Tregs, addressed separately below), and double-negative T cells (FIG. 6A). To better define HLA-DR⁺CD38⁺ T cell populations, a comprehensive analysis of marker expression was performed. In CyTOF analysis, T cell clusters co-expressing HLA-DR and CD38 (Node 4(−)) were increased in IBD mucosa (FIG. 6B) and this finding was validated by flow cytometry (FIG. 6C). Across almost all mucosa T cell subsets, HLA-DR and CD38 expression was associated with enhanced expression of CD45R isoforms (CD45RO and CD45RA), chemokine receptors (CCR4, CCR6, CCR7, and CXCR3) (e.g. clusters 9 and 15) (FIG. 4D). and IL-1β (FIG. 6D).

Automated, unbiased analysis of 20 clusters within the T cell compartment (FIGS. 6E to 611) using FlowSOM identified and confirmed expansion of HLA-DR⁺CD38⁺ T cell clusters in IBD (1-8 and 20) as well as co-expression of CD45R isoforms and chemokine receptors in these clusters (FIGS. 6E and 6F). Non-IBD tissue was characterized by expansion of several HLA⁻DR⁻/CD38⁻ T cell clusters (FIGS. 6E and 6F). Although these HLA-DR⁻CD38⁻ T cells expressed minimal chemokine receptors, they robustly expressed TNFα and/or IFNγ (FIGS. 6E-6F).

In contrast to prior reports (Joosse et al., Mucosal Immunol., 12:154-63 (2019)), expansion of HLA-DR⁺CD38⁺ T cells was not observed in the peripheral blood of IBD subjects (FIG. 2C). However, in the peripheral blood, HLA-DR⁺CD38⁺ T cells that were present were also characterized by increased expression of central memory T cell markers, markers of activation, and pro-inflammatory cytokines including CD45RO, CCR7, CTLA-4, and IL-1β respectively (FIG. 6A).

To better phenotype HLA-DR⁺CD38⁺ T cells, this population was manually gated and performed a dedicated clustering analysis (FIG. 6I, 6J). HLA-DR⁺CD38⁺ T cells were found to represent a mix of T cell subtypes (CD4, CD8 and DN T cells, T regulatory cells (Tregs)) and memory states (FIG. 6J). Similar to the findings in T cells at large, HLA-DR⁺CD38⁺ T cells co-expressing TNFα+IFNγ+ (Node 4(−) in FIG. 6J) were diminished in UCa mucosa whereas IL-17A⁺HLA-DR⁺CD38⁺CD161⁺ DN EM T cells (Node 4(+) in FIG. 6J) were enhanced (FIG. 6K). IL-1β⁺HLA-DR⁺CD38⁺ T cells (Node 11(+) in FIG. 6J) demonstrated a strong trend towards expansion in CDa mucosa (FIG. 6L).

### Example 6: FOXP3⁺ Tregs are Increased in Abundance in Active IBD Mucosa and Demonstrate Increased Expression of Pro-Inflammatory Cytokines (IL-17A, IL-1β)

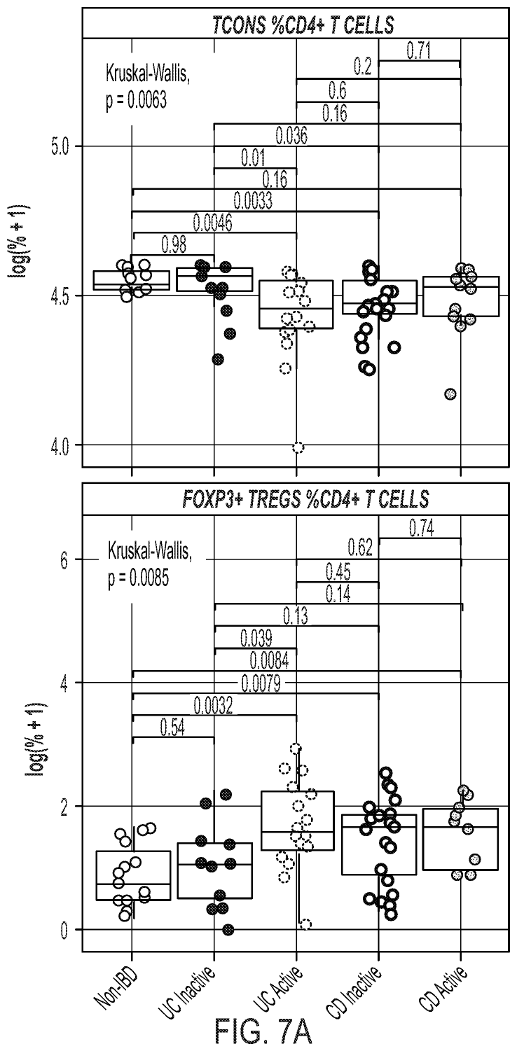

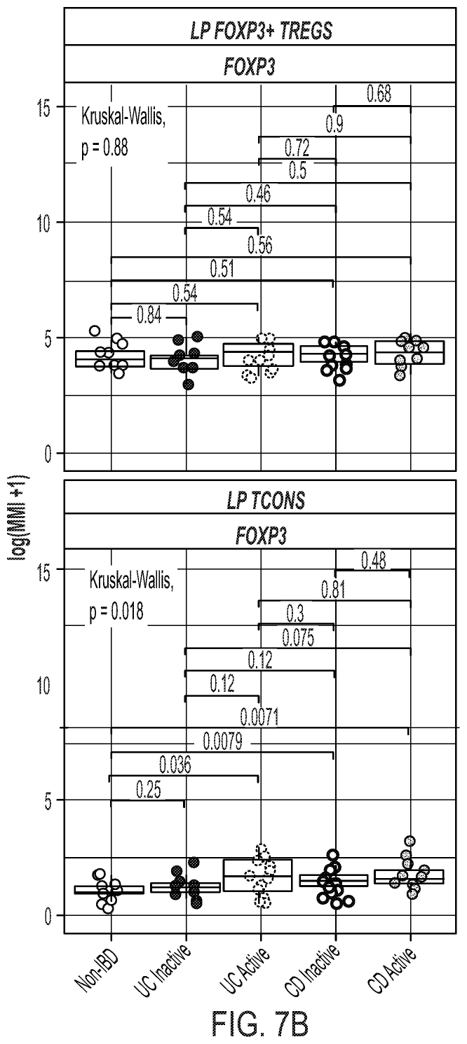

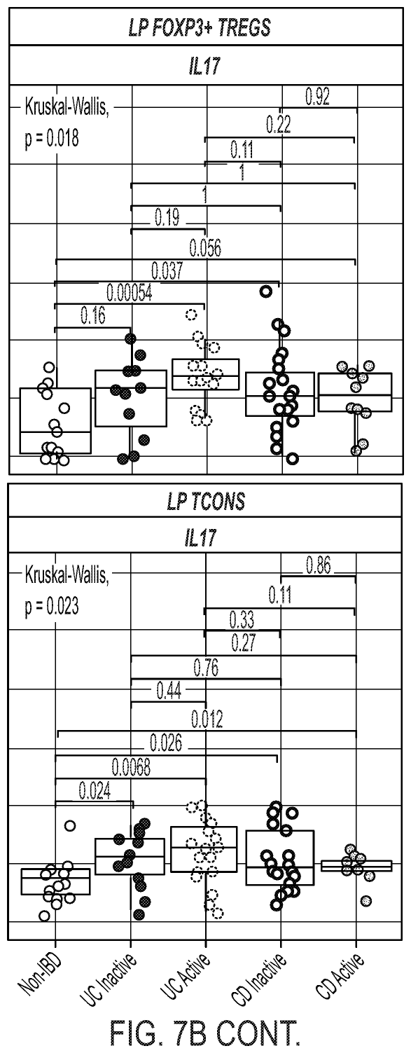

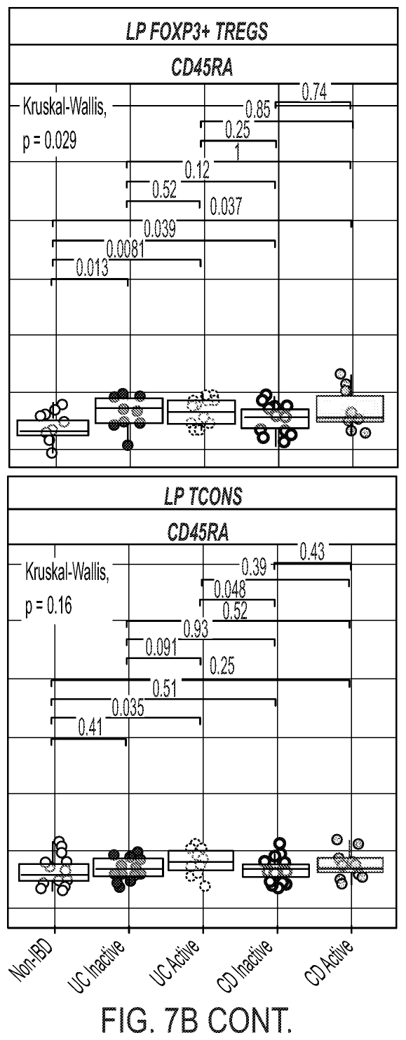

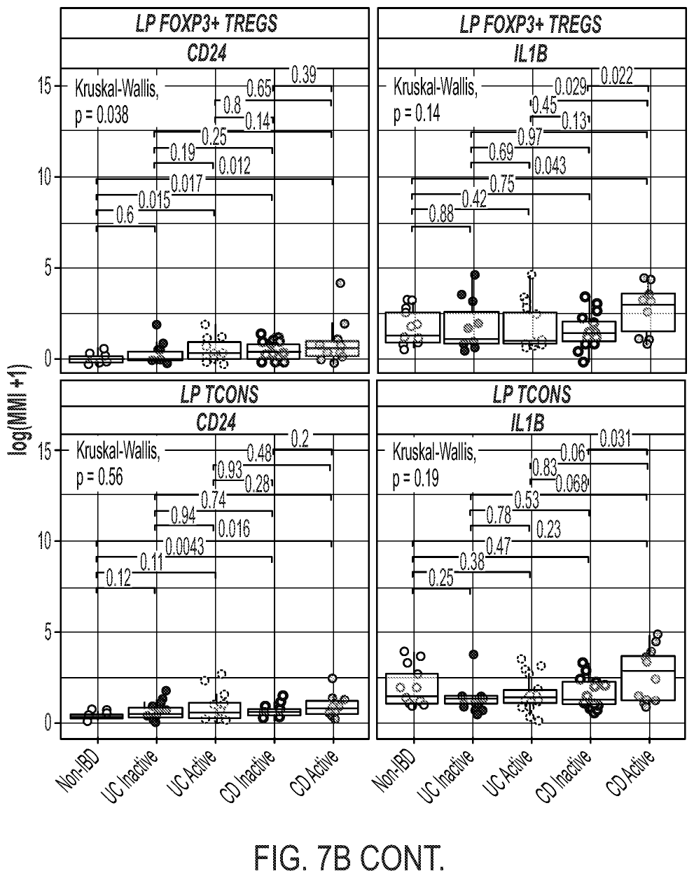

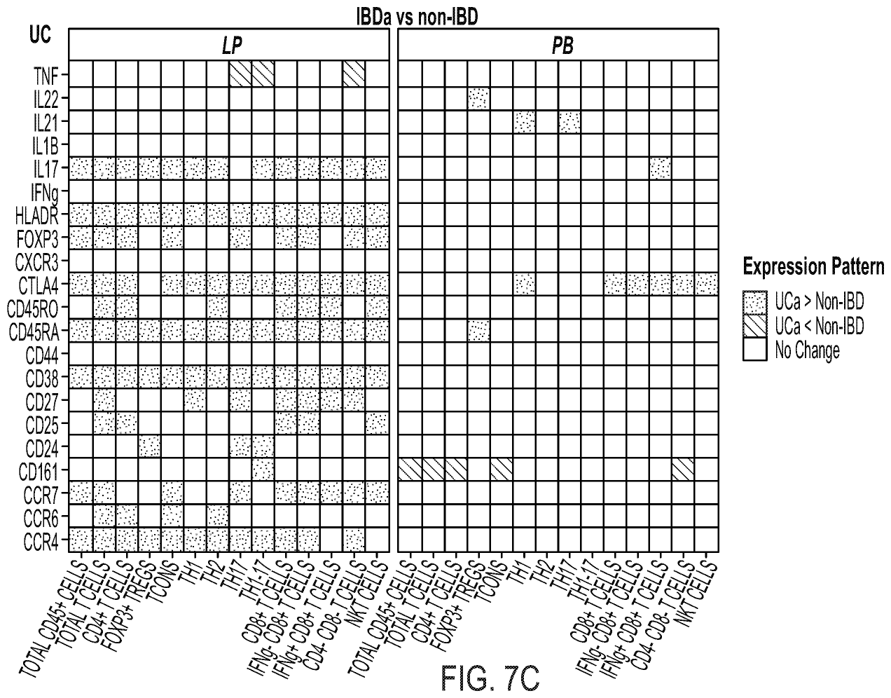

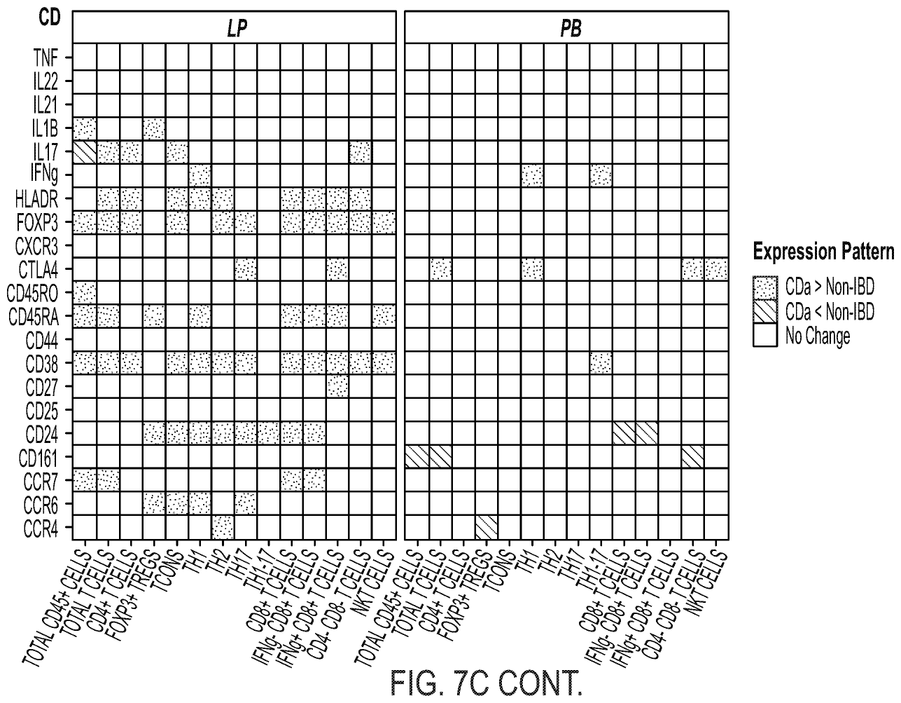

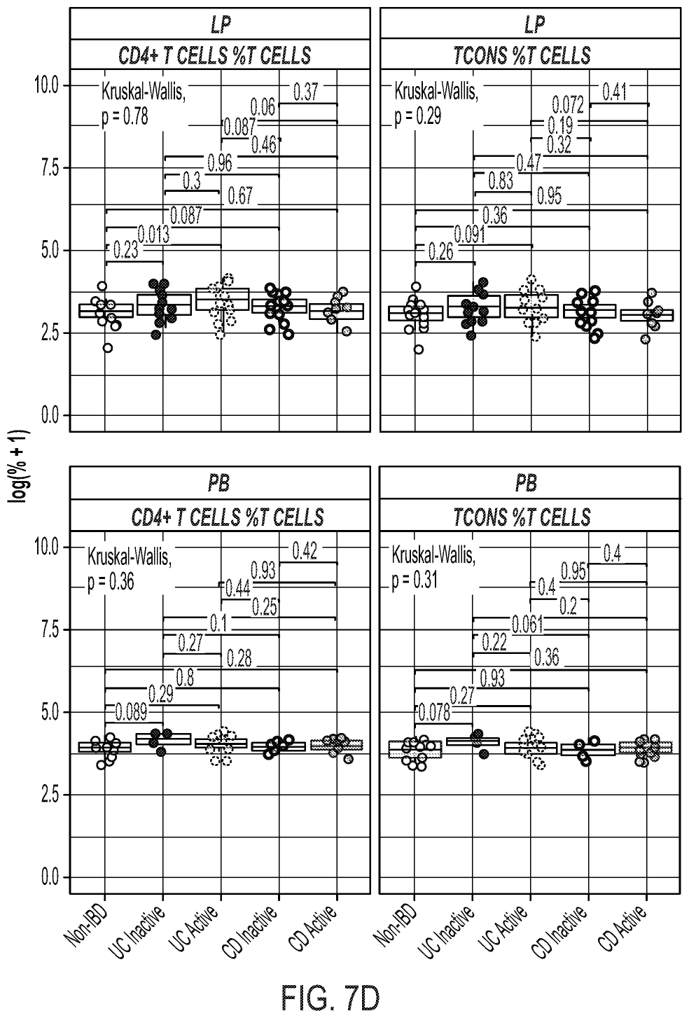

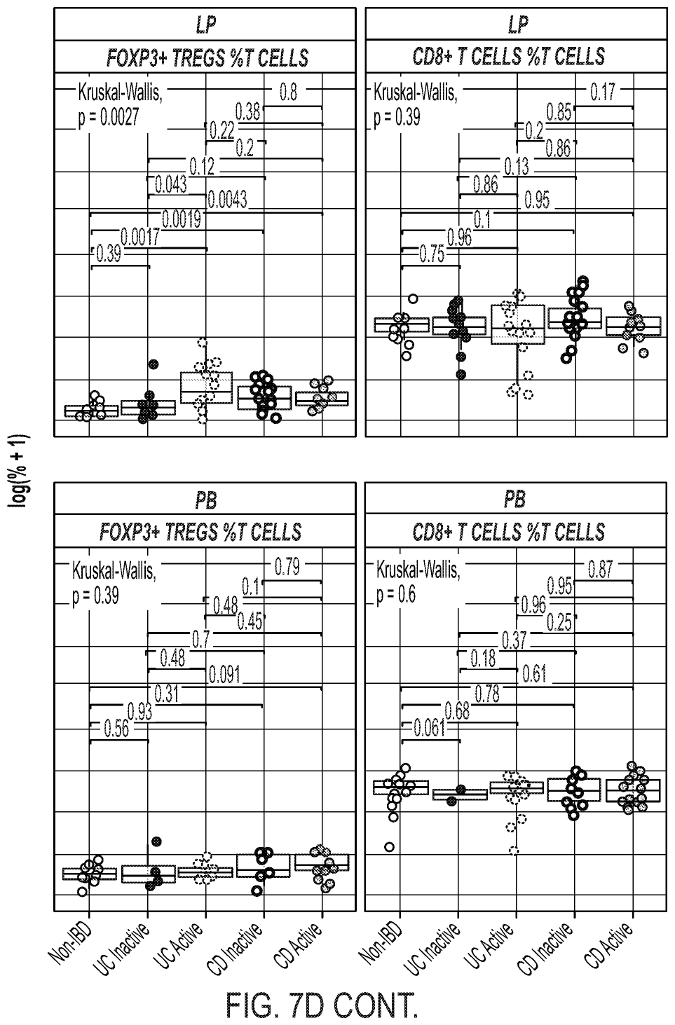

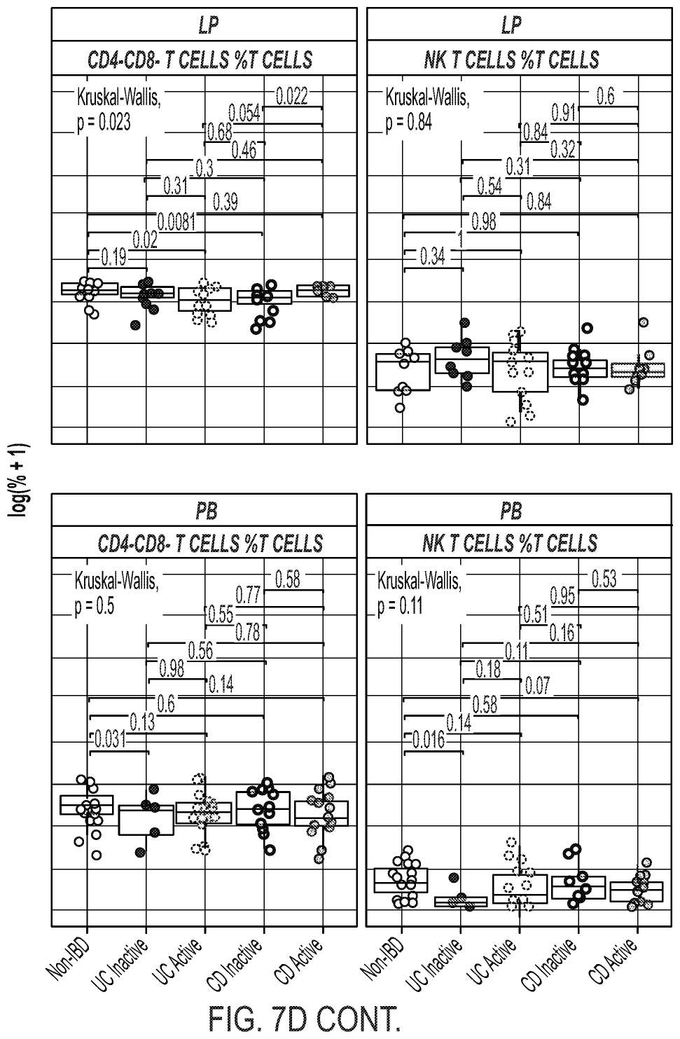

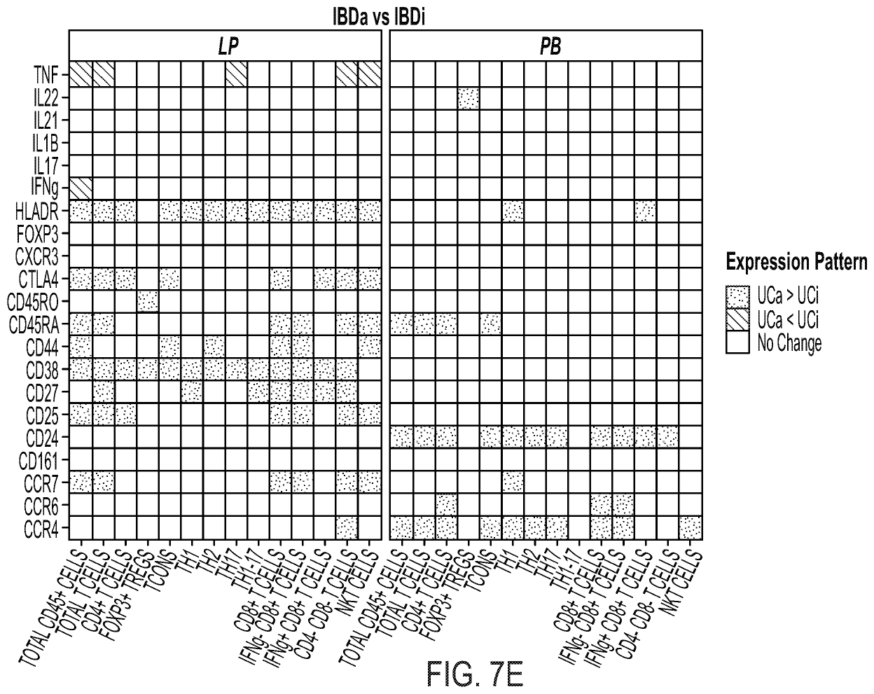

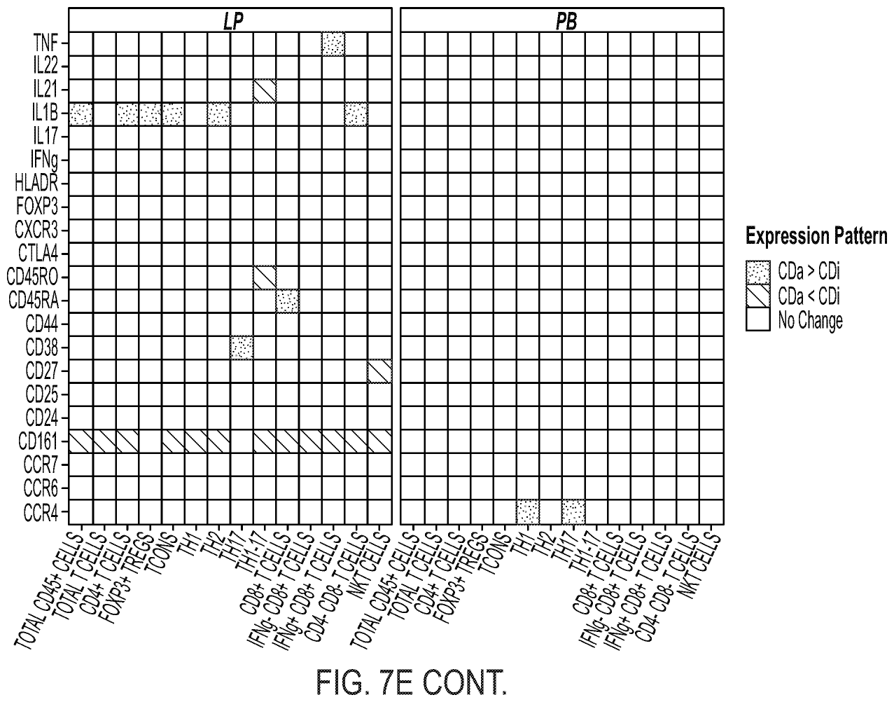

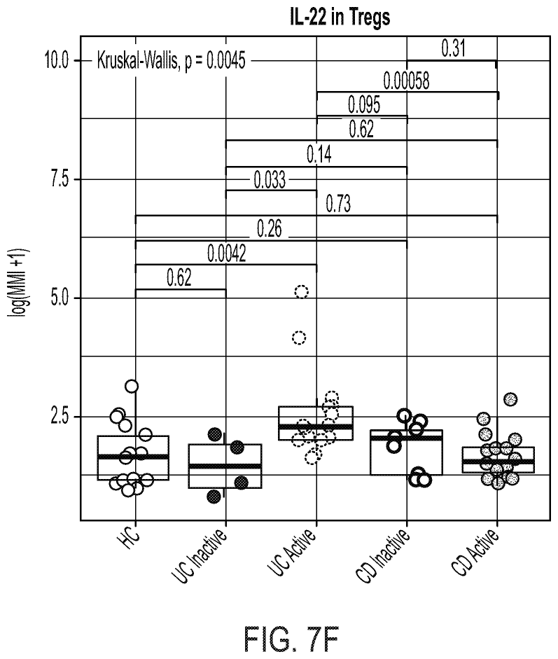

The abundance of FOXP3⁺ Tregs out of total CD4⁺ T cells was increased in IBD mucosa (active ulcerative colitis/active Crohn's disease/inactive Crohn's disease) compared to non-IBD controls (FIG. 7A), consistent with previous published data (Lord J D, World J Gastroenterol, 21:11236-45 (2015)). FOXP3⁺ regulatory T cells in active IBD tissue (active ulcerative colitis/active Crohn's disease) were characterized by enhanced expression of CD45RA and CD24, both markers of naïve/immature regulatory T cells (FIG. 7B). As has been previously described for CD27, IL-17A expression was increased in mucosa FOXP3⁺ regulatory T cells in IBD (active ulcerative colitis/inactive Crohn's disease, trend for active Crohn's disease) compared to non-IBD (FIG. 7B). Furthermore, active Crohn's disease FOXP3⁺ regulatory T cells had increased expression of IL-1β compared to both active ulcerative colitis and non-IBD (FIG. 7B). Although T cells do not classically express IL-1β, its expression by CD4⁺ human lymphocytes has been previously described (Arbore et al., Science, 352:aad1210 (2016)), but not in regulatory T cells. The data presented herein supports the possibility that regulatory T cells in active IBD tissue may be altered, possibly with pathogenic potential, as has been described for other inflammatory disorders (Esposito et al., J Immunol., 185:7467-73 (2010)).

Interestingly, active IBD (active ulcerative colitis/active Crohn's disease) mucosa could be distinguished from non-IBD by enhanced expression of FOXP3 in non-regulatory T cells, including CD25⁻CD4⁺ T cells (Tcons) and CD8⁺ T cells (FIGS. 7B, 7C). In contrast to prior reports (Maul et al., Gastroenterology, 128:1868-78 (2005); Saruta et al., Clin Immunol, 125:281-90 (2007)), no difference was observed in peripheral abundance of FOXP3⁺ Tregs in IBD compared to control (FIG. 7D). However, IL-22 expression was increased in peripheral active ulcerative colitis FOXP3⁺ Tregs compared to active Crohn's disease, inactive ulcerative colitis, and non-IBD (FIGS. 7C, 7E, and 7F), which has not previously been described and could reflect altered functionality of Tregs in disease.

### Example 7: Memory Tregs are Increased in Abundance in Active IBD Mucosa and Express Pro-Inflammatory Cytokines

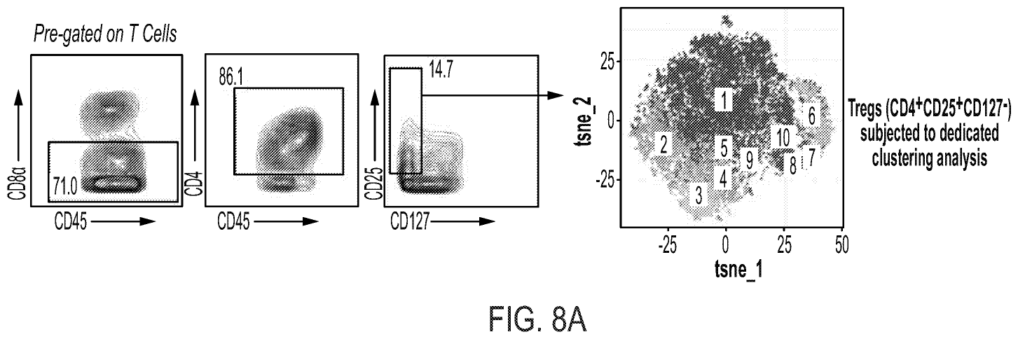

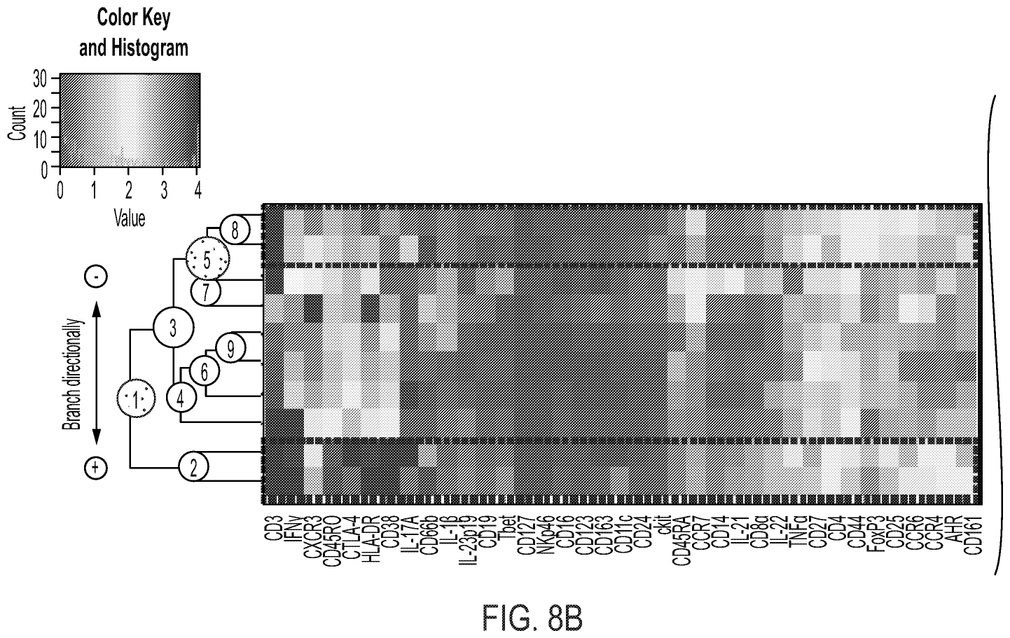

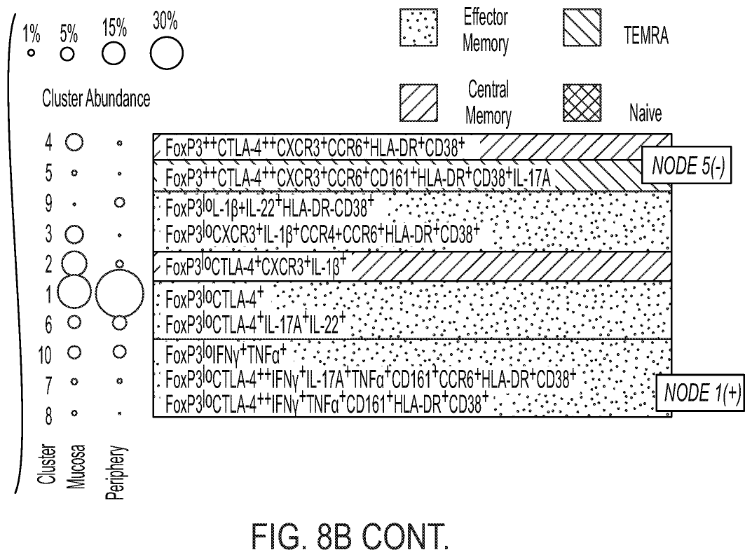

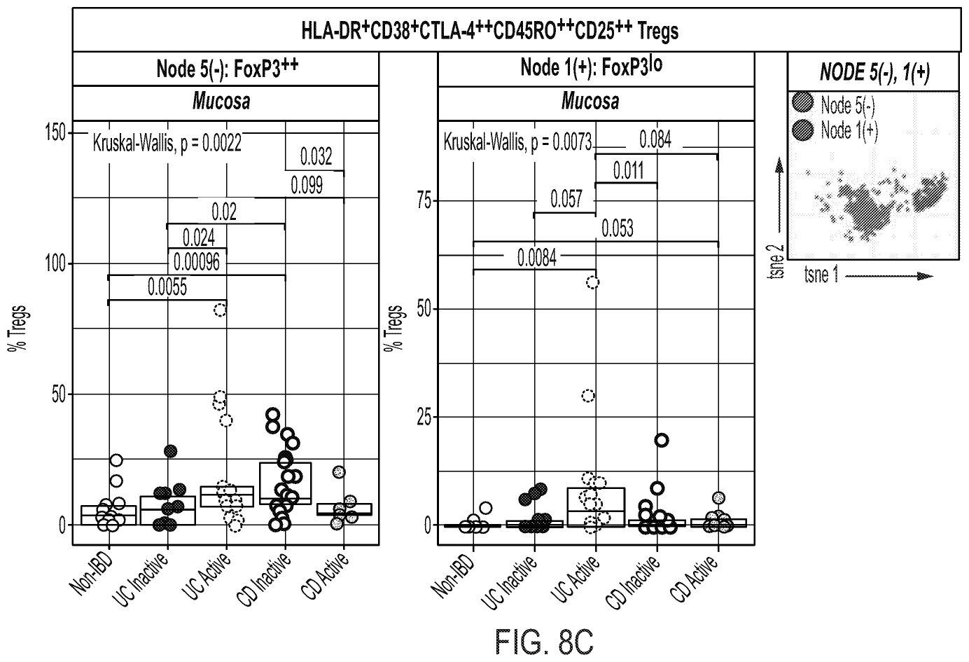

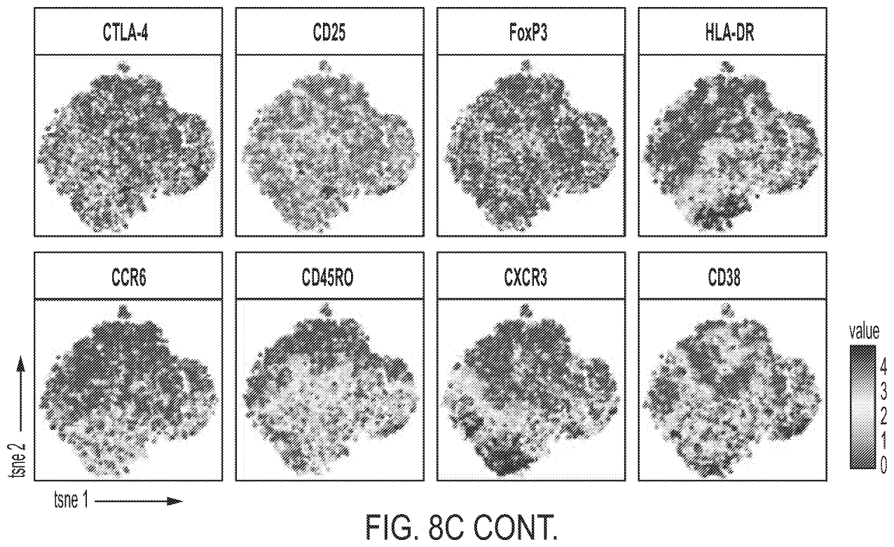

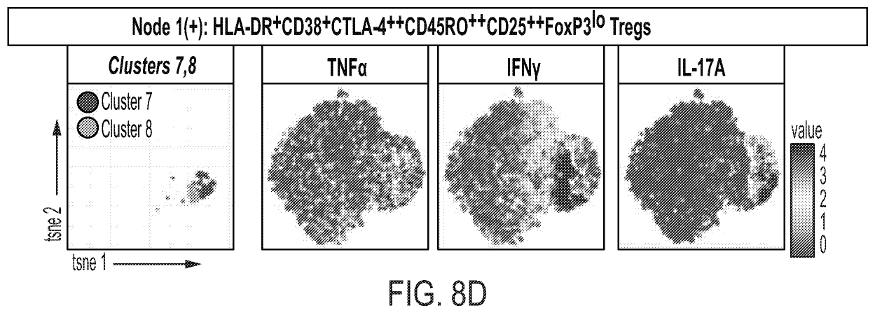

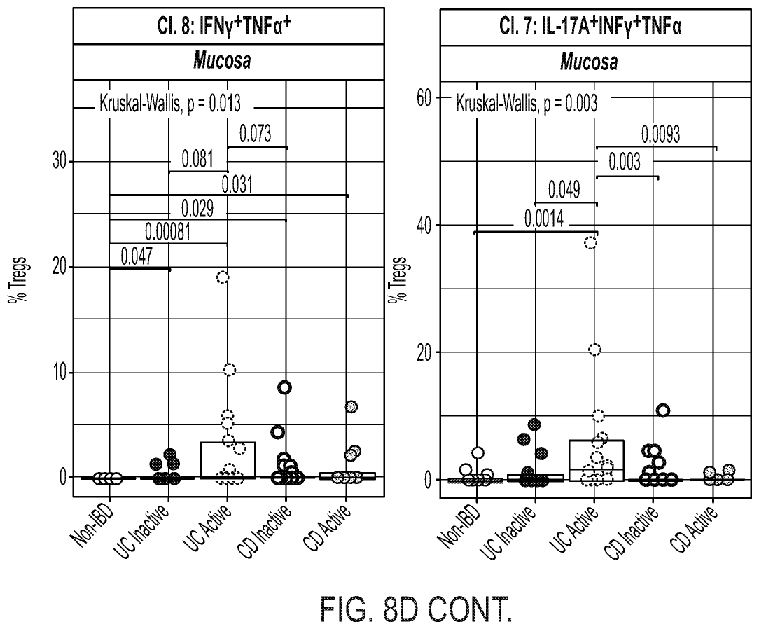

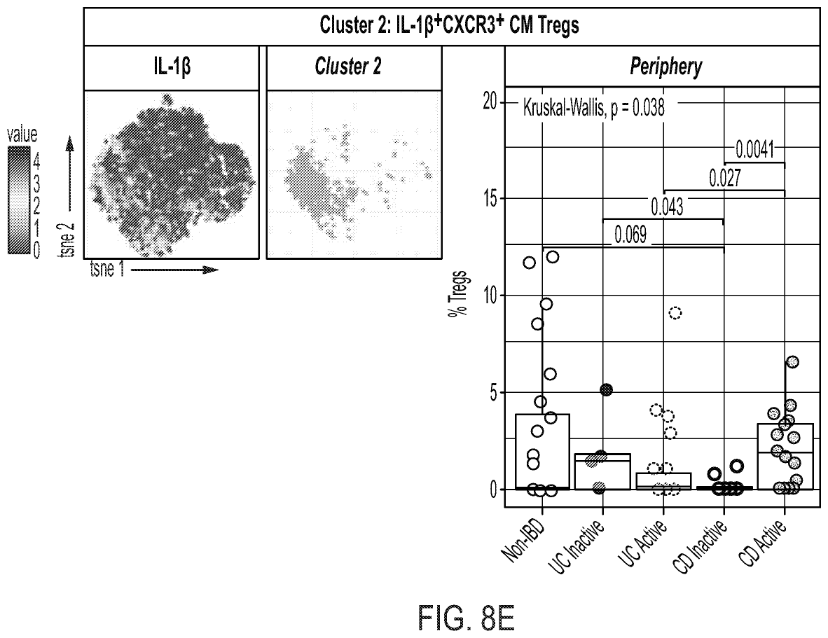

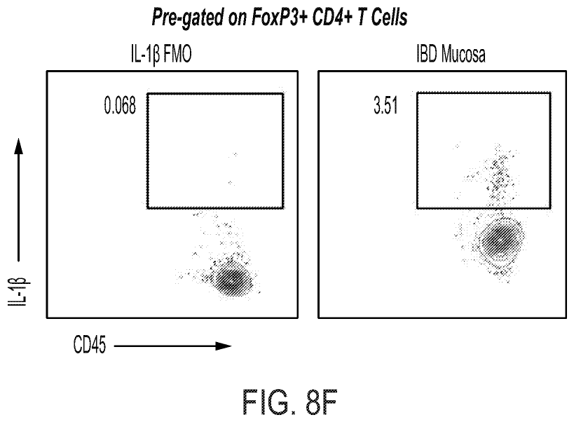

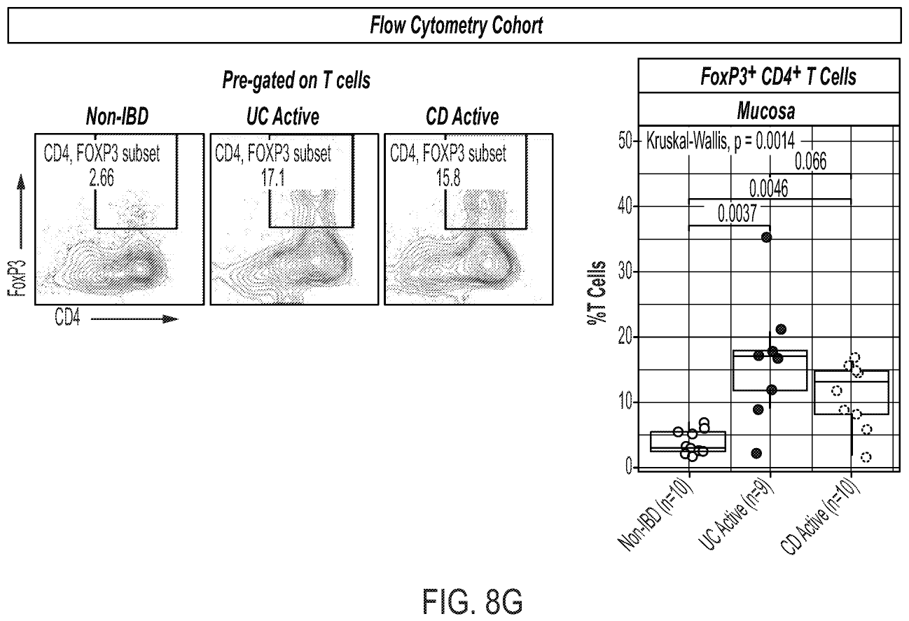

Regulatory T cells (Tregs) were manually gated (as CD4⁺CD25⁺CD127⁻ T cells) and subjected to dedicated automated analysis (FIGS. 8A, 8B). Memory Treg subsets were assigned according to the same algorithm detailed for other T cells. disease-specific signatures were identified in Tregs that mirrored findings in the preceding T cell analyses, including HLA-DR and CD38 co-expression in IBD mucosa and IL-17A expression in UCa mucosa.

Treg populations enhanced in IBD mucosa were HLA-DR⁺CD38⁺, especially in UCa (Node 5(−), Node 1(+) in FIGS. 8B, 8C), similar to the findings in preceding T cell analyses. HLA-DR⁺CD38⁺ IBD-associated Tregs co-expressed various chemokine receptors including CCR6 and CXCR3 and were uniformly CD25⁺⁺CTLA-4⁺⁺CD45RO⁺⁺, suggesting an activated memory phenotype (FIGS. 8B, 8C). Some branches were characterized by FoxP3⁺⁺ expression, perhaps signifying high suppressive capacity (Node 5(−)). Others, e.g., Clusters 7 and 8 (Node 1(+)), were FoxP3lo and expressed pro-inflammatory cytokines (TNFα⁺IFNγ⁺IL-17A^{+/-}), possibly indicating non-suppressive or even non-Treg properties (FIGS. 8B, 8C).

Cluster 8, which co-expressed IFNγ and TNFα, was increased in both UCa and CDa, whereas Cluster 7, which co-expressed IFNγ, TNFα, and IL-17A, was specifically increased in UCa mucosa (FIG. 8D). Only one Treg cluster differed in peripheral disease: Cluster 2, IL-1 β⁺CXCR3⁺ CM Tregs, enhanced in CDa compared to UCa and CDi (FIG. 8E). Given the novelty of identifying IL-1β expression in Tregs, this finding was confirmed using flow cytometry (FIG. 8F). The data supports the possibility that Tregs in active IBD tissue may be altered, possibly with pathogenic potential, as has been described in IBD and other inflammatory disorders (Koenecke C et al., J Immunol 2012; 189:2890-6; Esposito M et al., J Immunol 2010; 185:7467-73.

Finally, as has been previously reported (Maul J et al., Gastroenterology 2005, 128:1868-78; Holmen N et al., Inflammatory bowel diseases 2006, 12:447-56), manual gating of two independent cohorts with CyTOF or flow cytometry revealed increased Tregs in IBD compared to non-IBD mucosa (FIG. 4A, FIG. 8G). In contrast to some prior reports (Maul J et al., Gastroenterology 2005), no difference was observed in peripheral abundance of Tregs in IBD (FIG. 4A).

### Example 8: IL-17A Expressing T Cells Predominate Ulcerative Colitis Mucosa; IL-1β, IFNγ, and TNFα Expressing T Cells Predominate Crohn's Disease Mucosa

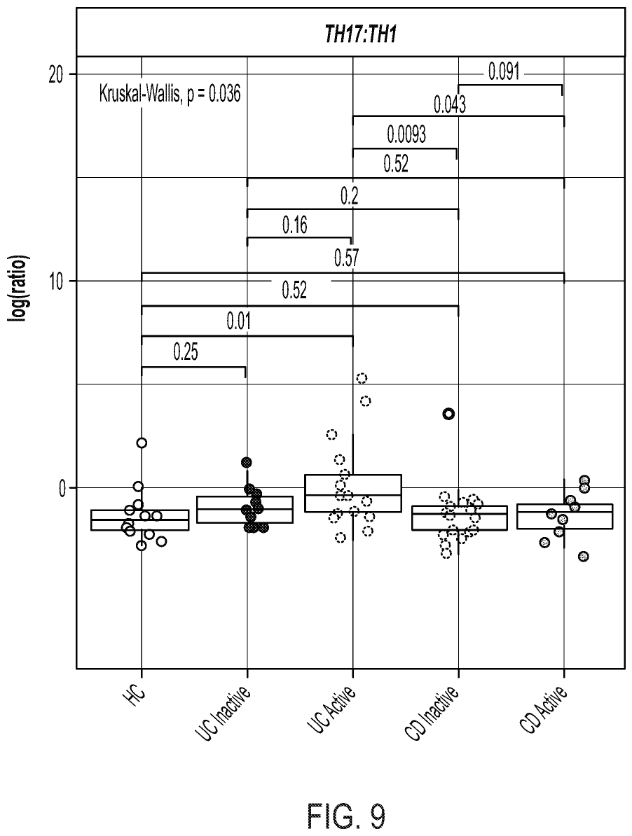

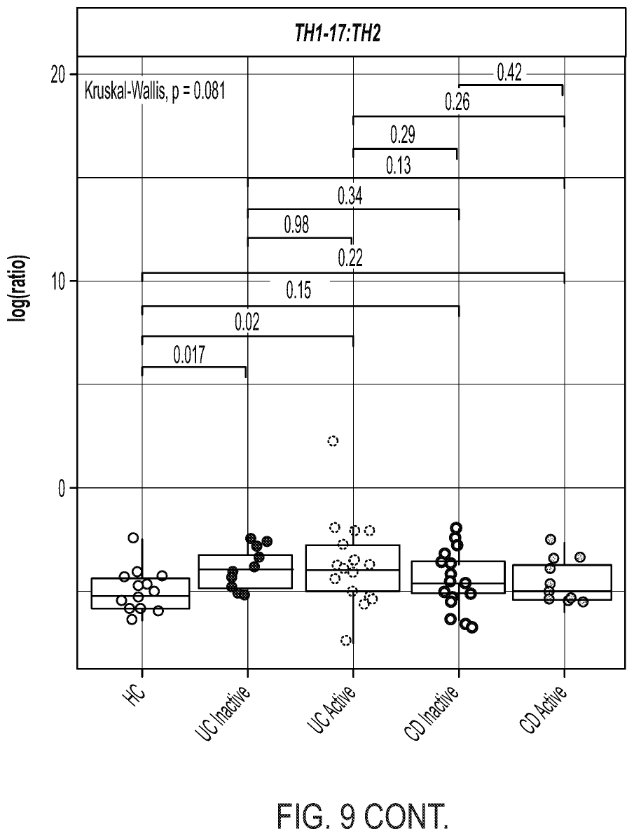

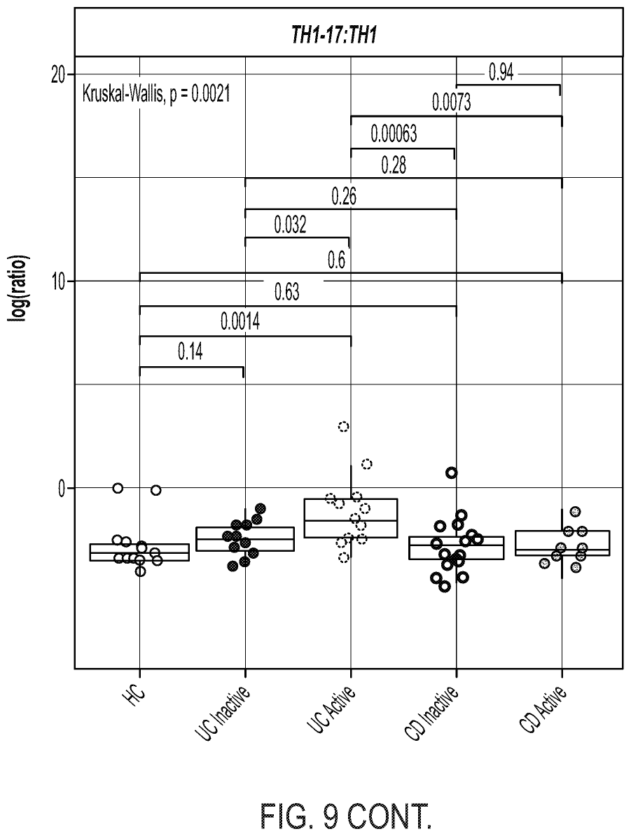

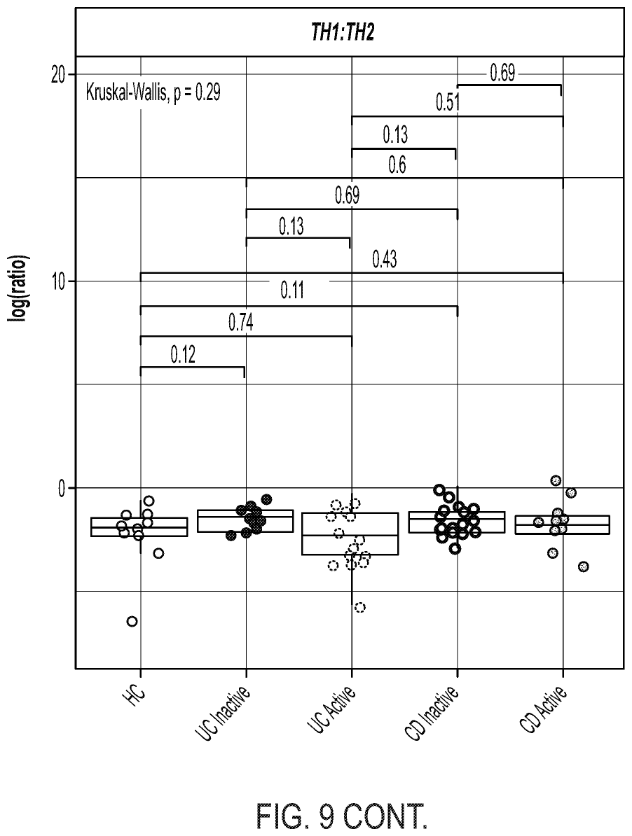

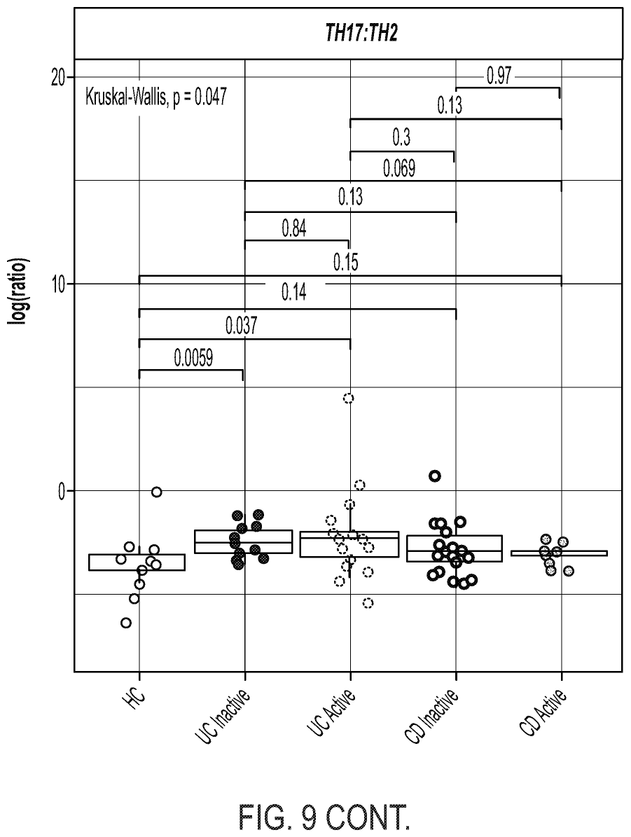

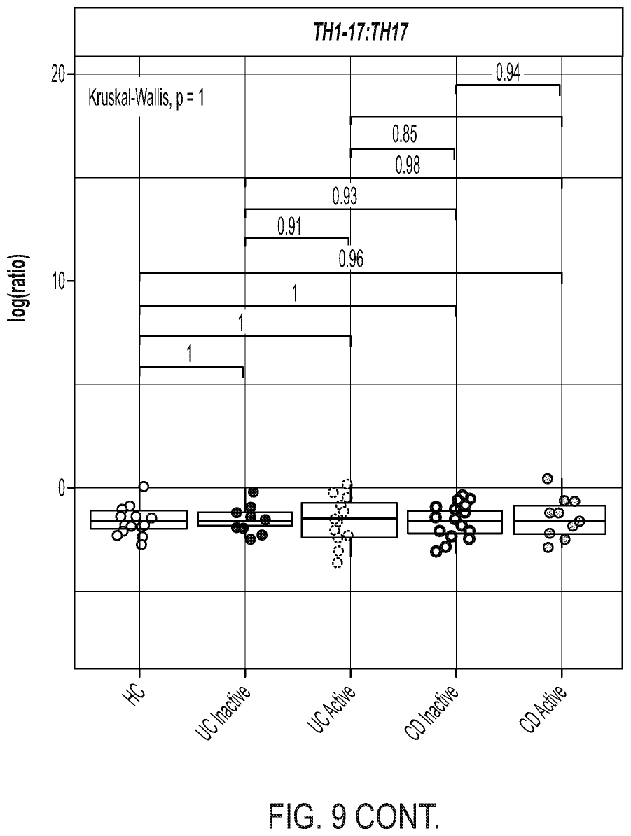

In contrast to the expansion of FOXP3⁺ regulatory T cell population in active IBD mucosa, the abundance of Tcons was diminished in IBD mucosa (active ulcerative colitis/inactive Crohn's disease) compared to non-IBD controls (FIG. 7A). To further characterize T helper (TH) cell subsets and their cytokine production, Tcons were subtyped according to their expression of IFNγ and IL-17A. Active ulcerative colitis mucosa was found to have increased IL-17A over IFNγ expression in TH cells, with an increased ratio of Th17:Th1 and Th1-17:Th1 compared to active Crohn's disease and non-IBD (FIG. 9). Although TH2 cytokines were not directly examined, this data expands upon the traditional understanding of ulcerative colitis as being “TH2-predominant” and Crohn's disease as “TH1-predominant.” This indicates that ulcerative colitis is dominated by IL-17A expressing TH cells.

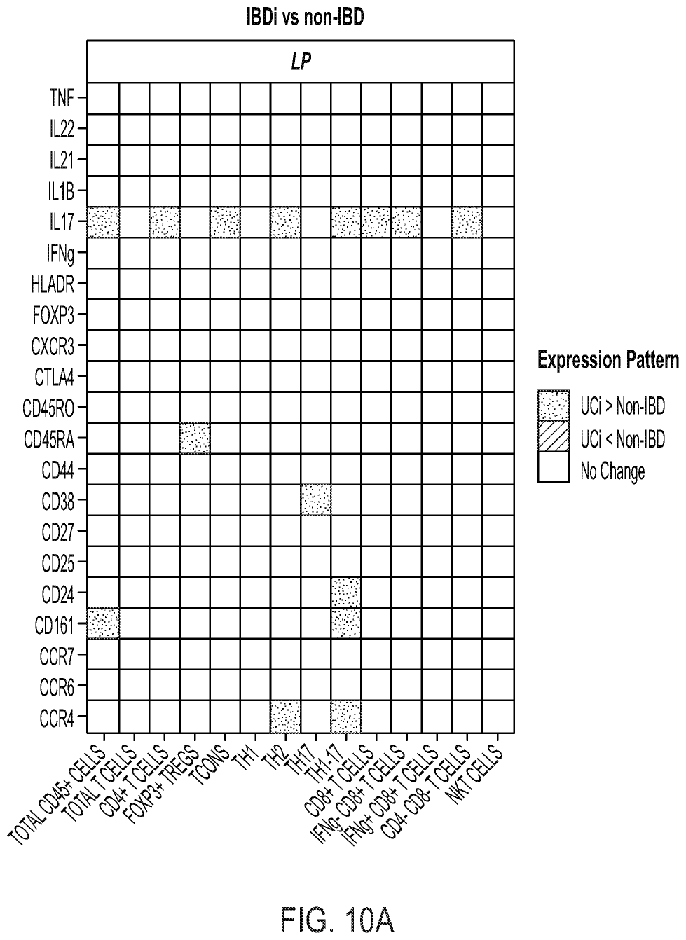

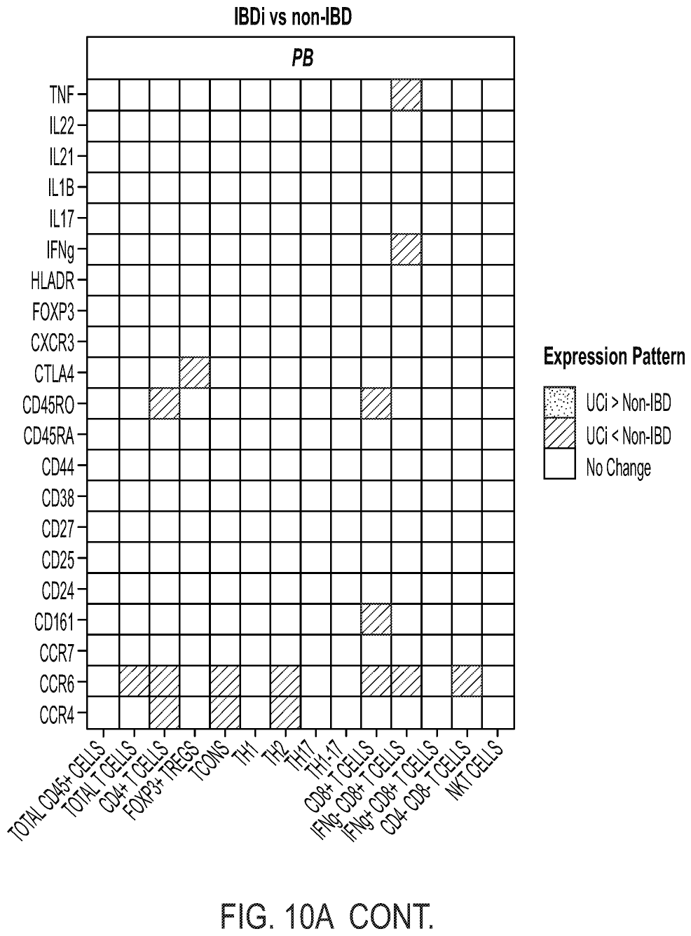

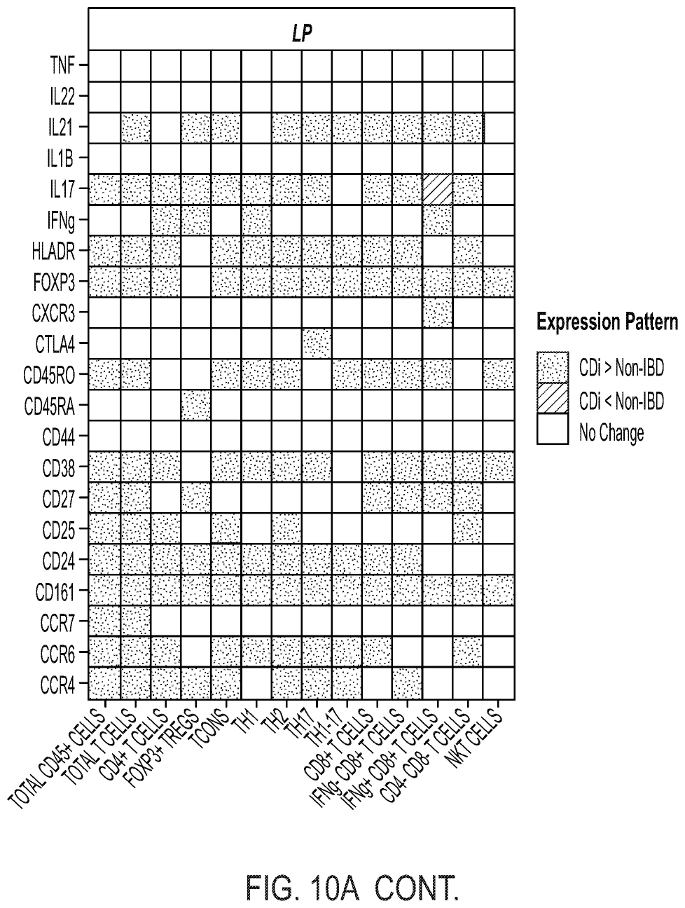

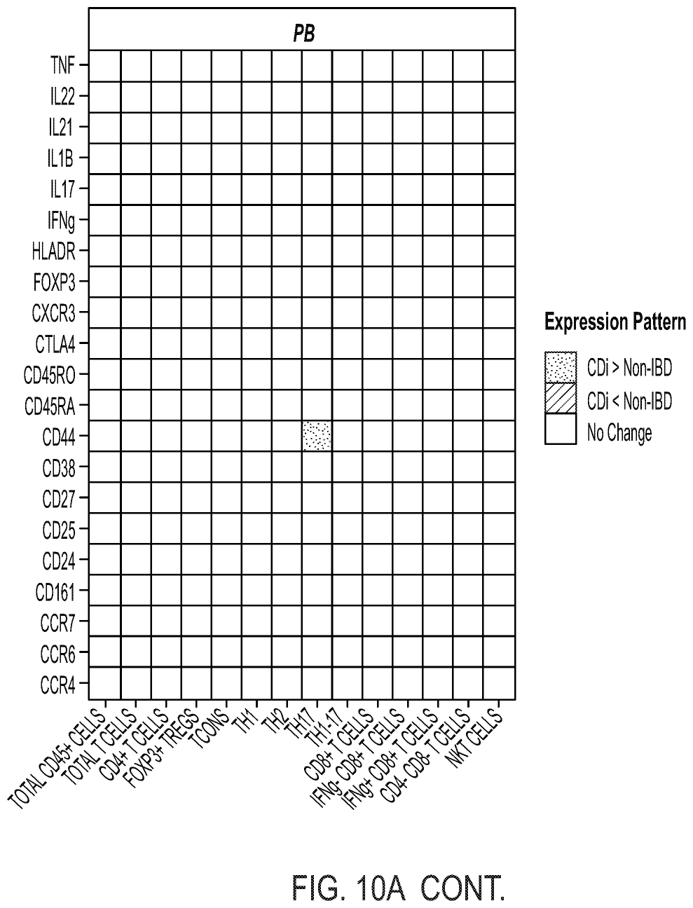

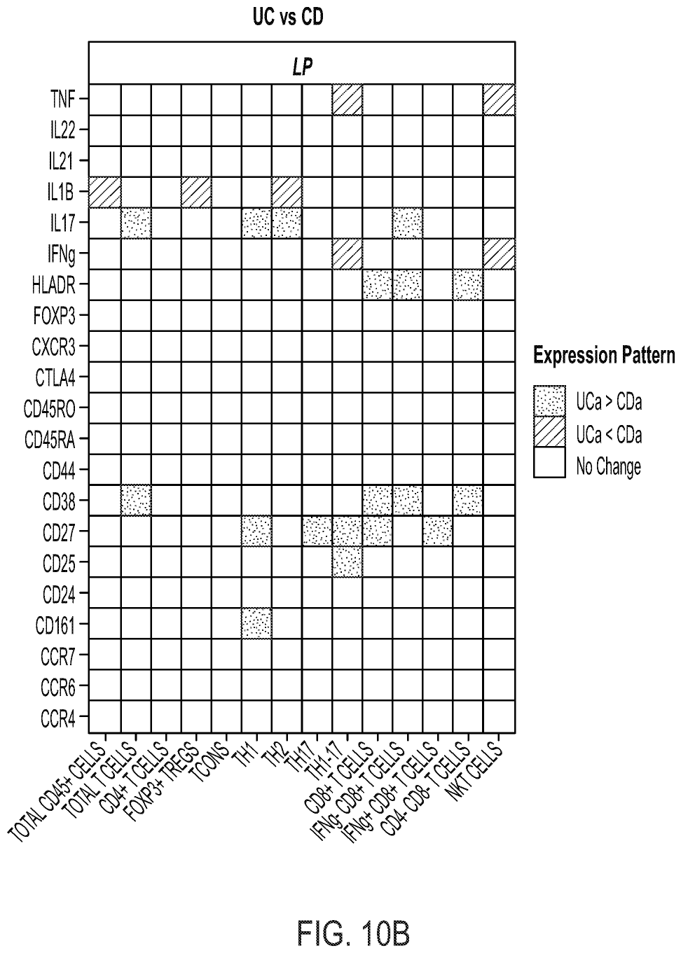

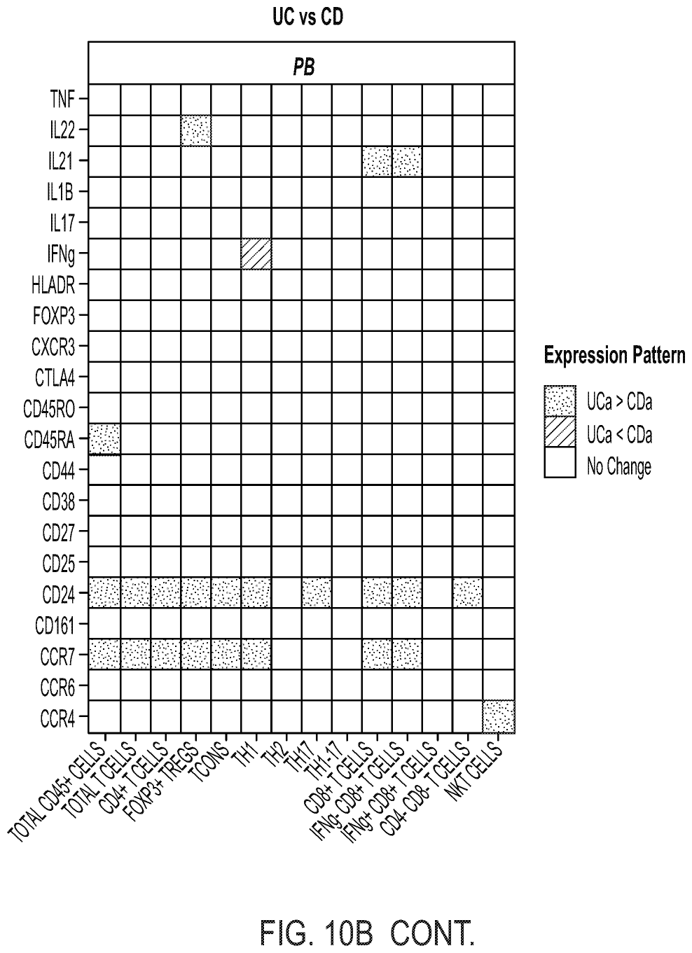

In addition to the findings described above, global trends in differential marker expression were observed in the T cell compartment across subject groups that could differentiate IBD patient types. This was derived from a comparison of marker expression across manually gated T cell subsets that was performed between active IBD and inactive IBD (ulcerative colitis/Crohn's disease), active IBD and non-IBD (active ulcerative colitis/active Crohn's disease), inactive IBD and non-IBD (inactive ulcerative colitis/inactive Crohn's disease) and between active ulcerative colitis and active Crohn's disease (FIGS. 7C, 7D, 10A, 10B). Hallmarks of active ulcerative colitis mucosa T cells other than those already mentioned (e.g., increased expression of HLA-DR, CD38, CD45RO, CD45RA, and IL-17A) included increased expression of CCR4, CTLA-4, and CD27, and decreased expression of TNFα in various T cell subsets. For example, when marker expression in active ulcerative colitis mucosa T cell subsets was compared to non-IBD controls, CCR4 expression was found to be enhanced in all CD4⁺ T cell subsets; CTLA-4 enhanced in almost all T cell subsets (except for FOXP3⁺ Tregs); CD27 enhanced in all CD8⁺ T cell subsets; and TNFα reduced in TH1 and TH1-17 and in double negative T cells (FIG. 7C). When compared to inactive ulcerative colitis mucosa T cells, TNFα expression was again found to be decreased in several T cell subsets, including total T cells, TH17 cells, double-negative T cells, and NK T cells (FIG. 7D).

In contrast, hallmarks of active Crohn's disease mucosa T cells other than those already mentioned (e.g., increased expression of HLA-DR, CD38, CD45RO, CD45RA, and IL-1β in FOXP3⁺ regulatory T cells) included increased expression of IL-1β, IFNγ, and TNFα in various T cell subsets. Specifically, IFNγ was increased in Th1 cells when compared to non-IBD (FIG. 7C); TNFα expression was increased in IFNγ⁺CD8⁺ T cells when compared to inactive Crohn's disease (FIG. 7D).

Direct comparison of active ulcerative colitis to active Crohn's disease mucosa T cell marker expression reinforced these signatures. Various active ulcerative colitis mucosa T cell subsets demonstrated increased IL-17A and CD27 expression, whereas various active Crohn's disease mucosa T cell subsets demonstrated increased TNFα, IL-1β, and IFNγ expression (e.g., Th1-17/natural killer T cells and FOXP3⁺ Tregs/Th2 cells and Th1-17/natural killer T cells, respectively) (FIG. 10A).

Interestingly, inactive ulcerative colitis and inactive Crohn's disease mucosa had T cell-specific hallmarks of their own when compared to non-IBD tissue (FIG. 10B). As observed for active ulcerative colitis, numerous inactive ulcerative colitis T cell subsets demonstrated increased expression of IL-17A compared to non-IBD, as well as increased CCR4 expression (TH2/TH1-17 cells) (FIG. 10B). There were extensive differences between inactive Crohn's disease T cells and non-IBD controls, including increased expression of cytokines (IL-17A, and IFNγ), activation markers (HLA-DR and CD38), FOXP3, chemokine receptors (CCR6 and CCR4), memory markers (CD45RO and CD161), and CD24 (FIG. 10B). These results support the conclusion that inactive IBD intestinal tissue, especially inactive Crohn's disease, has its own inflammatory signatures and that the tissue may not return to a normal baseline during periods of disease remission.

Notably, T cell-specific inflammatory signatures were detected in the peripheral blood, although these were less extensive than, and differed from, what was observed in the mucosa. For example, active ulcerative colitis could be distinguished from active Crohn's disease by enhanced CCR7 and CD24 expression in numerous T cell subsets, which indicates a more naïve T cell phenotype in active ulcerative colitis vs active Crohn's disease (FIG. 10A). Active ulcerative colitis could be distinguished from inactive ulcerative colitis by the increased expression of CD24 and CCR4 in most CD4⁺ and CD8⁺ T cells and increased expression of CD45RA in total T cells and Tcons (FIG. 7D). Minimal differences were detected between inactive Crohn's disease, active Crohn's disease, and non-IBD peripheral blood (FIGS. 7C, 10B).

### Example 9: IL-1β⁺IFNγ⁺ TNFα⁺ Naïve B Cells are Enriched in CDa Mucosa

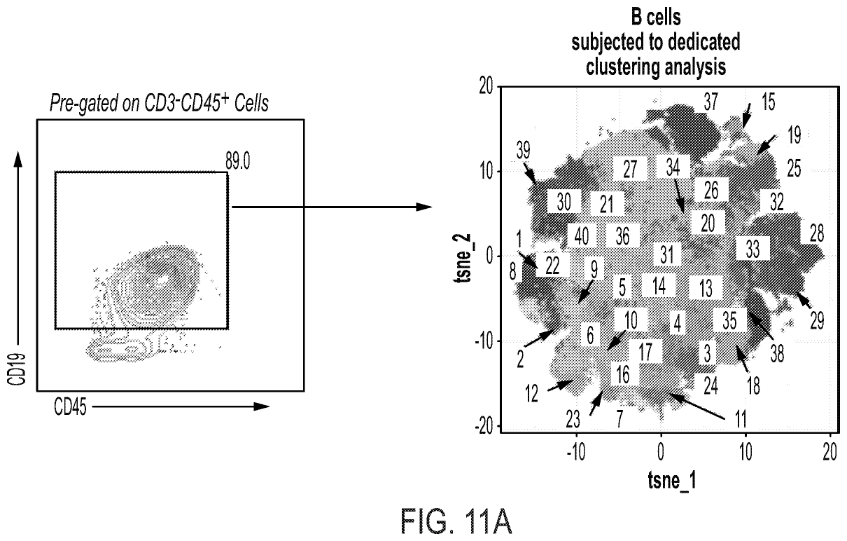

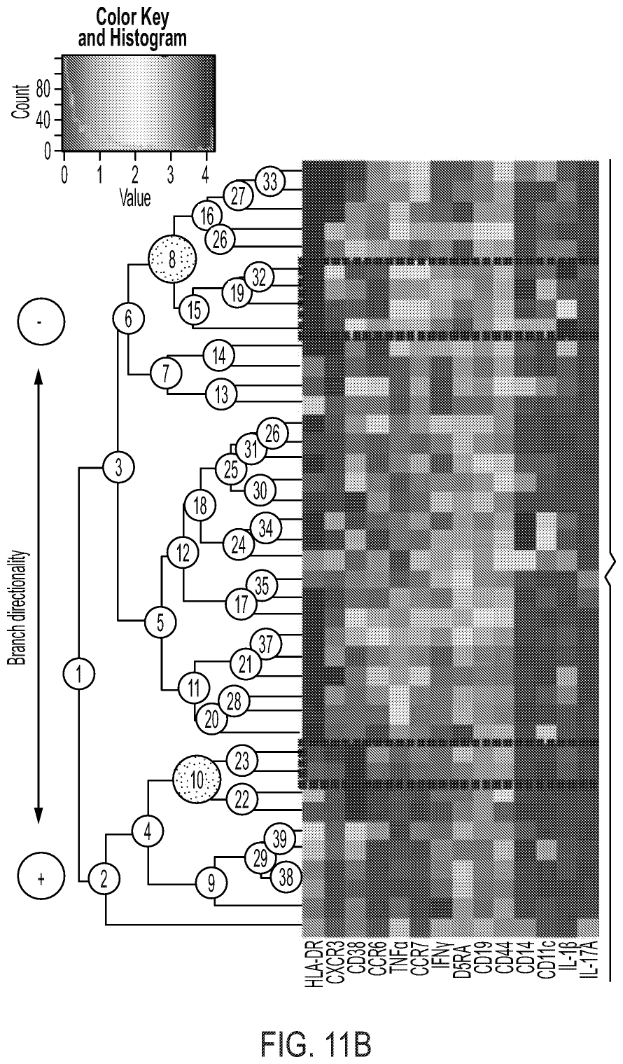

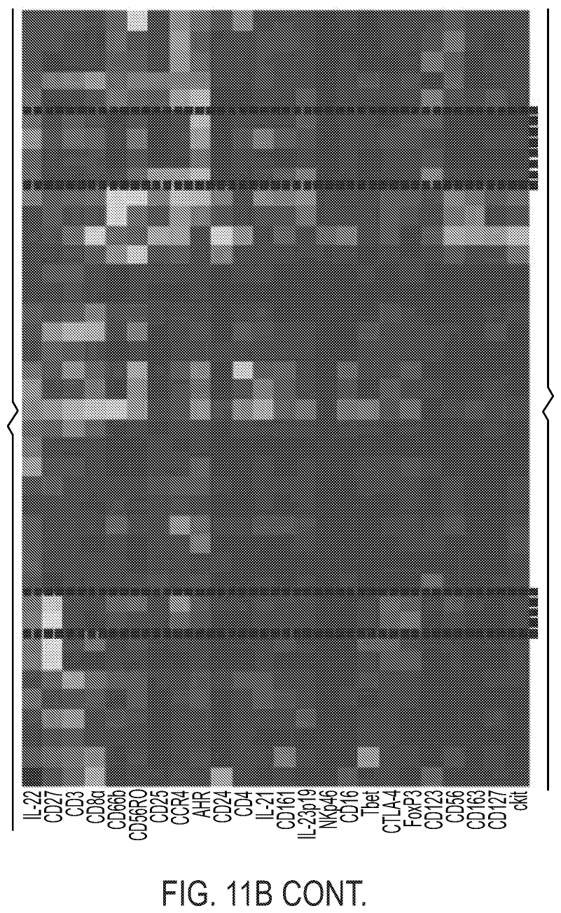

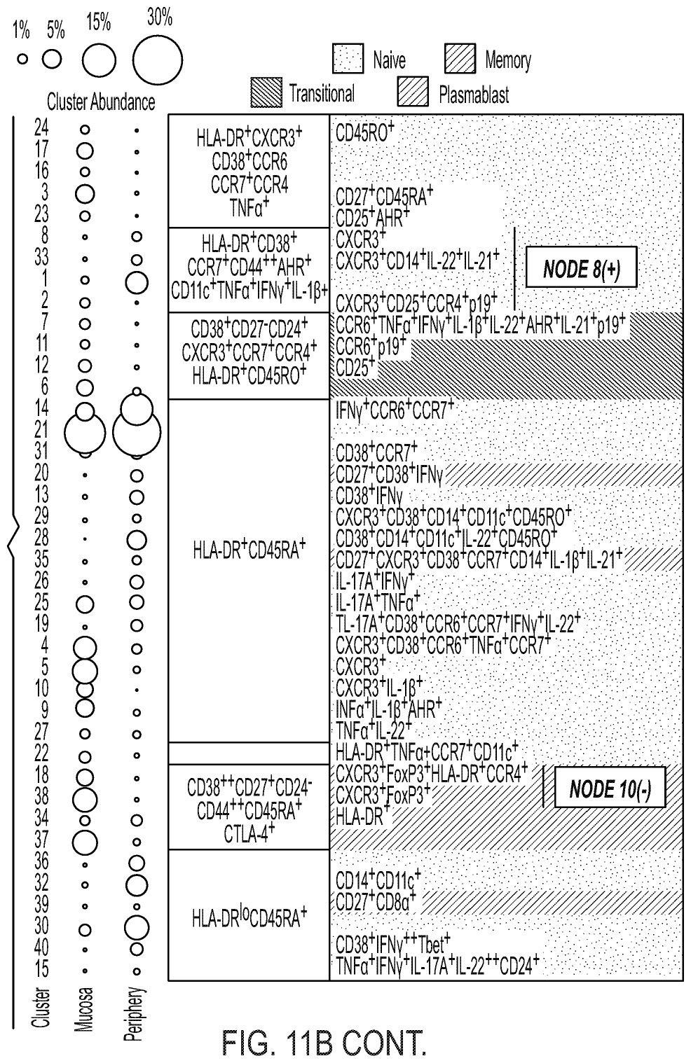

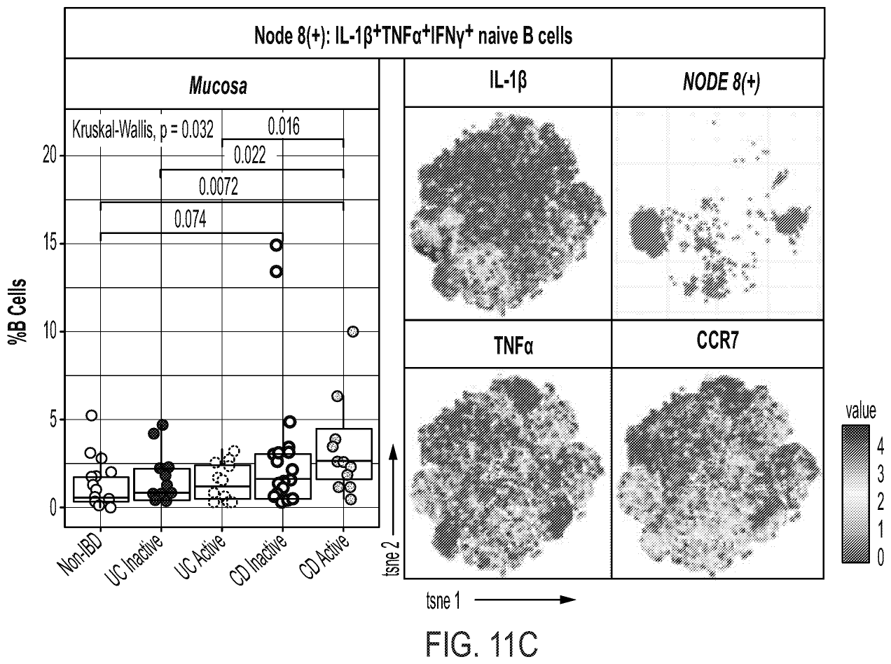

As noted, manual gating of CyTOF data demonstrated enrichment of B cells in UCa mucosa (FIG. 4A). To investigate differences within B cell populations across subject groups, CD19⁺ B cells were clustered in a dedicated analysis (FIGS. 11A, 11B). Multiple B cell subtypes including CD27⁻ naïve, CD27⁺ memory, CD27⁻CD24⁺CD38⁺ transitional, and CD27⁺CD24⁻CD38⁺⁺ plasmablasts were identified (FIG. 11B).

IL-1β⁺IFNγTNFα⁺ naïve B cell clusters (Node 8(+) in FIG. 11B) were increased in CDa mucosa (FIG. 11C). These clusters were CD44⁺⁺ (marker of activated B cells), CCR7⁺, AHR⁺, HLA-DR⁺, CD38⁺ and CD11c⁺, a marker expressed in B cells capable of antigen presentation associated with autoimmunity (FIGS. 11B, 11C). Cluster 33 within this node branch was found to express CD14, which could reflect a non-B cell macrophage/monocyte population or CD14⁻expressing B cells which have been previously reported (Ziegler-Heitbrock H W et al., Eur J Immunol 1994, 24:1937-40).

### Example 10: Plasmablasts/Regulatory B Cells are Increased in Abundance in Active Inflammatory Bowel Disease Mucosa

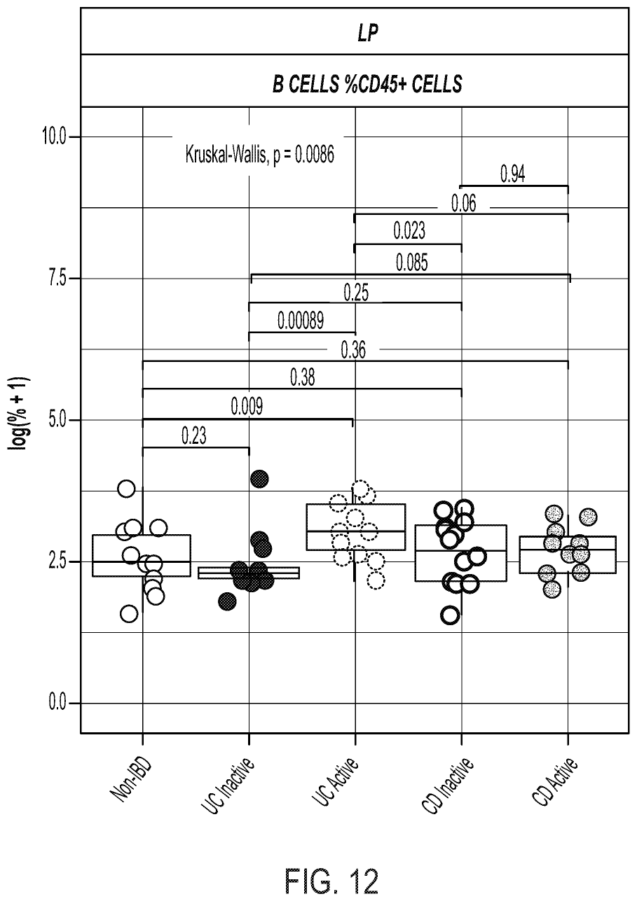

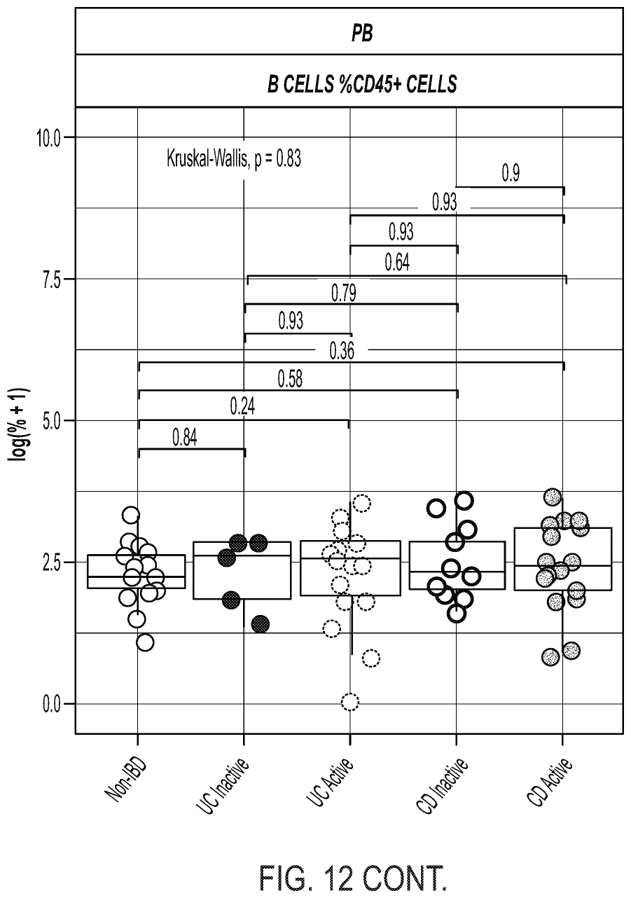

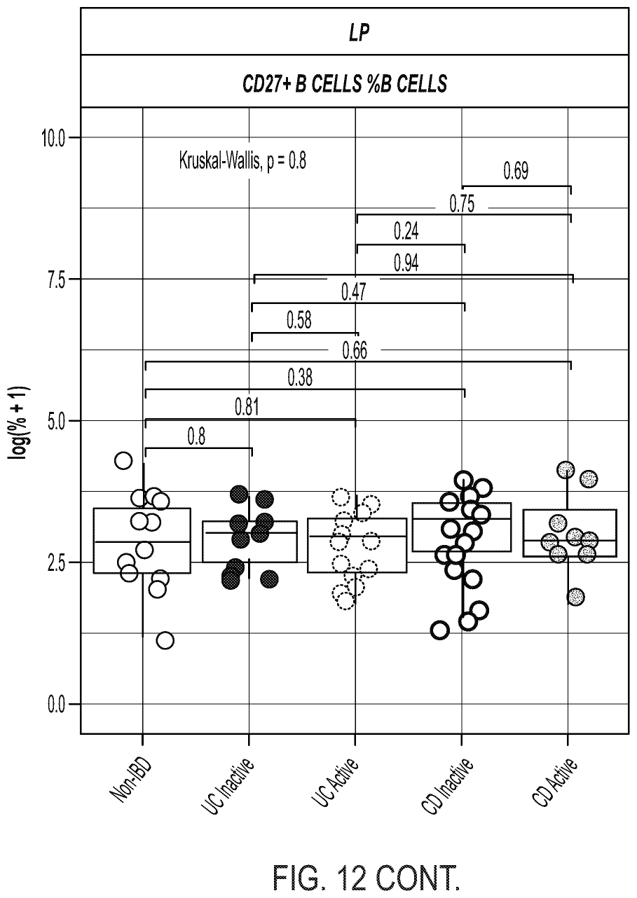

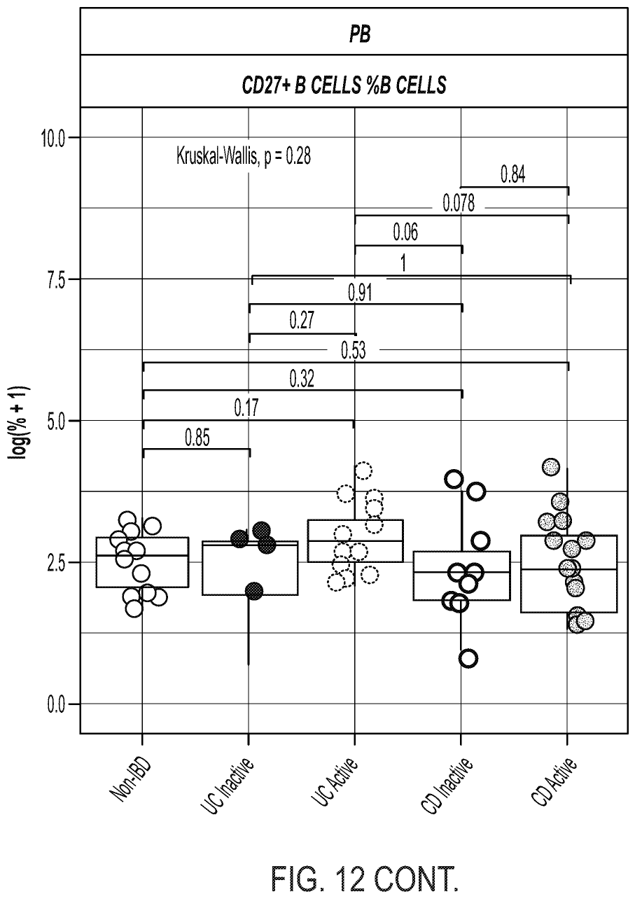

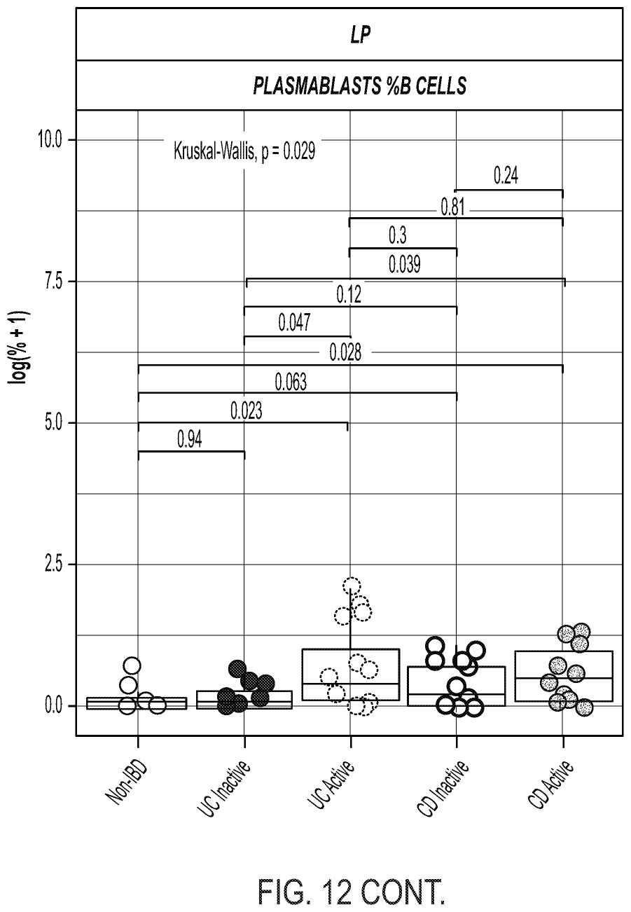

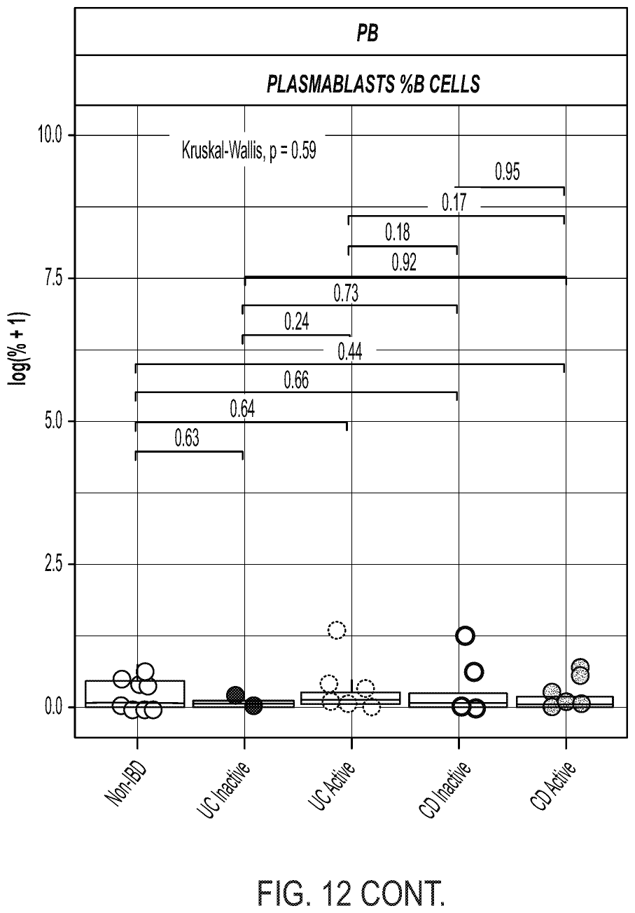

An analysis of B cell differences across subject groups in the mucosa and peripheral blood was also performed. To further characterize B cell populations, B cells were subtyped into CD27⁺ (mature) B cells, CD27⁻ (immature) B cells, and CD24⁺CD38⁺ (plasmablasts/regulatory B cells). No differences in abundance were observed for CD27^{+/-} B cells; however, in contrast to previously published results (Wang et al., J. Crohn's Colitis, 10:1212-23 (2016)) plasmablast/regulatory B cells abundance was enhanced in active IBD (active ulcerative colitis/active Crohn's disease) compared to non-IBD (FIG. 12). No B cell abundance differences were identified in the periphery (FIG. 12). Principal component analysis (PCA) was performed on all mucosa samples to differentiate plasmablasts/regulatory B cells from other B cell subtypes based on marker expression.

### Example 11: CXCR3+ Plasmablasts are Increased in IBD Mucosa

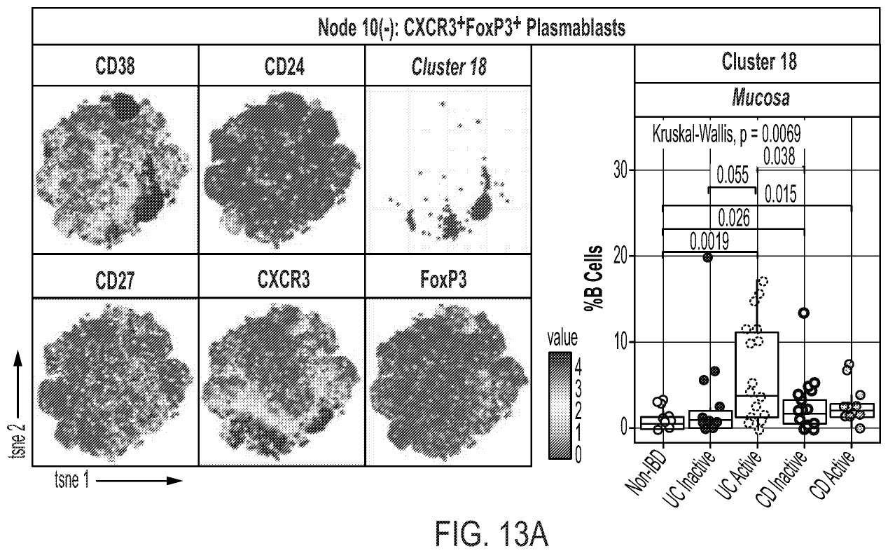

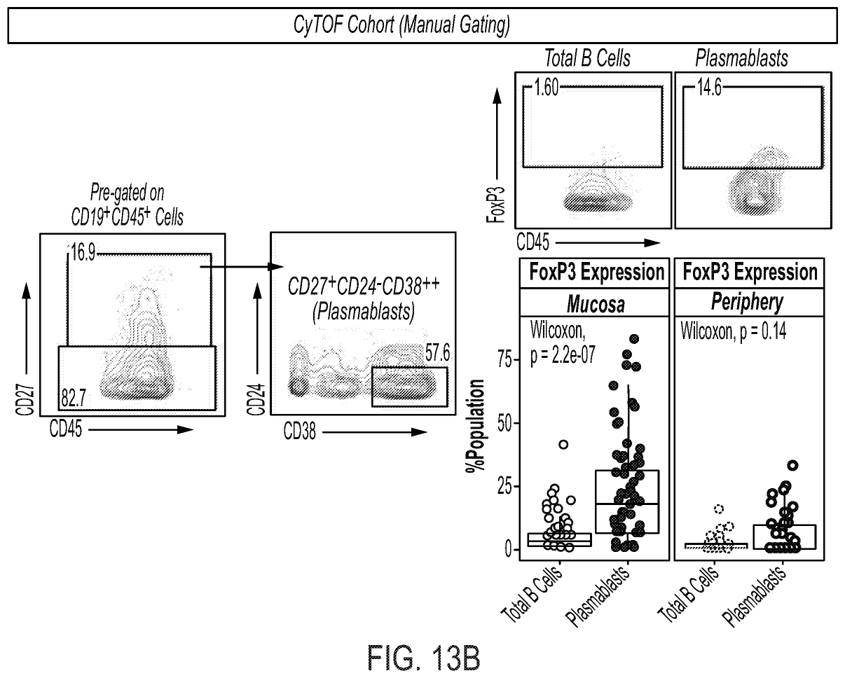

A population of CXCR3⁺ plasmablasts (Cluster 18 in FIG. 11B) was found to be increased in active IBD mucosa, especially in UCa (FIG. 13A). CXCR3⁺ plasmablast clusters (Node 10(−) in FIG. 11B) expressed FoxP3, which was further investigated through manual gating of CyTOF data (FIG. 13B), also supporting a FoxP3 signal in mucosal plasmablasts. Although FoxP3 expression in non-T cell lineages has been controversial, the findings presented herein indicate a regulatory function for mucosal plasmablasts.

### Example 12: IL-17A-Expressing B Cells Predominate Ulcerative Colitis Mucosa Whereas IL-1β-Expressing B Cells Predominate Crohn's Disease Mucosa

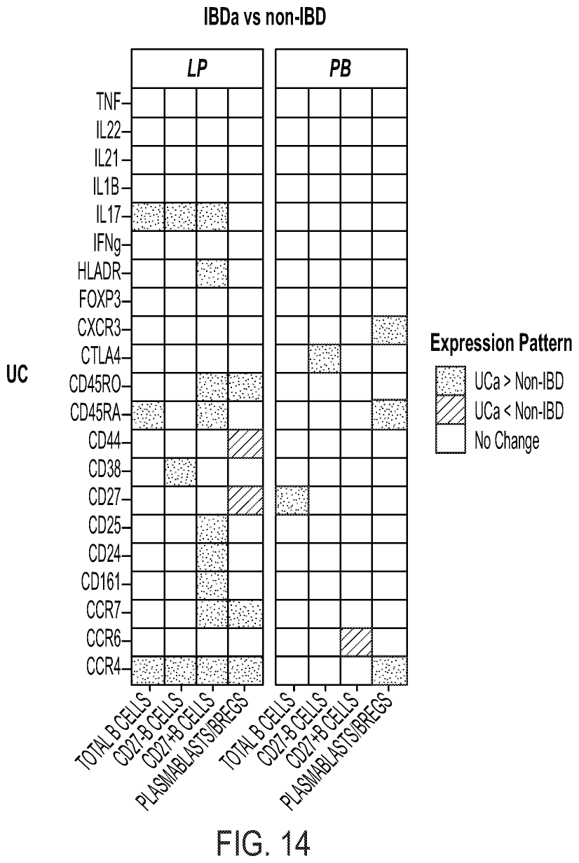

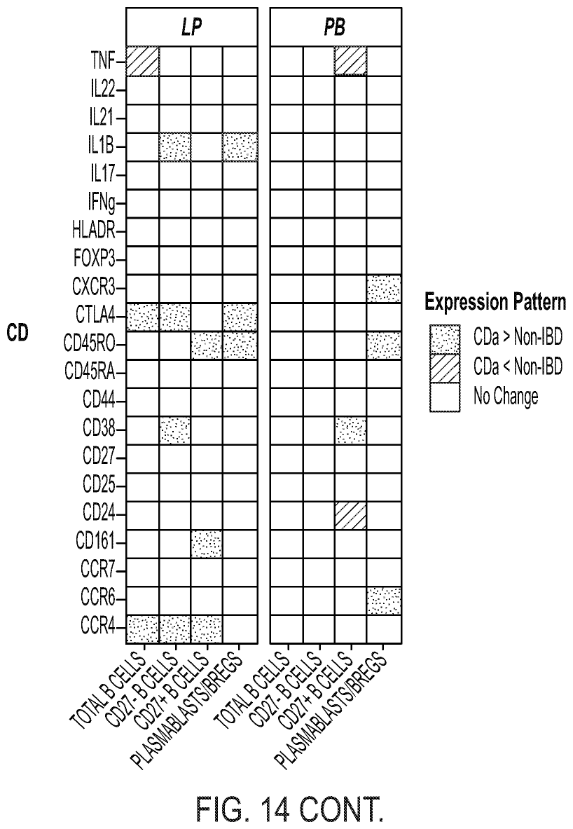

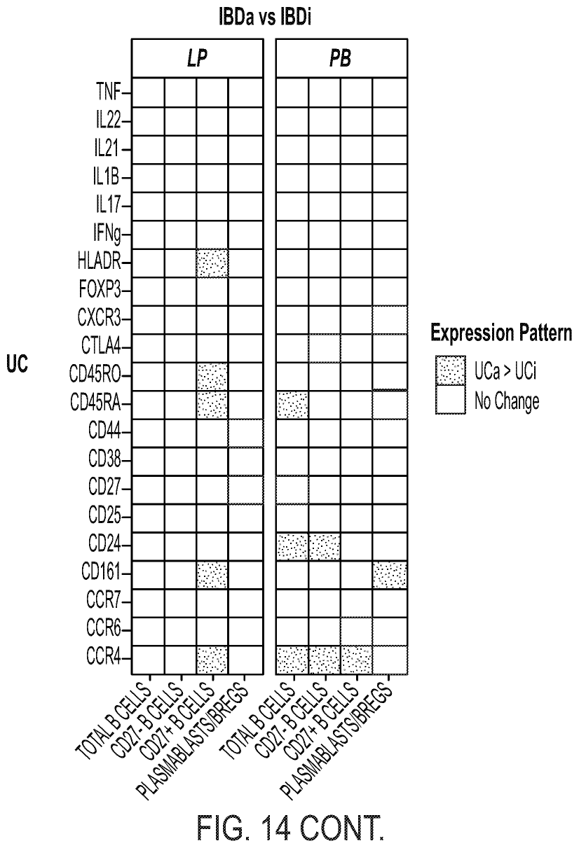

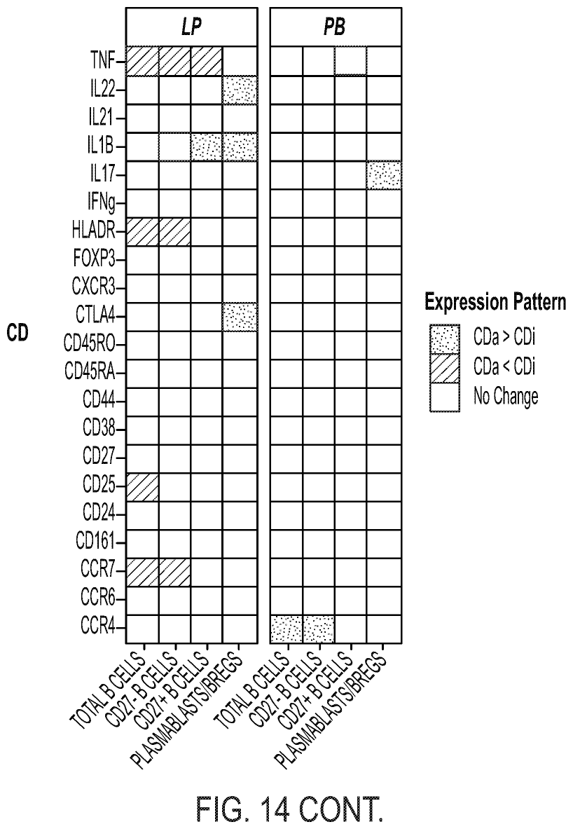

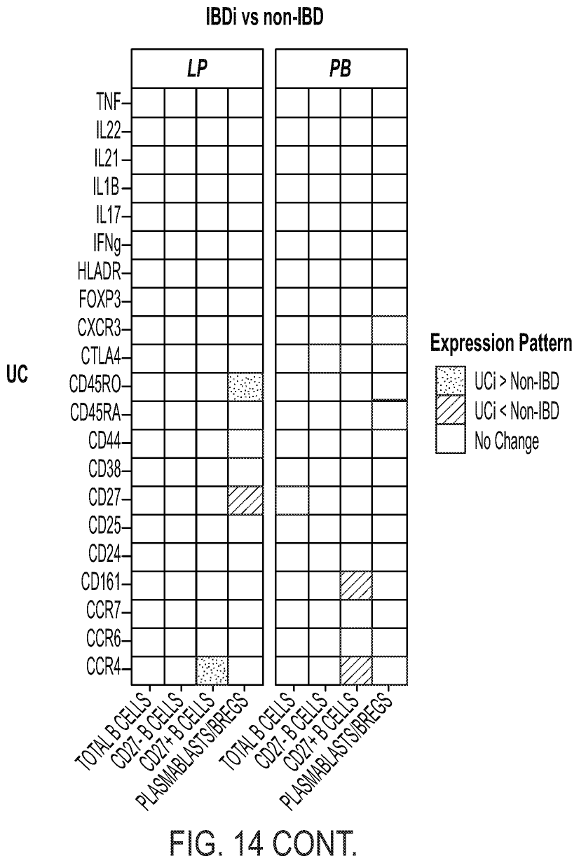

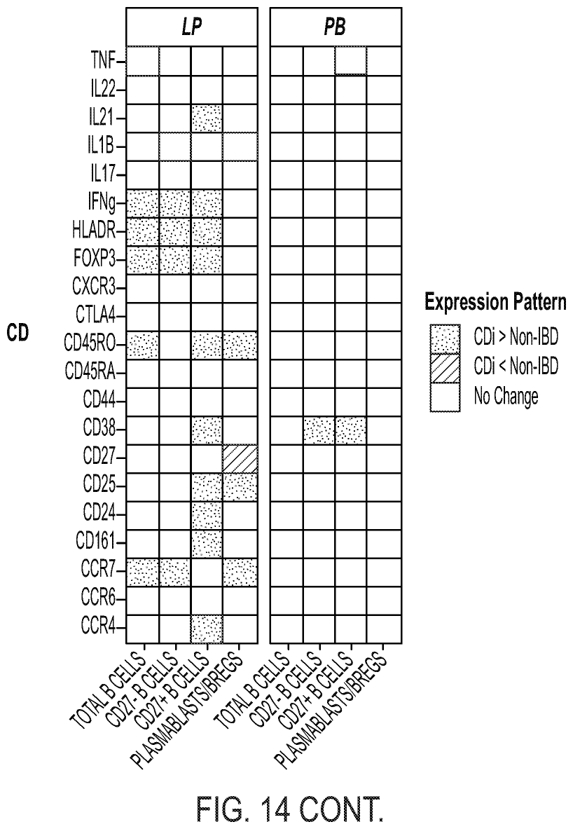

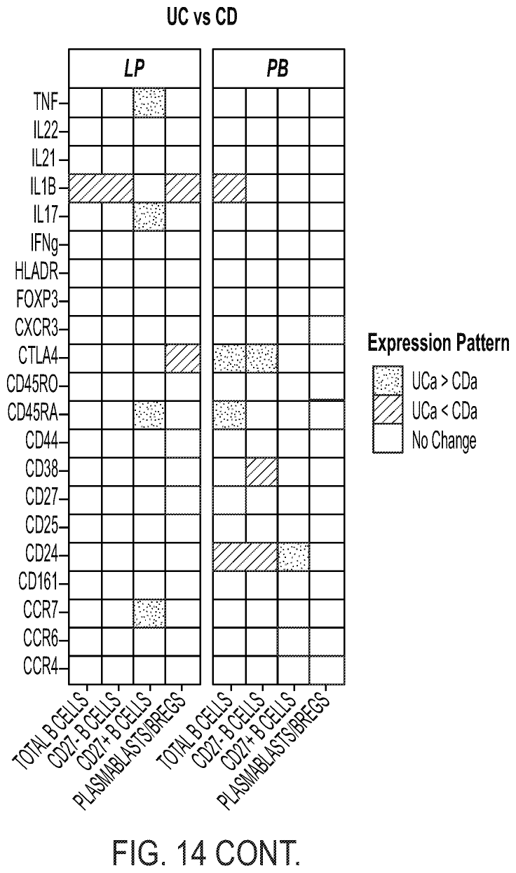

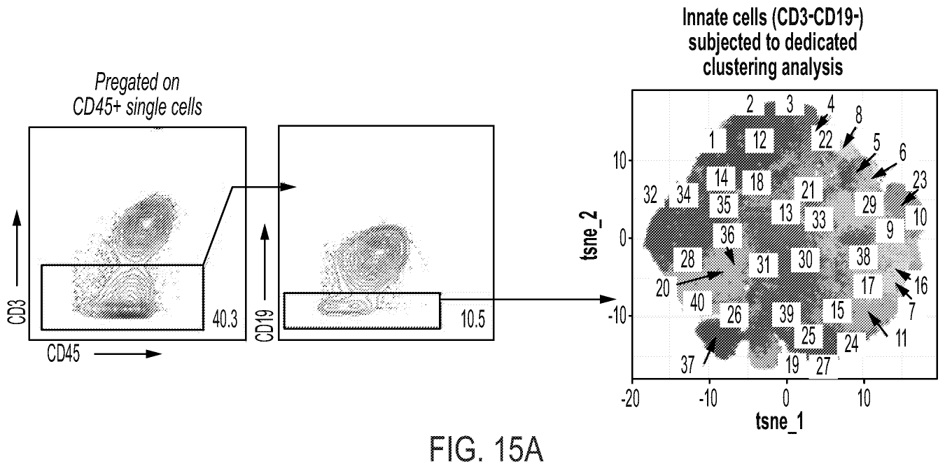

Almost all subsets of B cells had increased CCR4 expression in active IBD (active ulcerative colitis/active Crohn's disease) compared to non-IBD (FIG. 14). Consistent with the findings in T cells described above, B cell subsets (specifically mature B cells and plasmablasts/regulatory B cells) in active Crohn's disease had enhanced IL-1β expression compared to active ulcerative colitis while active ulcerative colitis B cells expressed more IL-17A than non-IBD and active Crohn's disease (immature B cells and mature B cells, respectively) (FIG. 14). Similar to the findings in T cells, inactive mucosa IBD tissue had B cell signatures that differentiated inactive Crohn's disease and inactive ulcerative colitis from active tissue as well as non-IBD mucosa. Once again, these differences were most robust for inactive Crohn's disease (rather than inactive ulcerative colitis) and included an increase in CCR7, CD45RO, IFNγ, and HLA-DR expression in inactive Crohn's disease compared to controls. Increases in CCR7, HLA-DR, and TNFα expression were observed in inactive Crohn's disease compared to active Crohn's disease across total B cells (FIG. 14).

In the periphery, differences were observed in B cell subset marker expression across patient groups. Increased IL-1β expression was observed in active Crohn's disease compared to active ulcerative colitis in total B cells and increased CXCR3 expression in active Crohn's disease and active ulcerative compared to control plasmablasts/regulatory B cells, as has been noted previously in ulcerative colitis (Hosomi et al., Clin. Exp. Immunol., 163:215-24 (2011), the contents of which are incorporated herein in their entirety) (FIG. 14).

### Example 13: UCa Mucosa is Enriched with Unconventional Granulocytes and CDa Mucosa with IL-1β+ Dendritic Cells

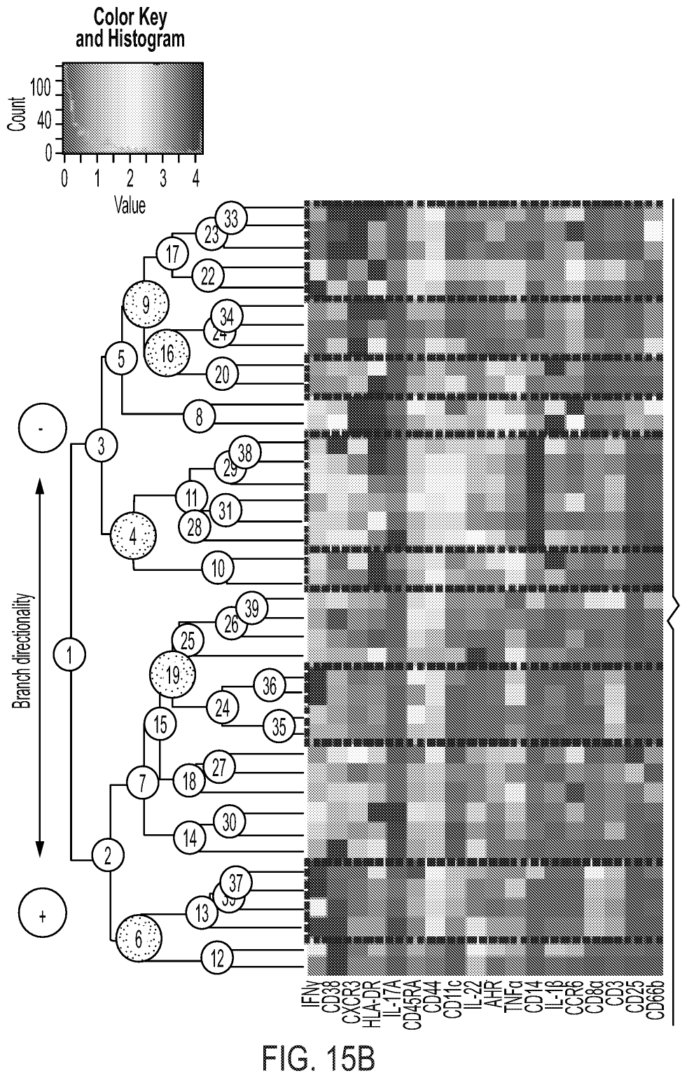

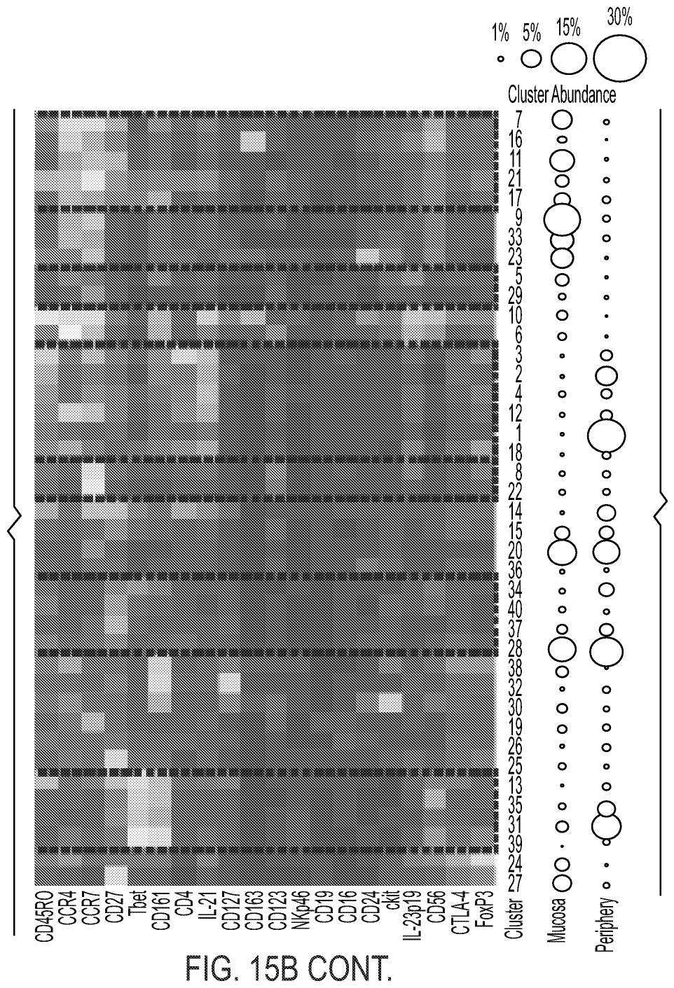

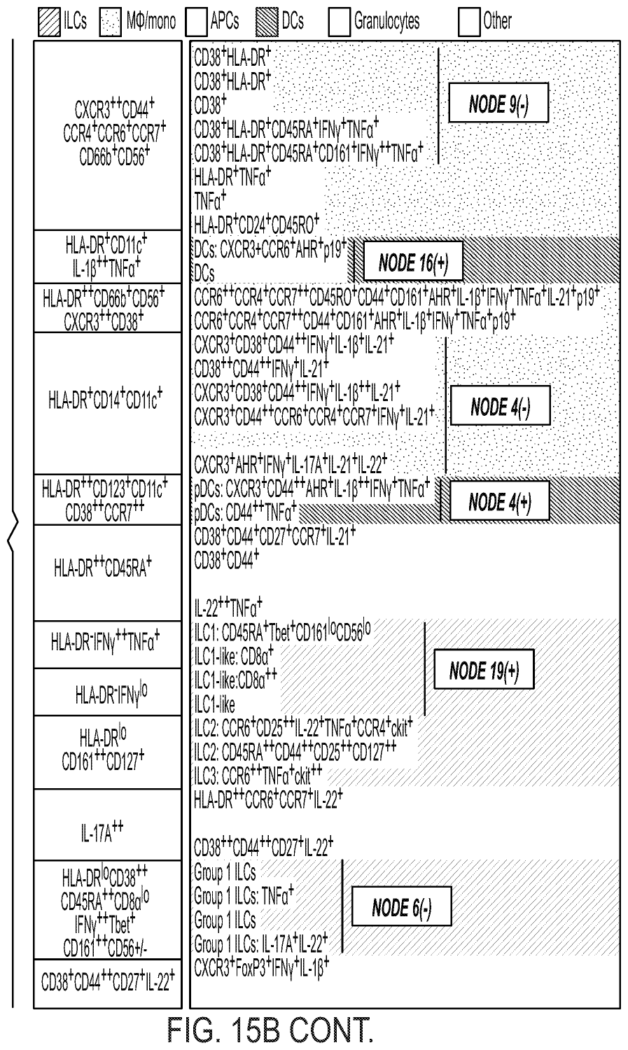

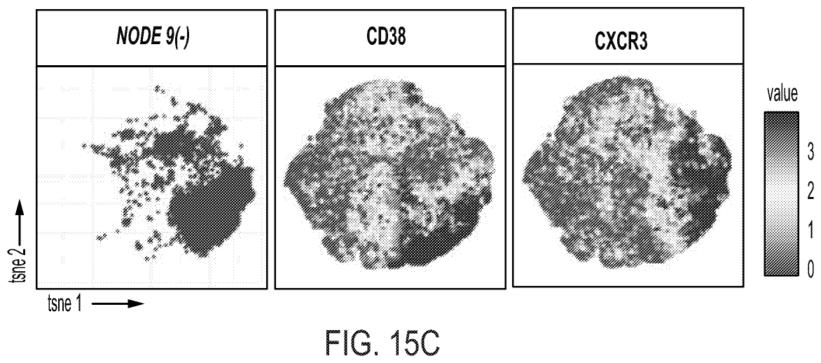

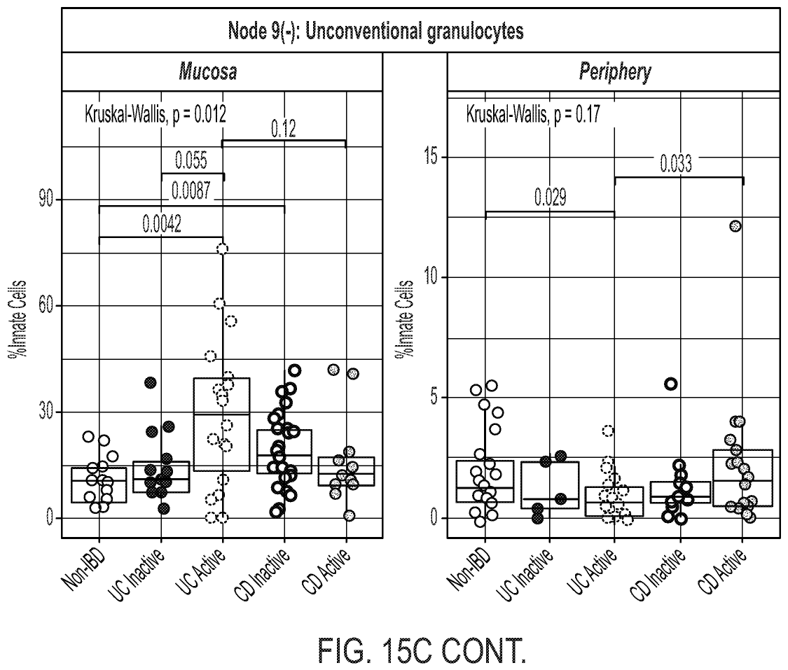

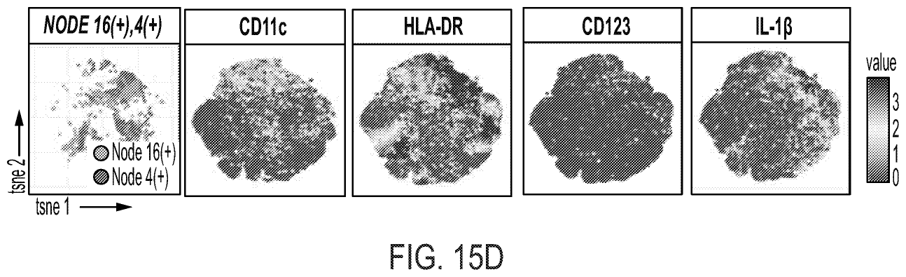

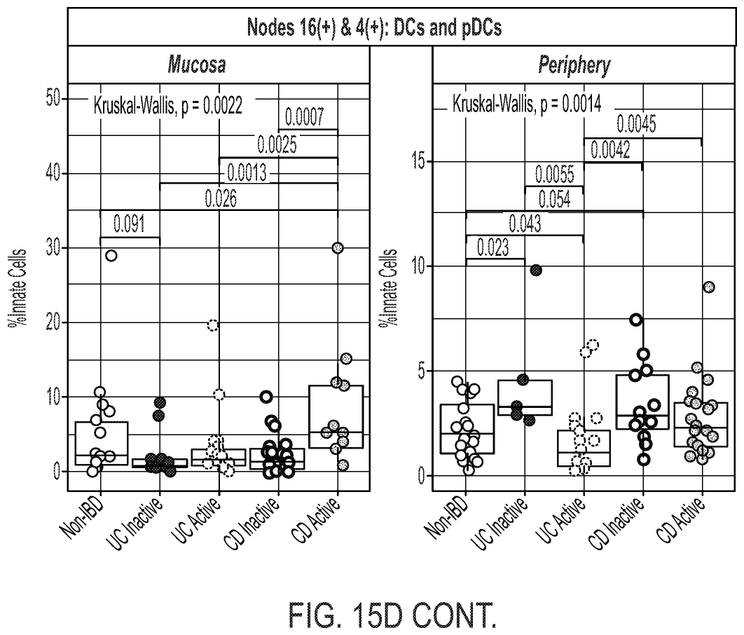

CD3⁻CD19⁻CD45⁺ cells (innate immune cells) were clustered in a dedicated analysis (FIGS. 15A, 15B). Multiple subsets of innate immune cells were identified, including granulocytes (CD66b⁺), macrophages/monocytes (Mϕ/mono; CD11c⁺CD14⁺), dendritic cells (DCs; HLA-DR⁺CD11c⁺CD14⁻), plasmacytoid DCs (pDCs; CD123⁺HLA-DR⁺CD11c⁺CD14⁻), and innate lymphoid cells (ILCs) groups 1 (CD56⁺Tbet⁺CD161⁺IFNγTNFα+), 2 (CD161⁺CD127⁺CD25⁺⁺ckit^{+/-}) and 3 (CD161⁺CD127⁺CCR6⁺⁺ckit⁺) (FIG. 15B).

UCa mucosa was characterized by increased granulocytes (Node 9(−) in FIG. 15B) with a concomitant decrease (trend) in the periphery (FIG. 15C). This population expressed chemokine receptors (CXCR3, CCR6), as well as unconventional granulocyte markers including HLA-DR, CD38, and CD56, which have been reported to be upregulated on granulocytes in other human diseases (Lok L S C et al., Proc Natl Acad Sci USA 2019; 116:19083-9; Lin A et al., Front Immunol 2017; 8:1781) (FIGS. 15B, 15C). CDa mucosa, on the other hand, was characterized by increased DCs and pDCs (Nodes 16(+) and 4(+) respectively in FIG. 5B), which were also increased in peripheral CDa>UCa (FIG. 15D). The majority of these DC and pDC clusters, notably, highly expressed IL-1β (FIGS. 15B, 15D).

### Example 14: Differential Abundance of Innate Immune Cells in the Tissue and the Periphery can Distinguish Inflammatory Bowel Disease Subtypes

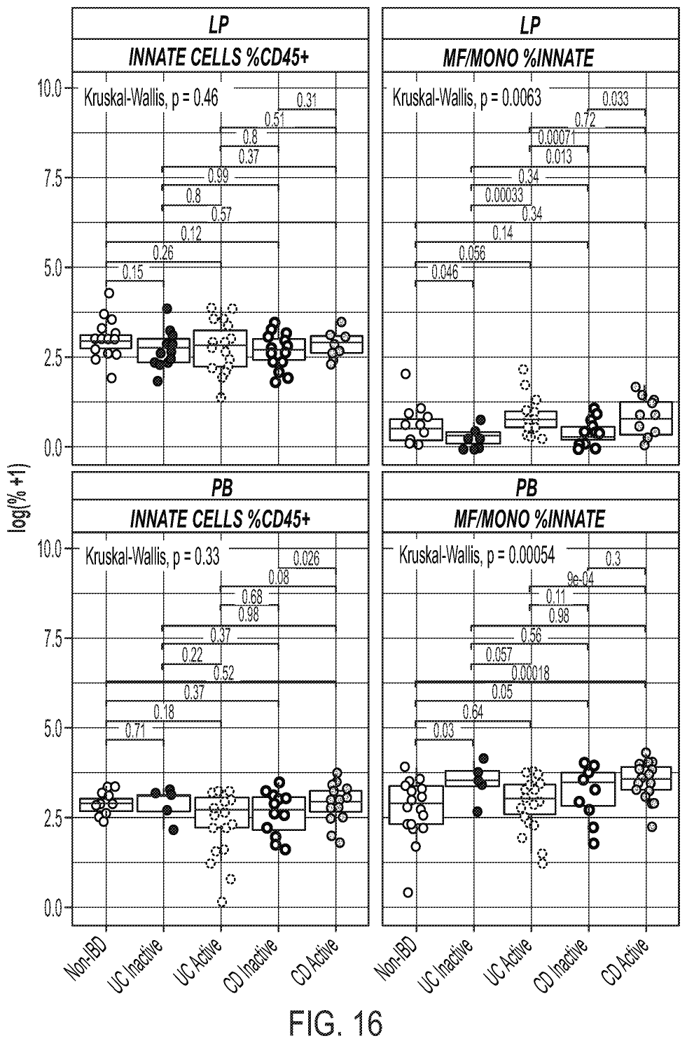

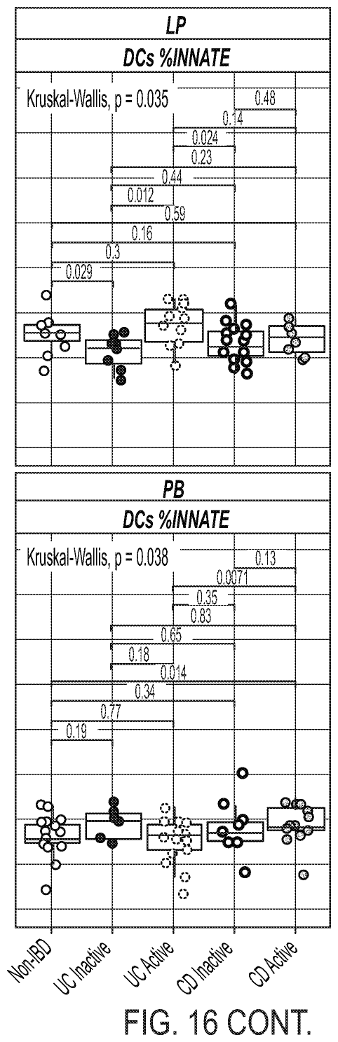

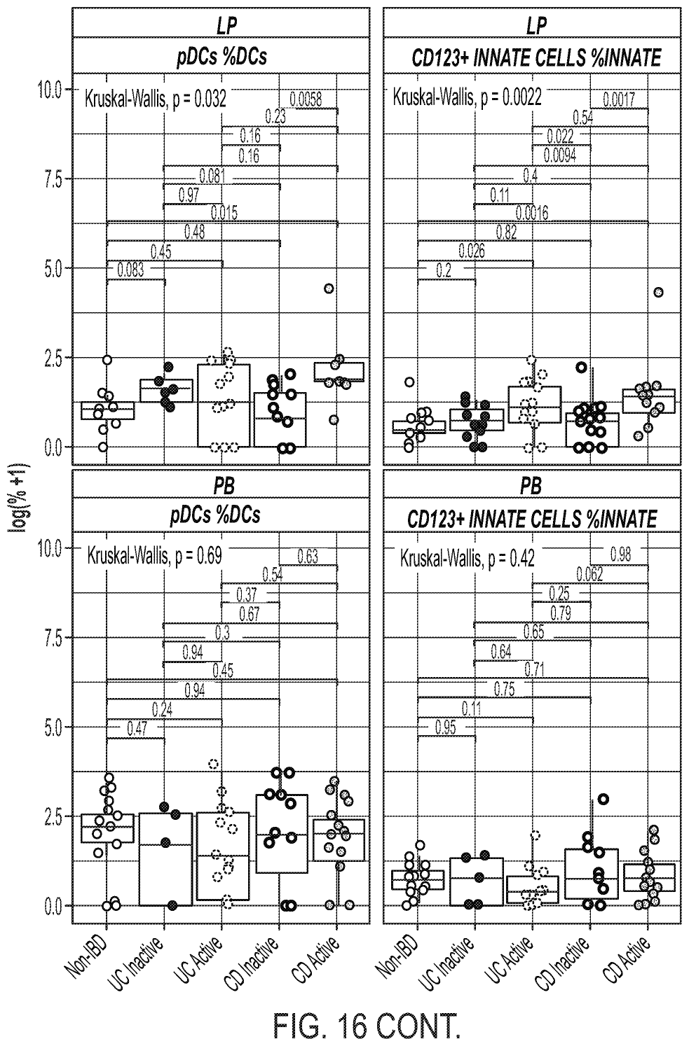

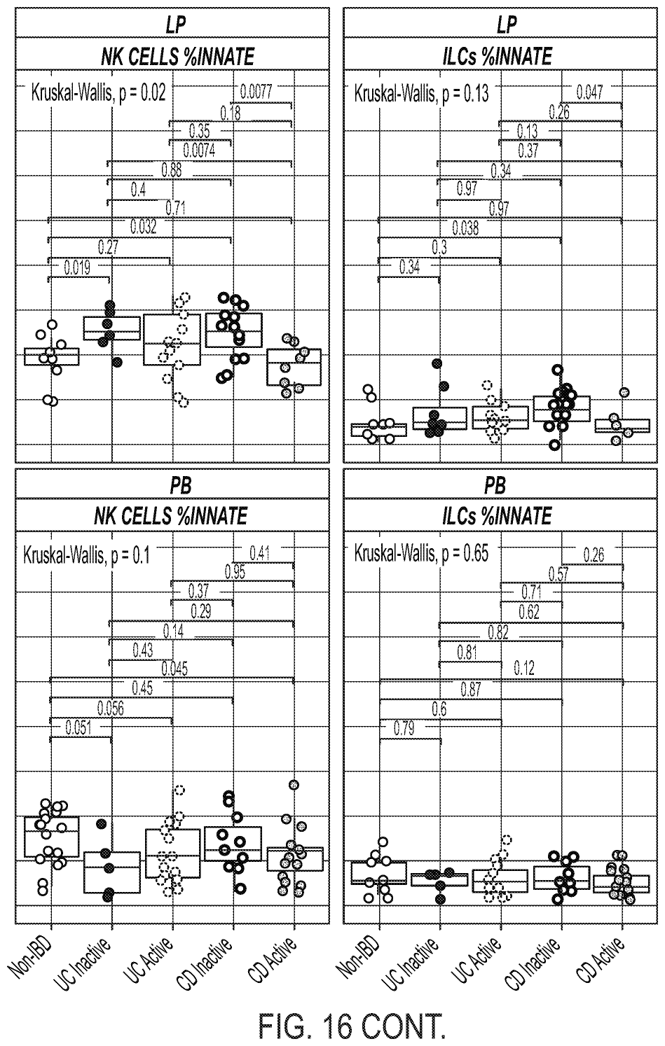

Innate immune cells were subtyped into macrophages and monocytes, CD14-antigen presenting cells (APCs) including dendritic cells, CD123⁺ dendritic cells (plasmacytoid dendritic cells), natural killer cells, innate lymphoid cells and CD123⁺ innate cells (a combination of plasmacytoid dendritic cells and basophils). Total mucosa innate immune cell abundance did not differ between patient groups and non-IBD subjects (FIG. 16). Consistent with prior reports (Kamada et al., J. Clin. Invest., 118:2269-80 (2008), the contents of which are incorporated by reference in their entirety), macrophages and monocytes were increased in abundance in all active IBD compared to inactive IBD mucosa (FIG. 16). Similarly, CD123⁺ innate cells were increased in all active IBD compared to inactive IBD mucosa (FIG. 16). Dendritic cells were also increased in active ulcerative colitis >inactive ulcerative colitis mucosa. Plasmacytoid dendritic cells, as a proportion of total dendritic cells, were increased in abundance in active Crohn's disease over both inactive Crohn's disease as well as non-IBD controls (FIG. 16). Again, indicative of unique signatures in inactive IBD tissue, both inactive ulcerative colitis and inactive Crohn's disease had greater abundance of natural killer cells than controls and active Crohn's disease mucosa. inactive Crohn's disease mucosa had a greater abundance of innate lymphoid cells than both active Crohn's disease and controls (FIG. 16).

In contrast to the findings in peripheral T and B cell subsets, and in addition to observed differences in tissue, innate immune cell subsets differed in abundance in the peripheral blood of patients and controls (FIG. 16). Monocytes were proportionally increased in Crohn's disease peripheral blood, including in active Crohn's disease >active ulcerative colitis and >non-IBD. Monocytes were proportionally increased in inactive Crohn's disease to a greater extent than in non-IBD (FIG. 16). These cells were also increased in inactive ulcerative colitis compared to non-IBD. Dendritic cells were also proportionally increased in active Crohn's disease compared to active ulcerative colitis and >controls (FIG. 16). No peripheral blood differences in abundance were observed for CD123⁺ innate cells, natural killer cells, or innate lymphoid cells (FIG. 16).

### Example 15: ILC Signatures Differentiate CD from UC in Mucosa and Periphery

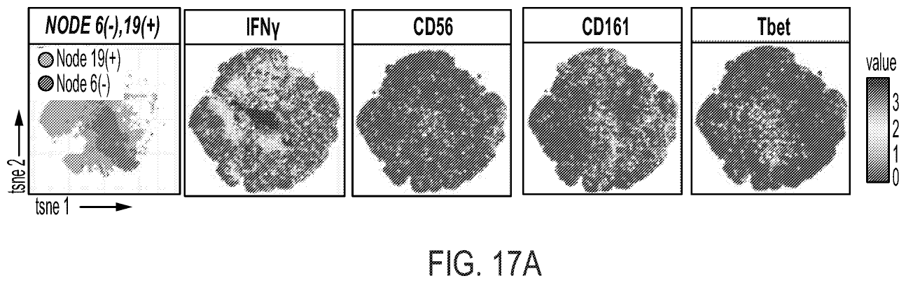

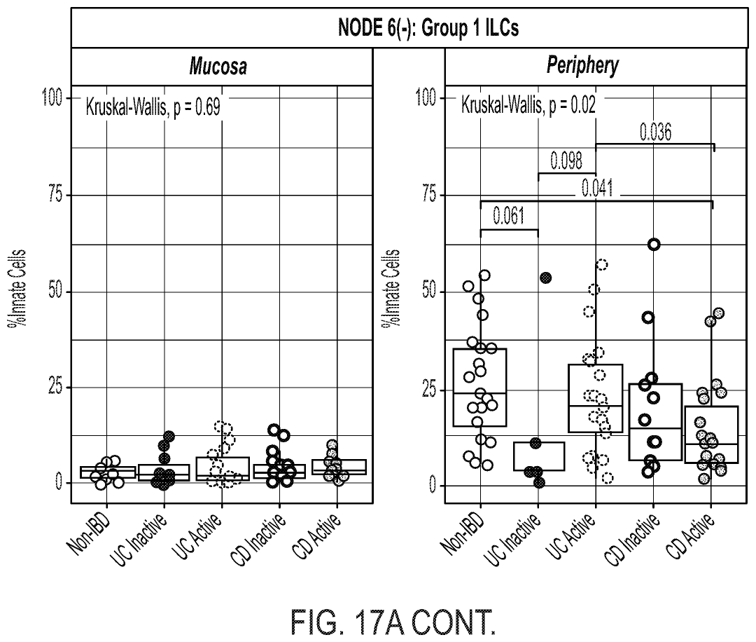

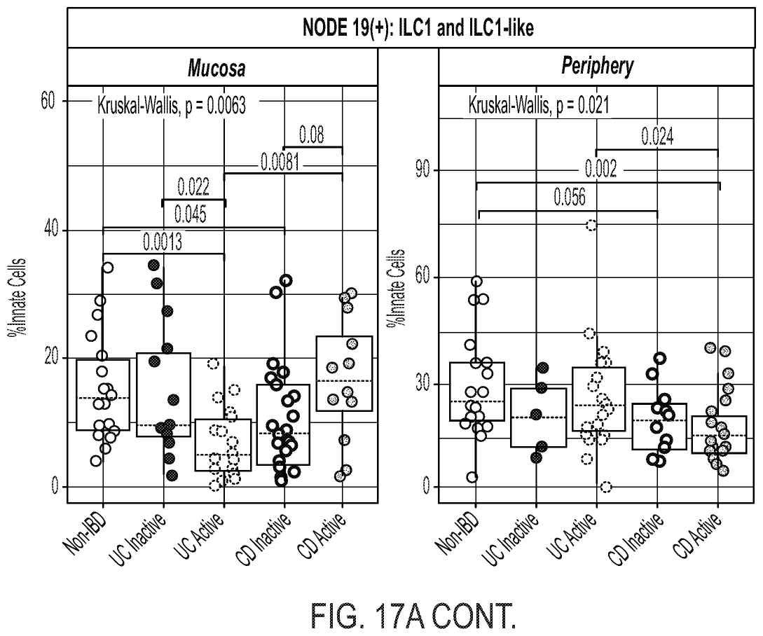

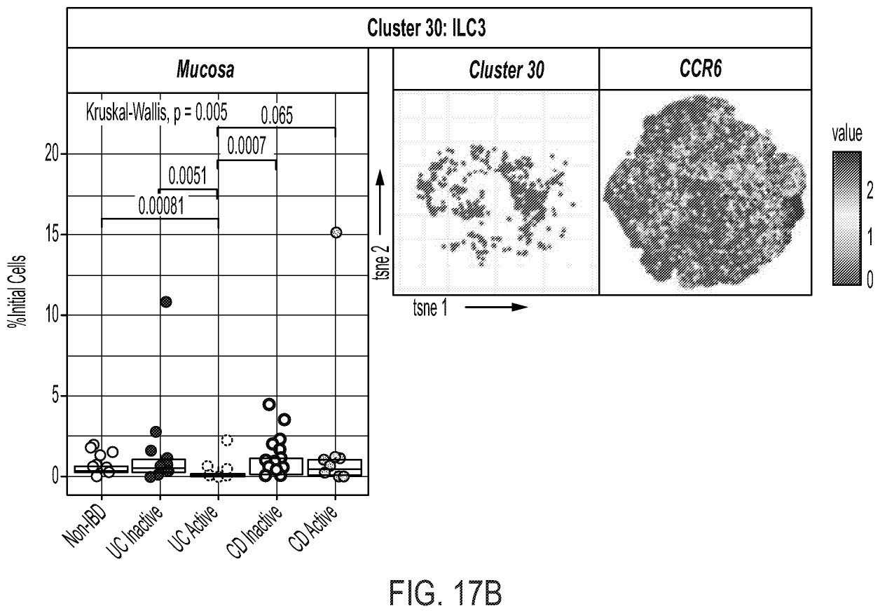

Group 1 ILCs (likely including both ILC1 and natural killer [NK] cells) (Node 6(−) in FIG. 15B) were decreased in peripheral CDa, without subject group differences in the mucosa (FIG. 17A). Node 19(+) (in FIG. 15B), which included both ILC1 (Cluster 34) as well as “ILC1-like” clusters (identified by lack of expression of lineage markers (e.g., CD14, CD11c) but with expression of the ILC1-hallmark cytokine IFNγ and by CD8α, which has been reported in ILCs (Roan F et al., J Immunol 2016; 196:2051-62), was also found to be decreased in peripheral CDa (FIG. 17A). ILC1 and ILC1-like clusters conversely were increased in the mucosa in CDa>UCa (FIG. 17A), while Cluster 30 (in FIG. 15B), representing ILC3s, was specifically reduced in UCa mucosa (FIG. 17B).

### Example 16: IL-1β Expression in Innate Immune Cells is Increased in Active Crohn's Disease Mucosa and Peripheral Blood

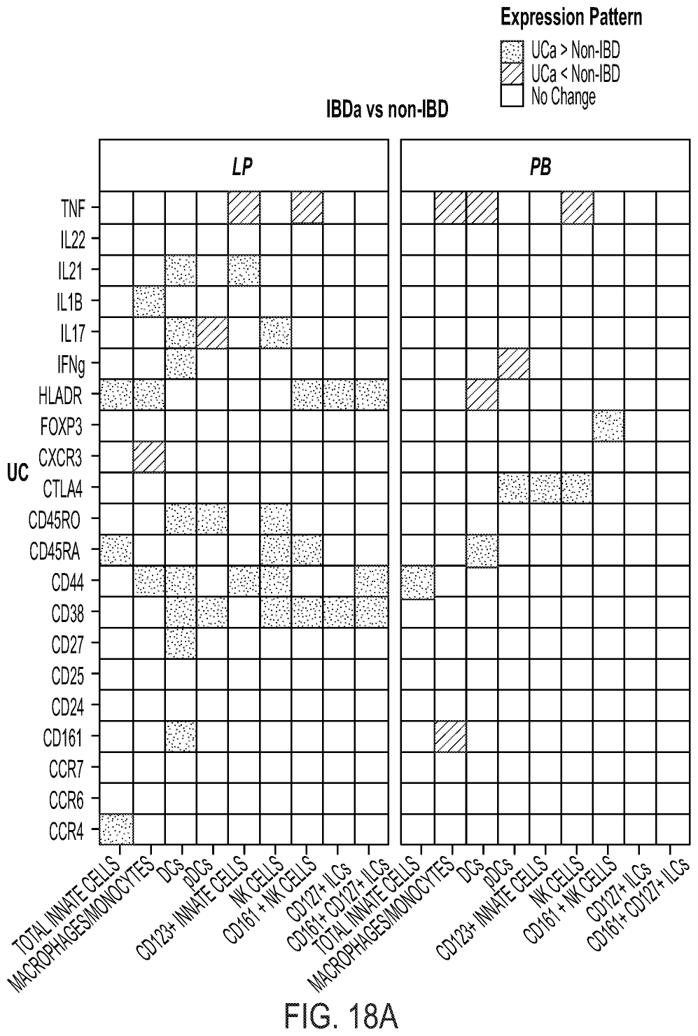

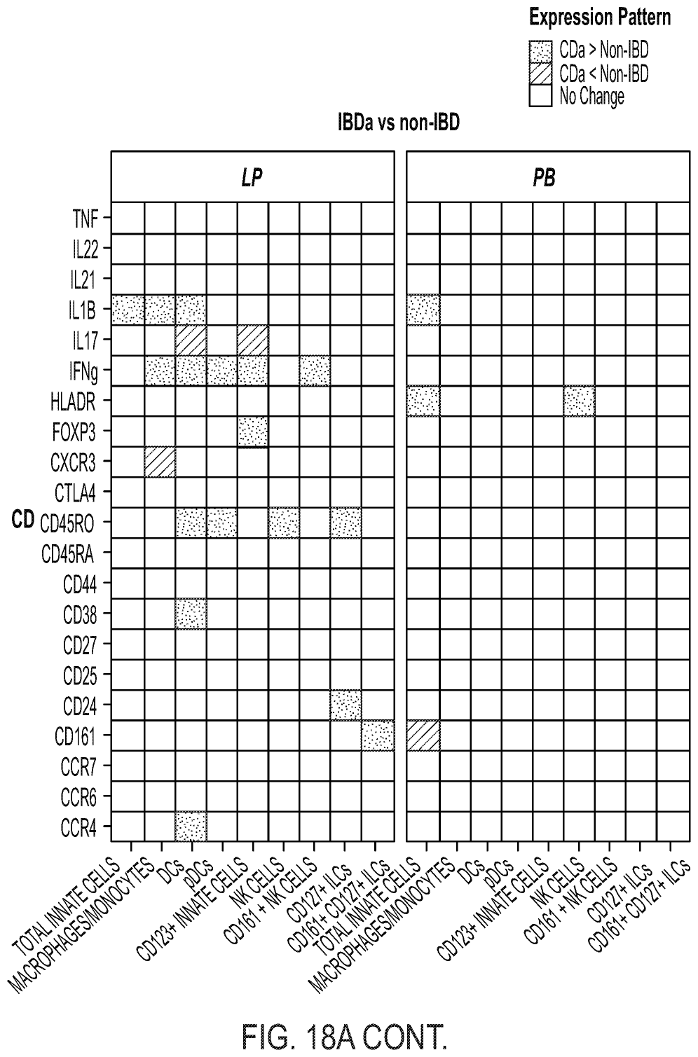

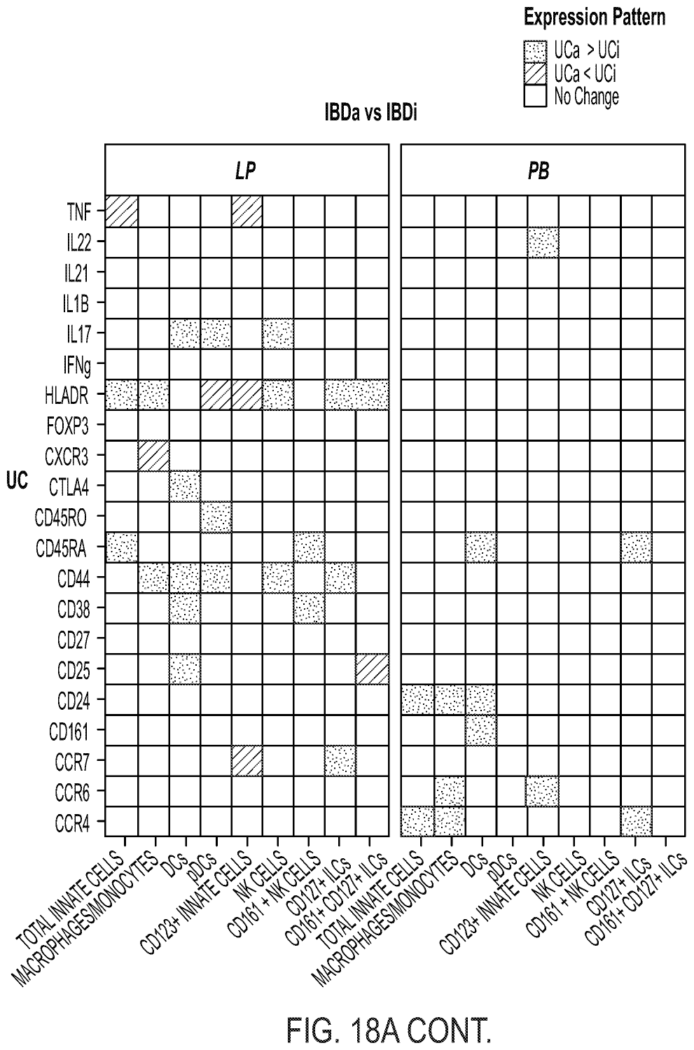

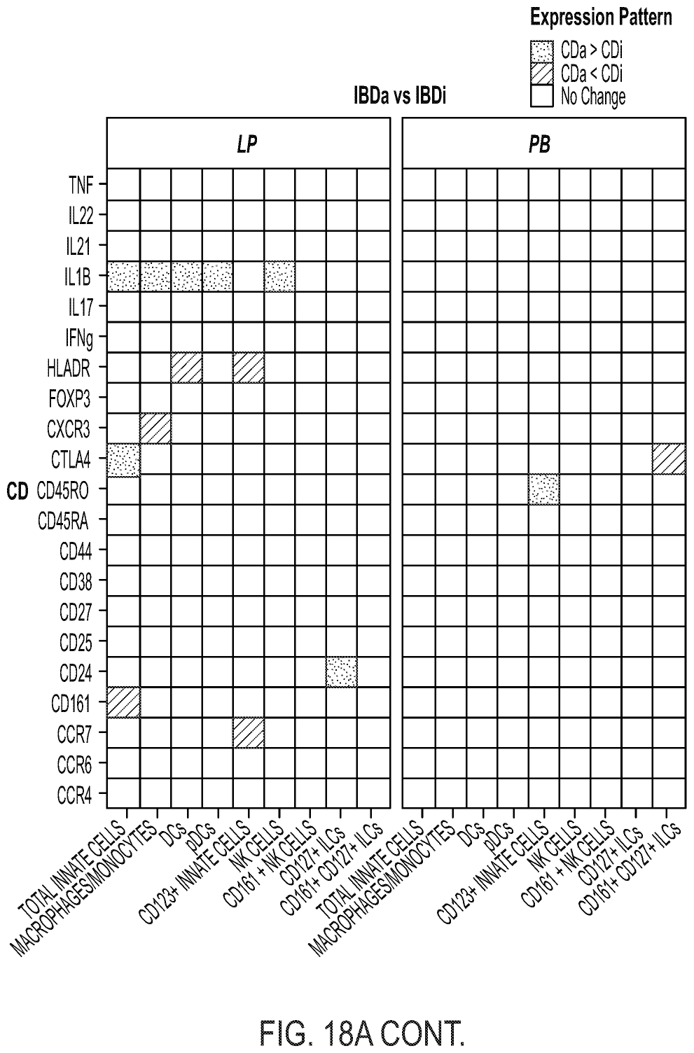

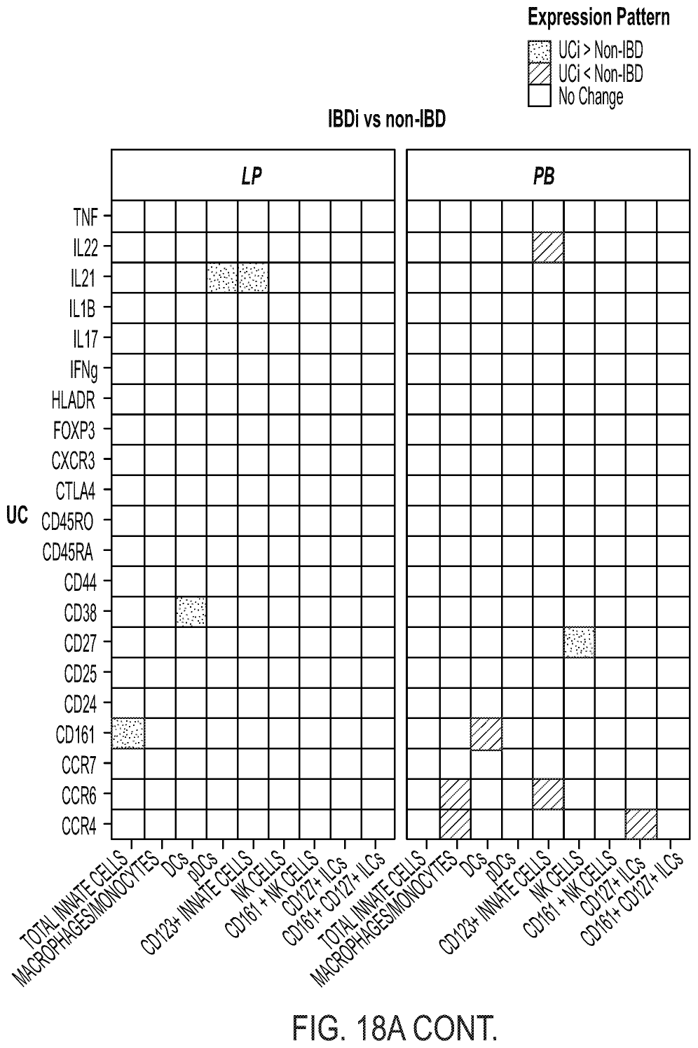

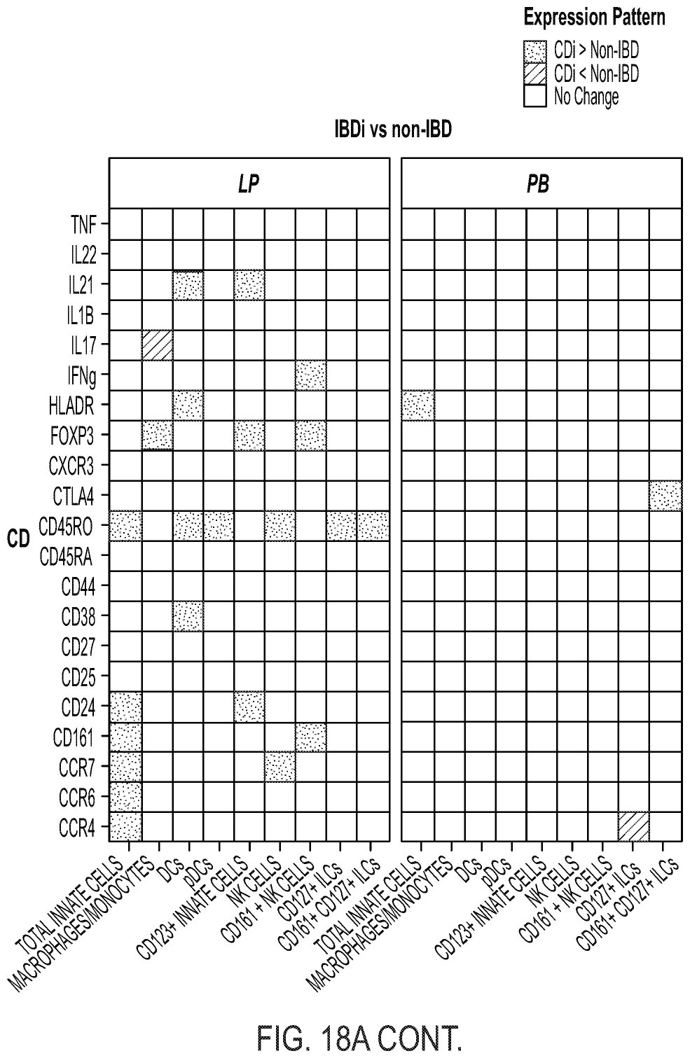

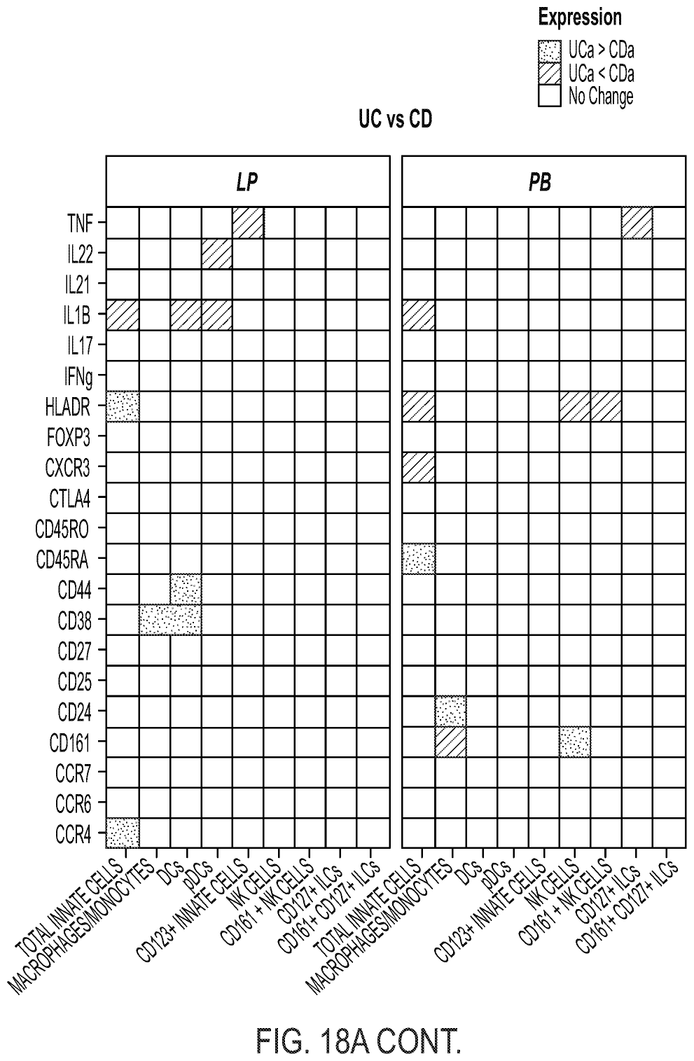

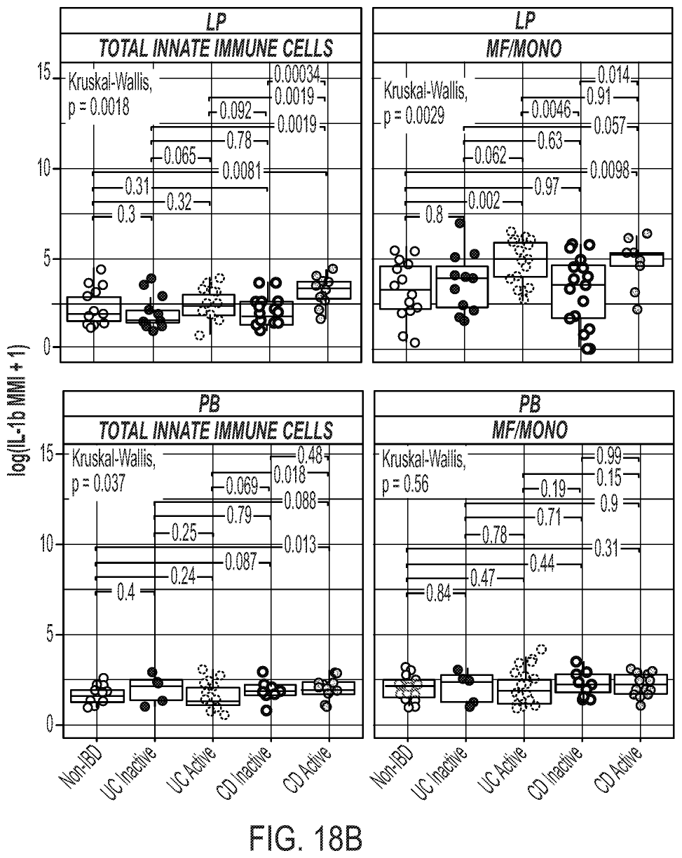

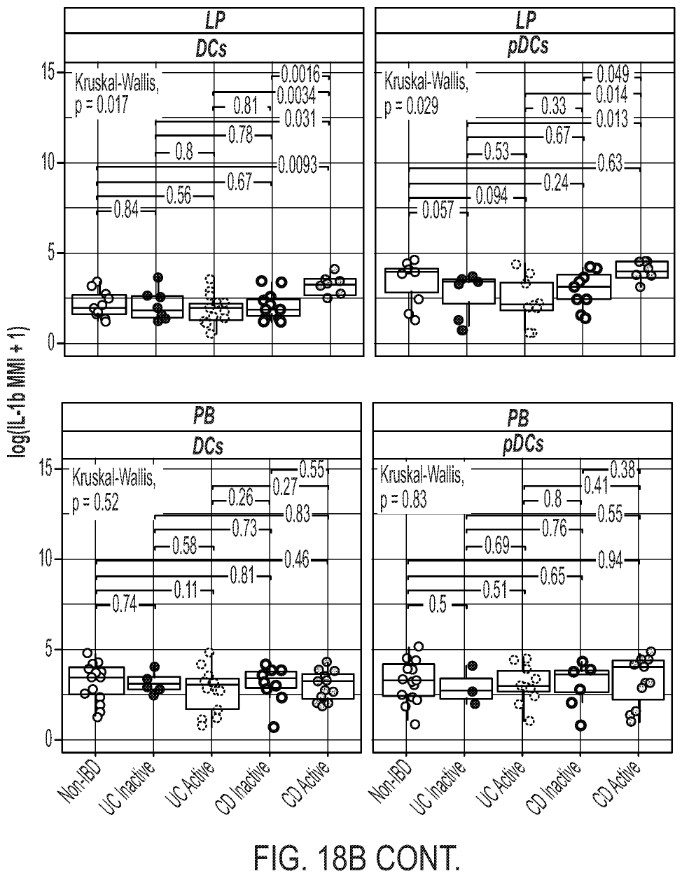

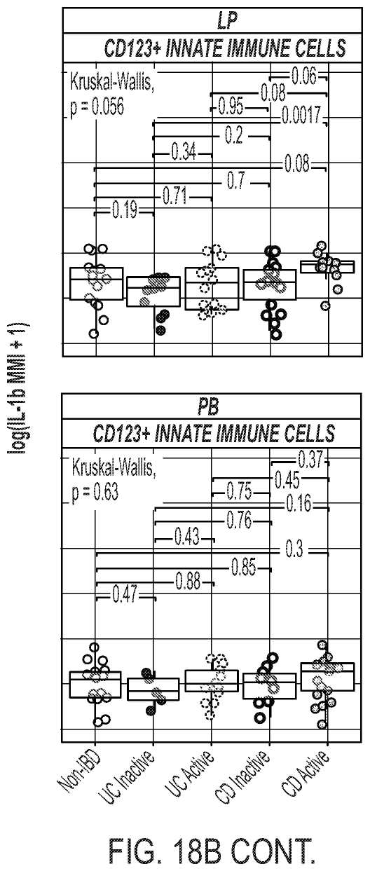

Similar to the findings in T cells and B cells, analysis of differential marker expression revealed differences in innate immune cell subsets that distinguished IBD mucosa and peripheral blood from non-IBD, as well as IBD patient subgroups (FIG. 18A).

Hallmarks of active ulcerative colitis mucosa innate immune cells mirrored the findings in T cells and B cells regarding IL-17A expression, which was increased in dendritic cells and natural killer cells compared to inactive ulcerative colitis and non-IBD mucosa. Similarly, TNFα expression was found to be reduced in various active ulcerative colitis innate immune cell subsets, including total innate immune cells (compared to inactive ulcerative colitis), CD161⁺ natural killer cells (compared to non-IBD) and CD123⁺ innate cells (compared to both inactive ulcerative colitis and non-IBD) (FIG. 18A).

Hallmarks of active Crohn's disease mucosa innate cells similarly included increased IL-1β, TNFα, and IFNγ expression in various subsets. For example, IL-1β and IFNγ expression were increased in macrophages and dendritic cells compared to non-IBD mucosa and TNFα expression was increased in CD123⁺ innate cells compared to active ulcerative colitis mucosa (FIG. 18A).

Direct comparison of active ulcerative colitis to active Crohn's disease mucosa further reinforced the IL-1β signature specific to Crohn's disease innate immune cells, with increased IL-1β expression in active Crohn's disease total innate immune cells, dendritic cells, and plasmacytoid dendritic cells. Notably, enhanced expression of IL-1β in macrophages was observed in both active ulcerative colitis and active Crohn's disease compared to non-IBD controls (FIGS. 18A and 18B).

Analysis of differential marker expression in peripheral blood innate immune cells across subject groups also identified increased IL-1β expression in total innate immune cells in active Crohn's disease >active ulcerative colitis (FIGS. 18A and 18B).

### Example 17: IL-1β⁺ Mϕ/Mono are Increased in Active IBD Mucosa and in Peripheral CDa

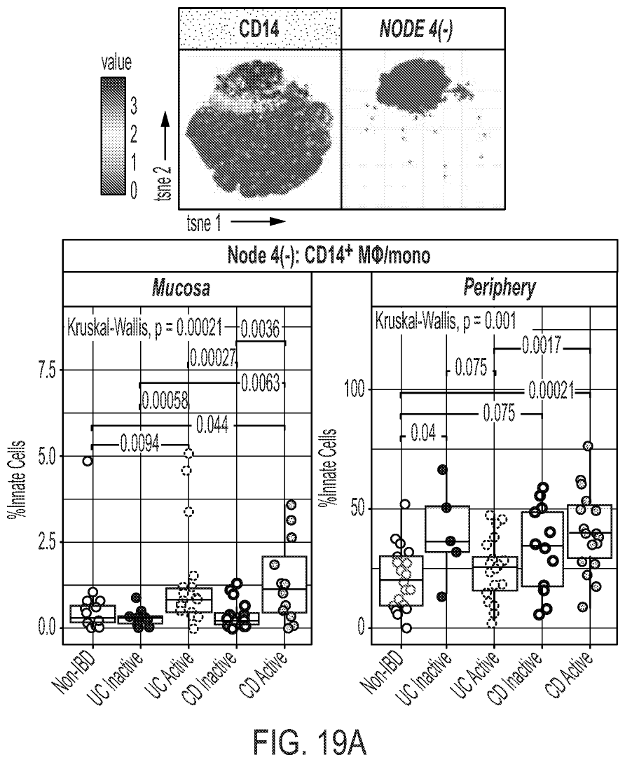

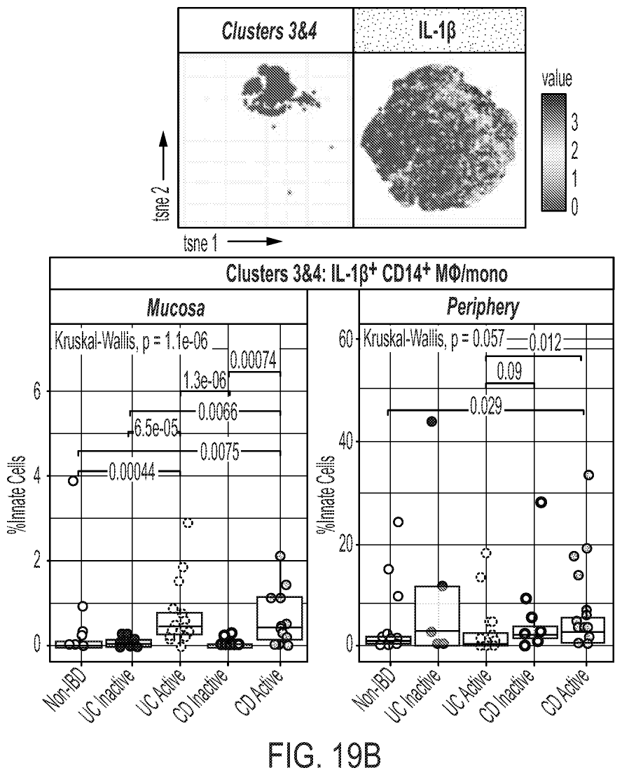

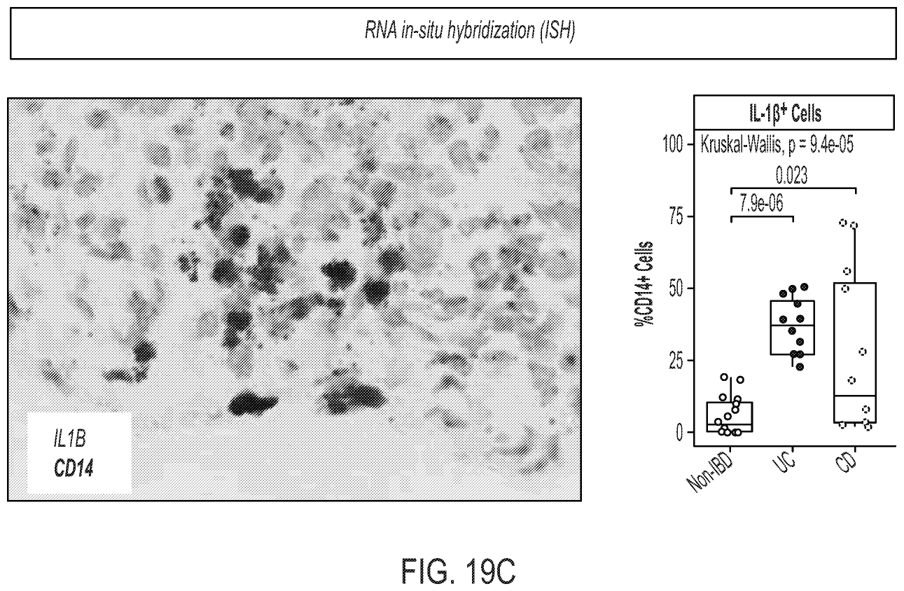

Total CD14+ Mϕ/mono, corresponding to Node 4(−) (in FIG. 15B), were increased in active IBD mucosa and in peripheral CDa (FIG. 19A) as were IL-1β⁺ Mϕ/mono clusters (clusters 3 and 4) (FIG. 19B). Expansion of IL-1β⁺ Mϕ/mono in active IBD mucosa was confirmed in an independent cohort through ISH (FIG. 19C). Markers used for dimensionality reduction and clustering of T cells comprised IL-1β, CXCR3, CCR6, CD45RA, CD3, IFNγ, CD19, HLA-DR, CD8a, CD14, IL-22, CD45RO, CD25, CD38, CCR7, IL-17A, CD45, CD127, IL-21, and CD4. Markers used for dimensionality reduction and clustering of HLA-DR+CD38+ T cells comprised IL-1β scRNA-seq analysis of colonic mucosa of five active UC subjects also identified a cluster of myeloid cells expressing IL1B, CD14, and FCGR3A (CD16). Additional cluster-defining genes included inflammatory mediators such as IL8, S100A8/A9 (calprotectin), CD163, SOD2, and LYZ.

### Example 18: Random Forest Modeling Discriminates CDa Vs UCa in Mucosa and Periphery

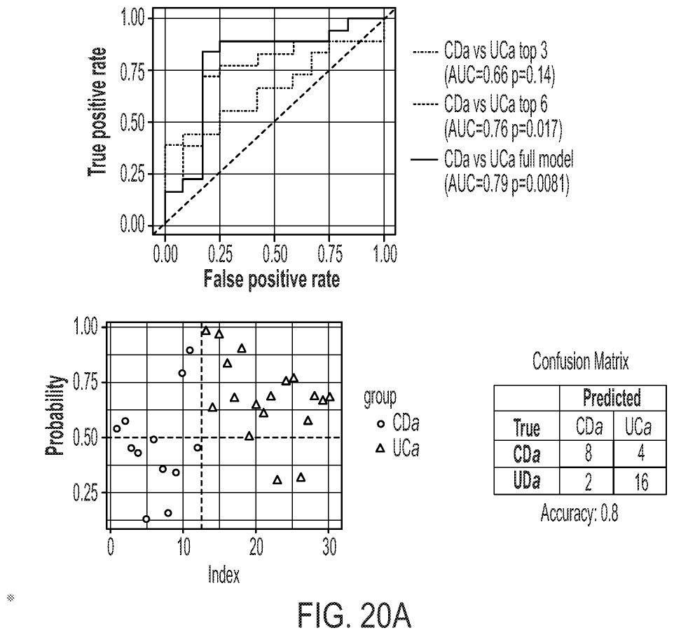

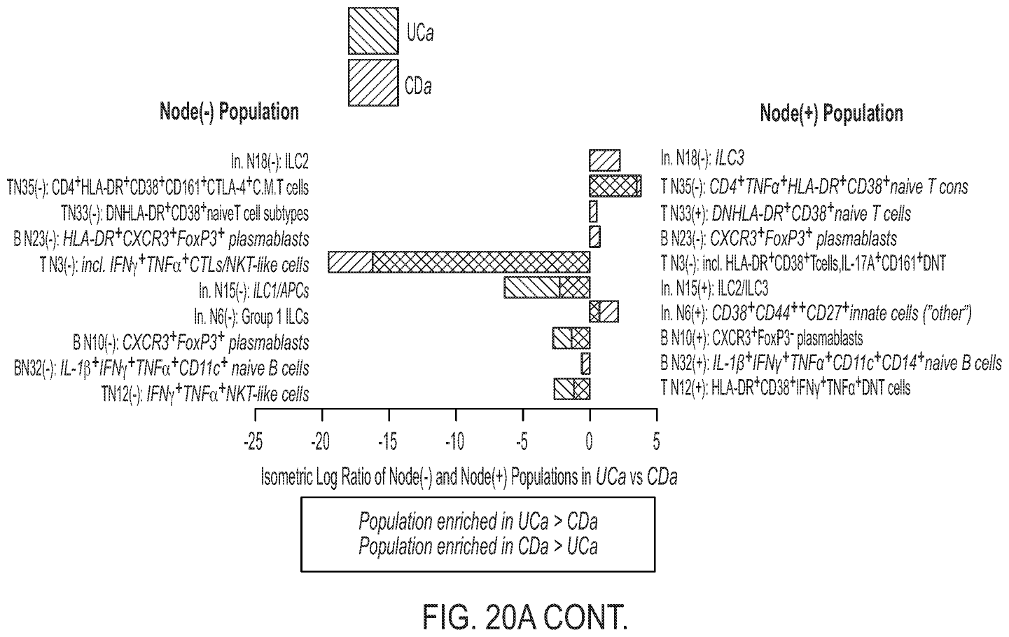

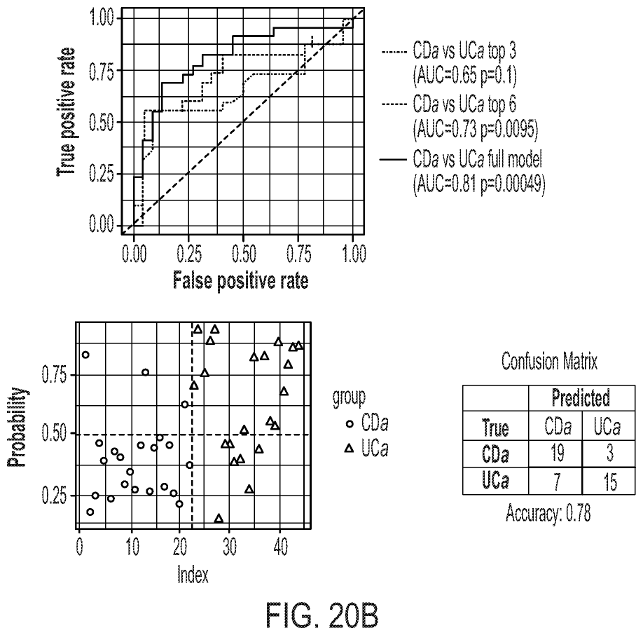

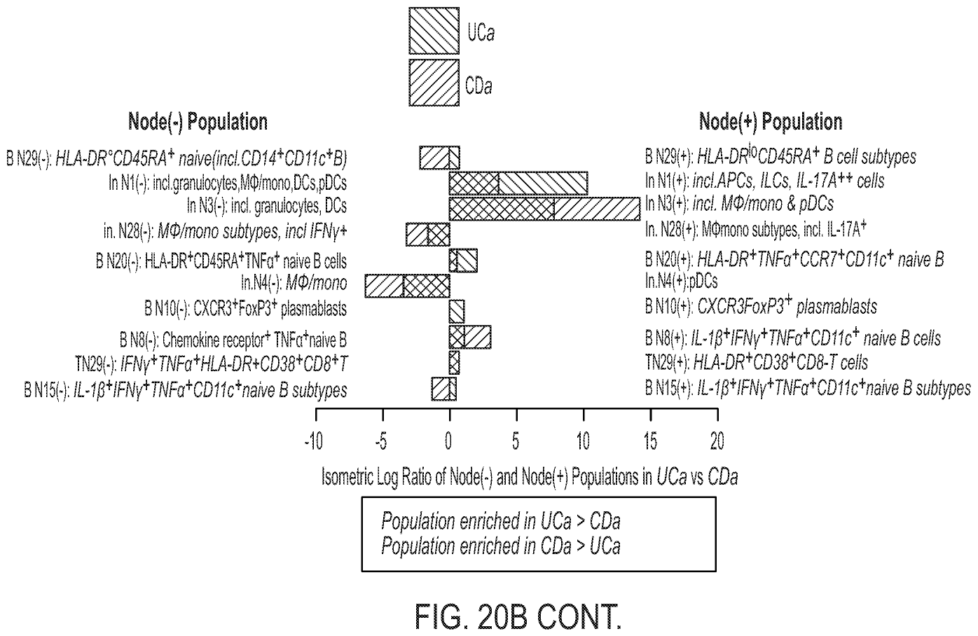

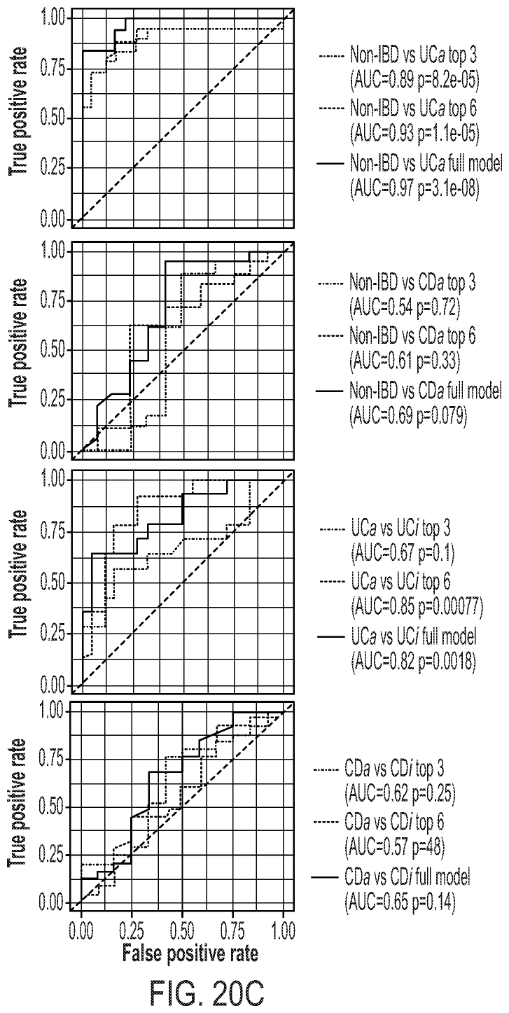

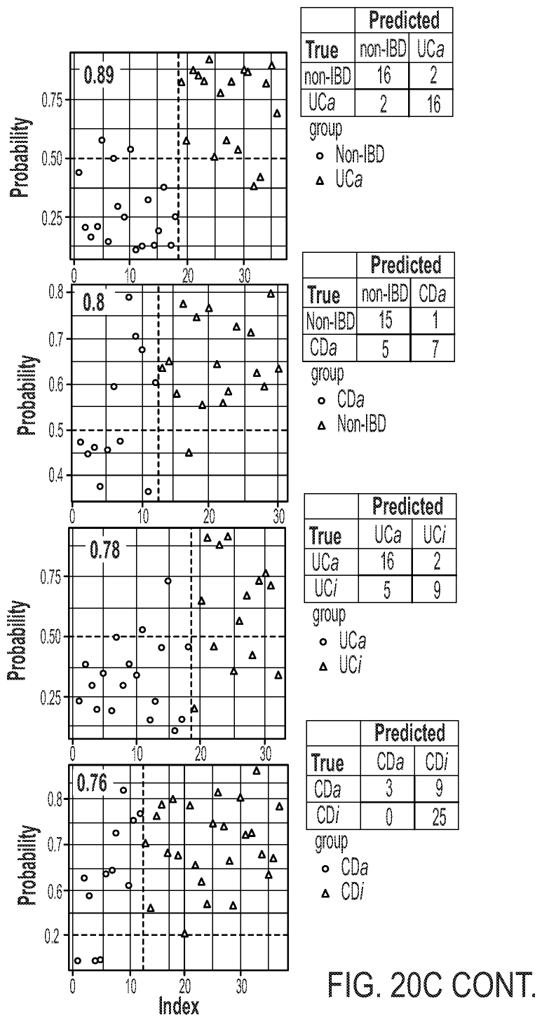

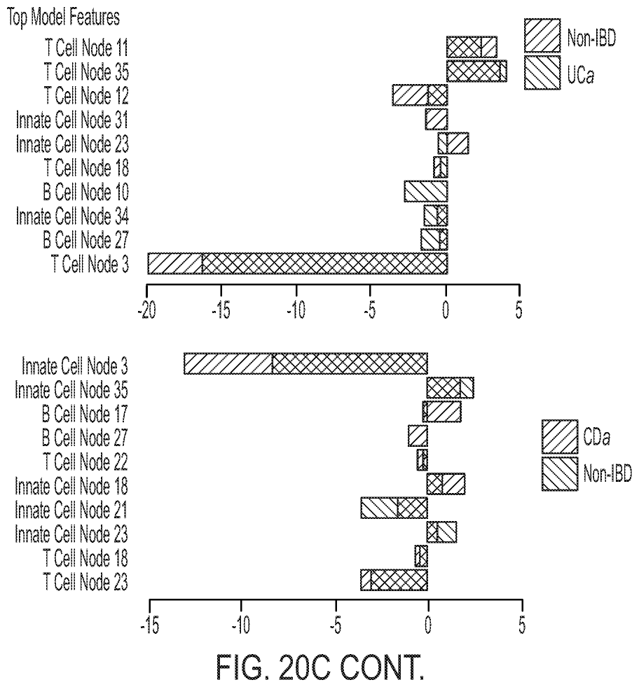

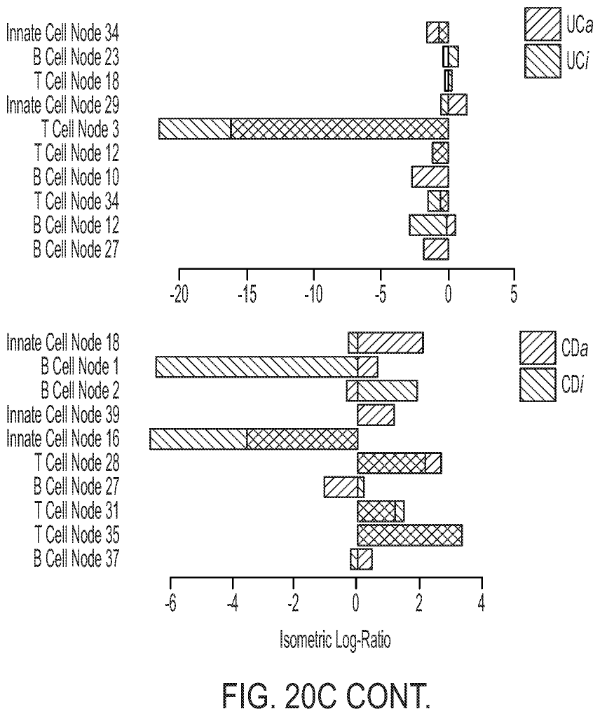

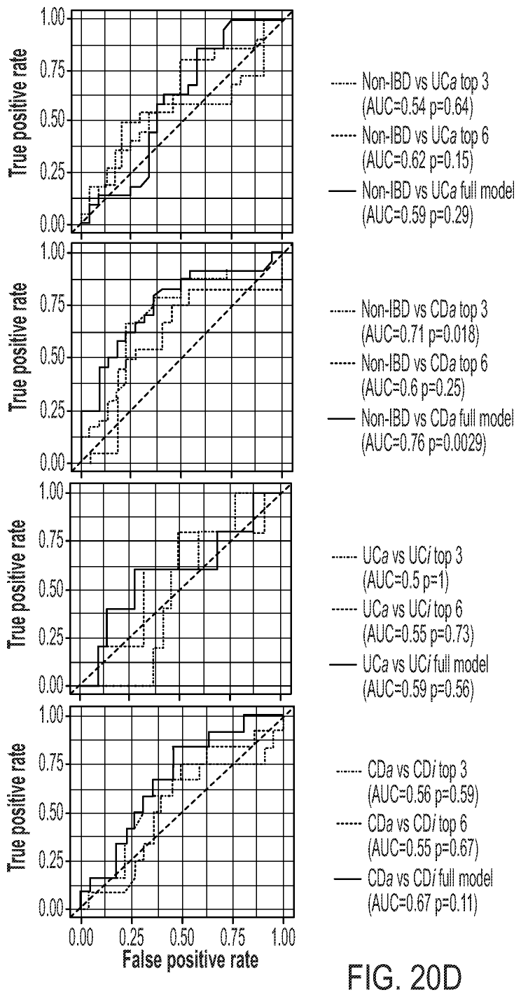

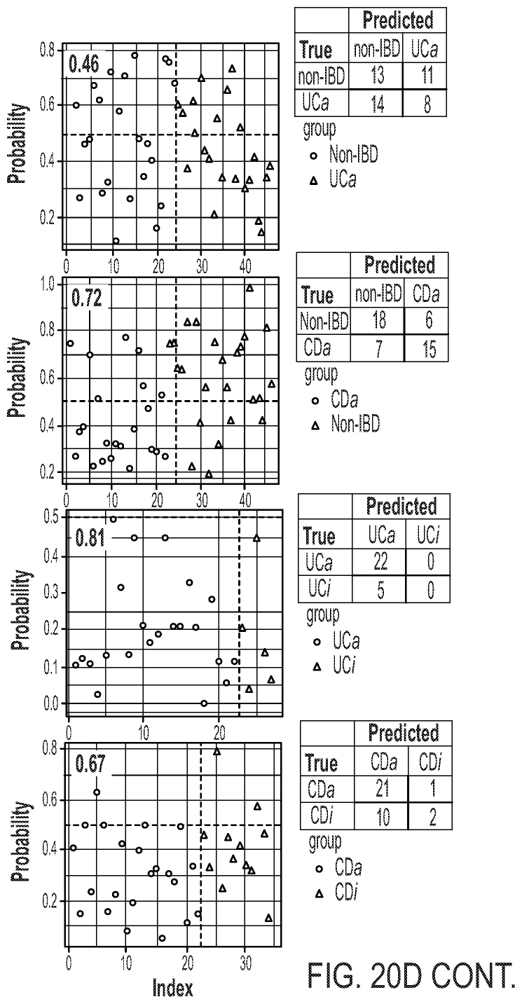

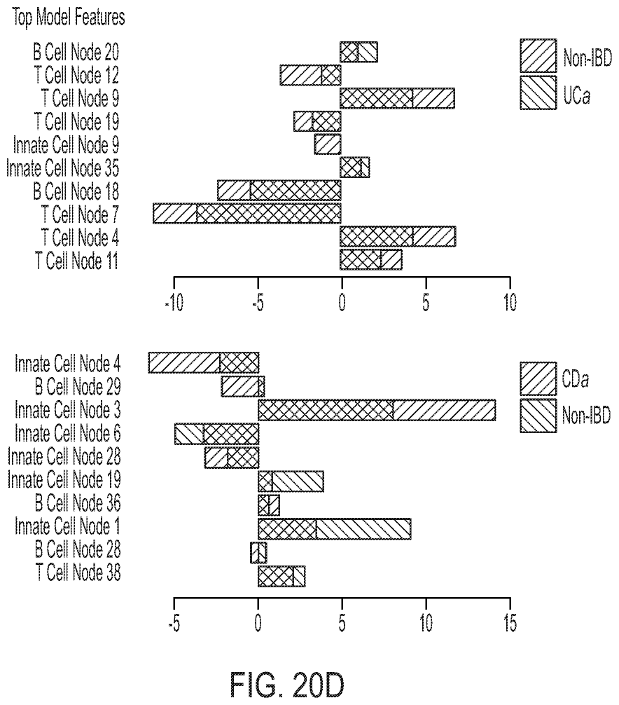

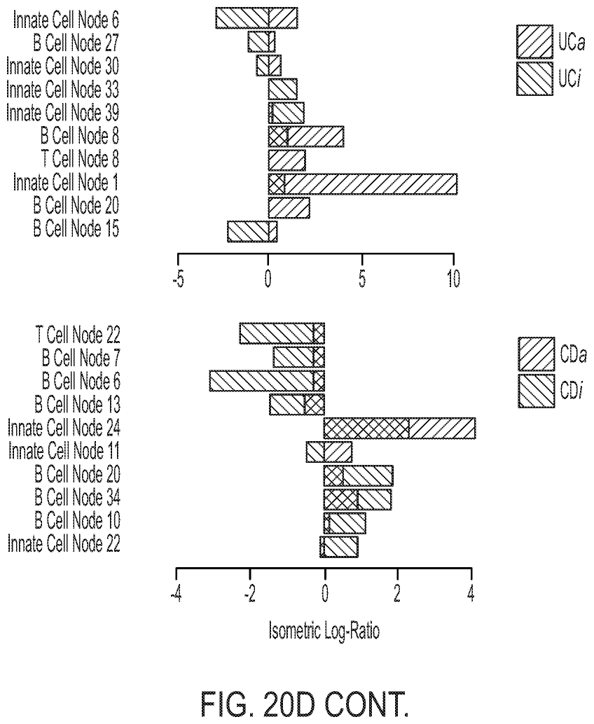

Machine learning with RF was applied to determine if differences between subject groups based on automated T, B and innate clustering could classify mucosal and peripheral disease. In brief, the relative abundance of the daughter branches of every node was computed in all samples, and RF analysis was used to model how these compositional differences vary across subject groups. RF models accurately differentiated CDa and UCa both in the mucosa and periphery (FIGS. 20A, 20B). Model receiver operating characteristic (ROC) curves associated with area under the curve (AUC) p-values demonstrate that a 6-feature model discriminates CDa vs UCa mucosa and periphery (p=0.017 and p=0.0095 respectively) (FIGS. 20A, 20B).

Interestingly, half of the top 6 nodes differentiating CDa from UCa mucosa were T cell nodes (FIG. 20A), while those in the periphery were predominantly innate cell nodes (FIG. 20B). The top 6 nodes differentiating CDa from UCa mucosa were T Cell Nodes 35 and 33, which both branch into clusters of HLA-DR⁺CD38⁺ T cells; T Cell Node 3, which has a daughter branch giving rise to HLA-DR⁺CD38⁺ T cells and IL-17A⁺CD161⁺ DN EM T cells; B Cell Node 23, which branches into clusters of CXCR3+ plasmablasts; and Innate Cell Nodes 15 and 18, which branch into ILC1/IL2/ILC3 populations. The ILC3 branch of Innate Cell Node 18 (the primary feature discriminating UCa from CDa mucosa) was enriched in CDa>UCa compared to the ILC2 branch (FIG. 20A). Among the top 6 nodes differentiating peripheral CDa from UCa were multiple innate cell nodes branching into IL-1β^{+/-} Mϕ/mono and DCs, complementing our findings demonstrating enrichment of IL-1β⁺ Mϕ/mono/DCs in peripheral CDa>UCa (in FIG. 15D, FIG. 19B).

RF was additionally used to differentiate other subject group pairings (FIGS. 20C, 20D). Based on AUC p-values, in the mucosa, RF modeling accurately classified non-IBD:UCa, non-IBD:CDa (strong trend with p=0.079), and UCa:UCi, but not CDa:CDi, perhaps suggesting that differences between active and inactive CD are more subtle (FIG. 20C). In the periphery, apart from UCa:CDa, only the comparison between non-IBD and CDa was significant, suggesting that perhaps CD is associated with more systemic inflammation than UC (FIG. 20B).

Inflammatory bowel disease (IBD) is a multifactorial disorder influenced by genetic predisposition, microbial dysbiosis, and immune dysregulation that can be subdivided into two main subtypes: ulcerative colitis and Crohn's disease. Recent studies from murine models and human data have led to improved understanding of the immunopathogenesis of IBD and the development of better targeted therapies including anti-cytokine antibodies, anti-trafficking antibodies, and JAK-inhibitors to treat these diseases. However, a significant proportion of patients either do not respond to the available therapies or become refractory to them, underscoring the need for a better understanding of immune dysregulation in IBD that can lead to more tailored therapies in the future.

To this end, a number of recent studies have applied cutting-edge single cell technologies, such as single cell RNAseq (scRNAseq) and mass cytometry (CyTOF) to evaluate, at an unprecedented resolution, cellular alterations associated with IBD. Here, data from CyTOF analysis of pediatric and adult IBD patient colonic mucosa and peripheral blood is presented that identified novel distinctions between Crohn's disease and ulcerative colitis immune landscapes. Many more differences between subject groups were identified in the tissue than in the peripheral blood, and the differences identified in the mucosa did not consistently match those found in the peripheral blood, suggesting distinct immune signatures in these compartments. A summary of these signatures is depicted in Table 3 and FIG. 1. Furthermore, machine learning was used to create models based on the data that can accurately classify disease type/activity in both tissue and periphery, potentially heralding new diagnostic avenues in this field.

Active colonic mucosa from IBD patients had significant alterations in both innate and adaptive immune cell populations from that of non-IBD controls. IBD patients had increased abundance of HLADR⁺CD38⁺ T cells in UCa and CDa mucosa; however, contrary to a recent report, altered abundance of these cells in the peripheral blood was not observed. CD38, nicotinamide adenine dinucleotide (NAD⁺) glycohydrolase, is expressed on a number of cell types, including T cells, and is involved in calcium mobilization, cell activation, and chemotaxis. CD38 has been implicated in colitis in mice and CD38+ effector T cells in the severity of pediatric IBD. The results reported herein indicate that CD38 could be targeted as an IBD therapeutic. Interestingly, HLA-DRA/CD38 transcripts were not reliably observed in T cells in scRNA-seq data (or in mining available published scRNA-seq data from human UC colonic mucosa), indicating that T cells may acquire HLA-DR/CD38 proteins through membrane fusion upon cellular interaction.

CXCR3⁺ plasmablasts were enriched in active IBD tissue. Plasmablasts, precursors to antibody-secreting plasma cells, migrate to sites of inflammation via the chemokine receptor CXCR3. Circulating CXCR3⁺ plasmablasts have been described in immune-mediated diseases including UC, as have increased plasma cells in UC colonic mucosa. The present findings of IBD-associated mucosal CXCR3⁺ plasmablasts complements and builds upon this knowledge. Classically associated with Tregs and suppression, FoxP3 expression in non-T cells is controversial; nevertheless, FoxP3 expression was detected in the CXCR3⁺ plasmablast population, indicating that these cells may play a regulatory role in inflamed mucosa.

Additionally, the results presented herein confirm previous observations that the abundance of regulatory T cells is enhanced in inflamed IBD tissue and is associated with an increase in regulatory T cell IL-17A expression. One explanation for this observation has been that even though overall there is an enrichment of regulatory T cells in the mucosal tissue, the ratio of regulatory T cells to conventional T cells is decreased, perhaps overwhelming the anti-inflammatory effect of the regulatory T cells present. However, in this cohort, both the abundance of regulatory T cells as well as the ratio of regulatory T cells to conventional T cells was elevated, suggesting that regulatory T cell function in IBD may be altered or compromised. This is further supported by the findings that regulatory T cells in active Crohn's disease had increased IL-1β expression. Interestingly, both regulatory T cells and conventional T cells from IBD patients had elevated expression of CD24. In T cells, CD24, heat-stable antigen, expression is downregulated upon exiting the thymus, but can be upregulated after TCR-CD3 stimulation. Additionally, CD24 expression on peripheral T cells has been shown to be necessary for homeostatic proliferation and essential for clonal T cell expansion and the development of experimental autoimmune encephalomyelitis. The increased T cell expression of CD24 in IBD may be diagnostic of disease.

Data from this cohort also suggest unique signatures of ulcerative colitis and Crohn's disease. T cells play a well-established role in the pathogenesis of IBD. The role of helper T cells (TH) in IBD has been controversial, with some studies suggesting pathogenic implication in both ulcerative colitis and Crohn's disease, while others suggesting an ulcerative colitis-specific signature. The results reported herein show that inflamed mucosa of ulcerative colitis patients is significantly enriched for IL-17A-producing T cells when compared to Crohn's disease and non-IBD controls, suggesting that ulcerative colitis may be driven by IL-17A expressing TH cells. Crohn's disease on the other hand, was dominated by enhanced IL-1β production across many T cell subtypes. Interestingly, IL-17A predominance in ulcerative colitis and IL-1β predominance in Crohn's disease was not restricted to T cells and was observed for other cell types such as B cells suggesting a more global pattern of dysregulation. Although IL-17 blockade was not efficacious in treating Crohn's disease, its use in ulcerative colitis has not been described and might be beneficial.

The results presented herein identified several disease-specific mucosal signatures related to differential cytokine expression. In T cells, effector memory subsets were stratified in UCa versus CDa mucosa along a cytokine axis, with IL-17A⁺ T cells increased in UCa and IFNγ⁺TNFα⁺ T cells increased in CDa. The role of Th17 cells in IBD has been controversial, with some studies suggesting pathogenic implication in CD, while others suggesting a UC-specific signature. MAIT cells, known to express pro-inflammatory cytokines including IFNγ and IL-17A, have not been rigorously studied in IBD although enrichment of MAIT cells in IBD mucosa has been reported. In this study, a UC-specific IL-17A⁺CD161⁺ EM T cell subset was identified, which could represent MAIT cells or T cells with a similar innate phenotype, and based on scRNA-seq analysis, could be characterized by the Th17-related transcription factor RORA. While IL-17A blockade was not efficacious in treating CD, its use in UC might be considered.

The present analysis related to Tregs similarly identified expansion of IL-17A⁺ memory subsets specific to UCa, belonging to umbrella populations of highly activated IBD-associated memory Tregs. Whereas one population of IBD-enriched, activated memory Tregs was FoxP3⁺⁺, the other was FoxP3^{lo} with intense pro-inflammatory cytokine expression (IFNγ⁺TNFα⁺IL-17A^{+/-}), suggesting that Treg function in IBD may be altered, as has been suggested by one prior study in CD. In addition to these mucosal Treg signatures, an IL-1β⁺ Treg population increased in peripheral CDa was an unusual finding.

Unlike T cells, B cell contribution to IBD pathogenesis is less understood. The data reported herein show a predominance of mucosal B cells in patients with ulcerative colitis. Additionally, elevated B:T cell ratio was observed in ulcerative colitis mucosa when compared to Crohn's disease patients and non-IBD controls. The increase in B cells is primarily driven by a compartment containing plasmablasts and/or regulatory B cells. Finally, it is shows that B cell marker expression, such as enhancement of CCR4 in IBD compared to non-IBD, could differentiate between cells from IBD patients and non-IBD controls. Taken together, these data suggest that the unique B cell IBD signatures could be used to tailor novel therapeutics.

Finally, there are significant differences in the innate cell composition between IBD patients and non-IBD controls both in the tissue and in the periphery, suggesting that the innate cell compartment might be the best peripheral biomarker of disease. Interestingly, Crohn's disease patients had an enhancement of CD14⁺ monocytes/MP in the periphery in both active and inactive states while in the tissue the concomitant increase in CD14⁺ monocytes/MP was only observed in active disease, indicating an ongoing systemic inflammation in Crohn's disease even in the absence of intestinal findings. Without wishing to be bound by theory, natural killer cells and innate lymphoid cells were enhanced in inactive Crohn's disease tissue, but not in active Crohn's disease, suggesting that these cells might participate in the recovery process or alternatively might be pathogenic by recruiting other cell types to the site of mucosal inflammation. Similar to B and T cells, mucosal innate cell's IL-1β production was also elevated in active Crohn's disease. Moreover, enhancement of innate cell IL-1β production in active Crohn's disease was also observed in peripheral blood, indicating that this might be a possible peripheral biomarker of Crohn's disease activity. Although IL-1β⁺ innate cells were more abundant in active Crohn's disease, the canonical producers of IL-1β, macrophages, were similarly elevated in active ulcerative colitis and active Crohn's disease. Increased expression of IL-1β⁺ in dendritic cells and plasmacytoid dendritic cells (pDCs) was restricted to active Crohn's disease, suggesting that not only the amount of IL-1β but also its source might be an important contributor to inflammation. Importantly, while the use of IL-1β blockade in controlled trials in Crohn's disease has not been reported, these data suggest that in a carefully chosen subgroup of patients, these approaches would be beneficial.

IL-1β signatures specific to CD also involved HLA-DR+CD38+ T cells, naïve B cells and DCs. IL-1β⁺ My/mono were increased in both CDa and UCa mucosa, which has been reported previously employing in vitro studies; however, the increase of IL-1β⁺ monocytes specific to peripheral CDa was identified. In a subgroup of CD patients, targeting IL-1β may be a promising strategy.

Peripheral immune population differences distinguishing UCa from CDa were primarily found in innate immune cells. DCs, pDCs, and CD14⁺ Mφ/mono were all increase while Group 1 ILCs were decreased in peripheral CDa. In peripheral UCa, a decrease (trend) was found in granulocytes expressing unconventional granulocyte markers including HLA-DR and CD56 with a concomitant increase in these cells in UCa mucosa, potentially reflective of tissue homing. This is consistent with previous data suggesting that intestinal neutrophil infiltration correlates with severity of UC.

Predictive modeling with RF built upon these findings. Many of the top RF nodes branched into clusters that were identified through the initial analysis as being hallmarks of disease, including HLA-DR⁺CD38⁺ T cells, FoxP3⁺CXCR3^{+/-} plasmablasts, and IL-1β^{+/-} My/mono and DCs. Other nodes identified through RF revealed important patterns related to specific clusters, such as the enrichment of ILC3>ILC2 in CDa mucosa. ILC1 (which was found to be increased in CDa>UCa mucosa) can transition to an ILC3 phenotype in an IL-1 dependent manner, complementing data presented herein that demonstrates IL-1-specific signatures in CDa. Importantly, RF models could distinguish nearly all mucosal subject group comparisons but in the periphery could only distinguish UCa:CDa and CDa:non-IBD. This indicates that although robust immunologic signatures are more likely to be mucosal, there are some that can be peripherally detected (especially in CDa) potentially of great biological and diagnostic relevance.

In summary, the results highlight that CyTOF analysis can be used to simultaneously define unique cellular profiles of mucosal tissue that can differentiate between IBD patients and healthy controls as well as between ulcerative colitis and Crohn's disease. A number of these signatures, such as IL-17A in UC, IL-1β in Crohn's disease and CD38 and CCR4 in MD, already have approved biologic inhibitors that can be amenable to targeted IBD therapeutics.

The results reported herein above were obtained using the following methods and materials.

### Human Sample Collection

Peripheral blood and/or colonic tissue from pediatric (age >6) and adult subjects were collected after providing written informed consent. Samples were collected peri-procedure (colectomy/colonoscopy) or during a clinical lab draw.

### Determination of Inflammation Status and Disease Phenotype

The inflammatory status of IBD tissue was based on blinded histopathological scoring by an experienced pathologist using the Nancy Index (FIG. 3A). An index of 0-1 was considered inactive disease while 2-4 was considered active. Nancy Index assignments and endoscopic assessments of inflammation were correlated (FIG. 3C). See FIG. 3C for further methodology for peripheral samples. Chart review was performed to obtain age, gender, treatment status, CRP values and disease phenotype per the Montreal Classification System (Table 1).

### Peripheral Blood Mononuclear Cell (PBMC) and Tissue Isolation/Cryopreservation

To isolate PMBCs, whole blood collected in K2 EDTA tubes (BD Vacutainer) was processed using density-gradient centrifugation with Lympholyte-H Cell Separation Media (Cedarlane). Biopsies and resected surgical specimens were collected in T-cell media (500 mL RPMI 1640 medium (ThermoFisher), 50 mL FBS plus 5 mL of: Pen/Strep (ThermoFisher), NEAA, Sodium pyruvate, Glutamax, and HEPES (Gibco)). PBMC suspensions (1 million cells per 100 μL) and intestinal samples were cryopreserved in freezing media (10% dimethyl sulfoxide (DMSO) (Sigma) and 90% fetal bovine serum (FBS) (Gibco)). Samples were transferred to liquid nitrogen for long-term storage.

Mucosal Cell Isolation from Cryopreserved Samples

For CyTOF, cryopreserved intestinal samples were thawed and digested overnight in digestion media (10 μg/mL collagenase I (Sigma)+1 μg/mL DNaseI (10,000 IU/mL) (Sigma) in 20 mL T-cell media/sample) at 37° C. at 200 rpm. For flow cytometry, samples were digested in 300 U/mL collagenase VIII (Sigma)+50 μg/mL DNAseI (Sigma) in 20 mL T-cell media/sample×45 min. Digested solutions were strained and pelleted to isolate cells.

### Peripheral Blood Mononuclear Cell (PBMC) and Mucosa Mononuclear Cell (MC) Cryopreservation

To isolate PBMCs, whole blood was collected in K2 EDTA tubes (BDVacutainer) and processed using density gradient centrifugation with Lympholyte-H Cell Separation Media (Cedarlane). Fresh biopsies and resected surgical specimens were collected in T cell media comprising 500 mL Roswell Park Memorial Institute (RPMI) 1640 medium (ThermoFisher), 50 mL fetal bovine serum (FBS), and 5 mL of penicillin streptomycin (Pen Strep) (ThermoFisher), non-essential amino acid (NEAA), sodium pyruvate, Glutamax, and 4-(2-hydroxyethyl)-1-piperazineethanesulfonic acid (HEPES) (Gibco). PBMC suspensions (1 million cells per 100 μL) and intestinal samples were slow frozen in freezing media comprising 10% dimethyl sulfoxide (DMSO)(Sigma) and 90% FBS (Gibco). Biopsies and tissue were cryopreserved.

Mucosal MCs were isolated from cryopreserved intestinal samples. Cryopreserved samples were thawed and suspended in digestion media (4 μl of collagenase I stock 50 mg/mL (Sigma) and 2 μL of DNase I stock 5 mg/mL (10,000 IU/mL) (Sigma) per 10 mL of T cell media). The samples were digested and centrifuged overnight at 37° C. at 200 rpm and then strained. The supernatant was pelleted to isolate the mucosal MCs.

### CyTOF Analysis

Thawed PBMCs and mucosal MCs were stimulated with Phorbol 12-myristate 13-acetate (PMA) (1:2000 of 100 μg/mL, Sigma), ionomycin (1:1000 of 1 mg/mL, Sigma), and GolgiStop (1:1500, BD Biosciences) for 4 hours. Cells were stained with Rh103 nucleic acid intercalator (500 μM, Fluidigm) to label non-viable cells. The samples were incubated with a panel of antibodies (Table 2). Each panel consisted of approximately 40 heavy metal-tagged surface and intracellular antibodies. All panels shared 22 markers at the same metal channels, and a subset of these markers were used for clustering. The markers in the subset included CD3, CD4, CD8, CD127, CD25, CD27, CXCR3, CCR6, CCR7, CD45RA, CD45RO, IFNγ, TNFα, IL-1β, IL-17A, IL-21, IL-22, HLA-DR, and CD38. Following incubation with antibodies, the samples were labeled with Ir191/193 nucleic acid intercalator (1:1000 of 125 μM, Fluidigm) to label DNA in individual cells. The samples were run on the CyTOF2 Mass Cytometer (Fluidigm) equipped with a SuperSampler fluidics system (Victorian Airships) at an event rate of <500 events/second. EQ4 Elemental beads (Fluidigm) were used for normalization.

### Quantification and Statistical Analysis

Normalized flow cytometry standard (FCS) files were uploaded to the Cytobank platform to define CD45⁺ single cell events and to manually gate immune cell populations (FIGS. 4C, 6I, 8A, 11A, 15A). CD45⁺CD3⁺ viable single cellular events from all mucosa samples were gated and exported from Cytobank, uploaded into FlowJo, a software suite for analyzing flow cytometry data. Extraneous channels were removed to ensure that all files contained the same channels. These files transferred to Cytofkit package on R (an integrated suite of software facilities for data manipulation, calculation and graphical display) and clustered using FlowSOM with a k=20 using channels/markers common to all files (k is a parameter that specifies the number of clusters to detect). Dimensionality reduction was achieved using t-stochastic neighbor embedding (T-SNE). CD45⁺ single cell events from mucosa and peripheral blood samples were further gated into a variety of cellular populations belonging to T cell, B cell, and innate immune cell lineages. The event counts as well as the mean metal intensity (MMI) of all available CyTOF panel markers were exported from Cytobank for further statistical analysis.

All statistical computations were performed in R, including Kruskal-Wallis tests (to compare cell population percentages and MIMI across multiple groups) and Mann Whitney U tests or Wilcoxon t-tests (to compare between two groups). Plots were generated using ggplot2, and box-and-whisker plots depict the median (heavy line) within the interquartile range (IQR, delimited by the box) with whiskers extending to largest observation less than or equal to +1.5*IQR and the smallest observation greater than or equal to −1.5*IQR.

### Flow Cytometry

Colonic mucosal samples from a separate cohort (demographic data in Table 4) were digested and stimulated as above. Cells were stained with fluorophore-conjugated antibodies in FCS buffer (PBS plus 2% FBS). Samples were acquired on a BD LSRFortessa. Further compensation and analysis were performed in FlowJo.

TABLE 4
FLOW CYTOMETRY COHORT

| | Non-IBD Control | Active Ulcerative Colitis | Active Crohn's Disease |
|---|---|---|---|
| | Tissue (n = 10 ) | Tissue (n = 9) | Tissue (n = 10) |
| Gender (n %) | 5 (50%) | 2 (22%) | 5 (50%) |
| Female | 5 (50%) | 7 (78%) | 5 (50%) |
| Male | | | |
| Age at collection, vears (mean ± SD) | (19.5 ± 11.6) | (15.0 ± 10.4) | (16.9 ± 5.3) |
| Age at diagnosis; years (mean ± SD) | (N/A) | (13.34 ± 7.5) | (114 ± 4.4) |
| Duration of disease years (mean ± SD) | (N/A) | (1.7 ± 3.1) | (5.5 ± 5.1) |
| Extent of disease (Montreal classification) a (%) | NA | NA | 0 |
| Ilea CD (LI) | NIA | N/A | 1 (1%) |
| Crohn's Colitis (12) | N/A | N/A | 9 (90%) |
| Ileo-colonic CD (L3) | N/A | N/A | 4 (40%) |
| Upper GI disease (L4) | NIA | 1 (11%) | N/A |
| Lett-sided colitis | N/A | 8 (89%) | N/A |
| Pancolitis | | | |
| Disease behavior (Montreal classification) n (%) | NA | NA | 7 (70%) |
| Non-stenolicinon-penetrating (B1) | NIA | N/A | 3 (30%) |
| Stenotic (B2) | N/A | N/A | 0 |
| Penetrating (B3) | N/A | N/A | 0 |
| Stenalicipenetrating(B2B3) | N/A | N/A | 2 (20%) |
| Perianal disease (p) | | | |
| Sample location n (%) | 8 (80%) | 5 (56%) | 8 (80%) |
| Left Colon | 2 (20%) | 4 (44%) | 2 (20%) |
| Transverse Colon | | | |
| CRP levels, mg/dL | not available | 0.16 | 2.84 |
| Median | N/A | [0.04, 1.06]; 7 | [0.88, 4.80], 4 |
| [min, max]; n patients with CRP | | | |
| value within 10d of sample collection | | | |
| Treatment n (%) | N/A | 2 (22%) | 0 |
| 5-Aminosalicylic acid | N/A | 2 (22%) | 3 (30%) |
| Corticosteroids | N/A | 2 (22%) | 6 (60%) |
| Immuanomodulators (AZA, MTX, SMP, tacrolimus) | N/A | 4 (44%) | 6 (60%) |
| anti-TNF inhibiors | N/A | 0 | 1 (10%) |
| ustekinusmab | N/A | 0 | 0 |
| vedolizumab | N/A | 3 (33%) | 2 (20%) |
| None | | | |

### RNA In Situ Hybridization (ISH)

Formalin-fixed tissue samples were paraffin-embedded and sectioned by the University of Pittsburgh Biospecimen Core. Tissue sections were deparaffinized using xylene and 100% ethanol. RNAscope® probes (ACD Bio) were used to stain targets, including IL1B (C1) and CD14 (C2). RNAscope® Duplex

Detection reagents (ACD Bio) were used according to manufacturer's instructions to amplify target RNA signal and bind probes to chromophores. Sections were stained with hematoxylin for detection of cell bodies. An Echo Revolve microscope was used to image stained sections at 40×. Cells positive for one or both probes were counted on 40× images using ImageJ 1.52 in a blinded fashion. Available demographic data of this cohort in Table 5.

TABLE 5
RNA IN SITU HYBRIDIZATION COHORT
More detailed clinical information unavailable

| | | | Age at Collection | | |
|---|---|---|---|---|---|
| Subject ID | Diagnosis | Gender | (years) | Sample Location | Extent of Disease |
| 1144 | Crohn's Disease | F | 40-43 | Colon (Non-Specified) | Ileo-colonic (L3) |
| 1176 | Crohn's Disease | F | 23-23 | Left Colon | Ileo-colonic (L3) |
| 2651 | Crohn's Disease | F | 18 | Cecum | Ile0-colonic (L3) |
| 1190 | Ulcerative Colitis | M | 60-69 | Left Colon | N/A |
| 1235 | Ulcerative Cottis | M | 50-59 | Right Colon | NA |
| 1203 | Uncerative Colitis | F | 50-53 | Left Colon | NA |
| 2773 | Colonic Dysmotility | F | 22 | Left Colon | NA |
| 1158 | Diverticulitis | M | 50-58 | Left Colon | NA |
| 1167 | Diverticulitis | M | 70-73 | Colon (Non-Specified) | NA |
| 1202 | Diverticulitis | M | 80-89 | Left Colon | NA |

scRNA-Seq: Tissue Preparation, Protocols, Analysis

In brief, processing of single-cell biopsies was based on a protocol adapted from Smilie et al (Smillie C S et al., Cell 2019, 178:714-30 e22). Cryopreserved mucosal samples (2-3 biopsies/sample, or equivalently-sized pieces of resected surgical tissue, as described in Konnikova et al., Mucosal immunology 2018, were thawed and separation of epithelial layer from mucosa was performed. The two cellular fractions were enzymatically digested and run on separate Seq-Well arrays as described in Ordovas-Montanes et al., Nature 2018, 560:649-54, with improved protocol for library preparation based on second-strand synthesis for cDNA (S\hat{ }3) from Hughes et al., bioRxiv 2019:689273. Two arrays were sequenced per sequencing run with an Illumina 75 Cycle NextSeq500/550v2.5 kit on an Illumina NextSeq. Read alignment was performed as done previously (Ordovas-Montanes J et al.; Hughes et al.). Quality-filtered base calls were converted to demultiplexed FASTQ files and aligned to the Hg19 genome using Cell Ranger on the Galaxy portal maintained by the Broad Institute.

Merging of datasets, clustering, and differential gene expression analyses were performed using Seurat v3.154 (satijalab.org/seurat/). More details regarding this analysis follows:

scRNA-Seq: Tissue Preparation

Cryopreserved mucosal samples (2-3 biopsies/sample, or equivalently-sized pieces of resected surgical tissue, as described in Konnikova et al.) were thawed in a 37° C. rock bath. Biopsy bites were rinsed in 30 mL of ice-cold PBS (ThermoFisher 10010-049) and allowed to settle. Each individual bite was then transferred to 10 mL epithelial cell solution (HBSS Ca/Mg-Free, 10 mM EDTA, 100 U/ml penicillin, 100 mg/mL streptomycin, 10 mM HEPES, and 2% FCS (all ThermoFisher)) freshly supplemented with 100 μL 0.5 M EDTA. Separation of the epithelial layer from the underlying lamina propria was performed for 15 min at 37° C. at 140 rpm in a dry shaking incubator. Samples were placed on ice for 10 min, then shaken vigorously 15 times. Visual macroscopic inspection of the tube at this point yielded visible epithelial sheets, and microscopic examination confirmed the presence of single-layer sheets and crypt-like structures. The remnant mucosa was carefully transferred into a large volume (30 mL) of ice-cold PBS to rinse, and then into 10 mL enzymatic digestion mix (Base: RPMI1640, 100 U/ml penicillin, 100 mg/mL streptomycin, 10 mM HEPES, 2% FCS, and 50 mg/mL gentamicin (all ThermoFisher)), supplemented with 100 mg/mL Liberase TM (Roche) and 100 mg/mL DNaseI (Roche)), at 37° C. at 120 rpm for 30 min. During this 30-minute mucosa digestion, the epithelial fraction was spun down at 400 g for 7 minutes and resuspended in 500 μL of epithelial cell solution. Cells were resuspended in ACK lysis buffer (ThermoFisher A1049201) with gentle resuspension, incubated on ice for 4 minutes, and spun down at 300 g for 4 minutes. This step removes red blood cells and was used even if no red blood cell contamination was visibly observed in order to maintain consistency across samples. Cells were resuspended in TrypLE express enzyme (ThermoFisher 12604013)] for 4 minutes in a 37° C. bath followed by gentle trituration with a P1000 pipette and filtering into a new conical tube through a 40 μm cell strainer (Falcon/VWR 21008-949). Filter was washed with 20 mL of ice-cold PBS. Cells were spun down at 300 g for 4 minutes and resuspended in 1 mL of epithelial cell solution and placed on ice while final steps of mucosa dissociation occurred. The mucosa enzymatic dissociation was quenched with 1 mL 100% FCS (ThermoFisher) and 80 μL 0.5M EDTA and placed on ice×5 min, followed by filtering through 40 μm cell strainer and rinsing with PBS to 30 mL total volume.

This tube was spun down at 400 g for 10 minutes and resuspended in 1 mL of ACK and placed on ice for 3 minutes. Cells were spun down at 400 g for 4 minutes and resuspended in 1 mL of epithelial cell solution and spun down at 400 g for 2 minutes and resuspended in 400 of epithelial cell solution and placed on ice.

scRNA-Seq: Seq-Well, Sequencing, Alignment

Epithelial and lamina propria samples were then run on separate Seq-Well arrays as described in Ordovas-Montanes et al. with the improved protocol for library preparation based on second-strand synthesis for cDNA (S\hat{ }3) from Hughes et al. Two arrays were sequenced per sequencing run with an Illumina 75 Cycle NextSeq500/550v2.5 kit on an Illumina NextSeq at a final concentration of 2.2 pM. The read structure was paired end with read 1 starting from a custom read 1 primer containing 20 bases with a 12-bp cell barcode and 8-bp unique molecular identified (UMI) and read 2 containing 50 bases of transcript information. Read alignment was performed as done previously. Quality-filtered base calls were converted to demultiplexed FASTQ files and aligned to the Hg19 genome using Cell Ranger on the Galaxy portal maintained by the Broad Institute.

scRNA-Seq: Data Analysis

Merging of datasets, clustering, and differential gene expression analyses were performed using Seurat v3.154 (https://satijalab.org/seurat/). In brief, the unique molecular identified (UMI) count matrices from the LP sequencing data of the 5 included subjects were read into Seurat objects. Each Seurat object was subsetted so that only those cells with at least 200 and no more than 2500 features (genes), and no more than 15% mitochondrial-specific genes, were included. Seurat objects were then merged. Count data was scaled using the default scale factor of 10000 and then log-normalized (natural log+1) as per Seurat v3.1 implementation. Then, the top 2000 differentially expressed genes were identified using the vst method and the data was scaled using a linear transformation to enable dimensionality reduction techniques such as PCA and t-SNE. PCA was applied and the top 50 PCs were used to construct a Shared Nearest Neighbor Graph (KNN) which was then used for clustering with the default Louvain algorithm per Seurat 3.1 with a resolution of 1.2 (total cells analyzed=3979) and generation of 20 clusters. The Wilcoxon Rank Sum Test was used to identify differentially expressed genes in each cluster compared to all others. To subset on T cells, a T cell feature list (comprised of all genes belonging to TRAV, TRB, TRG groups, in addition to other T cell-related genes TRDC, TRDJ1, CD8G, CD8D, CD3E, CD247, CD4, CD8A, CD8B) was applied to the data using the AddModuleScore feature. First, the complete Seurat object dataset was subsetted on all clusters that appeared to be T cells based on their T cell feature/Module score. Then, this new dataset was subsetted so that only those cells with a T cell feature/Module score >0 were included. The top 2000 differentially expressed genes in this new dataset were computed, data then scaled, PCA run, KNN neighbors identified using the first 20 PCs, and clustered with a resolution of 1.2. Two of the generated clusters were clearly B cells and plasma cells based on differential gene expression and the dataset was subsetted once again to exclude those two clusters. On this new Seurat object (total cells=718), the same pipeline was run, with generation of 9 clusters that appeared to all be T cells based on differential gene expression.

### Random Forest (RF) Predictive Modeling

RF classifiers were constructed to differentiate between two subject groups (pairwise comparisons depicted in FIGS. 20A-20D). RF modeling was performed using the leave-one-out cross-validation (LOOCV) strategy, removing one sample as a testing set and using the rest as training set. This strategy is repeated until each sample is left out at least once. The input features were the isometric log-ratios (ILRs) of branch abundance of each node in the hierarchical clustering dendrograms from T, B and innate cell analyses. Prediction accuracy (with a cutoff point equally weighing false positive and false negative predictions) and area under the curve (AUC) were calculated to evaluate model performance. To choose the proper number of features to include in classifiers, each time in LOOCV, top n (n=5, 10, 20, 30, 50 or 100) differential features were selected by Wilcoxon test based on training data to construct classifiers; the n that yielded the highest prediction accuracy was selected as the number of features for the full models.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A panel for characterizing inflammatory bowel disease, the panel comprising two or more capture molecules each bound to a substrate, wherein each capture molecule specifically binds a marker polypeptide selected from the group consisting of CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα, or a polynucleotide encoding said marker polypeptide.

2. The panel of claim 1, wherein the markers comprise one of the following sets:
CD3, CD4, CD8, CD127, CD25, CD27, CXCR3, CCR6, CCR7, CD45RA, CD45RO, IFNγ, TNFα, IL-1β, IL-17A, IL-21, IL-22, HLA-DR, and CD38;
CD38, HLA-DR, FOXP3, IL-17A, CCR4, CTLA-4, TNFα, IL-1β, CD161, IFNγ, CCR6, CCR4, CD45RO CD161, CD24, CCR7, CD44, IL-21, IL-22, CD45RA, CXCR3, and CD45RA;
CD45, CD3, CD4, CD8, FOXP3, CD127, CD25, CD27, CD56, CXCR3, CCR6, CCR7, CD45RA, CD45RO, CTLA-4, CD161, IFNγ, TNFα, IL-1β, IL-17A, IL-21, IL-22, HLA-DR, CD38.

3-4. (canceled)

5. The panel of claim 1, wherein the capture molecule is a polypeptide, polynucleotide probe, or fragment thereof.

6-10. (canceled)

11. A method for characterizing markers associated with a disease, the method comprising
detecting a marker in a sample derived from a subject suspected of having inflammatory bowel disease, the method comprising detecting two or more marker polypeptides selected from the group consisting of CD45, CD3, CD4, CD8, CD24, CD25, CD27, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, CD11c, IL23p19, CD66b, CD163, CD44, ckit, CD16, NKp46, AHR, and TNFα in the sample;
distinguishing active ulcerative colitis from a non-inflammatory bowel disease state, the method comprising detecting in a T cell derived from the mucosa of a subject having active ulcerative colitis an increase in a marker polypeptide selected from the group consisting of CD38, HLA-DR, FOXP3, IL-17A, CCR4, and CTLA-4 and a decrease in TNFα relative to a reference level;
distinguishing active ulcerative colitis from a non-inflammatory bowel disease state, the method comprising detecting in a T cell derived from the mucosa of a subject having active ulcerative colitis an increase in a marker polypeptide selected from the group consisting of IL-17A, HLA-DR, FOXP3, CTLA-4, CD45RO, CD45RA, CD38, CD27, CD25, CD24, CD161, CCR7, CCR6, and CCR4 and a decrease in TNFα relative to a reference level;
distinguishing active ulcerative colitis from a non-inflammatory bowel disease state, the method comprising detecting in a T cell derived from the mucosa or peripheral blood of a subject an increase or decrease in a marker polypeptide selected from the group consisting of a marker polypeptide present in FIG. 7C, 18A, or another figure;
distinguishing active Crohn's disease from a non-inflammatory bowel disease state, the method comprising detecting in a T cell derived from the mucosa of a subject having active Crohn's disease an increase in a marker polypeptide selected from the group consisting of CD38, HLA-DR, and FOXP3;
distinguishing active Crohn's disease from active ulcerative colitis, the method comprising detecting in a T cell derived from the mucosa of a subject an increase in marker polypeptide IL-1β and a decrease in IL-17A relative to a reference level;
distinguishing active ulcerative colitis from inactive ulcerative colitis, the method comprising detecting in a T cell derived from the mucosa of a subject an increase in HLA-DR, CD38, and CTLA-4 relative to a reference level.
distinguishing active Crohn's Disease from inactive Crohn's Disease, the method comprising detecting in a T cell derived from the mucosa of a subject an increase in polypeptide markers IL-1β and a decrease in CD161 relative to a reference level;
distinguishing inactive Crohn's Disease from non-inflammatory bowel disease, the method comprising detecting in a T cell derived from the mucosa of a subject an increase in polypeptide marker IL-17A relative to a reference level;
distinguishing active ulcerative colitis or Crohn's disease from non-inflammatory bowel disease, the method comprising detecting in a B cell derived from the peripheral blood of the subject an increase in polypeptide marker CXCR3 relative to a reference level;
distinguishing inactive ulcerative colitis from non-inflammatory bowel disease, the method comprising detecting in a T cell derived from the mucosa of a subject an increase in polypeptide markers IL-17A, CD45RA, CD38, CD24, CD161, and CCR4 relative to a reference level;
distinguishing active ulcerative colitis from active Crohn's disease, the method comprising detecting an increase in polypeptide markers CCR7, CD24, and IL-22 in a T cell derived from the peripheral blood of the subject relative to a reference level;
distinguishing active ulcerative colitis from inactive ulcerative colitis, the method comprising detecting an increase in polypeptide markers CD24, CD45RA, and CCR4 in a T cell derived from the peripheral blood of the subject relative to a reference level; and
distinguishing active ulcerative colitis from inactive ulcerative colitis, the method comprising detecting an increase in polypeptide markers IL-22, HLA-DR, CD45RA, CD24, CCR7, CCR6, and CCR4 in a T cell derived from the peripheral blood of the subject relative to a reference level.

12-84. (canceled)

85. A method of treating ulcerative colitis in a selected subject, the method comprising administering an agent that inhibits IL-17 or CD38 to a subject, wherein the subject is selected as having increased levels of IL-17A or CD38 in a T cell derived from the mucosa of the subject relative to a reference.

86. A method of treating Crohn's disease in a selected subject, the method comprising administering an agent that inhibits IL-1β blockade to a selected subject, wherein the subject is selected as having increased levels of IL-1β in a T cell derived from the mucosa of the subject relative to a reference.

87. A method of characterizing disease progression in a subject having or suspected of having inflammatory bowel disease, the method comprising:
detecting two or more markers selected from the group consisting of CD45, CD3, CD4, CD8, CD24, CD25, CD38, CD44, CD45RA, CD45RO, CD127, CD161, CTLA-4, CXCR3, CCR4, CCR6, CCR7, FOXP3, HLA-DR, IFNγ, IL-1β, IL-17A, IL-21, IL-22, and TNFα in a first sample derived from a subject at a first point in time; and
detecting the same markers in a second sample derived from the subject at a second point in time; thereby characterizing disease progression.

88. A method of distinguishing disease states, the method comprising
distinguishing active ulcerative colitis from other forms of inflammatory bowel disease, the method comprising detecting in a sample derived from the mucosa of a subject having active ulcerative colitis an increased B cell to T cell ratio;
distinguishing active ulcerative colitis from a non-inflammatory bowel disease state, the method comprising detecting an increase in CD4⁺ T cells in a sample derived from a subject having active ulcerative colitis relative to a reference level.
distinguishing active ulcerative colitis, active Crohn's disease, or inactive Crohn's disease from a non-inflammatory bowel disease state, the method comprising detecting an increased in HLA-DR⁺CD38⁺ T cells in a sample derived from a subject having or suspected of having active ulcerative colitis, active Crohn's disease, or inactive Crohn's disease relative to a reference.
distinguishing active ulcerative colitis, active Crohn's disease, or inactive Crohn's disease from a non-inflammatory bowel disease state, the method comprising detecting increased number of FOXP3⁺ regulatory T cells in a sample derived from active ulcerative colitis, active Crohn's disease, or inactive Crohn's disease relative to a reference;
distinguishing Crohn's disease from ulcerative colitis and from a non-inflammatory bowel disease state, the method comprising detecting increased expression of IL-1β in FOXP3⁺ regulatory T cells in a mucosa sample derived from a subject having Crohn's disease relative to a reference level;
distinguishing active inflammatory bowel disease from a non-inflammatory bowel disease state, the method comprising detecting in a mucosa sample derived from a subject having inflammatory bowel disease an increased FOXP3⁺ expression level in non-regulatory T cells relative to a reference level;
distinguishing active ulcerative colitis from active Crohn's disease and inactive ulcerative colitis, the method comprising detecting an increased IL-22 expression level in FOXP3⁺ regulatory T cells in peripheral blood relative to a reference level;
distinguishing active ulcerative colitis and inactive Crohn's disease from a non-inflammatory bowel disease state, the method comprising detecting a decreased number of conventional T cells in a mucosa sample derived from a subject having active ulcerative colitis or inactive Crohn's disease compared to a non-inflammatory bowel disease reference number;
distinguishing active ulcerative colitis from a non-inflammatory bowel disease state, the method comprising detecting one or more of the group consisting of increased CCR4 expression in all CD4⁺ T cell subsets; increased CTLA-4 expression in T cell subset excluding FOXP⁺ regulatory T cells; increased CD27 expression in CD8⁺ T cells; and decreased TNFα expression in TH1, T1-17, and double negative T cells relative to a reference level;
distinguishing active inflammatory bowel disease from a non-inflammatory bowel disease state, the method comprising detecting an increased number of plasmablasts and regulatory B cells in the mucosa sample derived from a subject having active inflammatory bowel disease relative to a reference number;
distinguishing active inflammatory bowel disease from a non-inflammatory bowel disease state, the method comprising detecting an increased number of CD123⁺ innate cells relative to a reference number in a mucosa sample derived from a subject having active inflammatory bowel disease;
distinguishing active ulcerative colitis from inactive ulcerative colitis, the method comprising detecting an increased number of dendritic cells relative to a reference number in a mucosa sample derived from a subject having active ulcerative colitis.
distinguishing active Crohn's disease from inactive Crohn's disease and from a non-inflammatory bowel disease state, the method comprising detecting an increased proportion of plasmacytoid dendritic cells relative to a reference proportion.

89-113. (canceled)

114. The method of claim 88, wherein the detecting is by mass cytometry Time-of-Flight (CyTOF), flow cytometry, bulk RNA sequencing, or single cell RNA sequencing.

115. A method of distinguishing a disease state, selected from among the following:
distinguishing active ulcerative colitis from anon-inflammatory bowel disease state and active Crohn's disease, the method comprising detecting an increased number of effector memory T cells in a mucosa sample derived from a subject having active ulcerative colitis compared to a non-inflammatory bowel disease state or Crohn's disease reference number, wherein the effector memory T cells are defined as of CD45RA⁺CD45RO⁺CCR7⁺CD27⁺CD3⁺CD45⁺CD4⁻CD8α⁻ and also express CD161, IL17A, TNFα, and IFNγ;
distinguishing active ulcerative colitis from a non-inflammatory bowel disease state, inactive ulcerative colitis, inactive Crohn's disease, or active Crohn's disease, the method comprising detecting a decreased number of effector memory T cells in a mucosa sample derived from a subject having active ulcerative colitis compared to a non-inflammatory bowel disease state, inactive ulcerative colitis, or inactive or active Crohn's disease reference number, wherein the effector memory T cells are defined as of CD45RA⁺CD45RO⁺CCR7⁺CD27⁺CD3⁺CD45⁺ and also express CD8α, TNFα, and IFNγ;
distinguishing active ulcerative colitis from a non-inflammatory bowel disease state, inactive ulcerative colitis, inactive Crohn's disease, or active Crohn's disease, the method comprising detecting a decreased number of effector memory T cells in a mucosa sample derived from a subject having active ulcerative colitis compared to a non-inflammatory bowel disease state, inactive ulcerative colitis, or inactive or active Crohn's disease reference number, wherein the effector memory T cells are defined as of CD45RA⁺CD45RO⁺CCR7⁺CD27⁺CD3⁺CD45⁺ and also express CD56, TNFα, and IFNγ;
distinguishing active Crohn's disease from active ulcerative colitis, the method comprising detecting an increased number of IL-1β⁺HLA-DR⁺CD38⁺CD45⁺CD3⁺ T cells in a mucosa sample derived from a subject having active Crohn's disease compared to an active ulcerative colitis reference number;
distinguishing active ulcerative colitis and inactive Crohn's disease, the method comprising detecting an increased number of FoxP3⁺HLA-DR⁺CD38⁺CTLA-4⁺CD45R0⁺ T regulatory cells in a mucosa sample derived from a subject having active ulcerative colitis compared to a non-inflammatory bowel disease state and active Crohn's disease reference number;
distinguishing inflammatory bowel disease from a non-inflammatory bowel disease state, the method comprising detecting an increased number of FoxP3^{lo}HLA-DR⁺CD38⁺CTLA-4⁺CD45RO⁺IFNγ⁺ TNFα⁺ T regulatory cells in a mucosa sample derived from a subject having inflammatory bowel disease compared to a non-inflammatory bowel disease state reference number;
distinguishing active ulcerative colitis from a non-inflammatory bowel disease state, inactive Crohn's disease and active Crohn's disease, the method comprising detecting an increased number of FoxP3^{lo}HLA-DR⁺CD38⁺CTLA-4⁺CD45RO⁺IFNγ⁺ TNFα⁺IL17A⁺ T regulatory cells in a mucosa sample derived from a subject having inflammatory bowel disease compared to a non-inflammatory bowel disease state reference number.
distinguishing active Crohn's disease from active ulcerative colitis, the method comprising detecting an increased number of CD45⁺CD3⁺CD25⁺CD127⁺CCR7⁺CD27⁺CD45RA⁺CD45RO⁺ central memory T cells regulatory cells that also express IL-1β and CXCR3 in a peripheral blood sample derived from a subject having active Crohn's disease compared to an active ulcerative colitis reference number;
distinguishing active Crohn's disease from a non-inflammatory bowel disease state, inactive ulcerative colitis, and active ulcerative colitis, the method comprising detecting an increased number of IL-1β⁺ TNFα⁺IFNγ⁺ naïve B cells in a mucosa sample derived from a subject having active Crohn's disease compared to a non-inflammatory bowel disease state, inactive ulcerative colitis, or active ulcerative colitis reference number;
distinguishing active ulcerative colitis, inactive Crohn's disease, and active Crohn's disease from a non-inflammatory bowel disease state, the method comprising detecting an increased number of CXCR3⁺ plasmablasts in a mucosa sample derived from a subject having active ulcerative colitis, inactive Crohn's disease, or active Crohn's disease compared to a non-inflammatory bowel disease state, reference number;
distinguishing active ulcerative colitis from a non-inflammatory bowel disease state, active Crohn's disease, inactive Crohn's disease, and inactive ulcerative colitis, the method comprising detecting an increased number of CD56⁺ granulocytes in a mucosa sample derived from a subject having active ulcerative colitis compared to a non-inflammatory bowel disease state, active Crohn's disease, inactive Crohn's disease, and inactive ulcerative colitis reference number, wherein the granulocytes are CD45⁺CXCR3⁺CD44⁺CCR4⁺CCR7⁺CD66b⁺CD56⁺CD3⁻CD19⁻;
distinguishing active Crohn's disease from a non-inflammatory bowel disease state, inactive Crohn's disease, inactive ulcerative colitis, and active ulcerative colitis, the method comprising detecting an increased number of dendritic cells or plasmacytoid dendritic cells in a mucosa sample derived from a subject having active Crohn's disease compared to a non-inflammatory bowel disease state, inactive ulcerative colitis, or active ulcerative colitis reference number, wherein the dendritic cells or plasmacytoid dendritic cells are CD11c⁺CD123⁺HLA-DR⁺IL-1β⁺;
distinguishing active Crohn's disease from active ulcerative colitis, the method comprising detecting an increased number of dendritic cells or plasmacytoid dendritic cells in a peripheral blood sample derived from a subject having active Crohn's disease compared to an active ulcerative colitis reference number, wherein the dendritic cells or plasmacytoid dendritic cells are CD11c⁺CD123⁺HLA-DR⁺IL-1β⁺;
distinguishing active Crohn's disease from a non-inflammatory bowel disease state and active ulcerative colitis, the method comprising detecting a decreased number of Group 1 innate lymphoid cells in a peripheral blood sample derived from a subject having active Crohn's disease compared to anon-inflammatory bowel disease state or active ulcerative colitis reference number, wherein the Group 1 innate lymphoid cells are CD45⁺CD3⁻CD19⁻CD38⁺CD45RA⁺IFNγ⁺ Tbet⁺CD161⁺;
distinguishing active Crohn's disease from active ulcerative colitis, the method comprising detecting an increased number of innate lymphoid cells (ILC) type 1 and ILC-like cells in a mucosa sample derived from a subject having active Crohn's disease compared to an active ulcerative colitis reference number, wherein the innate lymphoid cells (ILC) type 1 and ILC-like cells are CD45⁺CD3⁻CD19⁻IFNγ⁺ TNFα⁺ Tbet^{+/-}CD8α^{+/-};
distinguishing active ulcerative colitis from a non-inflammatory bowel disease state, the method comprising detecting a decreased number of innate lymphoid cells (ILC) type 1 and ILC-like cells in a mucosa sample derived from a subject having active ulcerative colitis compared to a non-inflammatory bowel disease state reference number, wherein the innate lymphoid cells (ILC) type 1 and ILC-like cells are CD45⁺CD3⁻CD19⁻IFNγ⁺ TNFα⁺ Tbet^{+/-}CD8α^{+/-};
distinguishing active Crohn's disease from active ulcerative colitis and non-inflammatory bowel disease state, the method comprising detecting a decreased number of innate lymphoid cells (ILC) type 1 and ILC-like cells in a peripheral blood sample derived from a subject having active Crohn's disease compared to active ulcerative colitis or non-inflammatory bowel disease state reference number, wherein the innate lymphoid cells (ILC) type 1 and ILC-like cells are CD45⁺CD3⁻CD19⁻IFNγ⁺ TNFα⁺ Tbet^{+/-}CD8α^{+/-};
distinguishing active ulcerative colitis from a non-inflammatory bowel disease state, inactive ulcerative colitis, active Crohn's disease, and inactive Crohn's disease, the method comprising detecting a decreased number of innate lymphoid cells type 3 in a mucosa sample derived from a subject having active ulcerative colitis compared to a non-inflammatory bowel disease state, inactive ulcerative colitis, active Crohn's disease, or inactive Crohn's disease reference number, wherein the innate lymphoid cells type 3 are CD45⁺CD3⁻CD19⁻CD161⁺CD127⁺CCR6⁺ckit⁺ TNFα⁺;
distinguishing active inflammatory bowel disease from inactive inflammatory bowel disease and a non-inflammatory bowel disease state, the method comprising detecting an increased number of macrophages/monocytes in a mucosa sample derived from a subject having active inflammatory bowel disease compared to inactive inflammatory bowel disease and a non-inflammatory bowel disease state reference number, wherein the macrophages/monocytes are CD45⁺CD3⁻CD19⁻CD1e CD11c⁺HLA-DR⁺CD14⁺; or
distinguishing active Crohn's disease from active ulcerative colitis and a non-inflammatory bowel disease state, the method comprising detecting an increased number of macrophages/monocytes in a peripheral blood sample derived from a subject having active Crohn's disease compared to active ulcerative colitis or a non-inflammatory bowel disease state reference number, wherein the macrophages/monocytes are CD45⁺CD3⁻CD19⁻CD14⁺CD11c⁺HLA-DR⁺CD14⁺.

116-136. (canceled)

137. The method of claim 115, wherein the macrophages/monocytes are CD45⁺CD3⁻CD19⁻CD14⁺CD11c⁺HLA-DR⁺CD14⁺IL-1β⁺.

138. A method of treating a selected patient, the method comprising administering a compound described herein, wherein the patient is characterized for a disease according to the method of claim 115.

139. The method of claim 115, wherein the T cell or B cell is derived from the peripheral blood of the subject.

140. The method of claim 115, wherein the method further comprises detecting increased expression of memory T cell markers, activation markers and pro-inflammatory cytokines.

141. The method of claim 115, further comprising characterizing T helper subsets.

142. The method of claim 115, wherein the detecting is by mass cytometry, flow cytometry, bulk RNA sequencing or single cell RNA sequencing.

143. The panel of claim 1, wherein the substrate is a bead or planar surface.

144. The panel of claim 143, wherein the planar surface is a membrane, filter, chip, glass slide or other solid support.

145. The panel of claim 1, wherein the capture molecule is a polynucleotide probe having a sequence at least partially complementary to the sequence of a polynucleotide encoding the marker polypeptide.
